(19) United States
(12) Patent Application Publication
(10) Pub. No.: US 2022/0275028 A1
(43) Pub. Date: Sep. 1, 2022

(54) Systems, Methods And Compositions For Recombinant In Vitro Transcription And Translation Utilizing Thermophilic Proteins

(71) Applicant: Nature's Toolbox, Inc., Rio Rancho, NM (US)

(72) Inventors: Michael Humbert, Santa Fe, NM (US); Alexander Koglin, Santa Fe, NM (US); Charlie Villanueva, Albuquerque, NM (US)

(21) Appl. No.: 17/603,276

(22) PCT Filed: Apr. 13, 2020
(86) PCT No.: PCT/US20/28005

§ 371 Date: Oct. 12, 2021

Related U.S. Application Data
(60) Provisional application No. 62/833,555, filed on Apr. 12, 2019.

(51) Int. Cl. C07K 14/195; C07K 14/245; C07K 14/32; C12N 9/00; C12N 9/10

(52) U.S. Cl. CPC C07K 14/195; C07K 14/245; C07K 14/32; C12N 9/93; C12N 9/1014; C12Y 201/02009

(57) ABSTRACT

Another aim of the current invention may include a recombinant cell-free expression system, the reaction mixture containing all the cell-free reaction components necessary for the in vitro biosynthesis of biological compounds, proteins, enzymes, biosimilars or chemical modification of small molecules.

This application claims the benefit of and priority to U.S. Provisional Application No. 62/833,555, filed Apr. 12, 2019. The entire specification and figures of the above-referenced application are hereby incorporated, in their entirety by reference.

### TECHNICAL FIELD

This invention relates to recombinant cell-free expression systems and methods of using the same for high yield in vitro production of biological materials.

### SEQUENCE LISTINGS

The instant application contains a Sequence Listing which has been submitted electronically in ASCII format and is hereby incorporated by reference in its entirety. Said ASCII copy, created on May 5, 2022, is named 90125-00097-Sequence-Listing_Amended.txt and is 427 Kbytes in size

### BACKGROUND

Cell-free expression systems (also known as in vitro transcription/translation, cell-free protein expression, cell-free translation, or cell-free biosynthesis) represent a molecular biology technique that enables researchers to express functional proteins or other target molecules in vitro. Such systems enable in vitro expression of proteins or other small molecules that are difficult to produce in vivo, as well as high-throughput production of protein libraries for protein evolution, functional genomics, and structural studies. Another advantage of such systems is that often the target protein to be expressed may be toxic to a host cell, or generally incompatible with cellular expression, making in vivo systems impractical if not wholly ineffective vehicles for protein expression. Compared to in vivo techniques based on bacterial or tissue culture cells, in vitro protein expression is considerably faster because it does not require gene transfection, cell culture or extensive protein purification.

More specifically, cell-free expression systems generate target molecules and complexes such as RNA species and proteins without using living cells. A typical cell-free expression system may utilize the biological components/machinery found in cellular lysates to generate target molecules from DNA containing one or more target genes. Common components of a typical cell-free expression system reaction may include a cell extract generally derived from a cell culture lysate, an energy source such as ATP, a supply of amino acids, cofactors such as magnesium, and the nucleic acid synthesis template with the desired genes, typically in the form of a plasmid synthesis template, or linear expression (or synthesis) template (LET or LST). A cell extract may be obtained by lysing the cell of interest and removing the cell walls, genomic DNA, and other debris through centrifugation or other precipitation methods. The remaining portions of the lysate or cell extract may contain the necessary cell machinery needed to express the target molecule.

A common cell-free expression system involves cell-free protein synthesis (CFPS). To produce one or more proteins of interest, typical CFPS systems harness an ensemble of catalytic components necessary for energy generation and protein synthesis from crude lysates of microbial, plant, or animal cells. Crude lysates contain the necessary elements for DNA to RNA transcription, RNA to protein translation, protein folding, and energy metabolism (e.g., ribosomes, aminoacyl-tRNA synthetases, translation initiation and elongation factors, ribosome release factors, nucleotide recycling enzymes, metabolic enzymes, chaperones, foldases, etc.). Common cell extracts in use today are made from Escherichia coli (ECE), rabbit reticulocytes (RRL), wheat germ (WGE), and insect cells (ICE), and even mammalian cells (MC).

Cell-free expression systems offer several advantages over conventional in vivo protein expression methods. Cell-free systems can direct most, if not all, of the metabolic resources of the cell towards the exclusive production of one protein. Moreover, the lack of a cell wall and membrane components in vitro is advantageous since it allows for control of the synthesis environment. For example, tRNA levels can be changed to reflect the codon usage of genes being expressed. The redox potential, pH, or ionic strength can also be altered with greater flexibility than in vivo since there is less concerned about cell growth or viability. Furthermore, direct recovery of purified, properly folded protein products can be easily achieved.

Despite many advantageous aspects of cell-free expression systems, several obstacles have previously limited their use as a protein production technology. These obstacles, which are especially present in the E. coli extract-based cell-free systems identified in U.S. Pat. No. 7,118,883, and the yeast extract-based cell-free systems identified in U.S. Pat. No. 9,528,137, include short reaction durations of active protein synthesis, low protein production rates, small reaction scales, a limited ability to correctly fold proteins containing multiple disulfide bonds, and its initial development as a “black-box” science. As a result, there exists a need for an economically viable commercial cell-free expression system that exhibits increased product yield, enhanced component stability, improved protein production rate, and extended reaction time.

As noted above, cell-free systems are not widely used for manufacturing of biologics because of their lack in consistency, yield and possibility to scale. The present inventors previously reported an extract-based cell-free system utilizing exemplary thermophiles to improve the application of such systems by replacing the E. coli machinery with thermostable proteins which led to improved production rates and higher yields, but also including a novel energy regeneration system. (Such novel energy regeneration systems being generally described in PCT Application No. PCT/US201 8/012121, the description, figures, examples, sequences and claims being incorporated herein by reference in their entirety.)

As detailed below, the present inventors have developed a fully recombinant in vitro transcription/translation system, which in some embodiments, incorporate peptide-based components from various exemplary thermophilic bacteria. As noted above, current commercially available cell-free systems are either based on adding necessary transcription/translation machinery from E. coli cell extracts or are based on recombinant E. coli enzymes. Various other sources for extracts have been reported including the use of thermophiles to improve in vitro protein production, but a fully recombinant expression system, including a fully-recombinant expression system based on thermophilic proteins has not been reported until now.

As will be discussed in more detail below, the current inventive technology overcomes the limitations of traditional cell-free expression systems while meeting the objectives of a truly energetically efficient and robust in vitro cell-free expression system that results in longer reaction durations and higher product yields. Specifically, the present invention includes a cell-free system based on thermophiles by recombinantly expressing each protein necessary for transcription/translation and thus enabling continuous flow with better control and fine tuning of the system without encountering huge variables as observed in extract-based batch systems. This system may be useful for small scale protein production in initial research applications as well as for mid-scale applications, such as small animal studies. The current invention allows for large scale manufacturing with the continuous flow approach in novel bioreactors described herein and can replace current manufacturing facilities with much larger footprints and personnel requirements.

### BRIEF SUMMARY OF THE INVENTION

One aim of the current invention relates to a recombinant cell-free expression system, the reaction mixture containing all the cell-free reaction components necessary for the in vitro transcription/translation mechanism, amino acids, nucleotides, metabolic components which provide energy, and which are necessary for protein synthesis. In a preferred embodiment, the enzymes identified herein may be sourced from different thermophile bacteria, as opposed to traditional cell-free systems that source components from E. coli or other eukaryotic systems, such as yeast. This thermophilic sourcing strategy provides higher stability during all steps during in vitro translation (tRNA loading, ribosomal peptide biosynthesis), as well as allows for improved performance and longer run-time of the recombinant expression system.

This present inventor's thermophilic sourcing strategy allows for the generation of a recombinant cell-free expression system that exhibits less sensitivity to variations in pH and salt concentrations and may be less affected by increasing phosphate concentration due to ATP hydrolysis. Another benefit of this thermophilic sourcing strategy is that it allows the inventive recombinant cell-free expression system to employ different sets of tRNAs, which are recognized by the thermophilic aminoacyl-tRNA synthetase enzymes, thus enabling full codon coverage for the first time in a cell-free system.

Another aim of the current invention may include a recombinant cell-free expression system, the reaction mixture containing all the cell-free reaction components necessary for the in vitro biosynthesis of biological compounds, proteins, enzymes, biosimilars or chemical modification of small molecules.

Another aim of the current invention may include methods, systems and apparatus for a continuous flow bioreactor system for in vitro transcription, in vitro translation and in vitro biosynthesis of vaccines, biologicals, proteins, enzymes, biosimilars and biosynthesis or chemical modification of small molecules using enzymes in a continuous flow operation.

Another aim of the invention may include one or more isolated nucleotide coding sequences that may form part of a recombinant cell-free expression reaction mixture. In a preferred embodiment, one or more nucleotide coding sequences may be from a thermophilic or other bacteria. In a preferred embodiment, a nucleotide coding sequences may include, but not be limited to: initiation factor nucleotide coding sequences, elongation factor nucleotide coding sequences, release factor nucleotide coding sequences, ribosome-recycling factor nucleotide coding sequences, aminoacyl-tRNA synthetase nucleotide coding sequences, and methionyl-tRNA transformylase nucleotide coding sequences. Additional nucleotide coding sequences may include RNA polymerase nucleotide coding sequences, as well as nucleotide coding sequences identified in the incorporated reference PCT Application No. PCT/US201 8/012121 (the “'121 Application”) related to the inorganic polyphosphate energy-regeneration system incorporated herein.

Another aim of the invention may include the generation of expression vectors having one or more isolated nucleotide coding sequences operably linked to promotor sequence(s) that may be used to transform a bacterial cell. In certain embodiments, nucleotide coding sequences may be optimized for expression in a select bacteria.

Another aim of the invention may include the expression of a nucleotide coding sequence identified herein generating a protein that may be further isolated and included in a recombinant cell-free expression reaction mixture. In a preferred embodiment, an expressed protein may include, but not be limited to: initiation factor proteins, elongation factor proteins, release factor proteins, ribosome-recycling factor proteins, aminoacyl-tRNA synthetase proteins, and methionyl-tRNA transformylase proteins. Additional nucleotide coding sequences may include RNA polymerase proteins, as well as proteins and compounds identified in the '121 Application related to the inorganic polyphosphate energy-regeneration system incorporated herein.

Another aim of the current invention may include a continuous flow recombinant cell-free expression apparatus. In this preferred embodiment, such a continuous flow recombinant cell-free expression apparatus may include the application of hollow fibers and hollow fiber-based bioreactors as an exchange medium for in vitro transcription, in vitro translation and in vitro biosynthesis of biological, proteins, enzymes, biosimilars and biosynthesis or chemical modification of small molecules using enzymes in a continuous flow operation.

Additional aims of the invention may include one or more of the following preferred embodiments:
1. A system for recombinant cell-free expression comprising:
   a core recombinant protein mixture having at least the following components:
      a plurality of initiation factors (IFs);
      a plurality of elongation factors (EFs);
      a plurality of peptide release factors (RFs);
      at least one ribosome recycling factor (RRF);
      a plurality of aminoacyl-tRNA-synthetases (RSs); and
      at least one methionyl-tRNA transformylase (MTF);
   at least one nucleic acid synthesis template;
   a reaction mixture having cell-free reaction components necessary for in vitro macromolecule synthesis; and
   wherein the above components are situated in a bioreactor configured for cell-free expression of macromolecules.
2. The system of embodiment 1, wherein the components of said core recombinant protein mixture comprises a core recombinant protein mixture derived from a bacteria.
3. The system of embodiment 2, wherein said core recombinant protein mixture derived from bacteria comprises a core recombinant protein mixture wherein at least one components is derived from a thermophilic bacteria.
4. The system of any one of embodiments 2, and 3, wherein said thermophilic bacteria comprises a thermophilic Bacillaceae bacteria, or Geobacillus thermophilic bacteria.
5. The system of embodiment 4, wherein said Geobacillus thermophilic bacteria is selected from the group consisting of: Geobacillus subterraneus, and Geobacillus stearothermophilus.
6. The system of embodiment 1, wherein said core recombinant protein mixture derived from bacteria comprises a core recombinant protein mixture wherein at least one components is derived from a non-thermophilic bacteria, or a combination of non-thermophilic and thermophilic bacteria.
7. The system of embodiment 6, wherein said non-thermophilic bacteria comprise Escherichia coli.
8. The system of embodiment 1, wherein said plurality of initiation factors (IFs) comprises a plurality of initiation factors derived from thermophilic bacteria.
9. The system of any one of embodiments 1, and 8, wherein said plurality of initiation factors derived from thermophilic bacteria comprise IF1, IF2, IF3, or a fragment or variant of any of the same.
10. The system of any one of embodiments 1, 8, and 9, wherein the plurality of initiation factors are selected from the group of amino acid sequences consisting of: SEQ ID NOs. 2, 4, 6, 70, 72, and 74, or a sequence having at least 90% sequence identity.
11. The system of embodiment 1, wherein said plurality of elongation factors (EFs) comprises a plurality of elongation factors derived from thermophilic bacteria.
12. The system of any one of embodiments 1, and 11, wherein said plurality of elongation factors derived from thermophilic bacteria comprise EF-G; EF-Tu; EF-Ts; EF-4; EF-P, or a fragment or variant of any of the same.
13. The system of any one of embodiments 1, 11, and 12, wherein the plurality of elongation factors are selected from the group of amino acid sequences consisting of: SEQ ID NOs. 8, 10, 12, 14, 16, 76, 78, 80, 82, and 84, or a sequence having at least 90% sequence identity.
14. The system of embodiment 1, wherein said plurality of peptide release factors (RFs) comprises a plurality of peptide release factors is derived from thermophilic bacteria, or a Bacillus bacteria.
15. The system of any one of embodiments 1, and 14, wherein said plurality of peptide release factors derived from a thermophilic bacteria comprise RF1, RF2, and RF3, or a fragment or variant of any of the same.
16. The system of any one of embodiments 1, 14, and 15, wherein the plurality of peptide release factors are selected from the group of amino acid sequences consisting of: SEQ ID NOs. 18, 20, 22, 86, 88, or a sequence having at least 90% sequence identity.
17. The system of embodiment 1, wherein said ribosome recycling factor (RRF) comprises a ribosome recycling factor derived from thermophilic bacteria.
18. The system of any one of embodiments 1, and 17, wherein said ribosome recycling factor is derived from Geobacillus.
19. The system of any one of embodiments 1, 17, and 18, wherein the ribosome recycling factor comprises a ribosome recycling factor according to amino acid sequences SEQ ID NOs. 14, and 90, or a sequence having at least 90% sequence identity.
20. The system of embodiment 1, wherein said plurality of aminoacyl-tRNA-synthetases (RSs) comprises a plurality of aminoacyl-tRNA-synthetases derived from thermophilic bacteria, or E. coli.
21. The system of any one of embodiments 1, and 20, wherein the plurality of aminoacyl-tRNA-synthetases comprises AlaRS; ArgRS; AsnRS; AspRS; CysRS; GlnRS; GluRS; GlyRS; HisRS; IleRS; LeuRS; LysRS; MetRS; PheRS (a); PheRS (b); ProRS; SerRS; ThrRS; TrpRS; TyrRS; and ValRS, or a fragment or variant of any of the same.
22. The system of any one of embodiments 1, 20, and 21, wherein said plurality of aminoacyl-tRNA-synthetases are selected from the group of amino acid sequences consisting of: SEQ ID NOs. 26, 28. 32, 34, 36, 38, 40, 42, 44, 46, 48, 50, 52, 54, 56, 58, 60, 62, 64, 66, 94, 96, 98, 100, 102, 104, 106, 108, 110, 112, 114, 116, 118, 120, 122, 124, 126, 128, and 130, or a sequence having at least 90% sequence identity.
23. The system of embodiment 1, wherein said methionyl-tRNA transformylase (MTF) comprises a methionyl-tRNA transformylase derived from thermophilic bacteria.
24. The system of embodiment 1, and 23, wherein said methionyl-tRNA transformylase is derived from Geobacillus.
25. The system of any one of embodiments 1, 23, and 24, wherein the methionyl-tRNA transformylase comprises a methionyl-tRNA transformylase according to amino acid sequences SEQ ID NOs. 68, and 132, or a sequence having at least 90% sequence identity.
26. The system of embodiment 1, wherein said nucleic acid synthesis template comprises a DNA template.
27. The system of embodiment 26, wherein said DNA template comprises a linear DNA template having:
   at least one target sequence operably linked to a promoter, and wherein said target sequence may optionally be codon optimized;
   at least one ribosome binding site (RBS);
   at least one expression product cleavage site; and
   at least one tag.
28. The system of embodiment 1, wherein said nucleic acid synthesis template comprises an RNA template.
29. The system of embodiment 1, wherein said reaction mixture comprises one or more of the following components:
   a quantity of ribosomes, and optionally a quantity of ribosomes derived from thermophilic bacteria;
   a quantity of RNase inhibitor;
   a quantity of RNA polymerase;
   a quantity of tRNAs, and optionally a quantity of tRNAs derived from thermophilic bacteria;
   a buffer; and
   a quantity of amino acids.
30. The system of embodiment 29, wherein said reaction mixture further comprises one or more of the following components:
   Tris-Acetate;
   Mg(OAc)2;
   K⁺-glutamate;
   amino-acetate;
   NaCl;
   KCl;
   MgCl₂;
   DTT;
   octyl-b-glycoside;
   NAD;
   NADP;
   sorbitol;
   FADH;
   CoA;
   PLP; and
   SAM.
31. The system of any of embodiments 1, and 29, and further comprising an energy source.
32. The system of embodiment 32, wherein said energy source comprises a quantity of nucleotide tri-phosphates (NTPs).
33. The system of embodiment 32, wherein said nucleotide tri-phosphates comprise one or more of the nucleotide tri-phosphates selected from the group consisting of: adenine triphosphate (ATP); guanosine triphosphate (GTP), Uridine triphosphate UTP, and Cytidine triphosphate (CTP)
34. The system of any of embodiments 31, 32, and 33, wherein said energy source comprises an inorganic polyphosphate-based energy regeneration system.
35. The system of embodiment 34, wherein said inorganic polyphosphate-based energy regeneration system comprises:
   a cellular adenosine triphosphate (ATP) energy regeneration system comprising:
      a quantity of Adenosyl Kinase (Gst AdK) enzyme;
      a quantity of Polyphosphate Kinase (TaqPPK) enzyme;
      a quantity of inorganic polyphosphate (PPi); and
      a quantity of adenosine monophosphate (AMP);
   wherein said AdK and PPK enzymes work synergistically to regenerate cellular ATP energy from PPi and AMP.
36. The system of embodiment 1, wherein said bioreactor comprises a continuous flow bioreactor.
37. A recombinant cell-free expression reaction mixture comprising:
   a plurality of initiation factors (IFs);
   a plurality of elongation factors (EF);
   a plurality of release factors (RF)
   at least one ribosome recycling factor (RRF);
   a plurality of aminoacyl-tRNA-synthetases (RSs); and
   at least one methionyl-tRNA transformylase (MTF);
38. The system of embodiment 37, wherein said plurality of initiation factors (IFs) comprise a plurality of initiation factors derived from thermophilic bacteria.
39. The system of any one of embodiments 37, and 38, wherein said plurality of initiation factors derived from thermophilic bacteria comprise IF1, IF2, IF3, or a fragment or variant of any of the same.
40. The system of any one of embodiments 37, 38, and 39, wherein the plurality of initiation factors are selected from the group of amino acid sequences consisting of: SEQ ID NOs. 2, 4, 6, 70, 72, and 74, or a sequence having at least 90% sequence identity.
41. The system of embodiment 37, wherein said plurality of elongation factors (EFs) comprise a plurality of elongation factors derived from thermophilic bacteria.
42. The system of any one of embodiments 37, and 41, wherein said plurality of elongation factors derived from a thermophilic bacteria comprises EF-G, EF-Tu, EF-Ts, EF-4, EF-P, or a fragment or variant of any of the same.
43. The system of any one of embodiments 37, 41, and 42, wherein the plurality of elongation factors are selected from the group of amino acid sequences consisting of: SEQ ID NOs. 8, 10, 12, 14, 16, 76, 78, 80, 82, and 84, or a sequence having at least 90% sequence identity.
44. The system of embodiment 37, wherein said plurality of peptide release factors (RFs) comprise a plurality of release factors derived from thermophilic bacteria, or a Bacillus sp. bacteria.
45. The system of any one of embodiments 37, and 44, wherein the plurality of peptide release factors comprises RF1, RF2, and RF3, or a fragment or variant of any of the same.
46. The system of any one of embodiments 37, 44, and 45, wherein the plurality of peptide release factors are selected from the group of amino acid sequences consisting of: SEQ ID NOs. 18, 20, 22, 86, 88, or a sequence having at least 90% sequence identity.
47. The system of embodiment 37, wherein said ribosome recycling factor (RRF) comprise a ribosome recycling factor derived from thermophilic bacteria.
48. The system of any one of embodiments 37, and 47, wherein said ribosome recycling factor derived from Geobacillus.
49. The system of any one of embodiments 37, 47, and 48, wherein the ribosome recycling factor comprise a ribosome recycling factor according to amino acid sequence SEQ ID NOs. 14, and 90, or a sequence having at least 90% sequence identity.
50. The system of embodiment 37, wherein said plurality of aminoacyl-tRNA-synthetases (RSs) comprise a plurality of aminoacyl-tRNA-synthetases wherein at least one is derived from thermophilic bacteria.
51. The system of any one of embodiments 37, and 50, wherein the plurality of aminoacyl-tRNA-synthetases comprise AlaRS; ArgRS; AsnRS; AspRS; CysRS; GlnRS; GluRS; GlyRS; HisRS; IleRS; LeuRS; LysRS; MetRS; PheRS (a); PheRS (b); ProRS; SerRS; ThrRS; TrpRS; TyrRS; and ValRS, or a fragment or variant of any of the same.
52. The system of any one of embodiments 37, 50, and 51, wherein said plurality of aminoacyl-tRNA-synthetases are selected from the group of amino acid sequences consisting of: SEQ ID NOs. 26, 28. 32, 34, 36, 38, 40, 42, 44, 46, 48, 50, 52, 54, 56, 58, 60, 62, 64, 66, 94, 96, 98, 100, 102, 104, 106, 108, 110, 112, 114, 116, 118, 120, 122, 124, 126, 128, and 130, or a sequence having at least 90% sequence identity
53. The system of any one of embodiments 37, wherein said methionyl-tRNA transformylase (MTF) comprises a methionyl-tRNA transformylase derived from thermophilic bacteria.
54. The system of any one of embodiments 37, and 53, wherein said methionyl-tRNA transformylase derived from Geobacillus.
55 The system of any one of embodiments 37, 53, and 54, wherein the methionyl-tRNA transformylase comprises a methionyl-tRNA transformylase according to amino acid sequence SEQ ID NOs. 68, and 132, or a sequence having at least 90% sequence identity.
56. An isolated nucleotide comprising a nucleotide selected from the group consisting of:
   SEQ ID NOs. 1, 3, 5 69, 71, and 73;
   SEQ ID NOs. 7, 9, 11, 13, 15, 75, 77, 79, 81, and 83;
   SEQ ID NOs. 17, 19, 21, 85, and 87;
   SEQ ID NOs. 23, and 89; and
   SEQ ID NO. 25, 27, 29, 31, 33, 35,37, 39, 41, 43, 45, 47, 49, 51, 53, 55, 57, 59, 61, 63, 65, 67, 91, 93, 95, 97, 99, 101, 103, 105, 107, 109, 111, 113, 115, 117, 119, 121, 123, 125, 127, 129 and 131.
57. An expression vector comprising at least one of the nucleotide sequences of embodiment 56, operably linked to a promoter.
58. A bacteria transformed by one of the expression vectors of embodiment 57.
59. The transformed bacteria of embodiment 58, wherein said bacteria comprises E. coli.
60. A peptide comprising an amino acid sequence selected from the group consisting of:
   SEQ ID NOs. 2, 4, 6, 70, 72 and 74;
   SEQ ID NOs. 8, 10, 12, 14, 16, 76, 78, 80, 82, and 84;
   SEQ ID NOs. 18, 20, 22, 86, 88;
   SEQ ID NOs. 14, and 90;
   SEQ ID NOs. 26, 28. 32, 34, 36, 38, 40, 42, 44, 46, 48, 50, 52, 54, 56, 58, 60, 62, 64, 66, 94, 96, SEQ ID NOs. 98, 100, 102, 104, 106, 108, 110, 112, 114, 116, 118, 120, 122, 124, 126, 128, and 130; and
   SEQ ID NOs. 68, and 132, or a fragment or variant of any of the same.
61. A cell-free expression system using at least one of the peptides of embodiment 60.

Additional aims of the inventive technology may become apparent from the detailed disclosure, figures and claims set forth below.

### BRIEF DESCRIPTION OF THE FIGURES

The accompanying figures, which are incorporated into and form a part of the specification, illustrate one or more embodiments of the present invention and, together with the description, serve to explain certain aspects of the inventive technology. The drawings are only for the purpose of illustrating one or more preferred embodiments of the invention and are not to be construed as limiting the invention.

FIG. 1: Demonstrates results of Aminoacyl-tRNA-Synthetase Kinetic Activity Assay for the following Synthetase enzymes: AlaRS, ArgRS, AsnRS, AspRS, CysRS, GlnRS (Ec), GluRS, GlyRS, HisRS, IleRS, and a no tRNA control.

FIG. 2: Demonstrates results of Aminoacyl-tRNA-Synthetase Kinetic Activity Assay for the following Synthetase enzymes: LeuRS, LysRS, MetRS, PheRS, ProRS, SerRS, ThrRS, TrpRS, TyrRS, and ValRS, and a no tRNA control.

FIG. 3A: Demonstrates results of Aminoacyl-tRNA-Synthetase Activity Assay utilizing exemplary tRNA from E. coli.

FIG. 3B: Demonstrates results of Aminoacyl-tRNA-Synthetase Activity Assay utilizing tRNA from the exemplary thermophilic bacteria Geobacillus stearothermophilus.

FIG. 4: Demonstrates the production of a Green Fluorescent Protein (muGFP, SEQ ID NO. 134)) cell-free expression product utilizing the recombinant cell-free expression system described herein.

FIG. 5: Diagram of a hollow fiber reactor for cell-free production and continuous exchange in one embodiment thereof.

FIG. 6A-B: Images of a hollow fiber reactor for cell-free production and continuous exchange in one embodiment thereof.

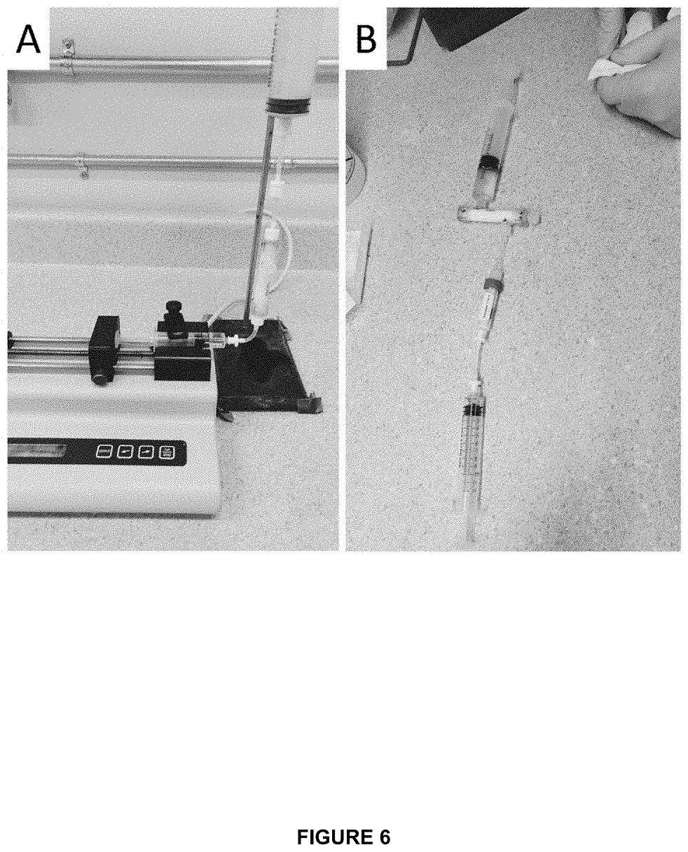

FIG. 7: A pET151/D-TOPO vector was used for select synthesized genes which add N-terminal tags to the expressed proteins. All genes expressed in this vector were reverse translated into DNA from the protein sequence and codon-optimized for expression in E. coli. N-terminal tags may be omitted from specific sequences identified below.

FIG. 8: A pET24a(+) vector was used for select synthesized genes which adds a C-terminal 6× His-tag to the expressed protein. All genes expressed in this vector were reverse translated into DNA from the protein sequence and codon-optimized for expression in E. coli. C-terminal tags may be omitted from specific sequences identified below.

FIG. 9: A pNAT vector was designed and used for select cloned and/or synthesized genes, which adds an N-terminal FLAG tag and/or a C-terminal 6× His tag to the expressed protein. All genes expressed in this vector were reverse translated into DNA from the protein sequence and codon-optimized for expression in E. coli. Tags may be omitted from specific sequences identified below.

FIG. 10: A pNAT 2.0 vector was designed and used for select cloned and/or synthesized genes, which adds an N-terminal or C-terminal 6× His tag to the expressed protein. All genes expressed in this vector were reverse translated into DNA from the protein sequence and codon-optimized for expression in E. coli. Tags may be omitted from specific sequences identified below.

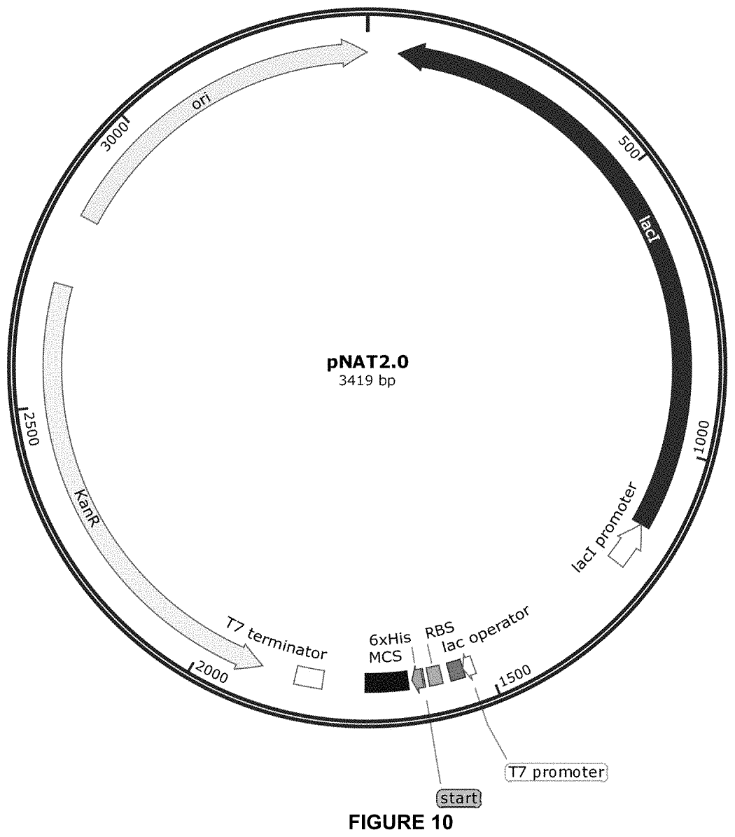

FIG. 11: Demonstrates SDS-PAGE results for the following purified Aminoacyl-tRNA-Synthetase (aaRS) enzymes: AlaRS, ArgRS, AsnRS, AspRS, CysRS, GlnRS (Ec), GluRS, GlyRS, HisRS, IleRS, and LeuRS.

FIG. 12: SDS-PAGE results for the following purified Aminoacyl-tRNA-Synthetase (aaRS) enzymes: LysRS, MetRS, PheBRS, ProRS, SerRS, ThrRS, TrpRS, TyrRS, ValRS, and the purified Methionyl-tRNA-Transformylase MTF.

FIG. 13: Demonstrates SDS-PAGE results for the following purified translation factors: IF-1, IF-2, IF-3, EF-G, EF-Ts, EF-Tu, EF-P, RF-1, RF-2, RF-3 and RRF.

FIG. 14: Demonstrates SDS-PAGE results for the purified translation factor EF-4.

FIG. 15: Demonstrates the real-time production of a fluorescent protein (muGFP; SEQ ID NO. 134) product utilizing the recombinant cell-free expression system described herein.

FIG. 16: shows a western blot with an anti-FLAG antibody of a cell-free protein expression reaction after reverse purification but without ribosomes filtered out. Demonstrates the specific detection of a protein cell-free expression product, specifically de-Green Fluorescent Protein (deGFP, SEQ ID NO. 135) utilizing the recombinant cell-free expression system described herein.

FIG. 17: (A) Demonstrates results of three independent Aminoacyl-tRNA-Synthetase AMP-Producing Activity Assay utilizing exemplary tRNA from E. coli. (B) Shows the AMP standard curve.

### MODE(S) FOR CARRYING OUT THE INVENTION(S)

The present invention is particularly suited for the on-demand manufacturing of therapeutic macromolecules, such as polypeptides, in a cell-free environment that are suitable for direct delivery to a patient. Therefore, the present invention will be primarily described and illustrated in connection with the manufacturing of therapeutic proteins. However, the present invention can also be used to manufacture any type of protein, including toxic proteins, proteins with radiolabeled amino acids, unnatural amino acids, etc. Further, the present invention is particularly suited for the on-demand manufacturing of proteins using cell-free expression, and thus the present invention will be described primarily in the context of cell-free protein expression.

The present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

The inventive technology described herein may include a novel recombinant cell-free expression system. In one preferred embodiment, the invention may include the generation of a reaction mixture that includes a plurality of core portions that may contribute to the in vitro expression activity. Exemplary core proteins may include the following:

In one embodiment, the recombinant cell-free expression system may include a reaction mixture having one or more initiation factors (IFs). Initiation factors may allow the formation of an initiation complex in the process of peptide synthesis. For example, IF1, IF2 and IF3 may be used in certain embodiments as initiation factors in the reaction mixture. For example, IF3 promotes the dissociation of ribosome into 30S and 50S subunits (i.e., the step being generally needed for initiating translation) and hinders the insertion of tRNAs other than formylmethionyl-tRNA into the P-position in the step of forming the initiation complex. IF2 binds to formylmethionyl-tRNA and transfers the formylmethionyl-tRNA to the P-position of 30S subunit, thereby forming the initiation complex. IF1 may potentiate the functions of IF2 and IF3. In the present invention, it may be preferable to use initiation factors derived from one or more bacteria, and more preferably thermophilic bacteria, for example, those obtained from the bacterial families Bacillaceae, and/or Geobacillus, such as Geobacillus subterraneus, or Geobacillus stearothermophilus. Exemplary amino acid sequences for one or more IFs of the invention may be selected from the group consisting of:

IF1 (SEQ ID NOs. 2, and 70)

IF2 (SEQ ID NOs. 4, and 72)

IF3 (SEQ ID NOs. 6, and 74)

In an embodiment of the invention, one or more of the above amino acid sequence thus comprises at least one IF comprising or consisting of an amino acid sequence encoded by the amino acid sequences according to SEQ ID NOs. 1-2, 4, 6 69-70, 72 and 74, or a fragment or variant of any one of these amino acid sequences. In this context, a fragment of a protein or a variant thereof encoded by the at least one coding region of the one or more IFs according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 1-2, 4, 6 69-70, 72 and 74 disclosed herein.

In the present invention, it may be preferable to use initiation factors expressed in, and/or isolated from one or more bacteria, and more preferably a bacteria configured to express high-levels of proteins, for example, E. coli. Exemplary nucleotide sequences for one or more IFs of the invention may be selected from the group consisting of:

IF1 (SEQ ID NOs. 1, and 69)

IF2 (SEQ ID NOs. 3, and 71)

IF3 (SEQ ID NOs. 5, and 73)

Notably, the nucleotide sequences may be codon-optimized for expression in one or more bacteria, or other protein expression system such as yeast or the like. For example, in this embodiment, exemplary nucleotide sequences SEQ ID NOs. 1, 3 and 5 have been codon-optimized for expression in E. coli.

In an embodiment of the invention, one or more of the above nucleotide sequence thus comprises at least one coding region encoding at least one IF comprising or consisting of a nucleotide sequence encoded by the nucleotide sequence according to SEQ ID NOs. 1, 3, 5, 69, 71, and 73, or a fragment or variant thereof. In this context, a fragment of a protein or a variant thereof encoded by the at least one coding region of the one or more IFs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 1, 3, 5, 69, 71, and 73 disclosed herein.

In one embodiment, the recombinant cell-free expression system may include a reaction mixture having one or more elongation factors. An elongation factor, such as EF-Tu, may be classified into 2 types, i.e., GTP and GDP types. EF-Tu of the GTP type binds to aminoacyl-tRNA and transfers it to the A-position of ribosome. When EF-Tu is released from ribosome, GTP is hydrolyzed into GDP. Another elongation factor EF-Ts binds to EF-Tu of the GDP type and promotes the conversion of it into the GTP type. Another elongation factor EF-G promotes translocation following the peptide bond formation in the process of peptide chain elongation. In the present invention, it is preferable to use EFs from bacterial and more preferably from and more preferably thermophilic bacteria, for example, those obtained from the bacterial families Bacillaceae, and/or Geobacillus, such as Geobacillus subterraneus, or Geobacillus stearothermophilus. Exemplary amino acid sequences for one or more EFs of the invention may be selected from the group consisting of:

EF-G (SEQ ID NOs. 8, and 76)

EF-Tu (SEQ ID NOs. 10, and 78)

EF-Ts (SEQ ID NOs. 12, and 80)

EF-4 (SEQ ID NOs. 14, and 82)

EF-P (SEQ ID NOs. 16, and 84)

In an embodiment of the invention, one or more of the above amino acid sequence thus comprises at least one EF comprising or consisting of an amino acid sequence encoded by the amino acid sequences according to SEQ ID NOs. 8, 10, 12, 14, 16, 76, 78, 80, 82, and 84 or a fragment or variant of any one of these amino acid sequences. In this context, a fragment of a protein or a variant thereof encoded by the at least one coding region of the one or more EFs according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 8, 10, 12, 14, 16, 76, 78, 80, 82, and 84 disclosed herein.

In the present invention, it may be preferable to use EFs expressed in, and/or isolated from one or more bacteria, and more preferably a bacteria configured to express high-levels of proteins, for example, E. coli. Exemplary nucleotide sequences for one or more EFs of the invention may be selected from the group consisting of:

EF-G (SEQ ID NOs. 7, and 75)

EF-Tu (SEQ ID NOs. 9, and 77)

EF-Ts (SEQ ID NOs. 11, and 79)

EF-4 (SEQ ID NOs. 13, and 81)

EF-P (SEQ ID NOs. 15, and 83)

Notably, the nucleotide sequences may be codon-optimized for expression in one or more bacteria, or other protein expression system such as yeast or the like. For example, in this embodiment, exemplary nucleotide sequences SEQ ID NOs. 7, 9, 11, 13, and 15 have been codon-optimized for expression in E. coli.

In an embodiment of the invention, one or more of the above nucleotide sequence thus comprises at least one coding region encoding at least one EF comprising or consisting of a nucleotide sequence encoded by the nucleotide sequence according to SEQ ID NOs. 7, 9, 11, 13, 15, 75, 77, 79 and 83 or a fragment or variant thereof. In this context, a fragment of a protein or a variant thereof encoded by the at least one coding region of the one or more EFs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 7, 9, 11, 13, 15, 75, 77, 79 and 83 disclosed herein.

In one embodiment, the recombinant cell-free expression system may include a reaction mixture having one or more peptide release factors (RFs). RFs may be responsible for terminating protein synthesis, releasing the translated peptide chain and recycling ribosomes for the initiation of the subsequent mRNA translation. When a protein is synthesized in a release factor-free reaction system, the reaction stops before the termination codon and thus a stable ternary complex (polysome display) composed of ribosome, peptide and mRNA can be easily formed. When a termination codon (UAA, UAG or UGA) is located at the A-position of ribosome, release factors RF1 and RF2 may enter the A-position and promote the dissociation of the peptide chain from peptidyl-tRNA at the P-position. RF1 recognizes UAA and UAG among the termination codons, while RF2 recognizes UAA and UGA. Another termination factor RF3 promotes the dissociation of RF1 and RF2 from ribosome after the dissociation of the peptide chain by RF1 and RF2.

In the present invention, it is preferable to use RFs from bacterial and more preferably from and more preferably thermophilic bacteria, for example, those obtained from the bacterial families Bacillaceae, and/or Geobacillus, such as Geobacillus subterraneus, or Geobacillus stearothermophilus. Exemplary amino acid sequences for one or more RFs of the invention may be selected from the group consisting of:

RF1 (SEQ ID NOs. 18, and 86)

RF2 (SEQ ID NOs. 20, and 88)

RF3 (SEQ ID NOs. 22)

In an embodiment of the invention, one or more of the above amino acid sequence thus comprises at least one RF comprising or consisting of an amino acid sequence encoded by the amino acid sequences according to SEQ ID NOs. 18, 20, 22, 86, and 88 or a fragment or variant of any one of these amino acid sequences. In this context, a fragment of a protein or a variant thereof encoded by the at least one coding region of the one or more RFs according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 18, 20, 22, 86, and 88 disclosed herein.

In the present invention, it may be preferable to use RFs expressed in, and/or isolated from one or more bacteria, and more preferably a bacteria configured to express high-levels of proteins, for example, E. coli. Exemplary nucleotide sequences for one or more RFs of the invention may be selected from the group consisting of:

RF1 (SEQ ID NOs. 17; and 85)

RF2 (SEQ ID NOs. 19; and 87)

RF3 (SEQ ID NO. 21)

Notably, the nucleotide sequences may be codon-optimized for expression in one or more bacteria, or other protein expression system such as yeast or the like. For example, in this embodiment, exemplary nucleotide sequences SEQ ID NOs. 17, 19, and 21 have been codon-optimized for expression in E. coli.

In an embodiment of the invention, one or more of the above nucleotide sequence thus comprises at least one coding region encoding at least one RF comprising or consisting of a nucleotide sequence encoded by the nucleotide sequence according to SEQ ID NOs. 17, 19, 21, 85, and 87 or a fragment or variant thereof. In this context, a fragment of a protein or a variant thereof encoded by the at least one coding region of the one or more RFs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 17, 19, 21, 85, and 87 disclosed herein.

In one embodiment, the recombinant cell-free expression system may include a reaction mixture having one or more ribosome recycling factor (RRF) which promotes the dissociation of tRNA remaining at the P-position after the protein synthesis and the recycling of ribosome for the subsequent protein synthesis. In the present invention, it is preferable to use RRFs from bacterial and more preferably from and more preferably thermophilic bacteria, for example, those obtained from the bacterial families Bacillaceae, and/or Geobacillus, such as Geobacillus subterraneus, or Geobacillus stearothermophilus. Exemplary amino acid sequences for one or more RRFs of the invention may be selected from the group consisting of:

RRF (SEQ ID NO. 24, and 90)

In an embodiment of the invention, one or more of the above amino acid sequence thus comprises at least one RRF comprising or consisting of an amino acid sequence encoded by the amino acid sequences according to SEQ ID NOs. 23 and 90 or a fragment or variant of any one of these amino acid sequences. In this context, a fragment of a protein or a variant thereof encoded by the at least one coding region of the one or more RRFs according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 23 and 90 disclosed herein.

In the present invention, it may be preferable to use RRFs expressed in, and/or isolated from one or more bacteria, and more preferably a bacteria configured to express high-levels of proteins, for example, E. coli. Exemplary nucleotide sequences for one or more RRFs of the invention may be selected from the group consisting of:

RRF (SEQ ID NOs. 23, and 89)

Notably, the nucleotide sequences may be codon-optimized for expression in one or more bacteria, or other protein expression system such as yeast or the like. For example, in this embodiment, exemplary nucleotide sequence SEQ ID NO. 23 has been codon-optimized for expression in E. coli.

In an embodiment of the invention, one or more of the above nucleotide sequence thus comprises at least one coding region encoding at least one RF comprising or consisting of a nucleotide sequence encoded by the nucleotide sequence according to SEQ ID NOs. 23, and 89 or a fragment or variant thereof. In this context, a fragment of a protein or a variant thereof encoded by the at least one coding region of the one or more RFs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 23, and 89 disclosed herein.

In one embodiment, the recombinant cell-free expression system may include a reaction mixture having one or more aminoacyl-tRNA synthetase (RS) enzymes. Aminoacyl-tRNA synthetase is an enzyme by which an amino acid is covalently bonded to tRNA in the presence of ATP to thereby synthesize aminoacyl-tRNA. In the present invention, it is preferable to use thermophile-origin aminoacyl-tRNA synthetase, for example, those obtained from the bacterial groups Bacillaceae, and/or Geobacillus, or more specifically from the species G. stearothermophilus, or Geobacillus stearothermophilus. Additional embodiments may include the use of an aminoacyl-tRNA synthetase enzymes from a non-thermophile, such as E. coli, such use being in conjunction with aminoacyl-tRNA synthetase enzymes of thermophile origin. Exemplary nucleotide and amino acid sequences for aminoacyl-tRNA synthetase enzymes selected from the group consisting of:

(SEQ ID NO. 26, and SEQ ID NO. 92)
AlaRS
(SEQ ID NO. 28, and SEQ ID NO. 94)
ArgRS
(SEQ ID NO. 30, and SEQ ID NO. 96)
AsnRS
(SEQ ID NO. 32, and SEQ ID NO. 98)
AspRS
(SEQ ID NO. 34, and SEQ ID NO. 100)
CysRS
(SEQ ID NO. 36)
GlnRS (Ec)
(SEQ ID NO. 38, and SEQ ID NO. 102)
GluRS
(SEQ ID NO. 40, and SEQ ID NO. 104)
GlyRS
(SEQ ID NO. 42, and SEQ ID NO. 106)
HisRS
(SEQ ID NO. 44, and SEQ ID NO. 108)
IleRS
(SEQ ID NO. 46, and SEQ ID NO. 110)
LeuRS
(SEQ ID NO. 48, and SEQ ID NO. 112)
LysRS
(SEQ ID NO. 50, and SEQ ID NO. 114)
MetRS
(SEQ ID NO. 52, and SEQ ID NO. 116)
PheRS (a)
(SEQ ID NO. 54, and SEQ ID NO. 118)
PheRS (b)
(SEQ ID NO. 56, and SEQ ID NO. 120)
ProRS
(SEQ ID NO. 58, and SEQ ID NO. 122)
SerRS
(SEQ ID NO. 60, and SEQ ID NO. 124)
ThrRS
(SEQ ID NO. 62, and SEQ ID NO. 126)
TrpRS
(SEQ ID NO. 64, and SEQ ID NO. 128)
TyrRS
(SEQ ID NO. 66, and SEQ ID NO. 130)
ValRS

In an embodiment of the invention, one or more of the above amino acid sequence thus comprises at least one RS comprising or consisting of an amino acid sequence encoded by the amino acid sequences according to SEQ ID NOs. 26, 28, 30, 32, 34, 36, 38, 40, 42, 44, 46, 48, 50, 52, 54, 56, 58, 60, 62, 64, 66, 92, 94, 96, 98, 100, 102, 104, 106, 108, 110, 112, 114, 116, 118, 120, 122, 134, 126, 128, and 130 or a fragment or variant of any one of these amino acid sequences. In this context, a fragment of a protein or a variant thereof encoded by the at least one coding region of the one or more RSs according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 26, 28, 30, 32, 34, 36, 38, 40, 42, 44, 46, 48, 50, 52, 54, 56, 58, 60, 62, 64, 66, 92, 94, 96, 98, 100, 102, 104, 106, 108, 110, 112, 114, 116, 118, 120, 122, 134, 126, 128, and 130 disclosed herein.

In the present invention, it may be preferable to use RSs expressed in, and/or isolated from one or more bacteria, and more preferably a bacteria configured to express high-levels of proteins, for example, E. coli. Exemplary nucleotide sequences for one or more RSs of the invention may be selected from the group consisting of:

(SEQ ID NO. 25, and SEQ ID NO. 91)
AlaRS
(SEQ ID NO. 27, and SEQ ID NO. 93)
ArgRS
(SEQ ID NO. 29, and SEQ ID NO. 95)
AsnRS
(SEQ ID NO. 31, and SEQ ID NO. 97)
AspRS
(SEQ ID NO. 33, and SEQ ID NO. 99)
CysRS
(SEQ ID NO. 35)
GlnRS
(Ec)
(SEQ ID NO. 37, and SEQ ID NO. 101)
GluRS
(SEQ ID NO. 39, and SEQ ID NO. 103)
GlyRS
(SEQ ID NO. 41, and SEQ ID NO. 105)
HisRS
(SEQ ID NO. 43, and SEQ ID NO. 107)
IleRS
(SEQ ID NO. 45, and SEQ ID NO. 109)
LeuRS
(SEQ ID NO. 47, and SEQ ID NO. 111)
LysRS
(SEQ ID NO. 49, and SEQ ID NO. 113)
MetRS
(SEQ ID NO. 51, and SEQ ID NO. 115)
PheRS (a)
(SEQ ID NO. 53, and SEQ ID NO. 117)
PheRS (b)
(SEQ ID NO. 55, and SEQ ID NO. 119)
ProRS
(SEQ ID NO. 57, and SEQ ID NO. 121)
SerRS
(SEQ ID NO. 59, and SEQ ID NO. 123)
ThrRS
(SEQ ID NO. 61, and SEQ ID NO. 125)
TrpRS
(SEQ ID NO. 63, and SEQ ID NO. 127)
TyrRS
(SEQ ID NO. 65, and SEQ ID NO. 129)
ValRS

Notably, the nucleotide sequences may be codon-optimized for expression in one or more bacteria, or other protein expression system such as yeast or the like. For example, in this embodiment, exemplary nucleotide sequence SEQ ID NOs. 25, 27, 29, 31, 33, 35, 37, 39, 41, 43, 45, 47, 49, 51, 53, 55, 57, 59, 61, 63, and 65 have been codon-optimized for expression in E. coli.

In an embodiment of the invention, one or more of the above nucleotide sequence thus comprises at least one coding region encoding at least one RS comprising or consisting of a nucleotide sequence encoded by the nucleotide sequence according to SEQ ID NOs. 25, 27, 29, 31, 33, 35,37, 39, 41, 43, 45, 47, 49, 51, 53, 55, 57, 59, 61, 63, 65, 91, 93, 95, 97, 99, 101, 103, 105, 107, 109, 111, 113, 115, 117, 119, 121, 123, 125, 127, and 129 or a fragment or variant thereof. In this context, a fragment of a protein or a variant thereof encoded by the at least one coding region of the one or more RSs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 25, 27, 29, 31, 33, 35,37, 39, 41, 43, 45, 47, 49, 51, 53, 55, 57, 59, 61, 63, 65, 91, 93, 95, 97, 99, 101, 103, 105, 107, 109, 111, 113, 115, 117, 119, 121, 123, 125, 127, and 129 disclosed herein.

In one embodiment, the recombinant cell-free expression system may include a reaction mixture having a methionyl-tRNA transformylase (MTF). N-Formylmethionine, carrying a formyl group attached to the amino group at the end of methionine, serves as the initiation amino acid in a prokaryotic protein synthesis system. This formyl group is attached to the methionine in methionyl-tRNA by MTF. Namely, MTF transfers the formyl group in Nlυ-formyltetrahydrofolate to the N-terminus of methionyl-tRNA corresponding to the initiation codon, thereby giving a formylmethionyl-tRNA. The formyl group thus attached is recognized by IF2 and acts as an initiation signal for protein synthesis. In the present invention, it is preferable to use an MTF from bacterial and more preferably from and more preferably thermophilic bacteria, for example, those obtained from the bacterial families Bacillaceae, and/or Geobacillus, such as Geobacillus subterraneus, or Geobacillus stearothermophilus. Exemplary amino acid sequences for one or more MTFs of the invention may be selected from the group consisting of:

MTF (SEQ ID NO. 68, and 132)

In an embodiment of the invention, one or more of the above amino acid sequence thus comprises at least one MTF comprising or consisting of an amino acid sequence encoded by the amino acid sequences according to SEQ ID NOs. 68, and 132 or a fragment or variant of any one of these amino acid sequences. In this context, a fragment of a protein or a variant thereof encoded by the at least one coding region of the one or more MTF s according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein or a variant thereof, preferably according to SEQ ID NOs. 68, and 132 disclosed herein.

In the present invention, it may be preferable to use an MTF expressed in, and/or isolated from one or more bacteria, and more preferably a bacteria configured to express high-levels of proteins, for example, E. coli. Exemplary nucleotide sequences for one or more MTFs of the invention may be selected from the group consisting of:

MTF (SEQ ID NO. 67, and 131)

Notably, the nucleotide sequences may be codon-optimized for expression in one or more bacteria, or other protein expression system such as yeast or the like. For example, in this embodiment, exemplary nucleotide sequence SEQ ID NO. 67 has been codon-optimized for expression in E. coli.

In an embodiment of the invention, one or more of the above nucleotide sequence thus comprises at least one coding region encoding at least one MTF comprising or consisting of a nucleotide sequence encoded by the nucleotide sequence according to SEQ ID NOs. 67, and 131 or a fragment or variant thereof. In this context, a fragment of a protein or a variant thereof encoded by the at least one coding region of the one or more MTFs according to the invention may typically comprise a nucleotide sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an nucleotide sequence of the respective naturally occurring full-length protein or a variant thereof, preferably according to SEQ ID NOs. 67, and 131 disclosed herein.

In one embodiment, the recombinant cell-free expression system may include a reaction mixture having a quantity of ribosomes. A ribosome is a particle where peptides are synthesized. It binds to mRNA and coordinates aminoacyl-tRNA to the A-position and formylmethionyl-tRNA or peptidyl-tRNA to the P-position, thereby forming a peptide bond. In the present invention, any ribosome can be used regardless of the origin, however, in a preferred embodiment, ribosomes may be isolated from thermophilic bacteria for use in the recombinant cell-free expression system, and preferably from cell lysates of thermophilic bacteria, such as from the bacterial families Bacillaceae, and/or Geobacillus, such as Geobacillus subterraneus, or Geobacillus stearothermophilus.

In one embodiment, the recombinant cell-free expression system may include a reaction mixture having a quantity of RNA polymerase or fragment or variant thereof which is an enzyme transcribing a DNA sequence into an RNA, occurs in various organisms. As an example, thereof, in one preferred embodiment, the invention may include a T7 RNA polymerase, for example according to amino acid sequence SEQ ID NO. 136. T7 RNA polymerase is derived from the in T7 phage which is an enzyme binding to a specific DNA sequence called T7 promoter and then transcribing the downstream DNA sequence into an RNA. In addition to T7 RNA polymerase, various RNA polymerases are usable in the present invention.

In one embodiment, the recombinant cell-free expression system may include a reaction mixture having a quantity of RNase inhibitor. RNase enzymes promoted the breakdown of RNA into oligonucleotides. RNase inhibitors are known in the art; as such, the type and quantity of RNase inhibitor to be included in a recombinant cell-free expression system is within the skill of those having ordinary skill in the art. Non-limiting examples of RNase inhibitors include mammalian ribonuclease inhibitor proteins [e.g., porcine ribonuclease inhibitor and human ribonuclease inhibitor (e.g., human placenta ribonuclease inhibitor and recombinant human ribonuclease inhibitor)], aurintricarboxylic acid (ATA) and salts thereof [e.g., triammonium aurintricarboxylate (aluminon)], adenosine 5′-pyrophosphate, 2′-cytidine monophosphate free acid (2′-CMP), 5′-diphosphoadenosine 3′-phosphate (ppA-3′-p), 5′-diphosphoadenosine 2′-phosphate (ppA-2′-p), leucine, oligovinysulfonic acid, poly(aspartic acid), tyrosine-glutamic acid polymer, 5′-phospho-2′-deoxyuridine 3′-pyrophosphate P′→5′-ester with adenosine 3′-phosphate (pdUppAp), and analogs, derivatives and salts thereof.

In one embodiment, the recombinant cell-free expression system may include a reaction mixture having a quantity of amino acids, a polynucleotide, such as an mRNA or DNA template encoding a target sequence typically in the form of a plasmid synthesis template, or linear expression (or synthesis) template (LET or LST), and other compounds and sequences identified in the '121 Application related to the inorganic polyphosphate energy-regeneration system, and preferably a coupled AdK/PPK energy regeneration system which may be necessary to energetically drive the in vitro expression reaction.

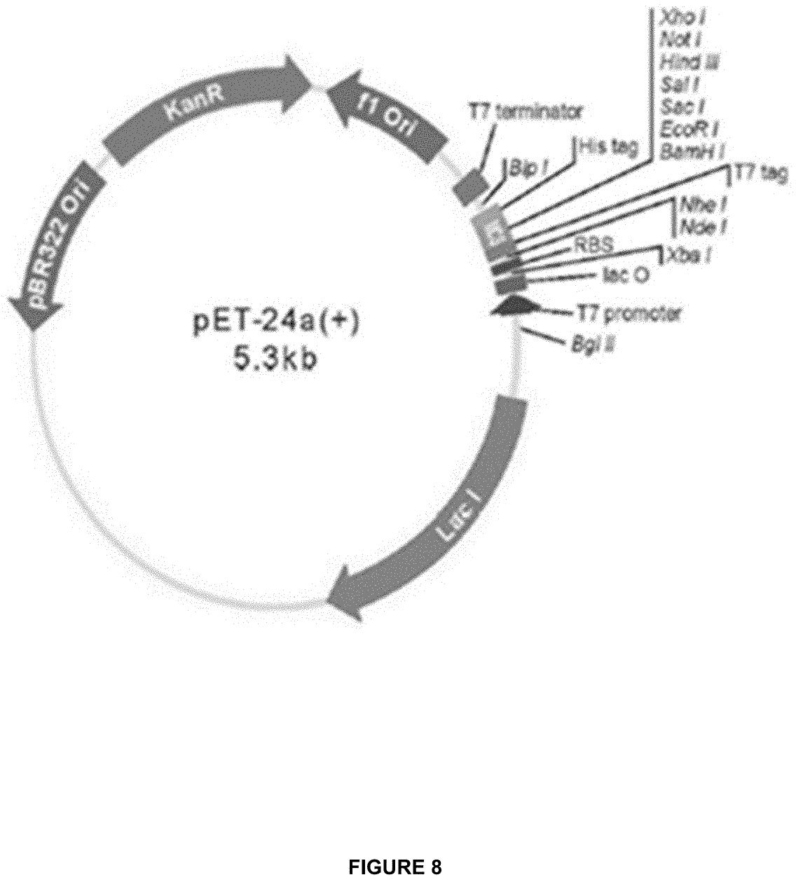

As generally shown in FIG. 8 of the '121 Application (incorporated herein by reference), in another preferred embodiment, isolated and purified Gst AdK (SEQ ID NO. 8 of the '121 application incorporated herein by reference) and/or TaqPPK (SEQ ID NO. 11 of the '121 application incorporated herein by reference) may be added to this cell-free expression system with a quantity of inorganic polyphosphate. In one embodiment, this quantity of inorganic polyphosphate may include an optimal polyphosphate concentration range. In this preferred embodiment, such optimal polyphosphate concentration range being generally, defined as the concentration of inorganic polyphosphate (PPi) that maintains the equilibrium of the reaction stable. In this preferred embedment, optimal polyphosphate concentration range may be approximately 0.2-2 mg/ml PPi.

As noted above, PPK can synthesize ADP from polyphosphate and AMP. In this preferred embodiment the coupled action of Gst AdK and PPK, may remove adenosine diphosphate (ADP) from the system by converting two ADP to one ATP and one adenosine monophosphate (AMP):

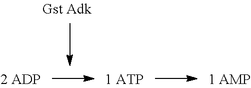

This reaction may be sufficiently fast enough to drive an equilibrium reaction of PPK towards production of ADP:

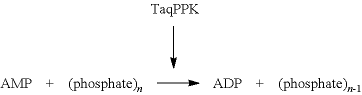

In this system, the presence of higher concentrations of AMP may further drive the TaqPPK reaction towards ADP.

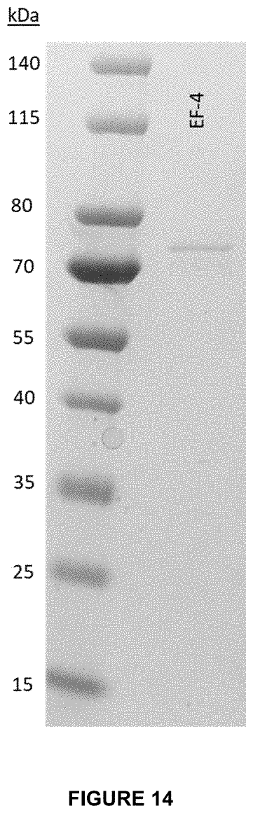

In a preferred embodiment, the production of macromolecules using the recombinant cell-free system of the invention may be accomplished in a bioreactor system. As used herein, a “bioreactor” may be any form of enclosed apparatus configured to maintain an environment conducive to the production of macromolecules in vitro. A bioreactor may be configured to run on a batch, continuous, or semi-continuous basis, for example by a feeder reaction solution. Referring to FIG. 14 of the '121 application (incorporated herein by reference), in this embodiment the invention may further include a cell-free culture apparatus. This cell culture apparatus may be configured to culture, in certain preferred embodiments thermophilic bacteria. A fermentation vessel may be removable and separately autoclavable in a preferred embodiment. Additionally, this cell-free culture apparatus may be configured to accommodate the growth of aerobic as well as anaerobic with organisms. Moreover, both the cell-free expression bioreactor and cell-free culture apparatus may accommodate a variety of cell cultures, such a microalgae, plant cells and the like.

In one embodiment, the present invention may be particularly suited for operation with a continuous exchange or flow bioreactor (1). In this preferred embodiment, this continuous exchange production apparatus may include a plurality of fibers and hollow fiber-based bioreactor as an exchange medium for in vitro transcription, in vitro translation and in vitro biosynthesis of biologicals, vaccines, proteins, enzymes, biosimilars and biosynthesis or chemical modification of small molecules using enzymes in a continuous flow operation.

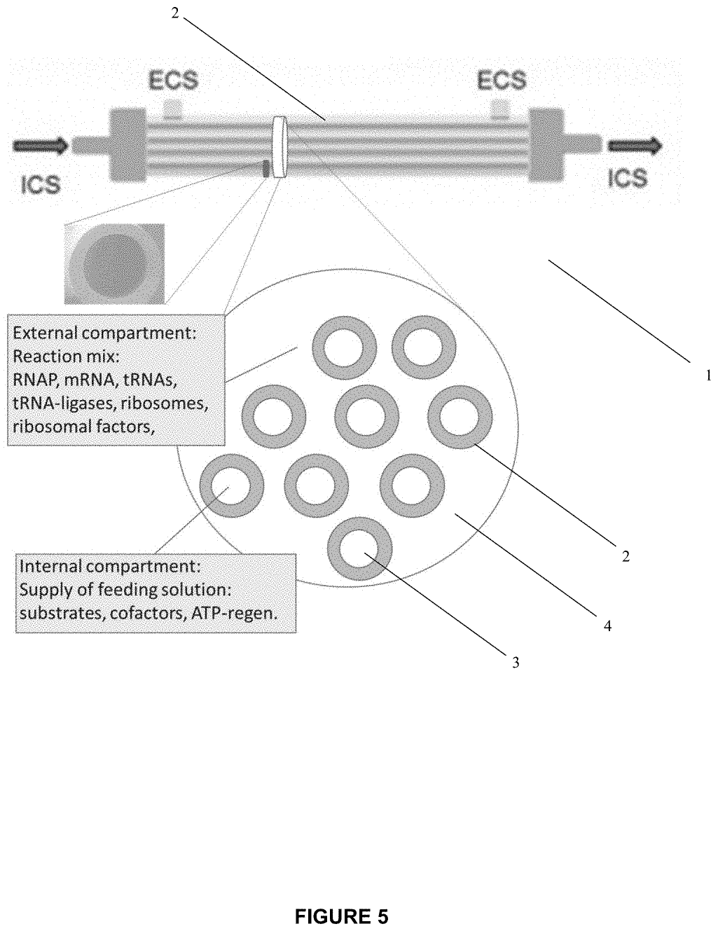

Generally referring to FIG. 5, a continuous flow bioreactor apparatus may include one or more hollow fibers (2) and hollow fiber-based bioreactors (2) as an exchange medium for in vitro transcription, in vitro translation and in vitro biosynthesis of biological, proteins, enzymes, biosimilars and biosynthesis or chemical modification of small molecules using enzymes in a continuous flow operation. In this embodiment, a continuous supply of substrates as described herein may be introduced to the apparatus, and may further be accompanied with the removal of a reaction product via a concentration gradient between the inner and out compartment of the hollow fiber reactors (2), allows for extend operational time and batch-independent production of biological and biologically modified materials, which may be isolated from the “flow-through” solution of the inner compartment.

As shown in FIGS. 5A and 5B, the operation of an exemplary hollow fiber reactor (2) is described. In this embodiment, while a feeding solution is pushed through the inner compartment of the reactor (3), the permeability of the fibers allow a continuous supply of substrates for mRNA synthesis (nucleotides), proteins in general (amino acids), substrates (for the in vitro biosynthesis or chemical modification of compounds) and the ATP regeneration system as incorporated herein from the '121 application to provide ATP and (via a nucleotide kinase, e.g. NDPK) GTP for the operation of the ribosome, the outer compartment (4) contains enzymes and factors to drive the in vitro transcription, in vitro translation, and in vitro biosynthesis reactions in a continuous exchange. Produced proteins, enzymes and larger biologicals are isolated and purified in a closed loop system as shown in FIG. 5B. This closed loop system prevents and/or reduces the risk of potential contaminations of the product, spillage or exposure, reducing the volume that needs to be processed and reducing the footprint of production spaces for biologicals of any kind. A straightforward increase of the volume of the reaction vessel, allows the adaptation from research scale biosynthesis to industrial scale production. Thus, reducing the development effort and costs for process scaling and development timelines.

In vitro recombinant cell-free expression, as used herein, refers to the cell-free synthesis of polypeptides in a reaction mixture or solution comprising biological extracts and/or defined cell-free reaction components. The reaction mix may comprise a template, or genetic template, for production of the macromolecule, e.g. DNA, mRNA, etc.; monomers for the macromolecule to be synthesized, e.g. amino acids, nucleotides, etc.; and such co-factors, enzymes and other reagents that are necessary for the synthesis, e.g. ribosomes, tRNA, polymerases, transcriptional factors, etc. The recombinant cell-free synthesis reaction, and/or cellular adenosine triphosphate (ATP) energy regeneration system components, incorporated by reference herein, may be performed/added as batch, continuous flow, or semi-continuous flow.

Some of the target proteins that may be expressed by the present invention may include, but not limited to: vaccines, eukaryotic peptides, prokaryotic peptides, bacterial related peptides, fungal related peptides, yeast-related, human related peptides, plant related peptides, toxin peptides, vasoactive intestinal peptides, vasopressin peptides, novel or artificially engineered peptides, virus related peptides, bacteriophage related proteins, hormones, antibodies, cell receptors, cell regulator proteins and fragments of any of the above-listed polypeptides.

Because this invention involves production of genetically altered organisms and involves recombinant DNA techniques, the following definitions are provided to assist in describing this invention.

The terms “isolated”, “purified”, or “biologically pure” as used herein, refer to material that is substantially or essentially free from components that normally accompany the material in its native state or when the material is produced. In an exemplary embodiment, purity and homogeneity are determined using analytical chemistry techniques such as polyacrylamide gel electrophoresis or high-performance liquid chromatography. A nucleic acid or particular bacteria that are the predominant species present in a preparation is substantially purified. In an exemplary embodiment, the term “purified” denotes that a nucleic acid or protein that gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

In preferred embodiments, the output of the cell-free expression system may be a product, such as a peptide or fragment thereof that may be isolated or purified. In the embodiment, solation or purification of a of a target protein wherein the target protein is at least partially separated from at least one other component in the reaction mixture, for example, by organic solvent precipitation, such as methanol, ethanol or acetone precipitation, organic or inorganic salt precipitation such as trichloroacetic acid (TCA) or ammonium sulfate precipitation, nonionic polymer precipitation such as polyethylene glycol (PEG) precipitation, pH precipitation, temperature precipitation, immunoprecipitation, chromatographic separation such as adsorption, ion-exchange, affinity and gel exclusion chromatography, chromatofocusing, isoelectric focusing, high performance liquid chromatography (HPLC), gel electrophoresis, dialysis, microfiltration, and the like.

As used herein, the term “activity” refers to a functional activity or activities of a peptide or portion thereof associated with a full-length (complete) protein. Functional activities include, but are not limited to, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide. Preferably, the activity of produced proteins retain at least 55%, 60%, 65%, 70%, 80%, 85%, 90%, 95% or more of the initial activity for at least 3 days at a temperature from about 0° C. to 30° C.

The term “nucleic acid” as used herein refers to a polymer of ribonucleotides or deoxyribonucleotides. Typically, “nucleic acid” polymers occur in either single- or double-stranded form but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein. For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

As used herein, the terms “target protein” refers generally to any peptide or protein having more than about 5 amino acids. The polypeptides may be homologous to, or preferably, may be exogenous, meaning that they are heterologous, i.e., foreign, to the bacteria from which the bacterial cell where they may be produced, such as a human protein or a yeast protein produced in the host bacteria, such as E. coli. Preferably, mammalian polypeptides, viral, bacterial, fungal and artificially engineered polypeptides are used.

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system.

All nucleotide sequences described in the invention may be codon optimized for expression in a particular organism, or for increases in production yield. Codon optimization generally improves the protein expression by increasing the translational efficiency of a gene of interest. The functionality of a gene may also be increased by optimizing codon usage within the custom designed gene. In codon optimization embodiments, a codon of low frequency in a species may be replaced by a codon with high frequency, for example, a codon UUA of low frequency may be replaced by a codon CUG of high frequency for leucine. Codon optimization may increase mRNA stability and therefore modify the rate of protein translation or protein folding. Further, codon optimization may customize transcriptional and translational control, modify ribosome binding sites, or stabilize mRNA degradation sites.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. Exemplary conservative amino acid substitutions are known by those of ordinary skill in the art. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Homology (e.g., percent homology, sequence identity+sequence similarity) can be determined using any homology comparison software computing a pairwise sequence alignment. As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

According to a specific embodiment, the homolog sequences are at least 60%, 65%, 70%, 75%, 80%, 85%, 90%, 95%, 99% or even identical to the sequences (nucleic acid or amino acid sequences) provided herein. Homolog sequences of SEQ ID Nos 1-22 of between 50%-99% may be included in certain embodiments of the present invention.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid but must be sufficiently complementary to hybridize with the template. The design of suitable primers for the amplification of a given target sequence is well known in the art and described in the literature cited herein.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “reaction mixture,” or “cell-free reaction mixture” or “recombinant cell-free reaction mixture” as used herein, refers to a solution containing reagents necessary to carry out a given reaction. A cell-free expression system “reaction mixture” or “reaction solution” typically contains a crude or partially-purified extract, (such as from a bacteria, plant cell, microalgae, fungi, or mammalian cell) nucleotide translation template, and a suitable reaction buffer for promoting cell-free protein synthesis from the translation template. In one aspect, the CF reaction mixture can include an exogenous RNA translation template. In other aspects, the CF reaction mixture can include a DNA expression template encoding an open reading frame operably linked to a promoter element for a DNA-dependent RNA polymerase. In these other aspects, the CF reaction mixture can also include a DNA-dependent RNA polymerase to direct transcription of an RNA translation template encoding the open reading frame. In these other aspects, additional NTPs and divalent cation cofactor can be included in the CF reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention. Moreover, those of ordinary skill will understand that some components in a reaction mixture, while utilized in certain embodiments, are not necessary to generate cell-free expression products. The term “cell-free expression products” may be any biological product produced through a cell-free expression system.

The term “about” or “approximately” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” or “approximately” will depend upon the particular system under study. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

The term “recombinant” or “genetically modified” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed, over-expressed, under-expressed or not expressed at all.

As used herein, the term “transformation” or “genetically modified” refers to the transfer of one or more nucleic acid molecule(s) into a cell. A microorganism is “transformed” or “genetically modified” by a nucleic acid molecule transduced into the bacteria or cell or organism when the nucleic acid molecule becomes stably replicated. As used herein, the term “transformation” or “genetically modified” encompasses all techniques by which a nucleic acid molecule can be introduced into a cell or organism, such as a bacteria.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor or binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “genome” refers to chromosomal DNA found within the nucleus of a cell, and also refers to organelle DNA found within subcellular components of the cell. The term “genome” as it applies to bacteria refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

The terms “expression product” as it relates to a protein expressed in a cell-free expression system as generally described herein, are used interchangeably and refer generally to any peptide or protein having more than about 5 amino acids. The polypeptides may be homologous to, or may be exogenous, meaning that they are heterologous, i.e., foreign, to the organism from which the cell-free extract is derived, such as a human protein, plant protein, viral protein, yeast protein, etc., produced in the cell-free extract. In some embodiment, the term “derived” means extracted from, or expressed and isolated from a bacteria. For example, in one embodiment a protein may be derived from a thermophilic bacteria may mean a protein that is endogenous to a thermophilic bacteria and isolated from said bacteria or expressed heterologously in a different bacteria and isolated as an individual protein or cell extract.

A “cell-free extract” or “lysate” may be derived from a variety of organisms and/or cells, including bacteria, thermophilic bacteria, thermotolerant bacteria, archaea, firmicutes, fungi, algae, microalgae, plant cell cultures, and plant suspension cultures.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1

### Synthesis and Cloning of Proteins for Recombinant Cell-Free Expression System

The present inventors synthesized and cloned into select expression vectors a plurality of core recombinant proteins, and preferably from a select thermophilic bacteria, for use in a recombinant cell-free expression system. In this embodiment, the present inventors synthesized and cloned into select expression vectors a plurality of core recombinant thermophilic initiation factors (IFs). In this embodiment, the present inventors synthesized and cloned into select expression vectors a plurality of core recombinant thermophilic elongation factors (EFs). In this embodiment, the present inventors synthesized and cloned into select expression vectors a plurality of core recombinant release factors (RFs). In this embodiment, the present inventors synthesized and cloned into select expression vectors at least one core recombinant ribosome recycling factor (RRFs). In this embodiment, the present inventors synthesized and cloned into select expression vectors a plurality of core recombinant aminoacyl-tRNA-synthetases (RSs). In this embodiment, the present inventors synthesized and cloned into select expression vectors at least one core recombinant methionyl-tRNA transformylase (MTF).

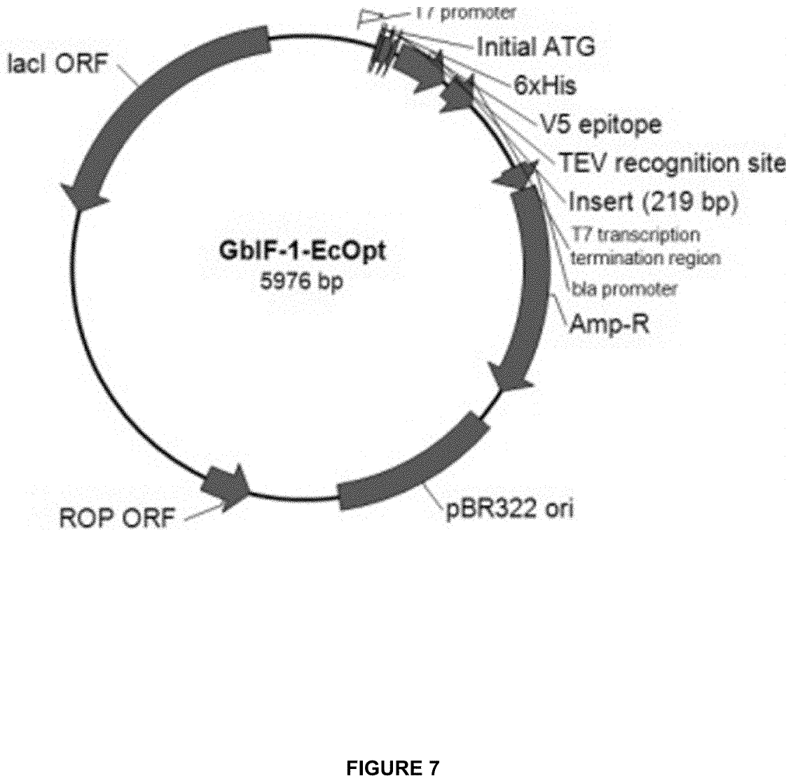

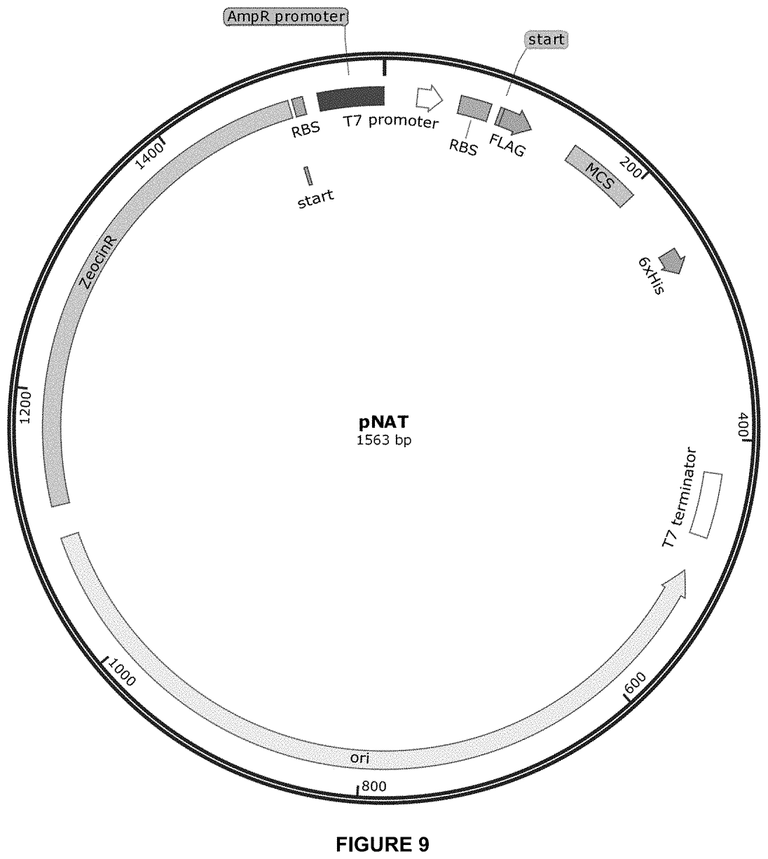

As shown generally in Table 1, in one preferred embodiment, the present inventors synthesized, cloned, expressed in E. coli and purified at least twelve (12) different recombinant factors, including nucleotide and/or amino acid sequences, and at least twenty-two (22) recombinant synthetases, including nucleotide and/or amino acid sequences (SEQ ID NOs. 1-132) that form an exemplary Core Recombinant Protein Mixture of at least thirty-four (34) proteins that may be applied to the inventive recombinant cell-free expression system. These core proteins were clone into an expression vector, for example the pET151/D-TOPO (pET151), pET24a(+), or pNAT, as shown in FIGS. 7-8 and 9.

The present inventors further generated a recombinant cell-free reaction mixture that incorporates one or more of the thirty-four (34) proteins identified, as well as select isolated ribosomes and tRNA from exemplary thermophilic bacteria. The present inventors next included in the recombinant cell-free reaction mixture a quantity of RNA polymerase, and in particular a T7 RNA polymerase enzyme, as well as exemplary amino acids, and buffers. As noted above, the present inventors further generated a recombinant cell-free reaction mixture that incorporates one or more of the components of the inorganic polyphosphate energy-regeneration system identified in the claims of in PCT Application No. PCT/US201 8/012121 ('121 Application).

### Example 2

### Generation of an Exemplary Recombinant Cell-Free Reaction Mixture

In one embodiment, the present inventors generated a recombinant cell-free reaction mixture capable of in vitro transcription and translation selected from the group consisting of:
a reaction mixture at least thirty-three (33) thermophilic core proteins identified in Table 1;
one (1) core protein from E. coli identified in Table 1;
tRNA from thermophiles
a quantity of ribosomes isolated from select thermophiles;
a quantity of amino acids;
a quantity of nucleotide tri-phosphates (NTPs) such as ATP, CTP, GTP, TTP;
a quantity of a reaction buffer; and
one or more components of the inorganic polyphosphate-based energy regeneration or energy regeneration system identified in the claims, figures, sequences, and specification of the '121 Application, which has been incorporated herein.

### Example 3

### Activity of Recombinant Aminoacyl-tRNA-Synthetases

The present inventors confirmed the activity of each purified aminoacyl-tRNA-synthetase (RS). Generally, the aminoacyl-tRNA-synthetase reaction is a two-step process:

Step 1: Activation amino acid+ATP=>aminoacyl-AMP+PPi

Step 2: Transfer aminoacyl-AMP+tRNA=>aminoacyl-tRNA+AMP

The resulting PPi can be measured using the EnzCheck pyrophosphate kit. Utilizing this outline, the present inventors performed kinetic assays using a commercial pyrophosphate assay kit (EnzCheck Pyrophosphate Assay Kit, Molecular Probes, E-6654, incorporated herein by reference). This commercially available assay spectrophotometrically measures indirectly the enzymatic production of pyrophosphate. Each RS reaction was set up in a total of 30 μl with the following final concentrations shown in Table 2. 12.5 μl of the RS reaction mix was used to set up a 50 μl reaction for the pyrophosphate assay as demonstrated in Table 3. Pyrophosphate assays were set up in a 96-well plate and automatically read in 2 min intervals on a plate reader set to read the absorbance at 360 nm. These kinetic measurements were used as a qualitative first test of the activity and functionality of all RS proteins.

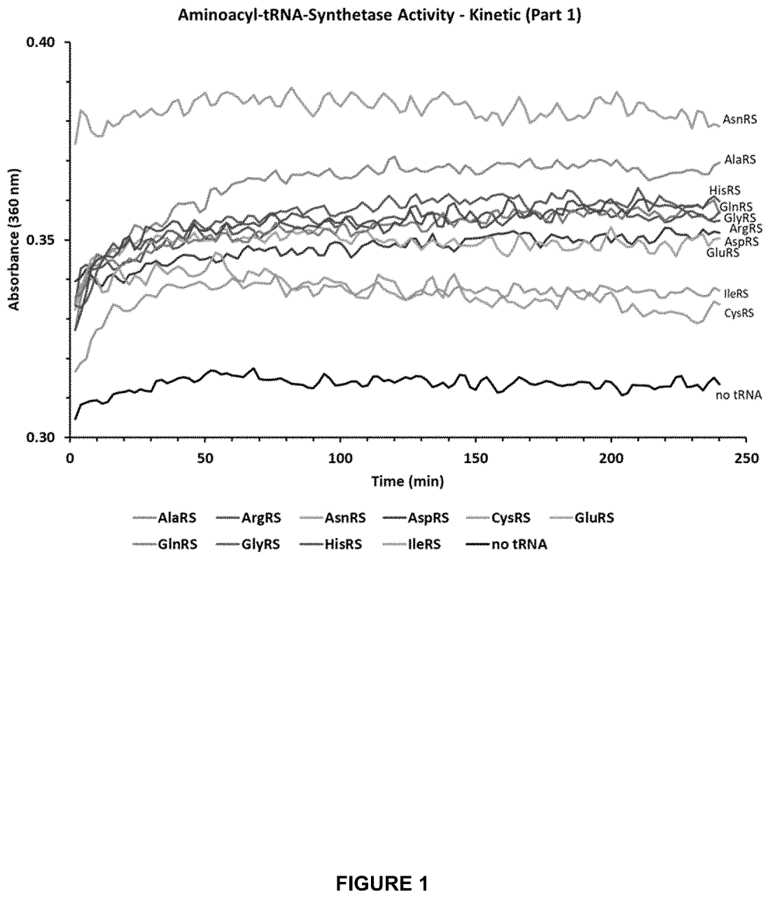

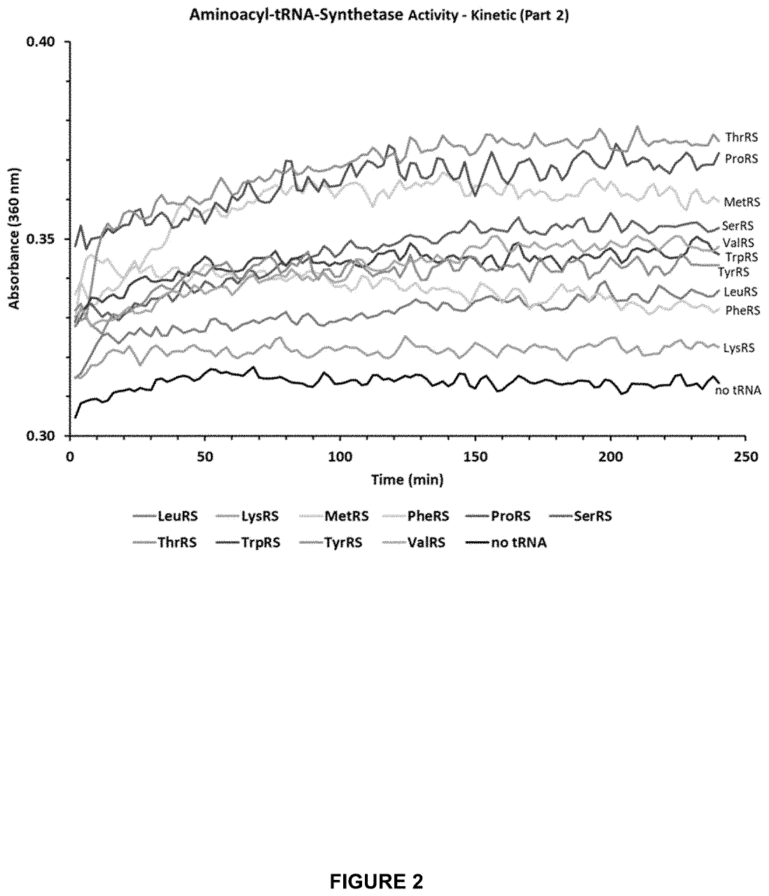

Assays were performed according to the manufacturer's instructions and the change in absorbance over time was plotted over time for each RS. As shown in FIGS. 1 and 2, each RS demonstrated good activity (no tRNA as control) and inorganic pyrophosphate is produced by hydrolysis of ATP to ADP+Pi and Pi can be detected indirectly using the EnzCheck assay kit. Even with low absorbance change, the data in FIGS. 1 and 2 is comparable to published reports regarding RS and graphs shown for other enzyme kinetics for ATP usage provided by the manufacture's guidelines. For clarity, for both FIGS. 1 and 2 only 10 RS were plotted on each graph but originated from the same experiment.

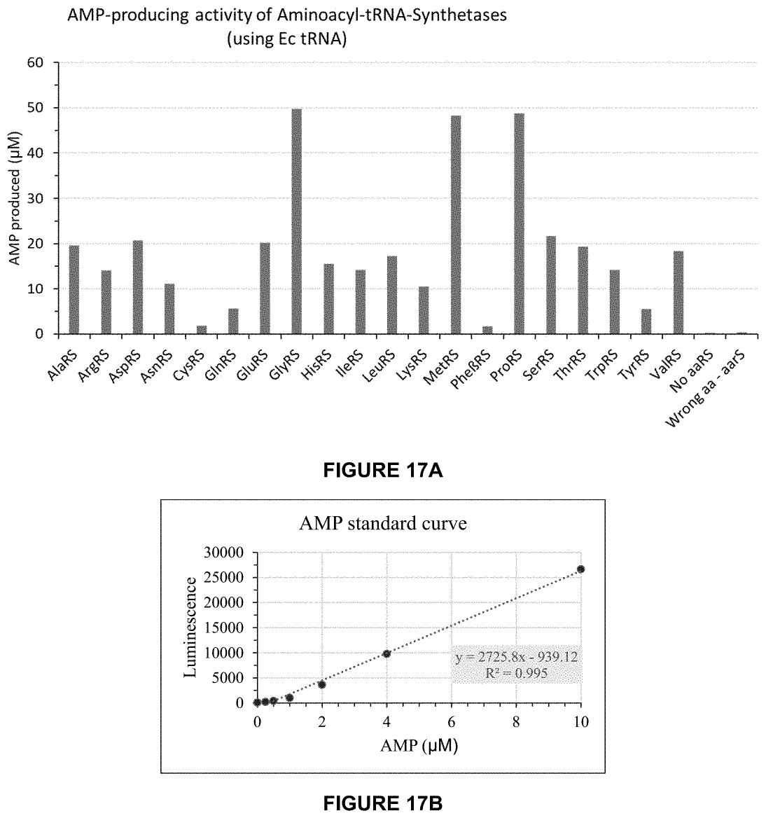

Resulting AMP from the aminoacyl-tRNA-synthetase reaction can be measured using the AMP-Glo™ kit. The present inventors performed assays using a commercial AMP detection kit (AMP-Glo™ assay, Promega V5012, incorporated herein by reference). This commercially available assay indirectly measures enzymatic production of AMP via a luminescence reaction. An included standard can be used for calibration and calculating the amount of produced AMP. This assay is a quantitative endpoint measurement assay. Each RS reaction was set up in a total of 100 μL with the final concentrations shown in Table 4, and run for one hour at 37° C. Subsequent AMP detection assays were performed in duplicate according to the manufacturer's instructions and produced AMP was calculated using the standard curve (FIG. 17B). FIG. 17A demonstrates results of three independent Aminoacyl-tRNA-Synthetase AMP-Producing Activity Assay utilizing exemplary tRNA from E. coli. A standard AMP curve is provided in FIG. 17B.

### Example 4

### Confirmation of Activity of Recombinant Aminoacyl-tRNA-Synthetases

As an additional confirmation of the activity of each cloned RS, the present inventors performed a malachite green phosphate assay using an available commercial kit (Cayman, Malachite Green Phosphate Assay Kit, #10009325, incorporated herein by reference). Produced pyrophosphate will form a complex with malachite green and lead to a color change which can be measured as absorbance. An included standard can be used for calibration and calculating the amount of produced PPi. This assay is a quantitative endpoint measurement assay. All reactions were performed according to the manufacturer's instructions and the produced PPi was calculated using the standard curve (shown as little inlet on graph).

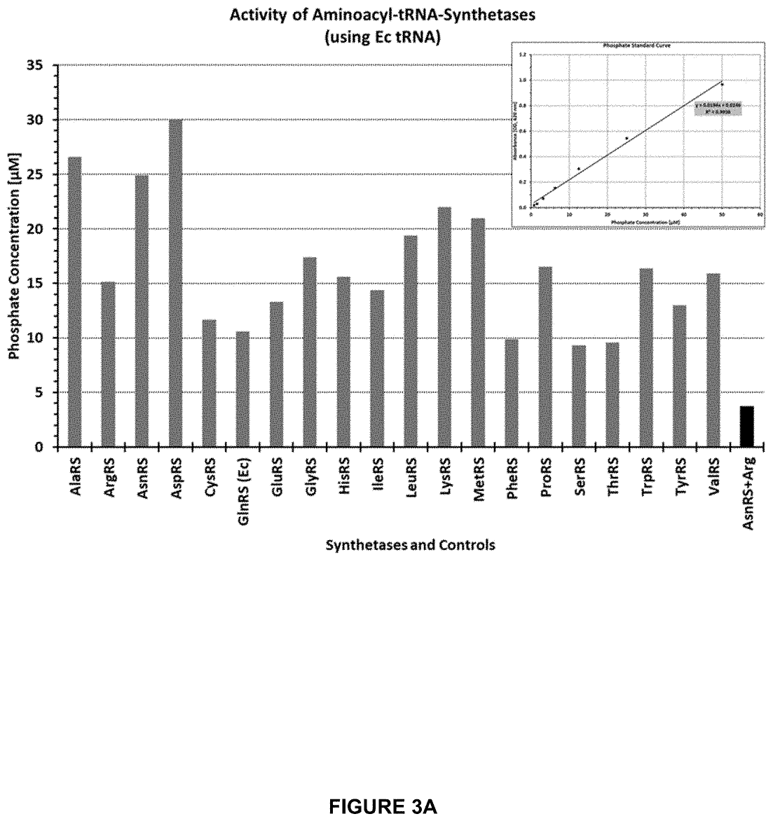

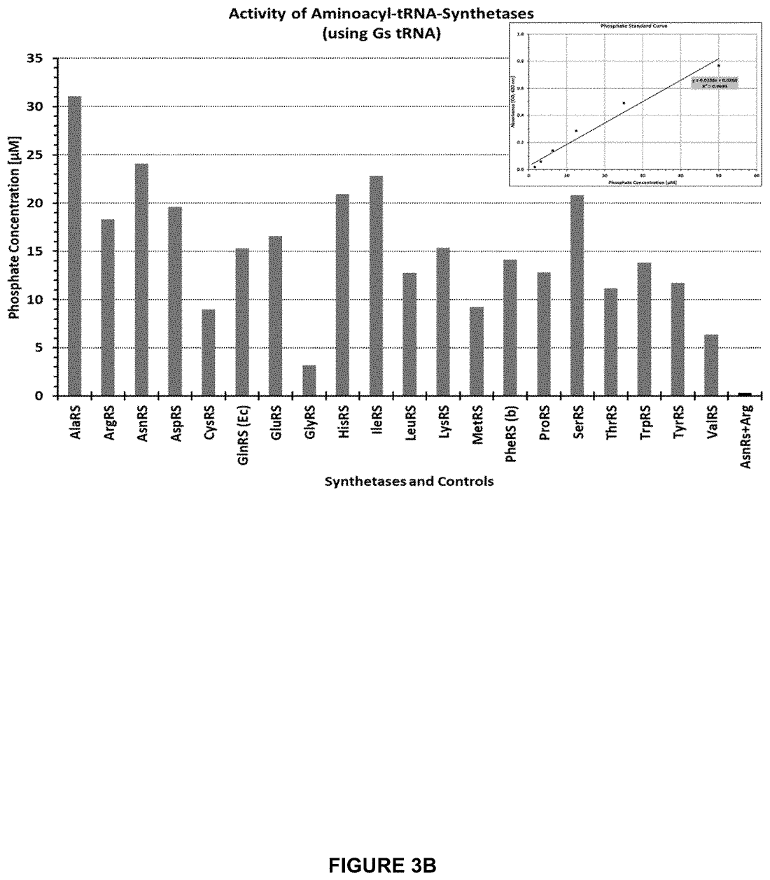

As shown in Table 4 below, the final concentrations for each RS reaction included a total volume of 150 μl. Exemplary tRNAs from E. coli were utilized in this assay. As shown in FIG. 3A, the graph demonstrated good activity for all RS compared to the controls without reaction buffer (no ATP) and the wrong amino acid for one of the RS (AsnRS+Arg). Each RS was used in the same molar concentration and incubated for 60 min before measuring the PPi concentration using the kit. Each bar was corrected for background/blank measurement) and represents the average value of a duplicate measurement. As shown in FIG. 3B, the same assay was replicated as generally described above utilizing tRNAs from a Geobacillus thermophile, such as Geobacillus subterraneus, or Geobacillus stearothermophilus.

### Example 5

### Recombinant Cell-Free Expression of Exemplary Protein

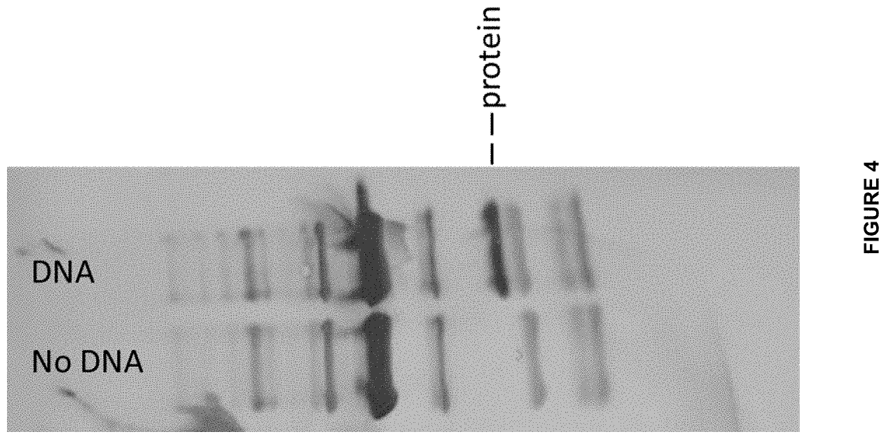

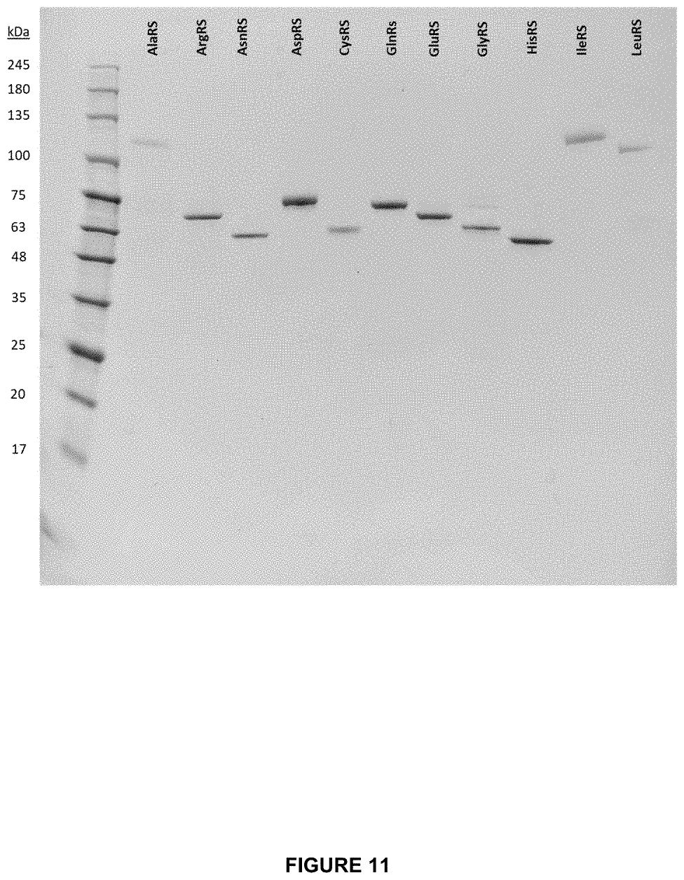

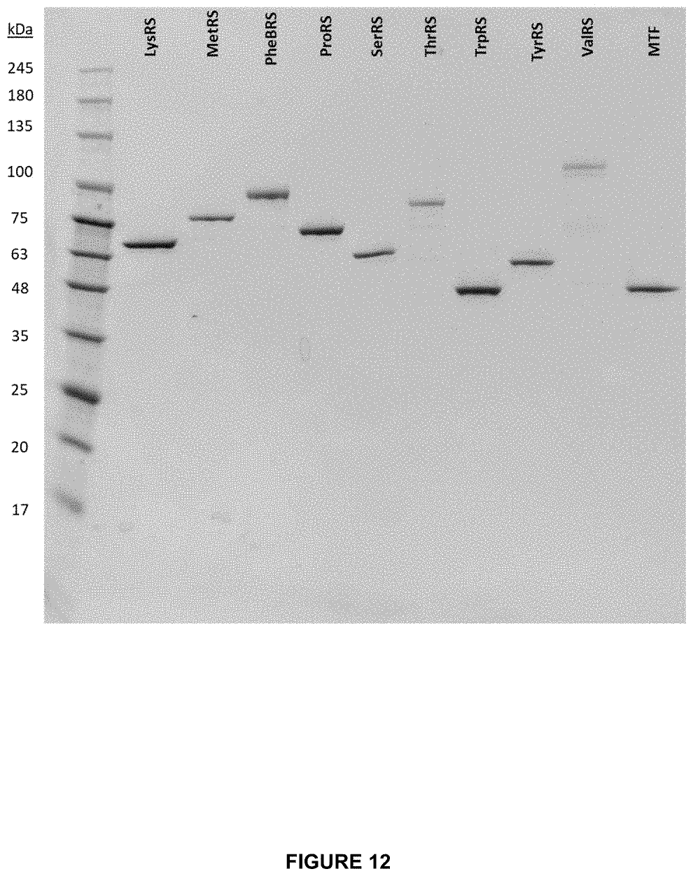

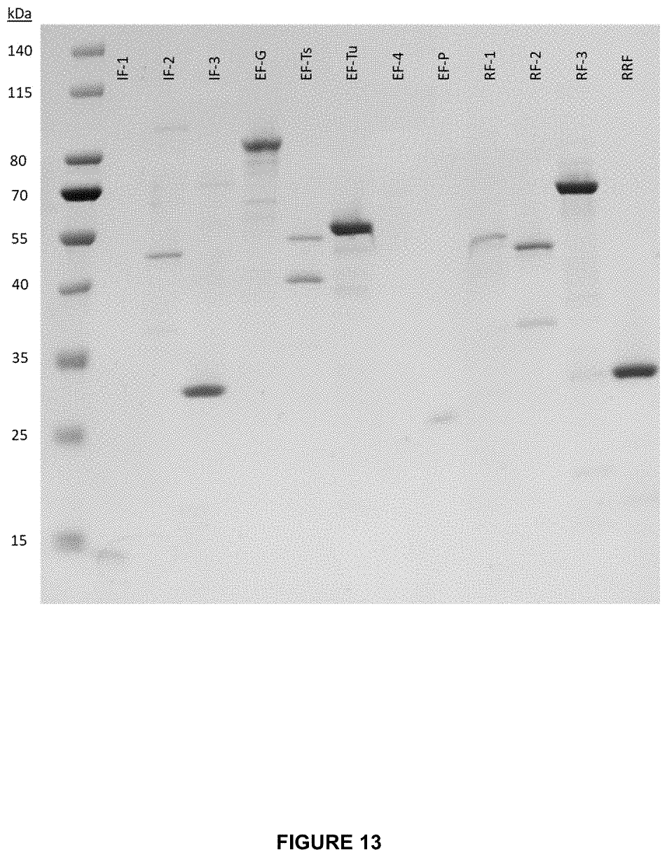

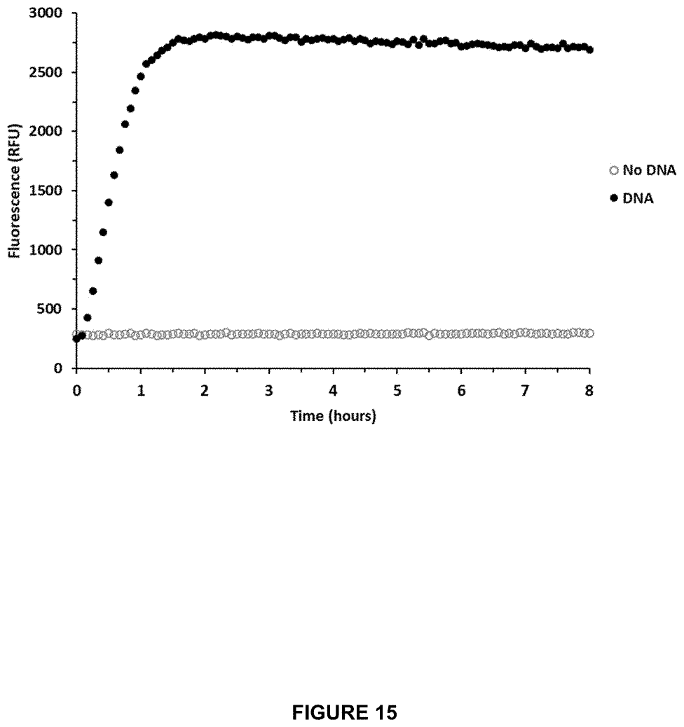

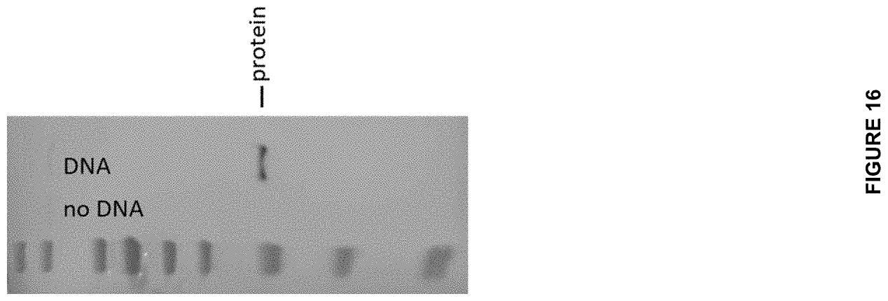

The present inventors demonstrated the production of two exemplary GFP peptides (SEQ ID NO. 134-135) in the invention's recombinant cell-free expression system. As identified in Table 6, a control and template recombinant cell-free expression mixture was generated. Isolation of core recombinant proteins identified in Table 6 below was demonstrated in FIGS. 11-14. As shown in FIG. 4, recombinant cell-free expression system transcribed the added template DNA and translates the resulting mRNA into the protein as indicated by the band in FIG. 4. As further demonstrated in FIG. 15, the present inventors showed real-time production of a fluorescent protein (muGFP; SEQ ID NO. 134) product utilizing the recombinant cell-free expression system described herein. As further shown in FIG. 16, the present inventors showed production of a fluorescent protein (deGFP; SEQ ID NO. 135) product utilizing the recombinant cell-free expression system described herein. Further, the present inventors demonstrated the removal of the recombinant cell-free expression system translation components from the produced GFP peptide via reverse purification. As specifically shown in FIG. 16, a western blot was performed with an anti-FLAG antibody of a cell-free protein expression reaction after reverse purification.

### Tables

TABLE 1
Exemplary core proteins for recombinant cell-free expression system
34 Core Recombinant Proteins

| | |
|---|---|
| 12 Recombinant Factors | initiation factors |
| | IF1 |
| | IF2 |
| | IF3 |
| | elongation factors |
| | EF-G |
| | EF-Tu |
| | EF-Ts |
| | EF-4 |
| | EF-P |
| | release factors |
| | RF1 |
| | RF2 |
| | RF3 |
| | ribosome-recycling factor |
| | RRF |
| 22 Recombinant Synthetases | aminoacyl-tRNA-synthetases |
| | AlaRS |
| | ArgRS |
| | AsnRS |
| | AspRS |
| | CysRS |
| | GlnRS (Ec) |
| | GluRS |
| | GlyRS |
| | HisRS |
| | IleRS |
| | LeuRS |
| | LysRS |
| | MetRS |
| | PheRS (a) |
| | PheRS (b) |
| | ProRS |
| | SerRS |
| | ThrRS |
| | TrpRS |
| | TyrRS |
| | ValRS |
| | methionyl-tRNA transformylase |
| | MTF |

TABLE 2
Pyrophosphate assay RS reaction mixture concentrations.

| Reaction buffer | RS reaction mix (30 μl) |
|---|---|
| 50 mM HEPES | 1 mM ATP |
| 150 mM NaCl | 20 μg tRNA |
| 10 mM KCl | 2 mM amino acid |
| 5 mM MgSO4 | 7 μg RS |
| 2 mM DTT | 1x reaction buffer |
| | ddH2O |

TABLE 3
50 μl pyrophosphate assay reaction.
Pyrophosphate assay (50 μl)

1x reaction buffer
0.4 mM MESG substrate
1 U purine nucleoside phosphorylase
0.03 U inorganic pyrophosphatase
12.5 μl RS reaction mix
ddH2O

TABLE 4
AMP assay RS reaction mixture concentrations

| Reaction buffer | RS reaction mix (100 μl) |
|---|---|
| 50 mM HEPES | 50 μM ATP |
| 150 mM NaCl | 100 μg tRNA |
| 10 mM KCl | 1 mM amino acid |
| 5 mM MgSO4 | 5 μg RS |
| 2 mM DTT | 1X reaction buffer |
| | ddH2O |

TABLE 5
Recombinant cell-free protein expression reaction mixture

| CONTROL REACTION | | | TEMPLATE REACTION | | |
|---|---|---|---|---|---|
| 2 | μl | Inorganic polyphosphate-based energy | 2 | μl | Inorganic polyphosphate-based energy |
| | | regeneration mixture | | | regeneration mixture |
| 1.33 | μl | Core Recombinant Protein Mix | 1.33 | μl | Core Recombinant Protein Mix |
| 0.9 | μl | Isolated Ribosomes - 100 mg/ml | 0.9 | μl | Isolated Ribosomes |
| 0.2 | μl | RNase Inhibitor | 0.2 | μl | RNase Inhibitor |
| 0.2 | μl | T7x polymerase | 0.2 | μl | T7x polymerase |
| 0.37 | μl | ddH2O | 0.45 | μl | DNA template |

TABLE 6
Protein, Vector and Tag Combination Listing

| Protein Name | Vector | Tag |
|---|---|---|
| IF-1 | pET151 | 6XHis |
| | pNAT | FLAG |
| IF-2 | pET151 | 6XHis |
| | pNAT | FLAG |
| IF-3 | pET151 | 6XHis |
| | pNAT | FLAG |
| EF-G | pET151 | 6XHis |
| | pNAT | FLAG |
| | pNAT | FLAG and |
| | | C-tag |
| EF-Tu | pNAT | C tag |
| EF-Ts | pET151 | 6XHis |
| | pNAT | FLAG |
| | pNAT | Ctag |
| EF-4 | pET24a(+) | 6XHis |
| | pNAT | FLAG |
| EF-P | pET24a(+) | 6XHis |
| | pNAT | FLAG |
| RF-1 | pET151 | 6XHis |
| | pNAT | FLAG |
| | pNAT | FLAG and |
| | | C-tag |
| | pNAT | C tag |
| RF-2 | pET151 | 6XHis |
| | pNAT | FLAG |
| RF-3 | pET24a(+) | 6XHis |
| | pNAT | FLAG |
| | pNAT | FLAG and |
| | | C-tag |
| | pNAT | C tag |
| RRF | pET151 | 6XHis |
| | pNAT | FLAG |
| | pNAT | FLAG and |
| | | C-tag |
| AlaRS | pET151 | 6XHis |
| | pNAT | FLAG |
| | pNAT | FLAG and |
| | | C-tag |
| | pNAT | C tag |
| ArgRS | pET151 | 6XHis |
| | pNAT | FLAG |
| AspRS | pET151 | 6XHis |
| | pNAT | FLAG |
| AsnRS | pET151 | 6XHis |
| | pNAT | FLAG |
| CysRS | pET151 | 6XHis |
| | pNAT | FLAG |
| GlnRS | pET151 | 6XHis |
| | pNAT | FLAG |
| GluRS | pET151 | 6XHis |
| | pNAT | FLAG |
| GlyRS | pET151 | 6XHis |
| | pNAT | FLAG |
| HisRS | pET151 | 6XHis |
| | pNAT | FLAG |
| | pNAT | FLAG and |
| | | C-tag |
| | pNAT | C tag |
| IleRS | pET151 | 6XHis |
| | pNAT | FLAG |
| LeuRS | pET151 | 6XHis |
| | pNAT | FLAG |
| LysRS | pET151 | 6XHis |
| | pNAT | FLAG |
| MetRS | pET151 | 6XHis |
| | pNAT | FLAG |
| | pNAT | FLAG and |
| | | C-tag |
| | pNAT | C tag |
| PheαRS | pET151 | 6XHis |
| | pNAT | FLAG |
| PheβRS | pET151 | 6XHis |
| | pNAT | FLAG |
| ProRS | pET151 | 6XHis |
| | pNAT | FLAG |
| SerRS | pET151 | 6XHis |
| | pNAT | FLAG |
| ThrRS | pET151 | 6XHis |
| | pNAT | FLAG |
| TrpRS | pET151 | 6XHis |
| | pNAT | FLAG |
| TyrRS | pET151 | 6XHis |
| | pNAT | FLAG |
| ValRS | pET151 | 6XHis |
| | pNAT | FLAG |
| MTF | pET151 | 6XHis |
| | pNAT | FLAG |

TABLE 7
Sequence Identity with Geobacillus subterraneus
91A1 strain sequences

| | | pET vector seqs - 91A1 | | |
|---|---|---|---|---|
| | | % identical | % positive | % gaps |
| Gs Aminoacyl | AlaRS | 92.72% | 96.64% | 1.57% |
| tRNA synthetases | ArgRS | 92.64% | 96.77% | 0.00% |
| | AsnRS | 95.70% | 98.19% | 0.23% |
| | AspRS | 70.39% | 72.93% | 23.18% |
| | CysRS | 94.29% | 96.83% | 1.48% |
| | GlnRS | No significant alignment | | |
| | GluRS | 93.78% | 96.39% | 1.61% |
| | GlyRS | 94.43% | 97.43% | 1.28% |
| | HisRS | 90.63% | 95.78% | 0.00% |
| | IleRS | 94.70% | 97.95% | 0.00% |
| | LeuRS | 94.58% | 97.66% | 0.74% |
| | LysRS | 96.16% | 98.38% | 0.00% |
| | MetRS | 95.08% | 98.16% | 0.00% |
| | MTF | 89.44% | 94.72% | 0.62% |
| | PheαRS | 91.64% | 93.87% | 3.90% |
| | PheβRS | 91.18% | 95.53% | 0.00% |
| | ProRS | 89.59% | 93.00% | 3.07% |
| | SerRS | 92.15% | 96.07% | 1.85% |
| | ThrRS | 92.96% | 96.94% | 0.46% |
| | TrpRS | 93.31% | 98.48% | 0.00% |
| | TyrRS | 90.00% | 95.24% | 0.00% |
| | ValRS | 93.96% | 95.60% | 3.19% |
| Gs Factors | EF-G | 95.09% | 98.27% | 0.00% |
| | EF-Ts | 94.92% | 97.29% | 0.00% |
| | EF-Tu | 98.23% | 99.49% | 0.00% |
| | EF-4 | 98.20% | 99.51% | 0.00% |
| | EF-P | 98.92% | 99.46% | 0.00% |
| | IF-1 | 84.52% | 85.71% | 14.29% |
| | IF-2 | 89.23% | 91.00% | 6.72% |
| | IF-3 | 63.79% | 65.52% | 34.48% |
| | RF-1 | 91.36% | 93.04% | 5.29% |
| | RF-2 | 96.34% | 98.48% | 0.00% |
| | RF-3 | No significant alignment | | |
| | RRF | 94.09% | 97.85% | 0.00% |

### REFERENCES

The following references are hereby incorporated in their entirety by reference:

[1] Carlson, Erik D. et al. “Cell-Free Protein Synthesis: Applications Come of Age.” Biotechnology advances 30.5 (2012): 1185-1194. PMC. Web. 1 Jan. 2018.

[2] Lloyd, A. J., Thomann, H. U., Ibba, M., & So11, D. (1995). A broadly applicable continuous spectrophotometric assay for measuring aminoacyl-tRNA synthetase activity. Nucleic acids research, 23(15), 2886-2892.

SEQUENCE LISTINGS

SEQ ID NO. 1
DNA
IF-1-GbIF-1-EcOpt
Geobacillus (codon-optimized for E. coli)
ATGGCCAAAGATGATGTGATTGAAGTTGAAGGCACCGTTATTGAAACCCTGCCGAATGCAATGTTTCGTG
TTGAACTGGAAAATGGTCATACCGTTCTGGCACATGTTAGCGGTAAAATTCGCATGCACTTTATTCGTAT
TCTGCCTGGTGATCGTGTTACCGTTGAACTGAGCCCGTACGATCTGACCCGTGGTCGTATTACCTATCGT
TATAAATGA
SEQ ID NO. 2
AMINO ACID
IF-1-GbIF-1-EcOpt
Geobacillus
MAKDDVIEVEGTVIETLPNAMFRVELENGHTVLAHVSGKIRMHFIRILPGDRVIVELSPYDLTRGRITYR
YK
SEQ ID NO. 3
DNA
IF-2-GsIF-2-EcOpt
Geobacillus stearothermophilus (codon-optimized for E. coli)
ATGAGCAAAATGCGCGTTTATGAGTACGCCAAAAAACAGAATGTTCCGAGCAAAGATGTGATCCACAAAC
TGAAAGAAATGAACATCGAAGTGAACAACCATATGGCAATGCTGGAAGCAGATGTTGTTGAAAAACTGGA
TCATCAGTATCGTCCGAATACCGGCAAAAAAGAAGAAAAAAAAGCCGAGAAGAAAACCGAGAAACCGAAA
CGTCCGACACCAGCAAAAGCAGCAGATTTTGCAGATGAAGAAATCTTCGATGATAGCAAAGAAGCAGCCA
AAATGAAACCGGCAAAGAAAAAAGGTGCACCGAAAGGTAAAGAAACCAAAAAAACCGAAGCACAGCAGCA
AGAGAAAAAACTGCTGCAGGCAGCGAAAAAGAAAGGCAAAGGTCCGGCAAAAGGGAAAAAACAGGCAGCA
CCGGCAGCCAAACAGGCACCGCAGCCTGCGAAAAAAGAAAAAGAACTGCCGAAAAAAATCACCTTTGAAG
GTAGCCTGACCGTTGCAGAACTGGCAAAAAAACTGGGTCGTGAACCGAGCGAAATTATCAAAAAACTGTT
TATGCTGGGTGTGATGGCCACCATTAATCAGGATCTGGATAAAGATGCCATTGAACTGATTTGCAGCGAT
TATGGTGTTGAGGTTGAAGAAAAAGTGACCATCGATGAAACCAACTTTGAAGCCATTGAAATTGTTGATG
CACCGGAAGATCTGGTTGAACGTCCGCCTGTTGTTACCATTATGGGTCATGTTGATCATGGTAAAACCAC
ACTGCTGGATGCAATTCGTCATAGCAAAGTTACCGAACAAGAAGCAGGCGGTATTACACAGCATATTGGT
GCATATCAGGTTACCGTGAACGATAAGAAAATCACGTTTCTGGATACACCGGGTCATGAAGCATTTACCA
CCATGCGTGCACGTGGTGCACAGGTGACCGATATTGTTATTCTGGTTGTTGCAGCAGATGATGGCGTTAT
GCCGCAGACCGTTGAAGCAATTAATCATGCAAAAGCCGCAAACGTTCCGATTATTGTTGCCATCAACAAA
ATCGATAAACCGGAAGCAAATCCGGATCGTGTTATGCAAGAACTGATGGAATATAATCTGGTTCCGGAAG
AATGGGGTGGTGATACCATTTTTTGTAAACTGAGCGCCAAAACCAAAGAAGGTCTGGACCATCTGCTGGA
AATGATTCTGCTGGTTAGCGAAATGGAAGAACTGAAAGCCAATCCGAATCGTCGTGCAGTTGGCACCGTT
ATTGAAGCCAAACTGGACAAAGGTCGTGGTCCGGTTGCGACCCTGCTGATTCAGGCAGGCACCCTGCGTG
TTGGTGATCCGATTGTTGTGGGCACCACCTATGGTCGTGTTCGTGCAATGGTTAATGATAGCGGTCGTCG
TGTTAAAGAAGCAACCCCGAGCATGCCGGTTGAAATTACCGGTCTGCATGAAGTTCCGCAGGCAGGCGAT
CGTTTTATGGTTTTTGAAGATGAGAAAAAGGCACGCCAGATTGCCGAAGCACGTGCACAGCGTCAGCTGC
AAGAACAGCGTAGCGTTAAAACCCGTGTTAGCCTGGATGACCTGTTTGAGCAGATTAAACAGGGTGAAAT
GAAAGAGCTGAACCTGATTGTTAAAGCCGATGTTCAGGGTAGCGTTGAAGCCCTGGTTGCAGCACTGCAG
AAAATTGATGTTGAAGGTGTTCGCGTGAAAATTATCCATGCAGCCGTTGGTGCAATTACCGAAAGCGATA
TTAGCCTGGCAACCGCAAGCAATGCAATTGTGATTGGTTTTAATGTTCGTCCGGATGCAAATGCAAAACG
TGCAGCAGAAAGTGAAAAAGTGGATATTCGTCTGCACCGCATTATCTATAACGTGATCGAAGAAATTGAG
GCAGCCATGAAAGGTATGCTGGATCCGGAATATGAAGAGAAAGTTATTGGTCAGGCAGAAGTTCGTCAGA
CCTTTAAAGTTAGCAAAGTGGGTACAATTGCCGGTTGTTATGTTACCGATGGTAAAATTACCCGTGATAG
TAAAGTTCGTCTGATTCGTCAGGGTATTGTTGTGTATGAAGGTGAAATTGATAGCCTGAAACGCTATAAA
GATGATGTTCGTGAAGTTGCCCAGGGTTATGAATGTGGTCTGACCATTAAAAACTTCAACGACATTAAAG
AGGGCGACGTTATCGAAGCCTATATCATGCAAGAAGTTGCACGCGCATAA
SEQ ID NO. 4
Amino Acid
IF-2-GsIF-2-EcOpt
Geobacillus stearothermophilus
MSKMRVYEYAKKQNVPSKDVIHKLKEMNIEVNNHMAMLEADVVEKLDHQYRPNTGKKEEKKAEKKTEKPK
RPTPAKAADFADEEIFDDSKEAAKMKPAKKKGAPKGKETKKTEAQQQEKKLLQAAKKKGKGPAKGKKQAA
PAAKQAPQPAKKEKELPKKITFEGSLTVAELAKKLGREPSEIIKKLFMLGVMATINQDLDKDAIELICSD
YGVEVEEKVTIDETNFEAIEIVDAPEDLVERPPVVTIMGHVDHGKTTLLDAIRHSKVTEQEAGGITQHIG
AYQVTVNDKKITFLDTPGHEAFTTMRARGAQVTDIVILVVAADDGVMPQTVEAINHAKAANVPIIVAINK
IDKPEANPDRVMQELMEYNLVPEEWGGDTIFCKLSAKIKEGLDHLLEMILLVSEMEELKANPNRRAVGTV
IEAKLDKGRGPVATLLIQAGTLRVGDPIVVGTTYGRVRAMVNDSGRRVKEATPSMPVEITGLHEVPQAGD
RFMVFEDEKKARQIAEARAQRQLQEQRSVKTRVSLDDLFEQIKQGEMKELNLIVKADVQGSVEALVAALQ
KIDVEGVRVKIIHAAVGAITESDISLATASNAIVIGFNVRPDANAKRAAESEKVDIRLHRIIYNVIEEIE
AAMKGMLDPEYEEKVIGQAEVRQTFKVSKVGTIAGCYVTDGKITRDSKVRLIRQGIVVYEGEIDSLKRYK
DDVREVAQGYECGLTIKNFNDIKEGDVIEAYIMQEVARA
SEQ ID NO. 5
DNA
IF-3-GbIF-3-EcOpt
Geobacillus (codon-optimized for E. coli)
ATGATCAGCAAGGACTTTATCATCAATGAGCAGATTCGTGCACGTGAAGTTCGTCTGATTGATCAGAATG
GTGAACAGCTGGGTATCAAAAGCAAACAAGAAGCACTGGAAATTGCAGCACGTCGTAATCTGGATCTGGT
TCTGGTGGCACCGAATGCAAAACCGCCTGTTTGTCGTATTATGGATTATGGCAAATTTCGCTTCGAGCAG
CAGAAAAAAGAAAAAGAGGCACGCAAAAAGCAGAAAGTGATCAATGTTAAAGAAGTGCGTCTGAGCCCGA
CCATTGAAGAACATGATTTTAACACCAAACTGCGCAACGCACGCAAATTTCTGGAAAAAGGTGATAAAGT
GAAAGCCACCATTCGTTTTAAAGGTCGTGCAATCACCCATAAAGAAATTGGTCAGCGTGTTCTGGATCGT
TTTAGCGAAGCATGTGCAGATATTGCAGTTGTTGAAACCGCACCGAAAATGGATGGTCGTAATATGTTTC
TGGTGCTGGCTCCGAAAAACGACAACAAATAA
SEQ ID NO. 6
Amino Acid
IF-3-GbIF-3-EcOpt
Geobacillus
MISKDFIINEQIRAREVRLIDQNGEQLGIKSKQEALEIAARRNLDLVLVAPNAKPPVCRIMDYGKFRFEQ
QKKEKEARKKQKVINVKEVRLSPTIEEHDFNTKLRNARKFLEKGDKVKATIRFKGRAITHKEIGQRVLDR
FSEACADIAVVETAPKMDGRNMFLVLAPKNDNK
SEQ ID NO. 7
DNA
EF-G-GsEF-G-EcOpt
Geobacillus (codon-optimized for E. coli)
ATGGCACGTGAATTCAGCCTGGAAAAAACCCGTAATATTGGTATTATGGCCCATATCGATGCAGGTAAAA
CCACCACCACCGAACGTATTCTGTTTTATACCGGTCGTGTGCATAAAATTGGTGAAGTTCATGAAGGTGC
AGCAACCATGGATTGGATGGAACAAGAACAAGAGCGTGGTATTACCATTACCAGCGCAGCCACCACCGCA
CAGTGGAAAGGTCATCGTATTAACATTATTGATACACCGGGTCACGTTGATTTTACCGTTGAAGTTGAAC
GTAGCCTGCGTGTTCTGGATGGTGCAATTACCGTGCTGGATGCACAGAGCGGTGTTGAACCGCAGACCGA
AACCGTTTGGCGTCAGGCAACCACCTATGGTGTTCCGCGTATTGTTTTTGTGAACAAGATGGATAAAATC
GGTGCCGATTTCCTGTATAGCGTTAAAACCCTGCATGATCGTCTGCAGGCAAATGCACATCCGGTTCAGC
TGCCGATTGGTGCAGAAGATCAGTTTAGCGGTATTATTGATCTGGTTGAAATGTGCGCCTATCACTATCA
TGATGAACTGGGCAAAAACATCGAACGCATTGATATTCCGGAAGAATATCGTGATATGGCCGAAGAGTAT
CACAACAAACTGATTGAAGCAGTTGCAGAACTGGATGAAGAACTGATGATGAAATATCTGGAAGGCGAAG
AAATTACCGCAGAGGAACTGAAAGCAGCAATTCGTAAAGCAACCATTAGCGTGGAATTTTTTCCGGTTTT
TTGTGGTAGCGCCTTCAAAAACAAAGGTGTGCAGCTGCTGCTGGATGGCGTTGTTGATTATCTGCCGAGT
CCGGTGGATATTCCTGCAATTCGTGGTGTTGTTCCGGATACCGAAGAAGAAGTTACACGCGAAGCAAGTG
ATGATGCACCGTTTGCAGCACTGGCCTTTAAAATCATGACCGATCCGTATGTTGGTAAGCTGACCTTTAT
TCGTGTTTATAGCGGCACCCTGGATAGCGGTAGCTATGTTATGAATACCACCAAAGGTAAACGTGAACGT
ATTGGTCGTCTGCTGCAGATGCATGCAAATCATCGTCAAGAAATCAGCAAAGTTTATGCCGGTGATATTG
CAGCAGCAGTTGGTCTGAAAGATACCACAACCGGTGATACCCTGTGTGATGAAAAACATCCGGTGATTCT
GGAAAGCATGCAGTTTCCGGAACCGGTTATTAGCGTTGCAATTGAACCGAAAAGCAAAGCCGATCAGGAT
AAAATGAGCCAGGCACTGCAGAAACTGCAAGAAGAGGATCCGACCTTTCGTGCACATACCGATCCGGAAA
CCGGTCAGACCATTATTAGTGGTATGGGTGAACTGCATCTGGATATCATTGTTGATCGTATGCGTCGCGA
ATTTAAAGTTGAAGCAAATGTTGGTGCACCGCAGGTTGCATATCGTGAAACCTTTCGTAAAAGCGCACAG
GTTGAAGGCAAATTTATCCGTCAGAGTGGTGGTCGTGGTCAGTATGGTCATGTTTGGATTGAATTTTCAC
CGAACGAACGCGGTAAAGGCTTTGAATTTGAAAATGCAATTGTTGGTGGTGTGGTGCCGAAAGAATATGT
TCCGGCAGTTCAGGCAGGTCTGGAAGAGGCAATGCAGAATGGTGTTCTGGCAGGTTATCCGGTTGTTGAT
ATTAAAGCCAAACTGTTCGATGGCAGCTATCACGATGTTGATAGCAGCGAAATGGCATTCAAAATTGCAG
CAAGCCTGGCACTGAAAAATGCCGCAACCAAATGTGATCCTGTTCTGCTGGAACCGATTATGAAAGTGGA
AGTTGTTATCCCTGAGGAATATCTGGGTGATATTATGGGCGATATTACCAGCCGTCGTGGTCGCATTGAA
GGTATGGAAGCACGTGGTAATGCCCAGGTTGTTCGTGCAATGGTTCCGCTGGCAGAAATGTTTGGTTATG
CAACCAGCCTGCGTAGCAATACCCAAGGTCGTGGCACCTTTAGCATGGTTTTTGATCATTATGAAGAGGT
GCCCAAAAACATTGCCGATGAGATCATCCAAGGGCGAATAA
SEQ ID NO. 8
Amino Acid
EF-G-GsEF-G-EcOpt
Geobacillus
MAREFSLEKTRNIGIMAHIDAGKTTTTERILFYTGRVHKIGEVHEGAATMDWMEQEQERGITITSAATTA
QWKGHRINIIDTPGHVDFTVEVERSLRVLDGAITVLDAQSGVEPQTETVWRQATTYGVPRIVFVNKMDKI
GADFLYSVKTLHDRLQANAHPVQLPIGAEDQFSGIIDLVEMCAYHYHDELGKNIERIDIPEEYRDMAEEY
HNKLIEAVAELDEELMMKYLEGEEITAEELKAAIRKATISVEFFPVFCGSAFKNKGVQLLLDGVVDYLPS
PVDIPAIRGVVPDTEEEVTREASDDAPFAALAFKIMTDPYVGKLTFIRVYSGILDSGSYVMNITKGKRER
IGRLLQMHANHRQEISKVYAGDIAAAVGLKDTTTGDTLCDEKHPVILESMQFPEPVISVAIEPKSKADQD
KMSQALQKLQEEDPTFRAHTDPETGQTIISGMGELHLDIIVDRMRREFKVEANVGAPQVAYRETFRKSAQ
VEGKFIRQSGGRGQYGHVWIEFSPNERGKGFEFENAIVGGVVPKEYVPAVQAGLEEAMQNGVLAGYPVVD
IKAKLFDGSYHDVDSSEMAFKIAASLALKNAATKCDPVLLEPIMKVEVVIPEEYLGDIMGDITSRRGRIE
GMEARGNAQVVRAMVPLAEMFGYATSLRSNTQGRGTFSMVFDHYEEVPKNIADEIIKKNKGE
SEQ ID NO. 9
DNA
EF-Tu-GsEF-Tu-EcOpt
Geobacillus (codon-optimized for E. coli)
ATGGCCAAAGCCAAATTTGAACGTACCAAACCGCATGTTAATATTGGCACCATTGGTCATGTTGATCATG
GTAAAACCACACTGACCGCAGCAATTACCACCGTTCTGGCAAAACAGGGTAAAGCCGAAGCAAAAGCATA
TGATCAGATTGATGCAGCACCGGAAGAACGTGAACGTGGTATTACCATTAGCACCGCACATGTTGAATAT
GAAACCGATGCACGTCATTATGCCCATGTTGATTGTCCGGGTCATGCAGATTATGTGAAAAATATGATTA
CCGGTGCAGCACAGATGGATGGTGCAATTCTGGTTGTTAGCGCAGCAGATGGTCCGATGCCGCAGACACG
TGAACATATTCTGCTGAGCCGTCAGGTTGGTGTTCCGTATATTGTTGTGTTTCTGAACAAATGCGATATG
GTGGATGATGAAGAACTGCTGGAACTGGTTGAAATGGAAGTTCGTGATCTGCTGTCCGAATATGATTTTC
CGGGTGATGAAGTTCCGGTTATTAAAGGTAGCGCACTGAAAGCACTGGAAGGTGATCCGCAGTGGGAAGA
AAAAATCATTGAACTGATGAATGCCGTGGATGAGTATATTCCGACACCGCAGCGTGAAGTTGATAAACCG
TTTATGATGCCGATCGAAGATGTGTTTAGCATTACCGGTCGTGGCACCGTTGCAACCGGTCGCGTTGAAC
GTGGCACCCTGAAAGTTGGTGATCCGGTTGAAATTATTGGTCTGAGTGATGAACCGAAAACCACCACCGT
TACCGGTGTTGAAATGTTTCGTAAACTGTTAGATCAGGCCGAAGCCGGTGATAATATTGGTGCACTGCTG
CGTGGTGTTTCACGTGATGAGGTGGAACGTGGTCAGGTTCTGGCGAAACCTGGTAGCATTACACCGCATA
CCAAATTCAAAGCACAGGTTTATGTTCTGACCAAAGAAGAAGGCGGTCGTCATACCCCGTTTTTTAGCAA
TTATCGTCCGCAGTTTTATTTCCGTACCACCGATGTTACCGGTATTATTACCCTGCCGGAAGGTGTGGAA
ATGGTTATGCCTGGTGATAACGTTGAAATGACCGTGGAACTGATTGCACCGATTGCAATTGAAGAAGGCA
CCAAATTTAGCATTCGTGAAGGTGGTCGTACCGTTGGTGCAGGTAGCGTTAGCGAAATTATCGAATAA
SEQ ID NO. 10
Amino Acid
EF-Tu-GsEF-Tu-EcOpt
Geobacillus
MAKAKFERTKPHVNIGTIGHVDHGKTTLTAAITTVLAKQGKAEAKAYDQIDAAPEERERGITISTAHVEY
ETDARHYAHVDCPGHADYVKNMITGAAQMDGAILVVSAADGPMPQTREHILLSRQVGVPYIVVFLNKCDM
VDDEELLELVEMEVRDLLSEYDFPGDEVPVIKGSALKALEGDPQWEEKIIELMNAVDEYIPTPQREVDKP
FMMPIEDVFSITGRGTVATGRVERGTLKVGDPVEIIGLSDEPKTTGVTGVEMFRKLLDQAEAGDNIGALL
RGVSRDEVERGQVLAKPGSITPHTKFKAQVYVLTKEEGGRHTPFFSNYRPQFYFRTTDVTGIITLPEGVE
MVMPGDNVEMTVELIAPIAIEEGTKFSIREGGRTVGAGSVSEIIE
SEQ ID NO. 11
DNA
EF-Ts-GsEF-Ts-EcOpt
Geobacillus (codon-optimized for E. coli)
ATGGCAATTACCGCACAGATGGTTAAAGAACTGCGTGAAAAAACCGGTGCAGGTATGATGGATTGTAAAA
AAGCACTGACCGAAACCAATGGCGATATGGAAAAAGCAATTGATTGGCTGCGCGAAAAAGGTATTGCAAA
AGCAGCAAAAAAAGCCGATCGTATTGCAGCAGAAGGTATGGCATATATTGCAGTTGAAGGTAATACCGCA
GTTATCCTGGAAGTTAATAGCGAAACCGATTTTGTGGCAAAAAACGAAGCATTTCAGACCCTGGTGAAAG
AGCTGGCAGCACATCTGCTGAAACAGAAACCGGCAAGCCTGGATGAAGCACTGGGTCAGACCATGGATAA
TGGTAGCACCGTTCAGGATTATATCAATGAAGCCATTGCCAAAATCGGCGAAAAAATCACCCTGCGTCGT
TTTGCAGTTGTTAATAAAGCAGATGGTGAAACCTTTGGTGCCTATCTGCATATGGGTGGTCGTATTGGTG
TTCTGACCCTGCTGGCAGGTAATGCAAGCGAAGATGTTGCAAAAGATGTGGCAATGCATATTGCAGCCCT
GCATCCGAAATATGTTAGCCGTGATGATGTTCCGCAAGAAGAAATTGCACACGAACGTGAAGTTCTGAAA
CAGCAGGCACTGAATGAAGGCAAACCGGAAAAAATTGTGGAAAAGATGGTTGAAGGTCGCCTGAACAAAT
TCTATGAAGATGTTTGTCTGCTGGAACAGGCCTTTGTTAAAAATCCGGATGTTACCGTTCGTCAGTATGT
TGAAAGCAATGGTGCCACCGTTAAACAGTTTATTCGTTATGAAGTTGGTGAGGGCTTAGAAAAACGCCAG
GATAATTTTGCCGAAGAAGTTATGAGCCAGGTTCGCAAACAGTAA
SEQ ID NO. 12
Amino Acid
EF-Ts-GsEF-Ts-EcOpt
Geobacillus
MAITAQMVKELREKTGAGMMDCKKALTETNGDMEKAIDWLREKGIAKAAKKADRIAAEGMAYIAVEGNTA
VILEVNSETDFVAKNEAFQTLVKELAAHLLKQKPASLDEALGQTMDNGSTVQDYINEAIAKIGEKITLRR
FAVVNKADGETFGAYLHMGGRIGVLTLLAGNASEDVAKDVAMHIAALHPKYVSRDDVPQEEIAHEREVLK
QQALNEGKPEKIVEKMVEGRLNKFYEDVCLLEQAFVKNPDVTVRQYVESNGATVKQFIRYEVGEGLEKRQ
DNFAEEVMSQVRKQ
SEQ ID NO. 13
DNA
EF-4-GsEF-4-EcOpt
Geobacillus (codon-optimized for E. coli)
ATGAACCGTGAGGAACGTCTGAAACGTCAGGAGCGTATTCGTAACTTCAGCATCATTGCGCACATCGACC
ACGGTAAAAGCACCCTGGCGGATCGTATCCTGGAGAAAACCGGTGCGCTGAGCGAGCGTGAACTGCGTGA
ACAGACCCTGGACATGATGGATCTGGAGCGTGAACGTGGTATCACCATTAAGCTGAACGCGGTGCAACTG
ACCTATAAGGCGAAAAACGGCGAGGAATACATCTTCCACCTGATTGACACCCCGGGCCACGTGGATTTTA
CCTATGAAGTTAGCCGTAGCCTGGCGGCGTGCGAAGGTGCGATTCTGGTGGTTGATGCGGCGCAGGGTAT
TGAGGCGCAAACCCTGGCGAACGTGTACCTGGCGATTGACAACAACCTGGAAATCCTGCCGGTTATCAAC
AAAATTGATCTGCCGAGCGCGGAGCCGGAACGTGTGCGTCAGGAGATCGAAGACGTTATTGGTCTGGATG
CGAGCGAGGCGGTGCTGGCGAGCGCGAAGGTTGGTATCGGCATTGAGGAAATCCTGGAGCAAATTGTGGA
AAAAATTCCGGCGCCGAGCGGTGACCCGGATGCGCCGCTGAAGGCGCTGATCTTTGACAGCCTGTACGAT
CCGTATCGTGGCGTGGTTGCGTACGTGCGTATTGTTGACGGTACCGTTAAGCCGGGCCAGCGTATCAAAA
TGATGAGCACCGGCAAGGAGTTCGAAGTGACCGAGGTGGGCGTTTTTACCCCGAAGCAAAAAATCGTTGA
CGAACTGACCGTGGGTGATGTTGGCTATCTGACCGCGAGCATTAAGAACGTGAAAGATACCCGTGTTGGT
GACACCATTACCGATGCGGAGCGTCCGGCGGCGGAACCGCTGCCGGGTTACCGTAAACTGAACCCGATGG
TTTTCTGCGGCATGTATCCGATCGACACCGCGCGTTACAACGATCTGCGTGAGGCGCTGGAAAAGCTGCA
GCTGAACGACGCGGCGCTGCACTTCGAGCCGGAAACCAGCCAAGCGCTGGGTTTCGGCTTTCGTTGCGGT
TTTCTGGGCCTGCTGCACATGGAGATCATTCAGGAACGTATCGAGCGTGAATTTCACATCGATCTGATTA
CCACCGCGCCGAGCGTGGTTTATAAAGTGCACCTGACCGACGGTACCGAGGTGAGCGTTGATAACCCGAC
CAACATGCCGGACCCGCAAAAAATCGATCGTATTGAGGAACCGTATGTGAAGGCGACCATTATGGTTCCG
AACGACTACGTGGGCCCGGTTATGGAACTGTGCCAGGGTAAACGTGGCACCTTCGTGGACATGCAATACC
TGGATGAGAAGCGTGTTATGCTGATCTATGACATTCCGCTGAGCGAAATCGTTTACGACTTCTTTGATGC
GCTGAAGAGCAACACCAAAGGTTACGCGAGCTTTGATTATGAGCTGATTGGCTACCGTCCGAGCAACCTG
GTGAAAATGGACATCCTGCTGAACGGTGAAAAGATTGATGCGCTGAGCTTCATCGTTCACCGTGAGGCGG
CGTATGAACGTGGCAAAGTGATTGTTGAGAAGCTGAAAGACCTGATCCCGCGTCAGCAATTTGAAGTGCC
GGTTCAGGCGGCGATTGGTAACAAAATCATTGCGCGTAGCACCATCAAGGCGCTGCGTAAAAACGTGCTG
GCGAAGTGCTACGGTGGCGATGTTAGCCGTAAGCGTAAACTGCTGGAGAAGCAGAAAGAAGGTAAGAAAC
GTATGAAACAGATTGGTAGCGTTGAGGTGCCGCAAGAAGCGTTCATGGCGGTGCTGAAGATCGACGATCA
AAAGAAA
SEQ ID NO. 14
Amino Acid
EF-4-GsEF-4-EcOpt
Geobacillus
MNREERLKRQERIRNFSIIAHIDHGKSTLADRILEKTGALSERELREQTLDMMDLERERGITIKLNAVQL
TYKAKNGEEYIFHLIDTPGHVDFTYEVSRSLAACEGAILVVDAAQGIEAQTLANVYLAIDNNLEILPVIN
KIDLPSAEPERVRQEIEDVIGLDASEAVLASAKVGIGIEEILEQIVEKIPAPSGDPDAPLKALIFDSLYD
PYRGVVAYVRIVDGTVKPGQRIKMMSTGKEFEVTEVGVFTPKQKIVDELTVGDVGYLTASIKNVKDTRVG
DTITDAERPAAEPLPGYRKLNPMVFCGMYPIDTARYNDLREALEKLQLNDAALHFEPETSQALGFGFRCG
FLGLLHMEIIQERIEREFHIDLITTAPSVVYKVHLTDGTEVSVDNPTNMPDPQKIDRIEEPYVKATIMVP
NDYVGPVMELCQGKRGTFVDMQYLDEKRVMLIYDIPLSEIVYDFFDALKSNTKGYASFDYELIGYRPSNL
VKMDILLNGEKIDALSFIVHREAAYERGKVIVEKLKDLIPRQQFEVPVQAAIGNKIIARSTIKALRKNVL
AKCYGGDVSRKRKLLEKQKEGKKRMKQIGSVEVPQEAFMAVLKIDDQKK
SEQ ID NO. 15
DNA
EF-P-GsEF-P-EcOpt
Geobacillus (codon-optimized for E. coli)
ATGATCAGCGTGAACGACTTCCGTACCGGTCTGACCATCGAAGTTGATGGCGAGATTTGGCGTGTGCTGG
AATTCCAGCACGTTAAGCCGGGTAAAGGCGCGGCGTTTGTGCGTAGCAAGCTGCGTAACCTGCGTACCGG
TGCGATCCAAGAACGTACCTTCCGTGCGGGCGAGAAGGTGAACCGTGCGCAGATTGACACCCGTAAAATG
CAATACCTGTATGCGAACGGTGACCAGCACGTTTTTATGGATATGGAGACCTACGAACAGATCGAGCTGC
CGGCGAAACAAATTGAGTATGAACTGAAGTTCCTGAAAGAAAACATGGAAGTGTTTATCATGATGTACCA
AGGTGAAACCATCGGCATTGAGCTGCCGAACACCGTTGAGCTGAAGGTGGTTGAGACCGAACCGGGTATT
AAAGGTGATACCGCGAGCGGTGGCAGCAAGCCGGCGAAACTGGAAACCGGCCTGGTGGTTCAGGTGCCGT
TCTTTGTTAACGAGGGTGACACCCTGATCATTAACACCGCGGATGGCACCTATGTTAGCCGTGCG
SEQ ID NO. 16
Amino Acid
EF-P-GsEF-P-EcOpt
Geobacillus
MISVNDFRTGLTIEVDGEIWRVLEFQHVKPGKGAAFVRSKLRNLRTGAIQERTFRAGEKVNRAQIDTRKM
QYLYANGDQHVFMDMETYEQIELPAKQIEYELKFLKENMEVFIMMYQGETIGIELPNTVELKVVETEPGI
KGDTASGGSKPAKLETGLVVQVPFFVNEGDTLIINTADGTYVSRA
SEQ ID NO. 17
DNA RF-1
Title: GsRF-1-Ec Opt
Origin: Geobacillus stearothermophilus (codon-optimized for E. coli)
ATGTTTGATCGTCTGGAAGCAGTTGAACAGCGTTATGAAAAACTGAATGAACTGCTGATGGAACCGGATG
TTATTAACGATCCGAAAAAACTGCGCGATTATAGCAAAGAACAGGCAGATCTGGAAGAAACCGTTCAGAC
CTATCGTGAGTATAAAAGCGTTCGTGAACAGCTGGCCGAAGCAAAAGCAATGCTGGAAGAGAAACTGGAA
CCTGAACTGCGTGAAATGGTGAAAGAAGAAATTGGCGAACTGGAAGAACGTGAAGAAGCACTGGTTGAGA
AACTGAAAGTTCTGCTGCTGCCGAAAGATCCGAATGATGAAAAAAACGTGATCATGGAAATTCGTGCAGC
AGCCGGTGGCGAAGAAGCAGCACTGTTTGCCGGTGATCTGTATCGTATGTATACCCGTTATGCAGAAAGC
CAAGGTTGGAAAACCGAAGTTATTGAAGCAAGCCCGACCGGTTTAGGTGGTTATAAAGAAATCATCTTCA
TGATCAATGGCAAGGGTGCATACAGCAAACTGAAATTTGAAAATGGTGCACATCGTGTTCAGCGTGTTCC
GGAAACCGAAAGCGGTGGTCGTATTCATACCAGCACCGCAACCGTTGCATGTCTGCCGGAAATGGAAGAA
ATCGAAGTGGAAATCAACGAGAAAGATATTCGCGTTGATACCTTTGCAAGCAGCGGTCCTGGTGGTCAGA
GCGTTAATACCACCATGAGCGCAGTTCGTCTGACCCATATTCCGACCGGTATTGTTGTTACCTGTCAGGA
TGAAAAATCCCAGATCAAAAACAAAGAAAAAGCCATGAAAGTGCTGCGTGCCCGTATCTATGATAAATAT
CAGCAAGAGGCACGTGCGGAATATGATCAGACCCGTAAACAGGCAGTTGGCACCGGTGATCGTAGCGAAC
GTATTCGTACCTATAACTTTCCGCAGAATCGTGTTACCGATCATCGTATTGGTCTGACCATTCAAAAACT
GGATCAGGTTCTGGATGGTCATCTGGATGAAATTATCGAAGCACTGATTCTGGATGACCAGGCAAAAAAG
CTGGAACAGGCAAATGATGCAAGCTAA
SEQ ID NO. 18
Amino Acid
RF-1-GsRF-1-EcOpt
Geobacillus stearothermophilus
MFDRLEAVEQRYEKLNELLMEPDVINDPKKLRDYSKEQADLEETVQTYREYKSVREQLAEAKAMLEEKLE
PELREMVKEEIGELEEREEALVEKLKVLLLPKDPNDEKNVIMEIRAAAGGEEAALFAGDLYRMYTRYAES
QGWKTEVIEASPTGLGGYKEIIFMINGKGAYSKLKFENGAHRVQRVPETESGGRIHTSTATVACLPEMEE
IEVEINEKDIRVDTFASSGPGGQSVNTTMSAVRLTHIPTGIVVTCQDEKSQIKNKEKAMKVLRARIYDKY
QQEARAEYDQTRKQAVGTGDRSERIRTYNFPQNRVTDHRIGLTIQKLDQVLDGHLDEIIEALILDDQAKK
LEQANDAS
SEQ ID NO. 19
DNA
RF-2-GsRF-2-EcOpt
Geobacillus stearothermophilus (codon-optimized for E. coli)
ATGGCAGCACCGAATTTTTGGGATGATCAGAAAGCAGCACAGGCAGTTATTAGCGAAGCAAATGCACTGA
AAGATCTGGTGGAAGAATTTAGCAGCCTGGAAGAACGTTTTGATAATCTGGAAGTTACCTACGAACTGCT
GAAAGAAGAACCGGACGACGAACTGCAGGCAGAACTGGTTGAAGAGGCAAAAAAACTGATGAAAGATTTT
AGCGAATTTGAACTGCAGCTGCTGCTGAATGAACCGTATGATCAAAATAATGCCATCCTGGAACTGCATC
CTGGTGCCGGTGGCACCGAAAGCCAGGATTGGGCAAGCATGCTGCTGCGTATGTATACCCGTTGGGCAGA
AAAAAAAGGCTTTAAAGTTGAAACCCTGGATTATCTGCCTGGTGAAGAAGCAGGTATTAAAAGCGTTACC
CTGCTGATTAAAGGCCATAATGCATATGGTTATCTGAAAGCCGAAAAAGGTGTTCATCGTCTGGTTCGTA
TTAGCCCGTTTGATGCAAGCGGTCGTCGTCATACCAGCTTTGTTAGCTGTGAAGTTGTGCCGGAACTGGA
TGATAACATTGAAATTGAAATTCGCCCTGAAGAACTGAAGATTGATACCTATCGTAGCAGCGGTGCAGGC
GGTCAGCATGTTAATACCACCGATAGCGCAGTGCGTATTACCCATCTGCCGACCGGTATTGTTGTTACCT
GTCAGAGCGAACGTAGCCAGATTAAAAACCGTGAAAAAGCCATGAATATGCTGAAAGCCAAACTGTACCA
GAAGAAATTAGAAGAACAGCAGGCCGAGCTGGCCGAACTGCGTGGTGAACAGAAAGAAATTGGTTGGGGT
AATCAGATTCGCAGCTATGTTTTTCATCCGTACAGCCTGGTTAAAGATCATCGTACCAATGTTGAAGTTG
GTAATGTTCAGGCCGTTATGGATGGTGAAATTGATGTTTTTATCGATGCATACCTGCGTGCCAAACTGAA
ATAA
SEQ ID NO. 20
Amino Acid
RF-2-GsRF-2-EcOpt
Geobacillus stearothermophilus
MAAPNFWDDQKAAQAVISEANALKDLVEEFSSLEERFDNLEVTYELLKEEPDDELQAELVEEAKKLMKDF
SEFELQLLLNEPYDQNNAILELHPGAGGTESQDWASMLLRMYTRWAEKKGFKVETLDYLPGEEAGIKSVT
LLIKGHNAYGYLKAEKGVHRLVRISPFDASGRRHTSFVSCEVVPELDDNIEIEIRPEELKIDTYRSSGAG
GQHVNTTDSAVRITHLPTGIVVTCQSERSQIKNREKAMNMLKAKLYQKKLEEQQAELAELRGEQKEIGWG
NQIRSYVFHPYSLVKDHRTNVEVGNVQAVMDGEIDVFIDAYLRAKLK
SEQ ID NO. 21
DNA
RF-3-BX1-RF-3-EcOpt
Bacillus sp. X1 (codon-optimized for E. coli)
ATGGGTAACGATTTCAAGAAAGAAGTGCTGAGCCGTCGTACCTTTGCGATCATTAGCCATCCGGATGCGG
GCAAGACCACCCTGACCGAGAAACTGCTGCTGTTCGGTGGCGCGATCCGTGATGCGGGTACCGTTAAGGC
GAAGAAAACCGGCAAATACGCGACCAGCGACTGGATGGAAATCGAGAAACAGCGTGGTATTAGCGTGACC
AGCAGCGTTATGCAATTCGATTACAACGGTTATCGTGTGAACATTCTGGACACCCCGGGCCACCAGGACT
TTAGCGAAGATACCTATCGTACCCTGATGGCGGTGGACAGCGCGGTTATGATCATTGATAGCGCGAAGGG
CATCGAGGACCAAACCATTAAGCTGTTCAAAGTGTGCCGTATGCGTGGTATCCCGATTTTCACCTTTATC
AACAAGCTGGACCGTCAGGGCAAACAACCGCTGGAGCTGCTGGCGGAACTGGAGGAAGTTCTGGGTATCG
AGAGCTACCCGATGAACTGGCCGATTGGTATGGGCAAAGAATTTCTGGGCATCTATGATCGTTACTATAA
CCGTATTGAGCAGTTCCGTGTGAACGAGGAAGAGCGTTTTATCCCGCTGAACGAAGACGGTGAAATTGAG
GGCAACCACAAGCTGGTTAGCAGCGGTCTGTACGAGCAGACCCTGGAAGAGATCATGCTGCTGAACGAGG
CGGGTAACGAATTTAGCGCGGAGCGTGTGGCGGCGGGTCAACTGACCCCGGTTTTCTTTGGTAGCGCGCT
GACCAACTTCGGCGTGCAGACCTTTCTGGAAACCTATCTGCAATTTGCTCCGCCGCCGAAGGCGCGTAAC
AGCAGCATCGGCGAGATTGATCCGCTGAGCGAAGAGTTTAGCGGCTTCGTTTTTAAAATTCAGGCGAACA
TGAACCCGGCGCACCGTGACCGTATCGCGTTCGTGCGTATTTGCAGCGGCAAGTTTGAGCGTGGCATGAG
CGTTAACCTGCCGCGTCTGGGCAAGCAGCTGAAACTGACCCAAAGCACCAGCTTCATGGCGGAAGAGCGT
AACACCGTGGAAGAGGCGGTTAGCGGTGACATCATTGGCCTGTACGATACCGGTACCTATCAGATCGGCG
ATACCCTGACCGTGGGCAAAAACGACTTCCAGTTTGAGCGTCTGCCGCAATTCACCCCGGAACTGTTTGT
GCGTGTTAGCGCGAAGAACGTTATGCGTCAGAAGAGCTTTTACAAAGGTCTGCACCAGCTGGTGCAAGAA
GGCGCGATTCAACTGTACAAGACCGTTAAAACCGATGAGTATCTGCTGGGTGCGGTGGGCCAGCTGCAAT
TCGAAGTTTTTGAGCACCGTATGAAGAACGAATATAACGCGGAAGTGCTGATGGAACGTCTGGGTAGCAA
AATCGCGCGTTGGATTGAAAACGACGAGGTTGATGAAAACCTGAGCAGCAGCCGTAGCCTGCTGGTGAAA
GACCGTTACGATCACTATGTTTTCCTGTTTGAGAACGACTTCGCGCTGCGTTGGTTTCAGGAAAAGAACC
CGACCATCAAACTGTACAACCCGATGGACCAACACGAT
SEQ ID NO. 22
Amino Acid
RF-3
BX1-RF-3-EcOpt
Bacillus sp. X1
MGNDFKKEVLSRRTFAIISHPDAGKTTLTEKLLLFGGAIRDAGTVKAKKTGKYATSDWMEIEKQRGISVT
SSVMQFDYNGYRVNILDTPGHQDFSEDTYRTLMAVDSAVMIIDSAKGIEDQTIKLFKVCRMRGIPIFTFI
NKLDRQGKQPLELLAELEEVLGIESYPMNWPIGMGKEFLGIYDRYYNRIEQFRVNEEERFIPLNEDGEIE
GNHKLVSSGLYEQTLEEIMLLNEAGNEFSAERVAAGQLTPVFFGSALTNFGVQTFLETYLQFAPPPKARN
SSIGEIDPLSEEFSGFVFKIQANMNPAHRDRIAFVRICSGKFERGMSVNLPRLGKQLKLIQSTSFMAEER
NTVEEAVSGDIIGLYDTGTYQIGDTLTVGKNDFQFERLPQFTPELFVRVSAKNVMRQKSFYKGLHQLVQE
GAIQLYKTVKTDEYLLGAVGQLQFEVFEHRMKNEYNAEVLMERLGSKIARWIENDEVDENLSSSRSLLVK
DRYDHYVFLFENDFALRWFQEKNPTIKLYNPMDQHD
SEQ ID NO. 23
DNA
RRF-GbRRF-EcOpt
Geobacillus (codon-optimized for E. coli)
ATGGCCAAACAGGTTATTCAGCAGGCCAAAGAAAAAATGGATAAAGCCGTTCAGGCATTTACCCGTGAAC
TGGCAAGCATTCGTGCAGGTCGTGCAAATGCAGGTCTGCTGGAAAAAGTTACCGTTGATTATTATGGTGT
TCCGACGCCGATTAATCAGCTGGCGAGCATTAGCGTTCCGGAAGCACGTCTGCTGGTGATTCAGCCGTAT
GATAAAAGCGCAATCAAAGAGATGGAAAAAGCAATTCTGGCAAGCGATCTGGGTCTGACCCCGAGCAATG
ATGGTAGCGTTATTCGTCTGGTTATTCCGCCTCTGACCGAAGAACGTCGTCGCGAACTGGCGAAACTGGT
GAAAAAATACAGCGAAGATGCAAAAGTTGCCGTGCGTAATATTCGTCGTGATGCAAATGATGAGCTGAAA
AAGCTGGAAAAGAATGGCGAAATTACCGAAGATGAACTGCGTAGCTATACCGATGAAGTTCAGAAACTGA
CCGATGATCATATCGCAAAAATTGACGCCATCACCAAAGAGAAAGAAAAAGAAGTCATGGAAGTTTAA
SEQ ID NO. 24
Amino Acid
RRF
GbRRF-EcOpt
Geobacillus
MAKQVIQQAKEKMDKAVQAFTRELASIRAGRANAGLLEKVTVDYYGVPTPINQLASISVPEARLLVIQPY
DKSAIKEMEKAILASDLGLTPSNDGSVIRLVIPPLTEERRRELAKLVKKYSEDAKVAVRNIRRDANDELK
KLEKNGEITEDELRSYTDEVQKLTDDHIAKIDAITKEKEKEVMEV
SEQ ID NO. 25
DNA
AlaRS-GsAlaRS-EcOpt
Geobacillus stearothermophilus (codon-optimized for E. coli)
ATGAAAAAACTGACCAGCGCACAGGTTCGTCGCATGTTTCTGGAATTTTTTCAAGAAAAAGGTCATGCCG
TTGAACCGAGCGCAAGCCTGATTCCGGTTGATGATCCGAGCCTGCTGTGGATTAATAGCGGTGTTGCAAC
CCTGAAAAAATACTTTGATGGTCGTATTGTTCCGGAAAATCCGCGTATTTGTAATGCCCAGAAAAGCATT
CGTACCAACGATATTGAAAATGTGGGTAAAACCGCACGCCATCACACCTTTTTTGAAATGCTGGGCAATT
TTAGCATCGGCGATTATTTCAAACGTGAAGCAATTCATTGGGCCTGGGAATTTCTGACCAGTGATAAATG
GATTGGTTTTGATCCGGAACGTCTGAGCGTTACCGTTCATCCGGAAGATGAAGAAGCATATAACATTTGG
CGCAATGAAATTGGTCTGCCGGAAGAACGTATTATTCGTCTGGAAGGTAACTTTTGGGATATTGGTGAAG
GTCCGAGCGGTCCGAATACCGAAATCTTTTATGATCGTGGTGAAGCCTTTGGTAATGATCCGAATGATCC
TGAACTGTATCCAGGTGGTGAAAATGATCGTTATCTGGAAGTTTGGAATCTGGTGTTTAGCCAGTTTAAT
CATAATCCGGATGGCACCTATACACCGCTGCCGAAAAAAAACATTGATACCGGCATGGGTTTAGAACGTA
TGTGTAGCATTCTGCAGGATGTTCCGACCAATTTTGAAACCGACCTGTTTCTGCCGATTATTCGTGCAAC
CGAGCAGATTGCCGGTGAACGTTATGGTGAAGATCCGGATAAAGATGTTGCCTTTAAAGTGATTGCCGAT
CATATTCGCGCAGTTACCTTTGCAATTGGTGATGGTGCACTGCCGAGCAATGAAGGTCGTGGTTATGTTC
TGCGTCGTCTGCTGCGTCGTGCAGTTCGTTATGCAAAACATATTGGTATTGAACGTCCGTTCATGTATGA
ACTGGTTCCGGTTGTTGGTGAAATCATGCACGATTATTATCCCGAGGTTAAAGAGAAAGCCGATTTTATT
GCACGTGTGATTCGTACCGAAGAAGAACGTTTTCACGAAACCCTGCATGAAGGTCTGGCAATTCTGGCAG
AAGTTATTGAAAAAGCAAAAGAACAGGGTTCCGATGTTATTCCGGGTGAAGAGGCATTTCGTCTGTATGA
TACCTATGGTTTTCCGATTGAACTGACCGAAGAATATGCAGCCGAAGCAGGTATGACCGTTGATCATGCA
GGTTTTGAACGTGAAATGGAACGTCAGCGTGAACGTGCCCGTGCAGCACGTCAGGATGTTGATAGTATGC
AGGTTCAAGGTGGTGTTCTGGGTGATATTAAAGATGAAAGTCGCTTTGTGGGCTATGATGAGCTGGTTGC
AGCAAGCACCGTTATTGCAATTGTTAAAGATGGTCGTCTGGTGGAAGAAGTTAAAGCAGGCGAAGAAGCA
CAGATTATTGTTGATGTTACCCCGTTTTATGCAGAAAGCGGTGGTCAGATTGCAGATCAGGGTGTTTTTG
AAAGCGAAACCGGCACCGCAGTTGTGAAAGATGTTCAGAAAGCACCGAATGGTCAGCATCTGCATGCAAT
TATTGTGGAACATGGCACCGTTAAAAAAGGTAGCCGTTATACCGCACGTGTTGATGAAGCAAAACGTATG
CGTATTGTGAAAAATCATACCGCAACACATCTGCTGCATCAGGCACTGAAAGACGTTCTGGGTCGTCATG
TTAATCAGGCAGGTAGCCTGGTTGCACCGGATCGTCTGCGTTTTGACTTTACCCATTTTGGTCAGGTTAA
ACCCGAAGAACTGGAACGTATTGAAGCGATTGTTAATGAGCAGATTTGGAAAAGCCTGCCGGTGGATATT
TTCTATAAACCGCTGGAAGAGGCAAAAGCAATGGGTGCAATGGCACTGTTTGGTGAAAAATATGGTGATA
TTGTGCGTGTGGTTAAAGTGGGTGATTATAGCCTGGAACTGTGTGGTGGTTGTCATGTGCCGAATACCAG
CGCCATTGGTCTGTTTAAAATCGTTAGCGAAAGCGGTATTGGTGCAGGCACCCGTCGCATTGAAGCAGTT
ACCGGTGAAGCAGCATATCGTTTTATGAGCGAACAGCTGGCCATTCTGCAAGAAGCAGCACAGAAACTGA
AAACCAGTCCGAAAGAACTGAATGCACGTCTGGATGGCCTGTTTGCAGAACTGAAAGAATTAGAACGCGA
AAATGAAAGCCTGGCAGCCCGTCTGGCACATATGGAAGCAGAACATCTGACCCGTCAGGTAAAAGATGTT
AATGGTGTTCCGGTTCTGGCAGCAAAAGTTCAGGCAAATGATATGAATCAGCTGCGTGCCATGGCCGATG
ATCTGAAACAAAAACTGGGTACAGCAGTTATTGTTCTGGCAAGCGCACAAGGTGGTAAAGTTCAGCTGAT
TGCAGCCGTTACAGATGACCTGGTAAAAAAAGGTTTTCATGCGGGTAAACTGGTTAAAGAAGTTGCAAGC
CGTTGCGGTGGTGGTGGCGGTGGTCGTCCGGATCTGGCACAGGCAGGCGGTAAAGATCCGAGCAAAGTTG
GTGAAGCACTGGGTTATGTTGAAACCTGGGTTAAAAGCGTGAGCTAA
SEQ ID NO. 26
Amino Acid
AlaRS-GsAlaRS-EcOpt
Geobacillus stearothermophilus
MKKLTSAQVRRMFLEFFQEKGHAVEPSASLIPVDDPSLLWINSGVATLKKYFDGRIVPENPRICNAQKSI
RINDIENVGKTARHHTFFEMLGNFSIGDYFKREAIHWAWEFLTSDKWIGFDPERLSVTVHPEDEEAYNIW
RNEIGLPEERIIRLEGNFWDIGEGPSGPNTEIFYDRGEAFGNDPNDPELYPGGENDRYLEVWNLVFSQFN
HNPDGTYTPLPKKNIDTGMGLERMCSILQDVPTNFETDLFLPIIRATEQIAGERYGEDPDKDVAFKVIAD
HIRAVIFAIGDGALPSNEGRGYVLRRLLRRAVRYAKHIGIERPFMYELVPVVGEIMHDYYPEVKEKADFI
ARVIRTEEERFHETLHEGLAILAEVIEKAKEQGSDVIPGEEAFRLYDTYGFPIELTEEYAAEAGMTVDHA
GFEREMERQRERARAARQDVDSMQVQGGVLGDIKDESRFVGYDELVAASTVIAIVKDGRLVEEVKAGEEA
QIIVDVTPFYAESGGQIADQGVFESETGTAVVKDVQKAPNGQHLHAIIVEHGTVKKGSRYTARVDEAKRM
RIVKNHTATHLLHQALKDVLGRHVNQAGSLVAPDRLRFDFTHFGQVKPEELERIEAIVNEQIWKSLPVDI
FYKPLEEAKAMGAMALFGEKYGDIVRVVKVGDYSLELCGGCHVPNTSAIGLFKIVSESGIGAGTRRIEAV
TGEAAYRFMSEQLAILQEAAQKLKTSPKELNARLDGLFAELKELERENESLAARLAHMEAEHLTRQVKDV
NGVPVLAAKVQANDMNQLRAMADDLKQKLGTAVIVLASAQGGKVQLIAAVTDDLVKKGFHAGKLVKEVAS
RCGGGGGGRPDLAQAGGKDPSKVGEALGYVETWVKSVS
SEQ ID NO. 27
DNA
ArgRS-GsArgRS-EcOpt
Geobacillus (codon-optimized for E. coli)
ATGAATATTGTGGGCCAGATCAAAGAAAAAATGAAAGAAGAAATTCGTCAGGCAGCAGTTCGTGCAGGTC
TGGCAAGCGCAGATGAACTGCCGGATGTTCTGCTGGAAGTTCCGCGTGATAAAGCACATGGTGATTATAG
CACCAATATTGCAATGCAGCTGGCACGTATTGCAAAAAAACCGCCTCGTGCAATTGCCGAAGCAATTGTT
GGTCAGCTGGATCGTGAACGTATGAGCGTTGCCCGTATTGAAATTGCAGGTCCGGGTTTTATCAACTTCT
ATATGGATAATCGTTACCTGACCGCAGTTGTTCCGGCAATTCTGCAGGCAGGTCAGGCATATGGTGAAAG
TAATGTTGGTAATGGTGAGAAAGTCCAGGTTGAATTTGTTAGCGCAAATCCGACCGGTGATCTGCATCTG
GGTCATGCACGTGGTGCAGCAGTTGGTGATAGCCTGTGTAATATTCTGGCAAAAGCAGGTTTTGATGTGA
CCCGTGAATACTATATTAATGATGCAGGCAAGCAGATCTACAATCTGGCCAAAAGCGTTGAAGCACGTTA
TTTTCAGGCACTGGGTGTTGATATGCCGCTGCCGGAAGATGGTTATTATGGTGATGATATTGTGGAAATC
GGCAAAAAACTGGCCGAAGAATATGGTGATCGTTTCGTTGAAATGGAAGAAGAGGAACGTCTGGCATTTT
TTCGTGATTATGGTCTGCGTTATGAGCTGGAAAAAATCAAAAAAGATCTGGCCGATTTTCGCGTTCCGTT
TGATGTTTGGTATAGCGAAACCAGCCTGTATGAAAGCGGTAAAATTGATGAAGCACTGAGCACCCTGCGT
GAACGTGGTTATATCTATGAACAGGATGGTGCAACCTGGTTTCGTAGCACCGCATTTGGAGATGATAAAG
ATCGTGTTCTGATTAAACAGGACGGCACCTATACCTATCTGCTGCCGGATATTGCATATCATCAGGATAA
ACTGCGTCGCGGTTTTAAGAAACTGATTAACATTTGGGGTGCCGATCATCATGGTTATATTCCTCGCATG
AAAGCAGCAATTGCAGCACTGGGTTATGATCCGGAAGCACTGGAAGTTGAAATTATTCAGATGGTGAATC
TGTATCAGAATGGCGAACGTGTGAAAATGAGCAAACGTACCGGTAAAGCAGTTACCATGCGTGAACTGAT
GGAAGAGGTTGGTGTTGATGCAGTTCGTTATTTCTTTGCAATGCGTAGCGGTGATACCCATCTGGATTTT
GATATGGATCTGGCAGTTAGCCAGAGCAATGAAAATCCGGTTTATTATGTTCAGTATGCCCATGCGCGTG
TTAGCAGCATTCTGCGTCAGGCGGAAGAACAGCATATTAGCTATGATGGTGATCTGGCACTGCATCATCT
GGTTGAAACCGAAAAAGAAATTGAGCTGCTGAAAGTGCTGGGTGATTTTCCGGATGTTGTTGCAGAAGCA
GCACTGAAACGTATGCCGCATCGTGTTACCGCATATGCATTTGACCTGGCCAGCGCACTGCATAGCTTTT
ATAACGCCGAAAAAGTTCTGGATCTGGACAACATCGAAAAAACCAAAGCACGTCTGGCCCTGGTTAAAGC
CGTTCAGATTACACTGCAGAATGCACTGGCCCTGATTGGTGTGAGCGCACCGGAACAAATGTAA
SEQ ID NO. 28
Amino Acid
ArgRS-GsArgRS-EcOpt
Geobacillus
MNIVGQIKEKMKEEIRQAAVRAGLASADELPDVLLEVPRDKAHGDYSTNIAMQLARIAKKPPRAIAEAIV
GQLDRERMSVARIEIAGPGFINFYMDNRYLTAVVPAILQAGQAYGESNVGNGEKVQVEFVSANPTGDLHL
GHARGAAVGDSLCNILAKAGFDVTREYYINDAGKQIYNLAKSVEARYFQALGVDMPLPEDGYYGDDIVEI
GKKLAEEYGDRFVEMEEEERLAFFRDYGLRYELEKIKKDLADFRVPFDVWYSETSLYESGKIDEALSTLR
ERGYIYEQDGATWFRSTAFGDDKDRVLIKQDGTYTYLLPDIAYHQDKLRRGFKKLINIWGADHHGYIPRM
KAAIAALGYDPEALEVEIIQMVNLYQNGERVKMSKRTGKAVTMRELMEEVGVDAVRYFFAMRSGDTHLDF
DMDLAVSQSNENPVYYVQYAHARVSSILRQAEEQHISYDGDLALHHLVETEKEIELLKVLGDFPDVVAEA
ALKRMPHRVTAYAFDLASALHSFYNAEKVLDLDNIEKTKARLALVKAVQITLQNALALIGVSAPEQM
SEQ ID NO. 29
DNA
AsnRS-GsAsnRS-EcOpt
Geobacillus (codon-optimized for E. coli)
ATGGATGTGAGCATTATTGGTGGTAATCAGTGTGTTAAAACCACCACCATTGCCGAAGTTAATCAGTATG
TTGGTCAGCAGGTTACCATTGGTGCATGGCTGGCAAATAAACGTAGCAGCGGTAAAATTGTTTTTCTGCA
GCTGCGTGATGGCACCGGTTTTATTCAGGGTGTTGTTGAAAAAGCCAATGTTAGCGAAGAGGTTTTTCAG
CGTGCAAAAACCCTGACACAAGAAACCAGCCTGTATGTGACCGGCACCGTTCGTATTGATGAACGTAGCC
CGTTTGGTTATGAACTGAGCGTTGCCGATCTGCAGGTTATTCAAGAAGCAGTTGATTATCCGATTACGCC
GAAAGAACATGGTGTTGAATTTCTGATGGATCATCGTCATCTGTGGCTGCGTAGCCGTCGTCAGCATGCA
ATTATGAAAATTCGCAACGAAATTATCCGTGCCACCTATGAATTTTTCAACGATCGTGGTTTTGTGAAAG
TGGATGCACCGATTCTGACCGGTAGCGCACCGGAAGGCACCACCGAACTGTTTCATACCAAATATTTCGA
TGAGGATGCATATCTGAGCCAGAGCGGTCAGCTGTATATGGAAGCAGCAGCAATGGCACTGGGTAAAGTT
TTTAGCTTTGGTCCGACCTTTCGTGCCGAAAAAAGCAAAACCCGTCGCCATCTGATTGAATTTTGGATGG
TTGAACCGGAAATGGCCTTTTATGAATTTGAAGATAATCTGCGCCTGCAAGAGGAATATGTTAGCTATCT
GGTTCAGAGCGTTCTGGAACGTTGTCGTCTGGAACTGGGTCGCCTGGGTCGTGATGTTAGCAAACTGGAA
TTAGTTAAACCGCCTTTTCCGCGTCTGACCTATGATGAAGCAATTAAACTGCTGCATGAAAAAGGCCTGA
CCGATATTGAATGGGGTGATGATTTTGGTGCACCGCATGAAACCGCAATTGCAGAAAGCTTTGATAAACC
GGTGTTTATCACCCATTATCCGACCAGCCTGAAACCGTTTTATATGCAGCCGGATCCGAATCGTCCGGAT
GTTGTTCTGTGTGCAGATCTGATTGCTCCGGAAGGTTATGGTGAAATTATTGGCGGTAGCGAACGCATCC
ATGATTATGAGCTGCTGAAACGTCGCCTGGAAGAACATCATCTGCCGCTGGAAGCATATGAATGGTATCT
GGATCTGCGTAAATATGGTAGCGTTCCGCATAGCGGTTTTGGTCTGGGTTTAGAACGTACCGTTGCATGG
ATTTGCGGTGTTGAACATGTGCGTGAAACCATTCCGTTTCCACGTCTGCTGAATCGTCTGTATCCGTAA
SEQ ID NO. 30
Amino Acid
AsnRS-GsAsnRS-EcOpt
Geobacillus
MDVSIIGGNQCVKTTTIAEVNQYVGQQVTIGAWLANKRSSGKIVFLQLRDGTGFIQGVVEKANVSEEVFQ
RAKTLIQETSLYVTGIVRIDERSPFGYELSVADLQVIQEAVDYPITPKEHGVEFLMDHRHLWLRSRRQHA
IMKIRNEIIRATYEFFNDRGFVKVDAPILTGSAPEGTTELFHTKYFDEDAYLSQSGQLYMEAAAMALGKV
FSFGPTFRAEKSKTRRHLIEFWMVEPEMAFYEFEDNLRLQEEYVSYLVQSVLERCRLELGRLGRDVSKLE
LVKPPFPRLTYDEAIKLLHEKGLTDIEWGDDFGAPHETAIAESFDKPVFITHYPTSLKPFYMQPDPNRPD
VVLCADLIAPEGYGEIIGGSERIHDYELLKRRLEEHHLPLEAYEWYLDLRKYGSVPHSGFGLGLERTVAW
ICGVEHVRETIPFPRLLNRLYP
SEQ ID NO. 31
DNA
AspRS-GsAspRS-EcOpt
Geobacillus (codon-optimized for E. coli)
ATGGAACGCACCTATTATTGTGGTGAAGTTCCGGAAACCGCAGTTGGTGAACGTGTTGTTCTGAAAGGTT
GGGTTCAGAAACGTCGTGATTTAGGTGGTCTGATTTTTATCGATCTGCGTGATCGTACCGGTATTGTTCA
GGTTGTTGCAAGTCCGGATGTTAGCGCAGAAGCACTGGCAGCAGCAGAACGTGTTCGTAGCGAATATGTT
CTGAGCGTTGAAGGCACCGTTGTTGCCCGTGCACCGGAAACAGTTAATCCGAATATTGCAACCGGTCGCA
TTGAAATTCAGGCAGAACGTATTGAAATTATCAACGAAGCAAAAACCCCTCCGTTTAGCATTAGTGATGA
TACCGATGCAGCCGAAGATGTTCGTCTGAAATATCGTTATCTGGATCTGCGTCGTCCGGTTATGTTTCAG
ACCCTGGCACTGCGTCATAAAATCACCAAAACCGTTCGTGATTTTCTGGATAGCGAACGCTTTCTGGAAA
TTGAAACCCCGATGCTGACCAAAAGCACACCGGAAGGTGCACGTGATTATCTGGTTCCGAGCCGTGTTCA
TCCGGGTGAATTTTATGCACTGCCGCAGAGTCCGCAGATCTTTAAACAGCTGCTGATGGTTGGTGGTGTG
GAACGTTATTATCAGATTGCACGTTGTTTTCGTGATGAGGACCTGCGTGCAGATCGTCAGCCGGAATTTA
CCCAGATTGATATTGAAATGAGCTTCATCGAGCAAGAGGATATCATTGATCTGACCGAACGTATGATGGC
AGCAGTTGTTAAAGCAGCAAAAGGTATTGATATTCCGCGTCCGTTTCCGCGTATTACCTATGATGAAGCA
ATGAGCTGTTATGGTAGCGATAAACCGGATATTCGTTTTGGTCTGGAACTGGTTGATGTGAGCGAAATTG
TTCGTGATAGCGCATTTCAGGTTTTTGCGCGTGCAGTTAAAGAAGGTGGTCAGGTTAAAGCAATTAATGC
AAAAGGTGCAGCACCGCGTTATAGCCGTAAAGATATTGATGCACTGGGCGAATTTGCAGGTCGTTATGGT
GCCAAAGGTCTGGCATGGCTGAAAGCAGAAGGTGAAGAACTGAAAGGTCCGATTGCAAAATTCTTTACCG
ATGAAGAACAGGCAGCCCTGCGTCGTGCACTGGCCGTTGAAGATGGTGACCTGCTGCTGTTTGTTGCAGA
TGAAAAAGCAATTGTTGCAGCAGCACTGGGTGCGCTGCGTCTGAAACTGGGTAAAGAACTGGGTCTGATT
GATGAAGCCAAACTGGCATTTCTGTGGGTTACCGATTGGCCTCTGCTGGAATACGATGAAGAGGAAGGTC
GCTATTACGCAGCACATCATCCGTTTACCATGCCGGTGCGTGATGATATCCCGCTGCTGGAAACCAATCC
GAGCGCAGTTCGTGCACAGGCATATGATCTGGTTCTGAATGGTTATGAATTAGGTGGTGGTAGCCTGCGT
ATTTTTGAACGTGATGTGCAAGAAAAAATGTTTCGTGCCCTGGGTTTTAGCGAAGAAGAAGCACGTCGTC
AGTTTGGTTTTCTGTTAGAAGCATTTGAATATGGCACCCCTCCGCATGGTGGTATTGCACTGGGTTTAGA
TCGTCTGGTTATGCTGCTGGCAGGTCGTACCAATCTGCGCGATACCATTGCATTTCCGAAAACCGCCAGC
GCAAGCTGTCTGCTGACCGAAGCACCGGGTCCTGTTAGCGACAAACAGCTGGAAGAACTGCATCTGGCAG
TTGTTCTGCCGGAAAATGAATAA
SEQ ID NO. 32
Amino Acid
AspRS-GsAspRS-EcOpt
Geobacillus
MERTYYCGEVPETAVGERVVLKGWVQKRRDLGGLIFIDLRDRTGIVQVVASPDVSAEALAAAERVRSEYV
LSVEGTVVARAPETVNPNIATGRIEIQAERIEIINEAKTPPFSISDDTDAAEDVRLKYRYLDLRRPVMFQ
TLALRHKITKTVRDFLDSERFLEIETPMLTKSTPEGARDYLVPSRVHPGEFYALPQSPQIFKQLLMVGGV
ERYYQIARCFRDEDLRADRQPEFTQIDIEMSFIEQEDIIDLTERMMAAVVKAAKGIDIPRPFPRITYDEA
MSCYGSDKPDIRFGLELVDVSEIVRDSAFQVFARAVKEGGQVKAINAKGAAPRYSRKDIDALGEFAGRYG
AKGLAWLKAEGEELKGPIAKFFTDEEQAALRRALAVEDGDLLLFVADEKAIVAAALGALRLKLGKELGLI
DEAKLAFLWVTDWPLLEYDEEEGRYYAAHHPFTMPVRDDIPLLETNPSAVRAQAYDLVLNGYELGGGSLR
IFERDVQEKMFRALGFSEEEARRQFGFLLEAFEYGTPPHGGIALGLDRLVMLLAGRTNLRDTIAFPKTAS
ASCLLTEAPGPVSDKQLEELHLAVVLPENE
SEQ ID NO. 33
DNA
CysRS-GsCysRS-EcOpt
Geobacillus (codon-optimized for E. coli)
ATGAGCAGCATTCGTCTGTATAATACCCTGACGCGTAAAAAAGAACCGTTTGAACCGCTGGAACCGAACA
AAGTTAAAATGTATGTTTGTGGTCCGACCGTGTATAACTATATTCATATTGGTAATGCCCGTGCAGCCAT
TGTGTTTGATACCATTCGTCGTTATCTGGAATTTCGCGGTTATGATGTTACCTATGTGAGCAATTTTACC
GACGTGGATGACAAACTGATTAAAGCAGCACGTGAACTGGGTGAAAGCGTTCCGGCAATTGCAGAACGTT
TTATTGAAGCCTATTTCGAAGATATTCAGGCCCTGGGTTGTAAAAAAGCAGATATTCATCCGCGTGTGAC
CGAAAATATCGATACCATTATTGAATTTATCCAGGCGCTGATCGATAAAGGCTATGCATATGAAGTTGAT
GGCGACGTTTATTATCGTACCCGTAAATTTCGCGAATATGGCAAACTGAGCCATCAGAGCATTGATGAAC
TGCAGGCAGGCGCACGTATTGAAATTGGTGAAAAAAAAGATGATCCGCTGGATTTTGCACTGTGGAAAGC
AGCAAAAGAAGGTGAAATTTGTTGGGATAGCCCGTGGGGTAAAGGTCGTCCTGGTTGGCATATTGAATGT
AGCGCAATGGCACGTAAATATCTGGGTGATACGATTGATATTCATGCCGGTGGTCAGGATCTGACCTTTC
CGCATCATGAAAATGAAATTGCACAGAGCGAAGCACTGACCGGTAAACCGTTTGCCAAATATTGGCTGCA
TAATGGCTATCTGAACATCAACAACGAGAAAATGAGCAAAAGCCTGGGTAATTTTGTTCTGGTGCATGAT
ATTATTCGCGAGATTGATCCGCAGGTTCTGCGCTTTTTTATGCTGAGCGTTCATTATCGTCATCCGATCA
ATTATAGCGAAGAACTGCTGGAAAGCGCACGTCGTGGTCTGGAACGTCTGAAAACCGCATATAGCAATCT
GCAGCACCGTCTGCAGGCAAGCACCAATCTGACCGATAATGATGAAGAATGGGTTAGCCGTATTGCCGAT
ATTCGTGCAAGCTTTATTCGTGAAATGGATGATGATTTTAACACCGCCAATGGTATTGCCGTTCTGTTTG
AACTGGCAAAACAGGCAAATCTGTATCTGCAAGAAAAAACCACCTCCGAAAAAGTGATTCATGCATTTCT
GCGTGAATTTGAACAGCTGGCAGATGTTCTGGGTCTGACCCTGAAACAGGATGAGCTGCTGGATGAAGAA
ATTGAAGCCCTGATTCAGAAACGTAATGAAGCCCGTAAAAATCGTGATTTTGCCCTGGCAGATCGTATTC
GTGATGAATTACGTGCGAAAAACATCATCCTGGAAGATACACCGCAGGGCACCCGTTGGAAACGTGGTTA
A
SEQ ID NO. 34
Amino Acid
CysRS-GsCysRS-EcOpt
Geobacillus
MSSIRLYNTLTRKKEPFEPLEPNKVKMYVCGPTVYNYIHIGNARAAIVFDTIRRYLEFRGYDVTYVSNFT
DVDDKLIKAARELGESVPAIAERFIEAYFEDIQALGCKKADIHPRVTENIDTIIEFIQALIDKGYAYEVD
GDVYYRTRKFREYGKLSHQSIDELQAGARIEIGEKKDDPLDFALWKAAKEGEICWDSPWGKGRPGWHIEC
SAMARKYLGDTIDIHAGGQDLTFPHHENEIAQSEALTGKPFAKYWLHNGYLNINNEKMSKSLGNFVLVHD
IIREIDPQVLRFFMLSVHYRHPINYSEELLESARRGLERLKTAYSNLQHRLQASTNLTDNDEEWVSRIAD
IRASFIREMDDDFNTANGIAVLFELAKQANLYLQEKTTSEKVIHAFLREFEQLADVLGLTLKQDELLDEE
IEALIQKRNEARKNRDFALADRIRDELRAKNIILEDTPQGTRWKRG
SEQ ID NO. 35
DNA
GlnRS-EcGlnRS-EcOpt
E. coli
ATGAGCGAAGCAGAAGCACGTCCGACCAACTTTATTCGTCAGATTATTGATGAAGATCTGGCCAGCGGTA
AACATACCACCGTTCATACCCGTTTTCCGCCTGAACCGAATGGTTATCTGCATATTGGTCATGCCAAAAG
CATTTGCCTGAATTTTGGTATTGCCCAGGATTATAAAGGTCAGTGCAATCTGCGTTTCGATGATACCAAT
CCGGTGAAAGAAGATATCGAATACGTCGAGAGCATCAAAAATGATGTTGAATGGCTGGGTTTTCATTGGA
GCGGTAATGTTCGTTATAGCAGCGATTATTTTGATCAGCTGCATGCCTATGCAATCGAACTGATTAACAA
AGGTCTGGCCTATGTTGATGAACTGACACCGGAACAAATTCGTGAATATCGTGGTACACTGACCCAGCCT
GGTAAAAATAGCCCGTATCGTGATCGTAGCGTTGAAGAAAATCTGGCCCTGTTTGAAAAAATGCGTGCCG
GTGGTTTTGAAGAAGGTAAAGCCTGTCTGCGTGCAAAAATTGATATGGCAAGCCCGTTTATTGTTATGCG
TGATCCGGTTCTGTATCGCATCAAATTTGCAGAACATCATCAGACCGGTAACAAATGGTGTATCTATCCG
ATGTATGATTTCACCCATTGCATTAGTGATGCCCTGGAAGGTATTACCCATAGCCTGTGTACCCTGGAAT
TTCAGGATAATCGTCGTCTGTATGATTGGGTGTTAGACAATATCACCATTCCGGTGCATCCGCGTCAGTA
TGAATTTAGCCGTCTGAATCTGGAATACACCGTTATGAGCAAACGTAAACTGAATCTGCTGGTGACCGAT
AAACATGTTGAAGGTTGGGATGATCCGCGTATGCCGACCATTAGCGGTCTGCGTCGTCGTGGTTATACCG
CAGCAAGCATCCGTGAATTTTGTAAACGTATTGGTGTGACCAAACAGGATAACACCATTGAAATGGCCAG
CCTGGAAAGCTGTATTCGCGAAGATCTGAATGAAAATGCACCGCGTGCAATGGCAGTTATCGATCCGGTT
AAACTGGTGATCGAAAATTATCAAGGTGAAGGTGAAATGGTGACCATGCCGAATCATCCGAATAAACCGG
AAATGGGTAGCCGTCAGGTTCCGTTTAGCGGTGAAATTTGGATTGATCGTGCAGATTTTCGTGAAGAAGC
CAACAAACAGTATAAACGTCTGGTTCTGGGTAAAGAAGTTCGTCTGCGTAACGCCTATGTTATTAAAGCA
GAACGTGTTGAAAAAGATGCCGAAGGCAATATTACCACCATTTTTTGTACCTATGACGCAGATACCCTGA
GCAAAGATCCGGCAGATGGTCGTAAAGTTAAAGGTGTTATTCATTGGGTTAGCGCAGCACATGCACTGCC
GGTTGAAATTCGCCTGTATGATCGTCTGTTTAGCGTTCCGAATCCGGGTGCAGCAGATGATTTTCTGAGC
GTTATTAATCCGGAAAGCCTGGTTATTAAACAGGGTTTTGCCGAACCGAGCCTGAAAGATGCAGTTGCAG
GTAAAGCATTTCAGTTTGAACGCGAAGGTTATTTTTGTCTGGATAGCCGTCATAGCACCGCAGAAAAACC
GGTGTTTAATCGTACCGTTGGTCTGCGTGATACCTGGGCAAAAGTTGGTGAATAA
SEQ ID NO. 36
Amino Acid
GlnRS-EcGlnRS-EcOpt
E. coli
MSEAEARPTNFIRQIIDEDLASGKHTTVHTRFPPEPNGYLHIGHAKSICLNFGIAQDYKGQCNLRFDDTN
PVKEDIEYVESIKNDVEWLGFHWSGNVRYSSDYFDQLHAYAIELINKGLAYVDELTPEQIREYRGTLIQP
GKNSPYRDRSVEENLALFEKMRAGGFEEGKACLRAKIDMASPFIVMRDPVLYRIKFAEHHQTGNKWCIYP
MYDFTHCISDALEGITHSLCTLEFQDNRRLYDWVLDNITIPVHPRQYEFSRLNLEYTVMSKRKLNLLVTD
KHVEGWDDPRMPTISGLRRRGYTAASIREFCKRIGVTKQDNTIEMASLESCIREDLNENAPRAMAVIDPV
KLVIENYQGEGEMVTMPNHPNKPEMGSRQVPFSGEIWIDRADFREEANKQYKRLVLGKEVRLRNAYVIKA
ERVEKDAEGNITTIFCTYDADTLSKDPADGRKVKGVIHWVSAAHALPVEIRLYDRLFSVPNPGAADDFLS
VINPESLVIKQGFAEPSLKDAVAGKAFQFEREGYFCLDSRHSTAEKPVFNRTVGLRDTWAKVGE
SEQ ID NO. 37
DNA
GluRS-GsGluRS-EcOpt
Geobacillus (codon-optimized for E. coli)
ATGGCCAAAGAAGTTCGCGTTCGTTACGCACCGAGTCCGACCGGTCATCTGCATATTGGTGGTGCACGTA
CCGCACTGTTTAATTACCTGTTTGCACGTCATCATGGTGGCAAAATGATTGTGCGTATTGAAGATACCGA
TATCGAACGTAATGTTGAAGGTGGTGAAAAAAGCCAGCTGGAAAATCTGAAATGGCTGGGCATTGATTAT
GATGAAAGCATTGATCAGGATGGTGGTTATGGTCCGTATCGTCAGACCGAACGTCTGGATATTTATCGCA
AATATGTGAACGAACTGCTGGAACAGGGTCATGCCTATAAATGTTTTTGTACACCGGAAGAACTGGAACG
TGAACGTGAAGCACAGCGTGCAGCAGGTATTGCAGCACCGCAGTATAGCGGTAAATGTCGTCATCTGACA
CCGGAACAGGTTGCCGAACTGGAAGCACAGGGTAAACCGTATACCATTCGTCTGAAAGTTCCGGAAGGTA
AAACCTATGAATTCTATGATCTGGTGCGTGGCAAAGTTGTGTTTGAAAGCAAAGATGTTGGTGGCGATTG
GGTTATTGTTAAAGCAAATGGTATTCCGACCTATAACTTTGCCGTTGTGATTGATGATCACCTGATGGAA
ATTTCACATGTGTTTCGTGGTGAAGAACATCTGAGCAATACCCCGAAACAGCTGATGGTGTATGAATATT
TTGGTTGGGAACCGCCTCAGTTTGCACATCTGACCCTGATTGTTAATGAACAGCGTAAAAAACTGAGCAA
ACGCGACGAAAGCATTATTCAGTTTGTGAGCCAGTATAAAGAACTGGGTTATCTGCCGGAAGCCATGTTT
AACTTTTTTGCACTGTTAGGTTGGTCACCGGAAGGTGAAGAAGAAATCTTTACCAAAGATGAACTGATCC
GCATGTTTGATGTTAGCCGTCTGAGCAAAAGCCCGAGTATGTTTGATACCAAAAAGCTGACCTGGATGAA
CAACCAGTACATCAAAAAACTGGATCTGGATCGTCTGGTTGAACTGGCACTGCCGCATCTGGTTAAAGCA
GGTCGTCTGCCTGCAGATATGACCGATGAGCAGCGTCAGTGGGCACGTGATCTGATTGCACTGTATCAAG
AGCAGATGAGCTATGGTGCAGAAATTGTTCCGCTGAGCGAACTGTTTTTCAAAGAAGAGATTGATTACGA
GGATGAAGCACGTCAGGTTCTGGCAGAAGAACAGGTTCCGGCAGTTCTGAGCACCTTTCTGGAAAGCGTT
CGTGAGCTGGAACCGTTTACCGCAGATGAAATTAAAGCAGCAATTAAAGCCGTTCAGAAAGCAACCGGTC
AGAAAGGGAAAAAACTGTTTATGCCGATTCGTGCAGCCGTTACAGGTCAGACCCATGGTCCGGAACTGCC
GTTTGCAATTCAGCTGCTGGGTAAAGAAAAAGTGATTGAACGCCTGGAACGCGCACTGCAAGAAAAATTC
TAA
SEQ ID NO. 38
Amino Acid
GluRS-GsGluRS-EcOpt
Geobacillus
MAKEVRVRYAPSPTGHLHIGGARTALFNYLFARHHGGKMIVRIEDTDIERNVEGGEKSQLENLKWLGIDY
DESIDQDGGYGPYRQTERLDIYRKYVNELLEQGHAYKCFCTPEELEREREAQRAAGIAAPQYSGKCRHLT
PEQVAELEAQGKPYTIRLKVPEGKTYEFYDLVRGKVVFESKDVGGDWVIVKANGIPTYNFAVVIDDHLME
ISHVFRGEEHLSNTPKQLMVYEYFGWEPPQFAHLTLIVNEQRKKLSKRDESIIQFVSQYKELGYLPEAMF
NFFALLGWSPEGEEEIFTKDELIRMFDVSRLSKSPSMFDTKKLTWMNNQYIKKLDLDRLVELALPHLVKA
GRLPADMTDEQRQWARDLIALYQEQMSYGAEIVPLSELFFKEEIDYEDEARQVLAEEQVPAVLSTFLESV
RELEPFTADEIKAAIKAVQKATGQKGKKLFMPIRAAVTGQTHGPELPFAIQLLGKEKVIERLERALQEKF
SEQ ID NO. 39
DNA
GlyRS-GsGlyRS-EcOpt
Geobacillus (codon-optimized for E. coli)
ATGGCAGTTACCATGGAAGAAATTGTTGCACATGCAAAACATCGTGGTTTTGTTTTTCCGGGTAGCGAAA
TTTATGGTGGTCTGGCAAATACCTGGGATTATGGTCCGCTGGGTGTTGAACTGAAAAATAACATTAAACG
TGCCTGGTGGAAAAAATTCGTTCAAGAAAGCCCGTATAATGTTGGTCTGGATGCAGCAATTCTGATGAAT
CCGCGTACCTGGGAAGCAAGCGGTCATCTGGGTAACTTTAATGATCCGATGGTTGATTGCAAACAGTGTA
AAGCACGTCATCGTGCAGATAAACTGATTGAAAAAGCCCTGGAAGAAAAAGGCATTGAGATGATTGTTGA
TGGTCTGCCGCTGGCAAAAATGGATGAACTGATTAAAGAATATGATATCGCCTGTCCGGAATGTGGTAGC
CGTGATTTTACCAATGTTCGTCAGTTTAACCTGATGTTCAAAACCTATCAGGGTGTTACCGAAAGCAGCG
CCAATGAAATTTATCTGCGTCCGGAAACCGCACAGGGTATTTTTGTTAATTTCAAAAATGTGCAGCGCAC
CATGCGTAAAAAACTGCCGTTTGGTATTGCACAGATTGGCAAAAGCTTTCGCAACGAAATTACCCCTGGT
AATTTTACCTTTCGCACCCGTGAATTTGAGCAGATGGAACTGGAATTTTTCTGTAAACCGGGTGAAGAAC
TGCAGTGGCTGGAATATTGGAAACAGTTTTGTAAAGAATGGCTGCTGAGCCTGGGTATGAAAGAAGATAA
TATTCGTCTGCGTGATCATGCCAAAGAAGAACTGAGCCATTATAGCAATGCAACCACCGATATCGAATAT
CATTTTCCGTTTGGTTGGGGTGAACTGTGGGGTATTGCAAGCCGTACCGATTATGATCTGAAACGCCATA
TGGAATATAGCGGTGAAGATTTCCATTACCTGGATCAAGAAACCAACGAACGTTATATTCCGTATTGTAT
TGAACCGAGTCTGGGTGCAGATCGTGTTACCCTGGCATTTATGATTGATGCCTATGATGAAGAGGAACTT
GAAGATGGTACAACCCGTACCGTGATGCATCTGCATCCGGCACTGGCACCGTATAAAGCAGCAGTGCTGC
CGTTAAGCAAAAAACTGGCAGATGGTGCACGTCGTATTTATGAGGAACTGGCAAAACACTTCATGGTGGA
TTATGATGAAACCGGTAGTATTGGTAAACGTTATCGTCGTCAGGATGAAATTGGCACCCCGTTTTGTATT
ACCTATGATTTTGAAAGCGAACAGGATGGTCAGGTTACCGTTCGTGATCGTGATACAATGGAACAGGTTC
GTCTGCCGATTGGCGAACTGAAAGCATTTCTGGAAGAGAAAATCGCCTTCTAA
SEQ ID NO. 40
Amino Acid
GlyRS-GsGlyRS-EcOpt
Geobacillus
MAVTMEEIVAHAKHRGFVFPGSEIYGGLANTWDYGPLGVELKNNIKRAWWKKFVQESPYNVGLDAAILMN
PRTWEASGHLGNFNDPMVDCKQCKARHRADKLIEKALEEKGIEMIVDGLPLAKMDELIKEYDIACPECGS
RDFTNVRQFNLMFKTYQGVTESSANEIYLRPETAQGIFVNFKNVQRTMRKKLPFGIAQIGKSFRNEITPG
NFTFRTREFEQMELEFFCKPGEELQWLEYWKQFCKEWLLSLGMKEDNIRLRDHAKEELSHYSNATTDIEY
HFPFGWGELWGIASRTDYDLKRHMEYSGEDFHYLDQETNERYIPYCIEPSLGADRVTLAFMIDAYDEEEL
EDGTTRTVMHLHPALAPYKAAVLPLSKKLADGARRIYEELAKHFMVDYDETGSIGKRYRRQDEIGTPFCI
TYDFESEQDGQVTVRDRDTMEQVRLPIGELKAFLEEKIAF
SEQ ID NO. 41
DNA
HisRS-GsHisRS-EcOpt
Geobacillus (codon-optimized for E. coli)
ATGGCATTTCAGATTCCGCGTGGCACCCAGGATGTTCTGCCTGGTGATACCGAAAAATGGCAGTATGTTG
AACATGTTGCACGTAATCTGTGTAGCCGTTATGGTTATCGTGAAATTCGTACCCCGATTTTTGAACACAC
CGAACTGTTTCTGCGTGGTGTGGGTGATACCACCGATATTGTTCAGAAAGAAATGTATACCTTCGAGGAT
AAAGGTGGTCGTGCACTGACCCTGCGTCCGGAAGGCACCGCACCGGTTGTTCGTGCATTTGTGGAACATA
AACTGTATGGTAGTCCGCATCAGCCGCTGAAACTGTATTATTCAGGTCCGATGTTTCGTTATGAACGTCC
TGAAGCAGGTCGTTTTCGTCAGTTTGTTCAGTTTGGTGTTGAAGCACTGGGTAGCAGCGATCCGGCAATT
GATGCAGAAGTTATGGCACTGGCAATGCATATTTATGAAGCCCTGGGTCTGAAACGTATTCGTCTGGTGA
TTAATAGCCTGGGTGATCTGGATAGCCGTCGTGCACATCGTGAAGCGCTGGTTCGTCATTTTAGCAGCCG
TATTCATGAACTGTGTCCGGATTGTCAGACCCGTCTGCATACCAATCCGCTGCGTATTCTGGATTGTAAA
AAAGATCGTGATCATGAGCTGATGGCAACCGCACCGAGCATCCTGGATTATCTGAATGAAGATAGCCGTG
CCTATTTCGAGAAAGTGAAACAGTATCTGACCAATCTGGGTATTCCGTTTGTTATTGATAGTCGTCTGGT
TCGTGGTCTGGATTATTACAATCATACCACCTTTGAAATCATGAGCGAAGCCGAAGGTTTTGGTGCAGCA
GCAACCCTGTGTGGTGGTGGTCGTTATAATGGTCTGGTTCAAGAAATTGGTGGTCCGGAAACACCTGGTA
TTGGTTTTGCACTGAGCATTGAACGTCTGCTGGCAGCACTGGATGCCGAAGGTGTTGAACTGCCGGTTGA
AAGTGGCCTGGATTGTTATGTTGTTGCAGTTGGTGAACGTGCAAAAGATGAAGCAGTGCGTCTGGTTTAT
GCCCTGCGTCGTAGCGGTCTGCGTGTTGATCAGGATTACCTGGGTCGTAAACTGAAAGCACAGCTGAAAG
CAGCAGATCGTCTGGGTGCAAGCTTTGTTGCAATTATTGGTGATGAGGAACTGGAACGTCAAGAAGCAGC
AGTTAAACATATGGCAAGCGGTGAACAGACCAATGTTCCGCTGGGTGAACTGGCACATTTTCTGCATGAA
CGTATTGGCAAAGAAGAATAA
SEQ ID NO. 42
Amino Acid
HisRS-GsHisRS-EcOpt
Geobacillus
MAFQIPRGTQDVLPGDTEKWQYVEHVARNLCSRYGYREIRTPIFEHTELFLRGVGDTTDIVQKEMYTFED
KGGRALTLRPEGTAPVVRAFVEHKLYGSPHQPLKLYYSGPMFRYERPEAGRFRQFVQFGVEALGSSDPAI
DAEVMALAMHIYEALGLKRIRLVINSLGDLDSRRAHREALVRHFSSRIHELCPDCQTRLHTNPLRILDCK
KDRDHELMATAPSILDYLNEDSRAYFEKVKQYLTNLGIPFVIDSRLVRGLDYYNHTTFEIMSEAEGFGAA
ATLCGGGRYNGLVQEIGGPETPGIGFALSIERLLAALDAEGVELPVESGLDCYVVAVGERAKDEAVRLVY
ALRRSGLRVDQDYLGRKLKAQLKAADRLGASFVAIIGDEELERQEAAVKHMASGEQTNVPLGELAHFLHE
RIGKEE
SEQ ID NO. 43
DNA
IleRS-GsIleRS-EcOpt
Geobacillus stearothermophilus (codon-optimized for E. coli)
ATGGACTACAAAGAAACCCTGCTGATGCCGCAGACCGAATTTCCGATGCGTGGTAATCTGCCGAAACGTG
AACCGGAAATGCAGAAAAAATGGGAAGAGATGGATATCTACCGCAAAGTTCAAGAACGTACCAAAGGTCG
TCCGCTGTTTGTTCTGCATGATGGTCCGCCTTATGCAAATGGTGATATTCATATGGGTCATGCCCTGAAC
AAAATCCTGAAAGATATTATCGTGCGCTATAAGAGCATGAATGGTTATTGTGCACCGTATGTTCCAGGTT
GGGATACCCATGGTCTGCCGATTGAAACCGCACTGGCAAAACAGGGTGTTGATCGTAAAAGCATGAGCGT
TGCAGAATTTCGTAAACGTTGTGAACAGTATGCCTATGAGCAGATTGATAATCAGCGTCGTCAGTTTAAA
CGTCTGGGTGTTCGTGGTGATTGGGATAATCCGTATATTACCCTGAAACCGGAATATGAAGCACAGCAGA
TTAAAGTGTTTGGCGAGATGGCAAAAAAAGGCCTGATCTATAAAGGTCTGAAACCTGTTTATTGGAGCCC
GAGCAGCGAAAGTGCACTGGCAGAAGCAGAAATTGAGTATAAAGATAAACGCTCCCCGAGCATTTATGTT
GCCTTTCCGGTTAAAGATGGTAAAGGTGTTCTGGAAGGTGATGAACGTATTGTGATTTGGACCACCACAC
CGTGGACCATTCCGGCAAATCTGGCAATTGCAGTTCATCCGGATCTGGATTATCATGTTGTTGATGTTAG
CGGTAAACGTTATGTTGTTGCAGCAGCACTGGCCGAAAGCGTTGCAAAAGAAATTGGTTGGGATGCATGG
TCAGTTGTGAAAACCGTTAAAGGTAAAGAACTGGAATATGTGGTTGCGAAACACCCGTTTTATGAACGTG
ATAGCCTGGTTGTTTGTGGTGAACATGTGACCACCGATGCAGGCACCGGTTGTGTTCATACCGCACCTGG
TCATGGTGAAGATGATTTTCTGGTTGGTCAGAAATATGGCCTGCCGGTTCTGTGTCCGGTGGATGAACGT
GGTTATATGACCGAAGAAGCACCGGGTTTTGAAGGTATGTTTTATGAGGATGCCAACAAAGCGATTACGC
AGAAACTGGAAGAAGTTGGCGCACTGCTGAAACTGGGTTTTATTACCCATAGCTATCCGCATGATTGGCG
TACCAAACAGCCGACCATTTTTCGTGCAACCACACAGTGGTTTGCAAGCATTGATAAAATTCGCAATGAA
CTGCTGCAGGCCATCAAAGAAACAAAATGGATCCCGGAATGGGGTGAAATTCGCATTCATAACATGGTTC
GTGATCGCGGTGATTGGTGTATTAGCCGTCAGCGTGCATGGGGTGTTCCGATTCCGGTGTTTTATGGTGA
AAATGGTGAACCGATTATCACCGATGAAACCATTGAACATGTTAGCAACCTGTTTCGTCAGTATGGTAGC
AATGTTTGGTTTGAACGTGAAGCAAAAGATCTGCTGCCGGAAGGTTTTACCCATCCGAGCAGCCCGAATG
GTATTTTTACAAAAGAAACCGATATCATGGACGTGTGGTTTGATAGCGGTAGCAGCCATCAGGCAGTTCT
GGTGGAACGTGATGATCTGATGCGTCCGGCAGATCTGTATCTGGAAGGCAGCGATCAGTATCGTGGTTGG
TTTAATAGCAGCCTGAGCACCGCAGTTGCAGTGACCGGTAAAGCACCGTATAAAGGTGTGCTGAGCCATG
GTTTTGTGCTGGATGGTGAAGGTCGTAAAATGAGCAAAAGCCTGGGTAATGTTGTTGTTCCTGCAAAAGT
TATGGAACAGTTTGGTGCAGATATTCTGCGTCTGTGGGTTGCCAGCGTTGATTATCAGGCAGATGTTCGT
ATTAGCGATCATATTCTGAAACAGGTGAGCGAAGTGTATCGCAAAATTCGTAATACCTTTCGCTTTATGC
TGGGTAACCTGTTTGATTTTGATCCGAATCAGAATGCAGTTCCGATTGGTGAACTGGGTGAAGTTGATCG
TTATATGCTGGCCAAACTGAATAAACTGATCGCCAAAGTGAAAAAAGCCTATGATAGCTACGATTTCGCA
GCCGTTTATCATGAAATGAACCATTTTTGTACCGTTGAACTGAGCGCCTTTTATCTGGATATGGCAAAAG
ATATCCTGTATATCGAAGCAGCAGATAGCCGTGCACGTCGTGCAGTTCAGACCGTTCTGTATGAAACCGT
TGTTGCACTGGCGAAACTGATTGCACCGATTCTGCCGCATACCGCAGATGAAGTTTGGGAACATATTCCG
AATCGTCGTGAAAATGTGGAAAGCGTTCAGCTGACCGATATGCCGGAACCGATTGCAATTGATGGCGAAG
AGGCACTGCTGGCAAAATGGGATGCCTTTATGGATGTTCGTGATGATATGCTGAAAGCACTGGAAAATGC
CCGTAACGAAAAAGTGATTGGTAAAAGCCTGACCGCAAGCGTTATTGTTTATCCGAAAGATGAAGCACGT
AAACTGCTGGCGAGCCTGGATGCCGATCTGCGTCAGCTGCTGATTGTTAGCGCATTTAGCATTGCAGATG
AACCGTATGATGCTGCCCCTGCAGAAGCCGAACGTCTGGATCATGTTGCCGTTCTGGTTCGTCCTGCCGA
AGGTGAAACCTGCGAACGTTGTTGGACCGTTACACCGGCAGTTGGTCAGGATCCGAGCCATCCGACCTTT
TGTCCGCGTTGTGCACATATTGTTAACGAACATTATAGCGCCTAA
SEQ ID NO. 44
Amino Acid
IleRS-GsIleRS-EcOpt
Geobacillus stearothermophilus
MDYKETLLMPQTEFPMRGNLPKREPEMQKKWEEMDIYRKVQERTKGRPLFVLHDGPPYANGDIHMGHALN
KILKDIIVRYKSMNGYCAPYVPGWDTHGLPIETALAKQGVDRKSMSVAEFRKRCEQYAYEQIDNQRRQFK
RLGVRGDWDNPYITLKPEYEAQQIKVFGEMAKKGLIYKGLKPVYWSPSSESALAEAEIEYKDKRSPSIYV
AFPVKDGKGVLEGDERIVIWTTTPWTIPANLAIAVHPDLDYHVVDVSGKRYVVAAALAESVAKEIGWDAW
SVVKTVKGKELEYVVAKHPFYERDSLVVCGEHVTTDAGTGCVHTAPGHGEDDFLVGQKYGLPVLCPVDER
GYMTEEAPGFEGMFYEDANKAITQKLEEVGALLKLGFITHSYPHDWRTKQPTIFRATTQWFASIDKIRNE
LLQAIKETKWIPEWGEIRIHNMVRDRGDWCISRQRAWGVPIPVFYGENGEPIITDETIEHVSNLFRQYGS
NVWFEREAKDLLPEGFTHPSSPNGIFTKETDIMDVWFDSGSSHQAVLVERDDLMRPADLYLEGSDQYRGW
FNSSLSTAVAVTGKAPYKGVLSHGFVLDGEGRKMSKSLGNVVVPAKVMEQFGADILRLWVASVDYQADVR
ISDHILKQVSEVYRKIRNTFRFMLGNLFDFDPNQNAVPIGELGEVDRYMLAKLNKLIAKVKKAYDSYDFA
AVYHEMNHFCTVELSAFYLDMAKDILYIEAADSRARRAVQTVLYETVVALAKLIAPILPHTADEVWEHIP
NRRENVESVQLTDMPEPIAIDGEEALLAKWDAFMDVRDDMLKALENARNEKVIGKSLTASVIVYPKDEAR
KLLASLDADLRQLLIVSAFSIADEPYDAAPAEAERLDHVAVLVRPAEGETCERCWTVTPAVGQDPSHPTF
CPRCAHIVNEHYSA
SEQ ID NO. 45
DNA
LeuRS-GsLeuRS-EcOpt
Geobacillus stearothermophilus (codon-optimized for E. coli)
ATGAGCTTTAACCACCGTGAAATCGAACAGAAATGGCAGGATTATTGGGAGAAGAATAAAACCTTTCGTA
CACCGGATGATGATGACAAACCGAAATTCTATGTGCTGGATATGTTTCCGTATCCGAGCGGTGCAGGTCT
GCATGTTGGTCATCCGGAAGGTTATACCGCAACCGATATTCTGGCACGTATGAAACGTATGCAGGGTTAT
AATGTTCTGCATCCGATGGGTTGGGATGCATTTGGTCTGCCTGCAGAACAGTATGCACTGGATACCGGTA
ATGATCCGGCAGAATTTACCCAGAAAAACATCGATAACTTTCGTCGCCAGATTAAAAGCCTGGGTTTTAG
CTATGATTGGGATCGTGAAATCAATACCACCGATCCGAATTATTACAAATGGACCCAGTGGATCTTCCTG
AAACTGTATGAAAAAGGTCTGGCCTATATGGATGAAGTTCCGGTTAATTGGTGTCCGGCACTGGGCACCG
TTCTGGCAAATGAAGAAGTTATTAACGGTCGTAGCGAACGTGGTGGCCATCCGGTTATTCGTAAACCGAT
GCGTCAGTGGATGCTGAAAATTACCGCATATGCAGATCGTCTGCTGGAAGATCTGGAAGAATTAGATTGG
CCTGAAAGCATCAAAGAAATGCAGCGTAATTGGATTGGTCGTAGTGAAGGTGCAGAAATTGAATTTGCAG
TTGATGGTCACGATGAAACCTTTACCGTTTTTACCACACGTCCGGATACACTGTTTGGTGCAACCTATAC
CGTGCTGGCACCGGAACATCCGCTGGTTGAAAAAATCACCACTCCGGAACAGAAACCTGCCGTTGATGCA
TATCTGAAAGAAATTCAGAGCAAAAGCGATCTGGAACGTACCGATCTGGCCAAAGAAAAAACCGGTGTGT
TTACCGGTGCATATGCCATTCATCCTGTTACCGGTGATCGCCTGCCGATTTGGATTGCAGATTATGTTCT
GATGAGCTATGGTACAGGTGCAATTATGGCAGTTCCGGCACATGATGAACGTGATTATGAATTCGCCAAA
AAATTCCATCTGCCGATGAAAGAAGTTGTTGCAGGCGGTAATATTGAGAAAGAAGCATATACAGGCGACG
GCGAACATATTAACAGCGAATTTCTGAATGGCCTGAATAAACAAGAGGCCATCGATAAAATGATTGCCTG
GCTGGAAGAACATGGTAAAGGTCGTAAAAAAGTTAGCTATCGTCTGCGTGATTGGCTGTTTAGCCGTCAG
CGTTATTGGGGTGAACCGATTCCGATTATTCATTGGGAAGATGGCACCATGACACCGGTTCCGGAAGAAG
AACTGCCGCTGGTTCTGCCGAAAACCGATGAAATTCGTCCGAGCGGCACCGGTGAAAGTCCGCTGGCAAA
TATTGAAGAATGGGTTAATGTTGTGGATCCGAAAACGGGTAAAAAAGGTCGTCGCGAAACCAATACCATG
CCGCAGTGGGCAGGTAGCTGTTGGTATTATCTGCGTTATATTGATCCGCACAACGATAAACAGCTGGCAG
ATCCGGAAAAACTGAAAAAATGGCTGCCGGTTGATGTGTATATTGGTGGTGCCGAACATGCAGTGCTGCA
TCTGCTGTATGCACGTTTTTGGCATAAATTTCTGTATGACCTGGGTATTGTTCCGACCAAAGAACCGTTT
CAGAAACTGTTTAATCAGGGTATGATTCTGGGCGAGAACAACGAAAAAATGAGCAAAAGTAAAGGCAATG
TGGTGAACCCGGATGATATTATTGAAAGCCATGGTGCAGATACCCTGCGTCTGTATGAGATGTTTATGGG
TCCGCTGGAAGCAAGCATTGCATGGTCAACCAAAGGCCTGGATGGTGCACGTCGTTTTCTGGATCGTGTT
TGGCGTCTGTTTGTTACCGAAAATGGTGAACTGAATCCGAACATTGTTGATGAACCGGCAAATGATACCC
TGGAACGCATTTATCATCAGACCGTTAAAAAAGTGACCGAGGATTATGAAGCCCTGCGTTTTAATACCGC
AATTAGCCAGCTGATGGTGTTTATTAACGAAGCCTATAAAGCCGAGCAGATGAAAAAAGAATATATGGAA
GGCTTCGTGAAACTGCTGAGTCCGGTTTGTCCGCATATTGGTGAAGAACTGTGGCAGAAACTGGGTCATA
CCGATACCATTGCATATGAACCGTGGCCGACCTATGATGAAACCAAACTGGTTGAAGATGTGGTGGAAAT
TGTTGTGCAGATTAATGGTAAAGTGCGTAGTCGCCTGCATGTGCCTGTTGATCTGCCTAAAGAAGCCTTA
GAAGAACGCGCACTGGCGGATGAAAAGATTAAAGAACAGCTGGAAGGTAAAACCGTGCGTAAAGTTATTG
CCGTTCCGGGTAAACTGGTTAATATTGTTGCCAACTAA
SEQ ID NO. 46
Amino Acid
LeuRS-GsLeuRS-EcOpt
Geobacillus stearothermophilus
MSFNHREIEQKWQDYWEKNKTFRTPDDDDKPKFYVLDMFPYPSGAGLHVGHPEGYTATDILARMKRMQGY
NVLHPMGWDAFGLPAEQYALDTGNDPAEFTQKNIDNFRRQIKSLGFSYDWDREINTTDPNYYKWTQWIFL
KLYEKGLAYMDEVPVNWCPALGTVLANEEVINGRSERGGHPVIRKPMRQWMLKITAYADRLLEDLEELDW
PESIKEMQRNWIGRSEGAEIEFAVDGHDETFTVFTTRPDTLFGATYTVLAPEHPLVEKITTPEQKPAVDA
YLKEIQSKSDLERTDLAKEKTGVFTGAYAIHPVTGDRLPIWIADYVLMSYGTGAIMAVPAHDERDYEFAK
KFHLPMKEVVAGGNIEKEAYTGDGEHINSEFLNGLNKQEAIDKMIAWLEEHGKGRKKVSYRLRDWLFSRQ
RYWGEPIPIIHWEDGTMTPVPEEELPLVLPKTDEIRPSGTGESPLANIEEWVNVVDPKTGKKGRRETNTM
PQWAGSCWYYLRYIDPHNDKQLADPEKLKKWLPVDVYIGGAEHAVLHLLYARFWHKFLYDLGIVPTKEPF
QKLFNQGMILGENNEKMSKSKGNVVNPDDIIESHGADTLRLYEMFMGPLEASIAWSTKGLDGARRFLDRV
WRLFVTENGELNPNIVDEPANDTLERIYHQTVKKVTEDYEALRFNTAISQLMVFINEAYKAEQMKKEYME
GFVKLLSPVCPHIGEELWQKLGHTDTIAYEPWPTYDETKLVEDVVEIVVQINGKVRSRLHVPVDLPKEAL
EERALADEKIKEQLEGKTVRKVIAVPGKLVNIVAN
SEQ ID NO. 47
DNA
LysRS-GsLysRS-EcOpt
Geobacillus stearothermophilus (codon-optimized for E. coli)
ATGAGCCATGAAGAACTGAATGATCAGCTGCGTGTTCGTCGTGAAAAACTGAAAAAAATCGAAGAACTGG
GCGTTGATCCGTTTGGTAAACGTTTTGAACGTACCCATAAAGCCCAAGAACTGTTTGAACTGTATGGTGA
TCTGAGCAAAGAGGAACTGGAAGAAAAACAAATTGAAGTTGCAGTTGCCGGTCGCATTATGACCAAACGT
GGTAAAGGTAAAGCAGGCTTTGCACATATTCAGGATGTTACCGGTCAGATTCAGATTTATGTGCGTCAGG
ATGATGTTGGTGAACAGCAGTATGAACTGTTCAAAATTAGCGATCTGGGTGATATTGTTGGTGTTCGTGG
CACCATGTTTAAAACCAAAGTGGGTGAACTGAGCATTAAAGTGAGCAGCTATGAATTTCTGACCAAAGCA
CTGCGTCCGCTGCCGGAAAAATATCATGGTCTGAAAGATATTGAACAGCGTTATCGTCAGCGCTATCTGG
ATCTGATTATGAATCCGGAAAGCAAAAAAACCTTTATTACCCGCTCACTGATTATCCAGAGCATGCGTCG
TTATCTGGATAGCCGTGGATATCTGGAAGTTGAAACCCCGATGATGCATGCCGTTGCCGGTGGTGCAGCA
GCACGTCCGTTTATTACACATCATAATGCACTGGATATGACCCTGTATATGCGTATTGCAATTGAACTGC
ATCTGAAACGTCTGATTGTTGGCGGTCTGGAAAAAGTGTATGAAATTGGTCGTGTGTTTCGCAATGAAGG
TATTAGCACCCGTCATAATCCGGAATTTACCATGCTGGAACTGTACGAAGCATATGCCGATTTTCACGAT
ATTATGGAACTGACCGAAAACCTGATTGCCCATATTGCAACCGAAGTTCTGGGCACCACCAAAATTCAGT
ATGATGAACATGTTGTTGACCTGACACCGGAATGGCGTCGTCTGCATATGGTTGATGCAATTAAAGAATA
TGTCGGCGTGGATTTTTGGCGTCAGATGAGTGATGAAGAAGCACGCGAACTGGCAAAAGAACATGGTGTG
GAAGTTGCACCGCATATGACCTTTGGCCATATTGTGAACGAATTCTTTGAGCAGAAAGTGGAAAGCCATC
TGATTCAGCCGACCTTTATCTATGGTCATCCGGTTGAAATTAGTCCGCTGGCCAAAAAAAACCCGGATGA
TCCTCGTTTTACCGATCGTTTTGAGCTGTTTATTGTGGGTCGTGAACATGCAAATGCCTTTACCGAACTG
AACGATCCGATTGATCAGCGTCAGCGTTTTGAAGCACAGCTGAAAGAACGTGAACAGGGTAATGATGAAG
CACACGAAATGGATGAAGATTTTCTGGAAGCACTGGAATATGGTATGCCTCCGACCGGTGGTTTAGGTAT
TGGTGTTGATCGTCTGGTTATGCTGCTGACCAATAGTCCGAGCATTCGTGATGTTCTGCTGTTTCCGCAG
ATGCGTCATAAATAA
SEQ ID NO. 48
Amino Acid
LysRS-GsLysRS-EcOpt
Geobacillus stearothermophilus
MSHEELNDQLRVRREKLKKIEELGVDPFGKRFERTHKAQELFELYGDLSKEELEEKQIEVAVAGRIMTKR
GKGKAGFAHIQDVTGQIQIYVRQDDVGEQQYELFKISDLGDIVGVRGTMFKTKVGELSIKVSSYEFLTKA
LRPLPEKYHGLKDIEQRYRQRYLDLIMNPESKKTFITRSLIIQSMRRYLDSRGYLEVETPMMHAVAGGAA
ARPFITHHNALDMTLYMRIAIELHLKRLIVGGLEKVYEIGRVFRNEGISTRHNPEFTMLELYEAYADFHD
IMELTENLIAHIATEVLGTTKIQYDEHVVDLTPEWRRLHMVDAIKEYVGVDFWRQMSDEEARELAKEHGV
EVAPHMTFGHIVNEFFEQKVESHLIQPTFIYGHPVEISPLAKKNPDDPRFTDRFELFIVGREHANAFTEL
NDPIDQRQRFEAQLKEREQGNDEAHEMDEDFLEALEYGMPPTGGLGIGVDRLVMLLTNSPSIRDVLLFPQ
MRHK
SEQ ID NO. 49
DNA
MetRS-GsMetRS-EcOpt
Geobacillus stearothermophilus (codon-optimized for E. coli)
ATGGAAAAAAAGACCTTCTATCTGACCACGCCGATCTATTATCCGAGCGATCGTCTGCATATTGGTCATG
CATATACCACCGTTGCCGGTGATGCAATGGCACGTTATAAACGTATGCGTGGTTATGATGTTATGTATCT
GACCGGCACCGATGAACATGGTCAGAAAATTCAGCGTAAAGCCGAAGAAAAAGGTGTTACACCGCAGCAG
TATGTTGATGAAATTGTTGCAGGTATTCAAGAACTGTGGAAAAAACTGGATATCAGCTATGATGATTTCA
TCCGTACCACACAAGAACGCCATAAAAAAGTTGTTGAGCAGATTTTTACCCGTCTGGTTGAACAGGGTGA
TATTTATCTGGGTGAATATGAAGGTTGGTATTGTACCCCGTGTGAAAGCTTTTATACCGAACGTCAGCTG
GTTGATGGTAATTGTCCGGATTGTGGTCGTCCGGTTGAAAAAGTTAAAGAGGAAAGCTATTTTTTCCGCA
TGAGCAAATATGTTGATCGCCTGCTGCAGTATTATGAAGAAAACCCGGATTTCATTCAGCCGGAAAGCCG
TAAAAATGAGATGATTAACAACTTTATCAAACCTGGCCTGGAAGATCTGGCAGTTAGCCGTACCACCTTT
GATTGGGGTATTAAAGTTCCGGGTAATCCGAAACATGTGATCTATGTTTGGATTGATGCACTGGCCAACT
ATATTACCGCATTAGGTTATGGCACCGATAACGATGAAAAATTCCGTAAATATTGGCCTGCCGATGTTCA
TCTGGTTGGTAAAGAAATTGTTCGCTTCCATACCATTTATTGGCCGATTATGCTGATGGCACTGGGTCTG
CCGCTGCCGAAAAAAGTTTTTGGTCATGGTTGGCTGCTGATGAAAGATGGTAAAATGAGCAAAAGCAAAG
GCAATGTTGTTGATCCGGTTACACTGATTGATCGTTATGGTCTGGATGCACTGCGTTATTATCTGCTGCG
TGAAGTTCCGTTTGGTGCAGATGGTGTTTTTACACCGGAAGGTTTTATTGAGCGCATCAATTATGATCTG
GCAAATGATCTGGGTAATCTGCTGCATCGTACCGTTGCAATGATCGAAAAATACTTTGATGGTGTGATTC
CGCCTTATCGTGGTCCGAAAACACCGTTTGATCAAGAGCTGGTTCAGACCGCACGTGAAGTTGTTCGTCA
GTATGAAGAGGCAATGGAAGGTATGGAATTTAGCGTTGCACTGGCAGCAGTTTGGCAGCTGATTAGTCGT
ACCAATAAATACATTGATGAAACCCAGCCGTGGGTGTTAGCAAAAGATGAACAGAAACGTGATGAACTGG
CAGCCGTTATGACCCATCTGGCAGAAAGCCTGCGTCATACCGCAGTTCTGCTGCAGCCGTTTCTGACCCG
CACACCGGAACGTATGCTGGCACAGCTGGGTATTACCGATCATAGCCTGAAAGAATGGGATAGCCTGTAT
GATTTTGGTCTGATTCCGGAAGGCACCAAAGTTCAGAAAGGTGAACCGCTGTTTCCGCGTCTGGATATTG
AAGCAGAAGTGGAATATATCAAAGCCCATATGCAAGGTGGTAAACCGGCAGCCGAACCGGTTAAAGAAGA
AAAAAAAGCAGCCGAAGCAGCGGAAATTAGCATCGATGAATTTGCAAAAGTTGATCTGCGTGTTGCCGAA
GTTATTCATGCAGAACGTATGAAAAACGCCGATAAACTGCTGAAACTGCAGCTGGATTTAGGTGGTGAAA
AACGTCAGGTTATTAGCGGTATTGCCGAATTCTATAAACCGGAAGAACTGGTGGGTAAAAAAGTGATTTG
TGTGGCAAATCTGAAACCGGCAAAACTGCGTGGTGAATGGTCTGAAGGCATGATTCTGGCAGGCGGTAGC
GGTGGTGAATTTAGCCTGGCAACCGTTGATCAGCATGTTCCGAATGGTACGAAAATCAAATAA
SEQ ID NO. 50
Amino Acid
MetRS-GsMetRS-EcOpt
Geobacillus stearothermophilus
MEKKTFYLTTPIYYPSDRLHIGHAYTTVAGDAMARYKRMRGYDVMYLTGTDEHGQKIQRKAEEKGVTPQQ
YVDEIVAGIQELWKKLDISYDDFIRTTQERHKKVVEQIFTRLVEQGDIYLGEYEGWYCTPCESFYTERQL
VDGNCPDCGRPVEKVKEESYFFRMSKYVDRLLQYYEENPDFIQPESRKNEMINNFIKPGLEDLAVSRTTF
DWGIKVPGNPKHVIYVWIDALANYITALGYGTDNDEKFRKYWPADVHLVGKEIVRFHTIYWPIMLMALGL
PLPKKVFGHGWLLMKDGKMSKSKGNVVDPVTLIDRYGLDALRYYLLREVPFGADGVFTPEGFIERINYDL
ANDLGNLLHRTVAMIEKYFDGVIPPYRGPKTPFDQELVQTAREVVRQYEEAMEGMEFSVALAAVWQLISR
INKYIDETQPWVLAKDEQKRDELAAVMTHLAESLRHTAVLLQPFLTRTPERMLAQLGITDHSLKEWDSLY
DFGLIPEGTKVQKGEPLFPRLDIEAEVEYIKAHMQGGKPAAEPVKEEKKAAEAAEISIDEFAKVDLRVAE
VIHAERMKNADKLLKLQLDLGGEKRQVISGIAEFYKPEELVGKKVICVANLKPAKLRGEWSEGMILAGGS
GGEFSLATVDQHVPNGTKIK
SEQ ID NO. 51
DNA
Phe-aRS-GsPhe-aRS-EcOpt
Geobacillus (codon-optimized for E. coli)
ATGAAAGAACGCCTGTATGAACTGAAACGTCAGGCACTGGAACAAATTGGTCAGGCACGTGATCTGCGTA
TGCTGAATGATGTTCGTGTTGCATATCTGGGTAAAAAAGGTCCGATTACCGAAGTTCTGCGTGGTATGGG
TGCACTGCCTCCGGAAGAACGTCCGAAAATTGGTGCACTGGCAAATGAAGTTCGTGAAGCAATTCAGCAG
GCCCTGGAAGCAAAACAGGCAAAACTTGAACAAGAAGAAGTGGAACGTAAACTGGCAGCCGAAGCAATTG
ATGTTACCCTGCCTGGTCGTCCGGTTAGCCTGGGTAATCCGCATCCGCTGACACGTGTTATTGAAGAAAT
TGAGGACCTGTTTATTGGCATGGGTTATACCGTTGCAGAAGGTCCGGAAGTTGAAACCGATTATTACAAT
TTTGAAGCCCTGAATCTGCCGAAAGGTCATCCGGCACGCGATATGCAGGATAGCTTTTATATCACCGAAG
AAATTCTGCTGCGTACCCATACCTCACCGATGCAGGCACGTACCATGGAAAAACATCGTGGTCGTGGTCC
GGTTAAAATCATTTGTCCGGGTAAAGTTTATCGTCGCGATACCGATGATGCAACCCATAGCCATCAGTTT
ACACAGATTGAAGGTCTGGTTGTGGATCGTAATATTCGTATGAGCGATCTGAAAGGCACCCTGCGTGAAT
TTGCCCGTAAACTGTTTGGTGAAGGTCGTGATATTCGTTTTCGTCCGAGCTTTTTTCCGTTTACCGAACC
GAGCGTTGAAGTTGATGTTAGCTGTTTTCGTTGTGAAGGCCGTGGTTGCGGTGTTTGTAAAGGCACCGGT
TGGATTGAAATTTTAGGTGCAGGTATGGTTCATCCGAATGTTCTGGAAATGGCAGGTTTTGATAGTAAAA
CCTATACCGGTTTTGCATTCGGTATGGGTCCTGAACGTATTGCAATGCTGAAATATGGCATTGATGATAT
CCGCCACTTCTATCAGAATGATCTGCGCTTTCTGCGTCAGTTTCTGCGTGTTTAA
SEQ ID NO. 52
Amino Acid
Phe-aRS-GsPhe-aRS-EcOpt
Geobacillus
MKERLYELKRQALEQIGQARDLRMLNDVRVAYLGKKGPITEVLRGMGALPPEERPKIGALANEVREAIQQ
ALEAKQAKLEQEEVERKLAAEAIDVTLPGRPVSLGNPHPLTRVIEEIEDLFIGMGYTVAEGPEVETDYYN
FEALNLPKGHPARDMQDSFYITEEILLRTHTSPMQARTMEKHRGRGPVKIICPGKVYRRDTDDATHSHQF
TQIEGLVVDRNIRMSDLKGTLREFARKLFGEGRDIRFRPSFFPFTEPSVEVDVSCFRCEGRGCGVCKGTG
WIEILGAGMVHPNVLEMAGFDSKTYTGFAFGMGPERIAMLKYGIDDIRHFYQNDLRFLRQFLRV
SEQ ID NO. 53
DNA
Phe-bRS-GsPhe-bRS-EcOpt
Geobacillus stearothermophilus (codon-optimized for E. coli)
ATGCTGGTTAGCTATCGTTGGCTGGGTGAATATGTTGATCTGACCGGTATTACCGCAAAAGAACTGGCAG
AACGTATTACCAAAAGCGGTATTGAAGTTGAACGTGTTGAAGCACTGGATCGTGGTATGAATGGTGTTGT
TATTGGTCATGTTCTGGAATGTGAACCGCATCCGAATGCAGATAAACTGCGTAAATGTCTGGTTGATTTA
GGTGAAGGTGAACCGGTGCGTATTATTTGTGGTGCACCGAATGTTGCAAAAGGTCAGAAAGTTGCAGTTG
CCAAAGTTGGTGCAGTTCTGCCTGGTAACTTTAAAATCAAACGTGCAAAACTGCGTGGCGAAGAAAGCAA
TGGTATGATTTGTAGCCTGCAAGAACTGGGTGTTGAAACCAAAGTTGTTCCGAAAGAATATGCCGATGGC
ATTTTTGTTTTTCCGAGTGATGCACCGGTTGGTGCCGATGCACTGGAATGGCTGGGTCTGCATGATGAAG
TTCTGGAACTGGCACTGACCCCGAATCGTGCAGATTGTCTGAGCATGATTGGTGTTGCCTATGAAGTTGC
AGCAATTCTGGGTCGTGATGTTAAACTGCCGGAAGCAGCAGTTAAAGAAAATAGCGAACATGTGCACGAA
TATATCAGCGTTCGTGTGGAAGCACCGGAAGATAATCCGCTGTATGCAGGTCGTATTGTTAAAAATGTTC
GTATTGGTCCGAGTCCGCTGTGGATGCAGGCACGTCTGATGGCAGCAGGTATTCGTCCGCATAATAATGT
TGTTGACATCACCAACTATATCCTGCTGGAATATGGTCAGCCGCTGCATGCATTTGATTATGATCGTCTG
GGTAGCAAAGAAATTGTTGTTCGTCGTGCAAAAGCCGGTGAAACCATTATTACCCTGGATGATGTTGAAC
GTAAACTGACCGAAAATCATCTGGTGATTACCAATGGTCGCGAACCGGTTGCACTGGCAGGCGTTATGGG
TGGTGCCAATAGCGAAGTTCGTGATGATACCACCACCGTTTTTATTGAAGCAGCCTATTTCACCAGTCCG
GTTATTCGTCAGGCCGTTAAAGATCATGGTCTGCGTAGCGAAGCGAGCACCCGTTTTGAAAAAGGTATTG
ATCCGGCACGTACCAAAGAGGCCCTGGATCGCGCAGCAGCACTGATGAGCGAATATGCAGGCGGTGAAGT
TGTTGGTGGTATTGTTGAAGCCAGCGTTTGGCGTCAGGATCCGGTTGTTGTTACCGTTACACTGGAACGC
ATTAATGGTGTTCTGGGCACCGCAATGACCAAAGAAGAAGTGGCTGCCATTCTGAGCAATCTGCAGTTTC
CGTTTACCGAAGATAATGGCACCTTTACCATTCATGTTCCGAGCCGTCGTCGTGATATTGCAATTGAAGA
AGATATTATTGAAGAGGCAGCCCGTCTGTATGGTTATGATCGCCTGCCTGCAACACTGCCGGTTGCCGAA
GCAAAACCTGGTGGTCTGACACCGCATCAGGCAAAACGTCGTCGCGTTCGTCGTTATCTGGAAGGCACCG
GTCTGTTTCAGGCAATTACCTATAGCCTGACCTCACCGGATAAAGCAACCCGCTTTGCCCTGGAAACCGC
AGAACCGATTCGTCTGGCACTGCCGATGAGTGAAGAACGTAGCGTTCTGCGTCAGAGCCTGATTCCGCAT
CTGCTGGAAGCCGCAAGCTATAATCGTGCACGTCAGGTTGAAGATGTTGCCCTGTATGAAATTGGTAGCG
TTTATCTGAGCAAAGGTGAACATGTACAGCCTGCAGAAAAAGAACGTTTAGCCGGTGTGCTGACAGGTCT
GTGGCATGCACATCTGTGGCAGGGTGAAAAAAAAGCCGTTGATTTTTATGTGGCCAAAGGTATTCTGGAT
GGTCTGTTTGATCTGCTGGGTTTAGCAGCACGTATTGAATATAAACCGGCAAAACGCGCTGATCTGCATC
CGGGTCGTACCGCAGATATTGTGCTGGATGGCCGTGTGATTGGTTTTGTTGGTCAGCTGCATCCTGCAGT
TCAGAAAGAGTATGATCTGAAAGAAACCTATGTGTTTGAGCTGGCCCTGACCGATCTGCTGAATGCAGAA
AGCGAAGCAATTCGTTATGAACCTATTCCGCGTTTTCCGAGCGTTGTGCGCGACATTGCACTGGTTGTTG
ATGAAAATGTTGAAGCGGGTGCACTGAAACAGGCAATCGAAGAAGCAGGTAAACCGCTGGTTAAAGATGT
TAGCCTGTTCGATGTTTATAAAGGCGATCGTCTGCCGGATGGTAAAAAAAGTCTGGCATTTAGCCTGCGT
TATTATGATCCGGAACGCACCCTGACAGATGAAGAGGTTGCAGCAGTGCATGAACGTGTGCTGGCAGCAG
TTGAAAAACAGTTTGGTGCCGTGCTGCGTGGTTAA
SEQ ID NO. 54
Amino Acid
Phe-bRS-GsPhe-bRS-EcOpt
Geobacillus stearothermophilus
MLVSYRWLGEYVDLTGITAKELAERITKSGIEVERVEALDRGMNGVVIGHVLECEPHPNADKLRKCLVDL
GEGEPVRIICGAPNVAKGQKVAVAKVGAVLPGNFKIKRAKLRGEESNGMICSLQELGVETKVVPKEYADG
IFVFPSDAPVGADALEWLGLHDEVLELALTPNRADCLSMIGVAYEVAAILGRDVKLPEAAVKENSEHVHE
YISVRVEAPEDNPLYAGRIVKNVRIGPSPLWMQARLMAAGIRPHNNVVDITNYILLEYGQPLHAFDYDRL
GSKEIVVRRAKAGETIITLDDVERKLTENHLVITNGREPVALAGVMGGANSEVRDDTTTVFIEAAYFISP
VIRQAVKDHGLRSEASTRFEKGIDPARTKEALDRAAALMSEYAGGEVVGGIVEASVWRQDPVVVTVTLER
INGVLGTAMTKEEVAAILSNLQFPFTEDNGTFTIHVPSRRRDIAIEEDIIEEAARLYGYDRLPAILPVAE
AKPGGLTPHQAKRRRVRRYLEGTGLFQAITYSLTSPDKATRFALETAEPIRLALPMSEERSVLRQSLIPH
LLEAASYNRARQVEDVALYEIGSVYLSKGEHVQPAEKERLAGVLTGLWHAHLWQGEKKAVDFYVAKGILD
GLFDLLGLAARIEYKPAKRADLHPGRTADIVLDGRVIGFVGQLHPAVQKEYDLKETYVFELALTDLLNAE
SEAIRYEPIPRFPSVVRDIALVVDENVEAGALKQAIEEAGKPLVKDVSLFDVYKGDRLPDGKKSLAFSLR
YYDPERTLTDEEVAAVHERVLAAVEKQFGAVLRG
SEQ ID NO. 55
DNA
ProRS-GsProRS-EcOpt
Geobacillus stearothermophilus (codon-optimized for E. coli)
ATGCGTCAGAGCCAGGCATTTATTCCGACACTGCGTGAAGTTCCGGCAGATGCAGAAGTTAAAAGCCATC
AGCTGCTGCTGCGTGCAGGTTTTATTCGTCAGAGCGCAAGCGGTGTTTATACCTTTCTGCCGCTGGGTCA
GCGTGTGCTGCAGAAAGTTGAAGCAATTATTCGCGAAGAAATGAATCGTATTGGTGCCATGGAACTGTTT
ATGCCTGCACTGCAGCCTGCAGAACTGTGGCAGCAGAGCGGTCGTTGGTATAGCTATGGTCCGGAACTGA
TGCGTCTGAAAGATCGTCATGAACGTGATTTTGCACTGGGTCCGACACATGAAGAGATGATTACCGCAAT
TGTTCGTGATGAGGTGAAAACCTATAAACGTCTGCCTCTGGTTCTGTATCAGATCCAGACCAAATTCCGT
GATGAAAAACGTCCGCGTTTTGGTCTGTTACGTGGTCGTGAATTTATGATGAAAGATGCCTATAGCTTCC
ATACCAGCAAAGAAAGCCTGGATGAAACCTACAACAATATGTATGAAGCCTACGCCAACATTTTTCGTCG
TTGCGGTCTGAATTTTCGTGCAGTTATTGCAGATAGCGGTGCAATTGGTGGTAAAGATACCCACGAATTC
ATGGTTCTGAGCGATATTGGTGAAGATACCATTGCATATAGTGATGCAAGCGATTATGCAGCCAATATTG
AAATGGCACCGGTTGTTGCAACCTATGAAAAAAGTGATGAACCTCCGGCAGAACTGAAGAAAGTTGCCAC
ACCGGGTCAGAAAACCATTGCCGAAGTTGCAAGCCATCTGCAAATTAGTCCGGAACGTTGTATTAAAAGC
CTGCTGTTTAATGTGGATGGTCGTTATGTTCTGGTGCTGGTTCGTGGTGATCATGAAGCAAATGAAGTGA
AAGTGAAAAATGTGCTGGATGCCACCGTTGTTGAACTGGCAAAACCGGAAGAAACCGAACGTGTTATGAA
TGCACCGATTGGTAGCCTGGGTCCTATTGGTGTTAGCGAAGATGTTACCGTTATTGCCGATCATGCAGTT
GCAGCAATTGTTAATGGTGTTTGTGGTGCCAATGAAGAGGGCTATCATTACATTGGTGTGAATCCGGGTC
GCGATTTTGCAGTTAGCCAGTATGCCGATCTGCGTTTTGTTAAAGAAGGTGATCCGAGTCCGGATGGTAA
AGGCACCATTCGTTTTGCACGTGGTATTGAAGTTGGCCATGTTTTTAAACTGGGCACCAAATATAGCGAA
GCCATGAATGCAGTTTATCTGGATGAGAATGGTCAGACCCAGACAATGATTATGGGTTGTTATGGTATTG
GCGTTAGCCGTCTGGTTGCAGCCATTGCAGAACAGTTTGCCGATGAACATGGTCTGGTTTGGCCTGCAAG
CGTTGCACCGTTTCATATTCATCTGCTGACCGCAAATGCCAAATCAGATGAACAGCGTGCACTGGCCGAA
GAATGGTATGAAAAACTGGGTCAAGCAGGTTTTGAAGTGCTGTATGATGATCGTCCAGAACGTGCCGGTG
TTAAATTTGCCGATAGCGATCTGATTGGTATTCCGCTGCGTGTTACCGTGGGTAAACGTGCAGGCGAAGG
TGTTGTTGAAGTTAAAGTTCGTAAAACCGGTGAAACCTTTGATGTTCCGGTTAGCGAACTGGTTGATACC
GCACGTCGTCTGCTGCAGAGCTAA
SEQ ID NO. 56
Amino Acid
ProRS-GsProRS-EcOpt
Geobacillus stearothermophilus
MRQSQAFIPTLREVPADAEVKSHQLLLRAGFIRQSASGVYTFLPLGQRVLQKVEAIIREEMNRIGAMELF
MPALQPAELWQQSGRWYSYGPELMRLKDRHERDFALGPTHEEMITAIVRDEVKTYKRLPLVLYQIQTKFR
DEKRPRFGLLRGREFMMKDAYSFHTSKESLDETYNNMYEAYANIFRRCGLNFRAVIADSGAIGGKDTHEF
MVLSDIGEDTIAYSDASDYAANIEMAPVVATYEKSDEPPAELKKVATPGQKTIAEVASHLQISPERCIKS
LLFNVDGRYVLVLVRGDHEANEVKVKNVLDATVVELAKPEETERVMNAPIGSLGPIGVSEDVTVIADHAV
AAIVNGVCGANEEGYHYIGVNPGRDFAVSQYADLRFVKEGDPSPDGKGTIRFARGIEVGHVFKLGTKYSE
AMNAVYLDENGQTQTMIMGCYGIGVSRLVAAIAEQFADEHGLVWPASVAPFHIHLLTANAKSDEQRALAE
EWYEKLGQAGFEVLYDDRPERAGVKFADSDLIGIPLRVTVGKRAGEGVVEVKVRKTGETFDVPVSELVDT
ARRLLQS
SEQ ID NO. 57
DNA
SerRS-GsSerRS-EcOpt
Geobacillus stearothermophilus (codon-optimized for E. coli)
ATGCTGGATGTGAAAATTCTGCGTACCCAGTTTGAAGAGGTGAAAGAAAAACTGATGCAGCGTGGTGGTG
ATCTGACCAATATTGATCGTTTTGAACAGCTGGATAAAGATCGTCGTCGTCTGATTGCAGAAGTTGAAGA
ACTGAAAAGCAAACGCAATGATGTTAGCCAGCAGATTGCAGTTCTGAAACGCGAAAAAAAAGATGCAGAA
CCGCTGATTGCACAGATGCGTGAAGTTGGTGATCGTATTAAACGTATGGATGAGCAGATTCGTCAGCTGG
AAGCAGAACTGGATGATCTGCTGCTGAGCATTCCGAATGTTCCGCATGAAAGCGTTCCGATTGGCCAGAG
CGAAGAAGATAACGTTGAAGTTCGTCGTTGGGGTGAACCGCGTAGCTTTAGCTTTGAACCGAAACCGCAT
TGGGAAATTGCAGATCGTCTGGGTCTGCTGGATTTTGAACGTGCAGCAAAAGTTGCAGGTAGCCGTTTTG
TTTTCTATAAAGGTCTGGGTGCACGTCTGGAACGTGCACTGATTAACTTTATGCTGGATATTCACCTGGA
TGAGTTTGGCTATGAAGAAGTTCTGCCTCCGTATCTGGTTAATCGTGCAAGCATGATTGGCACCGGTCAG
CTGCCGAAATTTGCAGAAGATGCATTTCATCTGGATAGCGAGGATTATTTTCTGATTCCGACCGCAGAAG
TTCCGGTTACCAATCTGCATCGTGATGAAATTCTGGCAGCAGATGACCTGCCGATCTATTATGCAGCATA
TAGCGCATGTTTTCGTGCAGAAGCAGGTAGCGCAGGTCGTGATACCCGTGGTCTGATTCGCCAGCATCAG
TTCAATAAAGTTGAACTGGTGAAATTCGTGAAGCCGGAAGATAGCTATGATGAACTGGAAAAGCTGACCC
GTCAGGCAGAAACCATTCTGCAGCGTCTGGGCCTGCCGTATCGTGTTGTTGCACTGTGTACCGGTGATCT
GGGTTTTAGCGTTGCAAAAACCTATGATATTGAAGTTTGGCTGCCGAGCTATGGCACCTATCGTGAAATT
AGCAGCTGTAGCAATTTTGAAGCATTTCAGGCACGTCGTGCCAATATTCGTTTTCGTCGTGATCCGAAAG
CAAAACCGGAATATGTTCATACCCTGAATGGTAGCGGTCTGGCAATTGGTCGTACCGTTGCAGCAATTCT
GGAAAATTATCAGCAAGAAGATGGCAGCGTTATTGTTCCGGAAGCACTGCGTCCGTATATGGGCAATCGT
GATGTTATTCGTTAA
SEQ ID NO. 58
Amino Acid
SerRS-GsSerRS-EcOpt
Geobacillus stearothermophilus
MLDVKILRTQFEEVKEKLMQRGGDLTNIDRFEQLDKDRRRLIAEVEELKSKRNDVSQQIAVLKREKKDAE
PLIAQMREVGDRIKRMDEQIRQLEAELDDLLLSIPNVPHESVPIGQSEEDNVEVRRWGEPRSFSFEPKPH
WEIADRLGLLDFERAAKVAGSRFVFYKGLGARLERALINFMLDIHLDEFGYEEVLPPYLVNRASMIGTGQ
LPKFAEDAFHLDSEDYFLIPTAEVPVTNLHRDEILAADDLPIYYAAYSACFRAEAGSAGRDTRGLIRQHQ
FNKVELVKFVKPEDSYDELEKLTRQAETILQRLGLPYRVVALCTGDLGFSVAKTYDIEVWLPSYGTYREI
SSCSNFEAFQARRANIRFRRDPKAKPEYVHTLNGSGLAIGRTVAAILENYQQEDGSVIVPEALRPYMGNR
DVIR
SEQ ID NO. 59
DNA
ThrRS-GsThrRS-EcOpt
Geobacillus (codon-optimized for E. coli)
ATGCCGGATGTTATTCGTATTACCTTTCCGGATGGTGCCGAAAAAGAATTTCCGAAAGGCACCACCACCG
AAGATGTTGCAGCAAGCATTAGTCCGGGTCTGAAAAAAAAGGCAATTGCGGGTAAACTGAATGGTCGTTT
TGTTGATCTGCGTACACCGCTGCATGAAGATGGTGAACTGGTGATTATTACCCAGGATATGCCGGAAGCA
CTGGATATTCTGCGTCATAGCACCGCACATCTGATGGCACAGGCAATTAAACGTCTGTATGGCAATGTGA
AATTAGGTGTTGGTCCGGTGATTGAAAACGGCTTCTATTATGATATCGACATGGAACATAAACTGACACC
GGATGATCTGCCGAAAATTGAAGCAGAAATGCGCAAAATCGTGAAAGAGAACCTGGATATTGTTCGCAAA
GAAGTTAGTCGCGAAGAGGCAATTCGCCTGTATGAAGAAATTGGTGATGAACTGAAACTGGAACTGATTG
CAGATATTCCGGAAGGTGAACCGATTAGCATTTATGAACAGGGCGAATTTTTTGATCTGTGCCGTGGTGT
TCATGTTCCGAGCACCGGTAAAATCAAAGAATTTAAACTGCTGAGCATCAGCGGTGCATATTGGCGTGGT
GATAGCAATAACAAAATGCTGCAGCGTATTTATGGCACCGCGTTTTTCAAAAAAGAAGATCTGGATCGTT
ATCTGCGTCTGCTGGAAGAAGCAAAAGAACGCGATCATCGTAAACTGGGTAAAGAGCTGGAACTGTTTAC
CACCAGTCAGCAGGTTGGTCAGGGTCTGCCGCTGTGGCTGCCGAAAGGTGCAACCATTCGTCGTATTATT
GAACGCTATATCGTGGATAAAGAAGTTGCACTGGGTTACGATCATGTTTATACACCGGTTCTGGGTAGCG
TTGAACTGTATAAAACCAGCGGTCATTGGGATCACTACAAAGAAAATATGTTTCCGCCTATGGAAATGGA
CAATGAAGAACTGGTTCTGCGTCCGATGAATTGTCCGCATCACATGATGATCTATAAAAGCAAACTGCAC
AGCTATCGTGAACTGCCGATTCGTATTGCAGAACTGGGCACCATGCATCGTTATGAAATGAGCGGTGCAC
TGACCGGTCTGCAGCGTGTTCGTGGTATGACCCTGAATGATGCACATATCTTTGTTCGTCCGGATCAGAT
CAAAGATGAATTCAAACGTGTGGTGAACCTGATCCTGGAAGTGTATAAAGATTTTGGCATCGAAGAATAC
AGCTTCCGTCTGAGTTATCGTGATCCGCATGATAAAGAAAAATACTATGATGACGATGAAATGTGGGAAA
AAGCACAGCGTATGCTGCGTGAAGCAATGGATGAATTAGGTCTGGATTATTATGAAGCCGAAGGTGAAGC
AGCCTTTTATGGTCCGAAACTGGATGTTCAGGTTCGTACCGCACTGGGAAAAGATGAAACCCTGAGCACC
GTTCAGCTGGATTTTCTGCTGCCGGAACGTTTCGATCTGACCTATATTGGTGAAGATGGCAAACCGCATC
GTCCGGTTGTTATTCATCGTGGTGTTGTTAGCACCATGGAACGTTTTGTGGCATTTCTGATCGAAGAGTA
TAAAGGTGCATTTCCGACCTGGCTGGCACCGGTTCAGGTTAAAGTTATTCCGGTTAGTCCGGAAGCGCAC
CTGGATTATGCATATGATGTTCAGCGTACCCTGAAAGAACGTGGTTTTCGTGTTGAAGTTGATGAACGCG
ACGAAAAAATCGGCTATAAAATCCGTGAAGCACAGATGCAGAAAATCCCGTATATGCTGGTTGTTGGTGA
TAAAGAGGTTAGCGAACGCGCAGTTAATGTTCGTCGTTATGGTGAAAAAGAAAGCCGTACCATGGGCCTT
GATGAATTTATGGCCCTGCTGGCAGATGATGTTCGTGAAAAACGTACCCGTCTGGGCAAAGCACAGTAA
SEQ ID NO. 60
Amino Acid
ThrRS-GsThrRS-EcOpt
Geobacillus
MPDVIRITFPDGAEKEFPKGTTTEDVAASISPGLKKKAIAGKLNGRFVDLRTPLHEDGELVIITQDMPEA
LDILRHSTAHLMAQAIKRLYGNVKLGVGPVIENGFYYDIDMEHKLTPDDLPKIEAEMRKIVKENLDIVRK
EVSREEAIRLYEEIGDELKLELIADIPEGEPISIYEQGEFFDLCRGVHVPSTGKIKEFKLLSISGAYWRG
DSNNKMLQRIYGTAFFKKEDLDRYLRLLEEAKERDHRKLGKELELFTTSQQVGQGLPLWLPKGATIRRII
ERYIVDKEVALGYDHVYTPVLGSVELYKTSGHWDHYKENMFPPMEMDNEELVLRPMNCPHHMMIYKSKLH
SYRELPIRIAELGTMHRYEMSGALTGLQRVRGMTLNDAHIFVRPDQIKDEFKRVVNLILEVYKDFGIEEY
SFRLSYRDPHDKEKYYDDDEMWEKAQRMLREAMDELGLDYYEAEGEAAFYGPKLDVQVRALGKDETLSTV
QLDFLLPERFDLTYIGEDGKPHRPVVIHRGVVSTMERFVAFLIEEYKGAFPTWLAPVQVKVIPVSPEAHL
DYAYDVQRTLKERGFRVEVDERDEKIGYKIREAQMQKIPYMLVVGDKEVSERAVNVRRYGEKESRTMGLD
EFMALLADDVREKRTRLGKAQ
SEQ ID NO. 61
DNA
TrpRS-GsTrpRS-EcOpt
Geobacillus stearothermophilus (codon-optimized for E. coli)
ATGAAAACCATCTTTAGCGGTATTCAGCCGAGCGGTGTTATTACCCTGGGTAACTATATTGGTGCACTGC
GTCAGTTTATTGAACTGCAGCATGAATATAACTGCTATTTCTGCATTGTTGATCAGCATGCAATTACCGT
TTGGCAGGATCCGCATGAACTGCGCCAGAATATTCGTCGTCTGGCAGCACTGTATCTGGCAGTTGGTATT
GATCCGACACAGGCAACCCTGTTTATTCAGAGCGAAGTTCCGGCACATGCACAGGCAGCATGGATGCTGC
AATGTATTGTTTATATTGGCGAACTGGAACGCATGACCCAGTTTAAAGAAAAAAGCGCAGGTAAAGAAGC
AGTTAGCGCAGGTCTGCTGACCTATCCGCCTCTGATGGCAGCCGATATTCTGCTGTATAACACCGATATT
GTTCCGGTTGGTGATGATCAGAAACAGCATATCGAACTGACCCGTGATCTGGCAGAACGTTTTAACAAAC
GTTATGGTGAGCTGTTTACCATTCCGGAAGCACGTATTCCGAAAGTTGGTGCACGTATTATGAGCCTGGT
GGATCCGACCAAAAAAATGAGCAAAAGCGATCCGAATCCGAAAGCCTATATTACACTGCTGGATGATGCA
AAAACCATCGAGAAAAAAATCAAAAGTGCCGTGACCGATAGCGAAGGCACCATTCGTTATGATAAAGAAG
CCAAACCGGGTATTAGCAACCTGCTGAACATTTATAGCACCCTGAGCGGTCAGAGCATTGAAGAATTAGA
ACGTAAATATGAAGGCAAAGGCTACGGTGTTTTTAAAGCAGATCTGGCACAGGTTGTTATTGAAACCCTG
CGTCCGATTCAAGAACGTTATCATCATTGGATGGAAAGCGAAGAACTGGATCGTGTTCTGGATGAAGGTG
CAGAAAAAGCAAATCGTGTTGCAAGCGAAATGGTGCGTAAAATGGAACAGGCAATGGGTCTGGGTCGTCG
TCGTTAA
SEQ ID NO. 62
Amino Acid
TrpRS-GsTrpRS-EcOpt
Geobacillus stearothermophilus
MKTIFSGIQPSGVITLGNYIGALRQFIELQHEYNCYFCIVDQHAITVWQDPHELRQNIRRLAALYLAVGI
DPTQATLFIQSEVPAHAQAAWMLQCIVYIGELERMTQFKEKSAGKEAVSAGLLTYPPLMAADILLYNTDI
VPVGDDQKQHIELTRDLAERFNKRYGELFTIPEARIPKVGARIMSLVDPTKKMSKSDPNPKAYIILLDDA
KTIEKKIKSAVTDSEGTIRYDKEAKPGISNLLNIYSTLSGQSIEELERKYEGKGYGVFKADLAQVVIETL
RPIQERYHHWMESEELDRVLDEGAEKANRVASEMVRKMEQAMGLGRRR
SEQ ID NO. 63
DNA
TyrRS-GsTyrRS-EcOpt
Geobacillus stearothermophilus (codon-optimized for E. coli)
ATGGATCTGCTGGCAGAACTGCAGTGGCGTGGTCTGGTGAATCAGACCACCGATGAAGATGGTCTGCGTG
AACTGCTGAAAGAAGAACGCGTTACCCTGTATTGTGGTTTTGATCCGACCGCAGATAGCCTGCATATTGG
TAATCTGGCAGCAATTCTGACCCTGCGTCGTTTTCAGCAGGCAGGTCATCAGCCGATTGCACTGGTTGGT
GGTGCAACCGGTCTGATTGGTGATCCGAGCGGTAAAAAAAGCGAACGTACCCTGAATGCAAAAGAAACCG
TTGAAGCATGGTCAGCACGTATTCAAGAACAGCTGAGCCGTTTTCTGGATTTTGAAGCACATGGTAATCC
GGCAAAAATCAAGAACAACTATGATTGGATTGGTCCGCTGGATGTTATTACCTTTCTGCGTGATGTTGGC
AAACATTTCAGCGTGAATTATATGATGGCCAAAGAAAGCGTTCAGAGCCGTATTGAAACCGGTATTAGCT
TTACCGAATTCAGCTATATGATGCTGCAGGCCTATGATTTTCTGCGTCTGTATGAAACCGAAGGTTGTCG
TCTGCAGATTGGTGGTAGCGATCAGTGGGGCAATATTACCGCAGGTCTGGAACTGATTCGTAAAACCAAA
GGTGAAGCACGTGCATTTGGTCTGACCATTCCGCTGGTTACCAAAGCAGATGGTACAAAATTTGGTAAAA
CCGAAAGCGGCACCATTTGGCTGGATAAAGAAAAAACCAGTCCGTATGAGTTCTACCAGTTTTGGATTAA
TACCGATGATCGTGATGTGATCCGCTACCTGAAATACTTTACATTTCTGAGCAAAGAAGAGATCGAAGCC
TTTGAACAAGAACTGCGTGAAGCACCGGAAAAACGTGCAGCACAGAAAGCACTGGCAGAAGAAGTTACCA
AACTGGTTCATGGTGAAGAAGCACTGCGTCAGGCAGTTCGTATTAGCGAAGCACTGTTTAGCGGTGATAT
TGGCAACCTGACCGCAGCAGAAATTGAACAGGGTTTTAAAGATGTTCCGAGCTTTGTTCATGAAGGTGGT
GATGTGCCGCTGGTCGAACTGCTGGTTAGCGCAGGTATTAGCCCGAGCAAACGTCAGGCACGTGAAGATA
TTCAGAATGGTGCCATTTATGTGAATGGTGAACGTCTGCAGGATGTTGGTGCGATTCTGACAGCAGAACA
TCGTCTGGAAGGTCGTTTTACCGTTATTCGTCGTGGCAAGTATTACCTGATTCGCTATGCCTAA
SEQ ID NO. 64
Amino Acid
TyrRS-GsTyrRS-EcOpt
Geobacillus stearothermophilus
MDLLAELQWRGLVNQTTDEDGLRELLKEERVTLYCGFDPTADSLHIGNLAAILTLRRFQQAGHQPIALVG
GATGLIGDPSGKKSERTLNAKETVEAWSARIQEQLSRFLDFEAHGNPAKIKNNYDWIGPLDVITFLRDVG
KHFSVNYMMAKESVQSRIETGISFTEFSYMMLQAYDFLRLYETEGCRLQIGGSDQWGNITAGLELIRKTK
GEARAFGLTIPLVTKADGTKFGKTESGTIWLDKEKTSPYEFYQFWINTDDRDVIRYLKYFTFLSKEEIEA
FEQELREAPEKRAAQKALAEEVTKLVHGEEALRQAVRISEALFSGDIGNLTAAEIEQGFKDVPSFVHEGG
DVPLVELLVSAGISPSKRQAREDIQNGAIYVNGERLQDVGAILTAEHRLEGRFTVIRRGKKKYYLIRYA
SEQ ID NO. 65
DNA
ValRS-GsValRS-EcOpt
Geobacillus (codon-optimized for E. coli)
ATGGCACAGCATGAAGTTAGCATGCCTCCGAAATATGATCATCGTGCAGTTGAAGCAGGTCGTTATGAAT
GGTGGCTGAAAGGTAAATTCTTTGAAGCAACCGGTGATCCGAATAAACGTCCGTTTACCATTGTTATTCC
GCCTCCGAATGTGACCGGTAAACTGCATCTGGGTCATGCATGGGATACCACACTGCAGGATATTATCACC
CGTATGAAACGTATGCAGGGTTATGATGTTCTGTGGCTGCCTGGTATGGATCATGCAGGTATTGCAACCC
AGGCAAAAGTTGAAGAAAAACTGCGTCAGCAGGGTCTGAGCCGTTATGATCTGGGTCGTGAAAAATTTCT
GGAAGAAACCTGGAAATGGAAAGAAGAATACGCAGGTCATATTCGTAGCCAGTGGGCAAAATTAGGTCTG
GGTTTAGATTATACCCGTGAACGTTTTACCCTGGATGAAGGTCTGAGCAAAGCAGTTCGTGAAGTTTTTG
TTAGCCTGTATCGTAAAGGTCTGATTTATCGCGGTGAGTATATCATTAATTGGGACCCTGTTACCAAAAC
CGCACTGAGCGATATTGAAGTGGTTTACAAAGAAGTTAAAGGCGCACTGTATCATCTGCGTTATCCGCTG
GCAGATGGTAGCGGTTGTATTGAAGTTGCAACCACACGTCCGGAAACCATGCTGGGTGATACCGCAGTTG
CAGTTCATCCTGATGATGAACGTTATAAACATCTGATCGGCAAAATGGTGAAACTGCCGATTGTTGGTCG
CGAAATTCCGATTATTGCAGATGAATATGTGGACATGGAATTTGGTAGTGGTGCCGTGAAAATTACACCG
GCACATGATCCGAACGATTTTGAAATTGGTAATCGCCATAATCTGCCTCGTATTCTGGTGATGAATGAAG
ATGGCACCATGAATGAAAATGCCATGCAGTATCAAGGTCTGGATCGTTTTGAATGCCGTAAACAAATTGT
TCGCGATCTGCAAGAACAGGGTGTTCTGTTTAAAATCGAAGAACATGTGCATAGCGTTGGTCATAGCGAA
CGTAGCGGTGCAGTTATTGAACCGTATCTGAGCACCCAGTGGTTTGTTAAAATGAAACCGCTGGCCGAAG
CAGCAATTAAACTGCAGCAGACCGATGGTAAAGTTCAGTTTGTGCCGGAACGCTTTGAAAAAACCTATCT
GCATTGGCTGGAAAACATTCGTGATTGGTGTATTAGCCGTCAGCTGTGGTGGGGTCATCGTATTCCGGCA
TGGTATCATAAAGAAACCGGTGAAATTTATGTGGATCACGAACCGCCTAAAGATATCGAAAATTGGGAAC
AAGATCCGGATGTTCTGGATACCTGGTTTAGCAGCGCACTGTGGCCGTTTAGCACCATGGGTTGGCCTGA
TGTTGAAAGTCCGGATTATAAACGTTATTATCCGACCGATGTGCTGGTTACCGGTTATGATATTATCTTT
TTTTGGGTGAGCCGCATGATTTTTCAAGGCCTGGAATTTACCGGCAAACGCCCTTTTAAAGATGTTCTGA
TTCATGGTCTGGTGCGTGATGCACAGGGTCGTAAAATGAGCAAAAGCTTAGGTAATGGTGTTGATCCGAT
GGATGTGATTGATCAGTATGGTGCAGATGCACTGCGTTATTTTCTGGCAACCGGTAGCAGCCCTGGTCAG
GATCTGCGTTTTAGCACCGAAAAAGTGGAAGCAACGTGGAATTTTGCCAACAAAATTTGGAATGCAAGCC
GTTTTGCACTGATGAACATGGGTGGTATGACCTATGAAGAACTGGATCTGAGCGGTGAAAAAACAGTTGC
GGATCATTGGATTCTGACCCGTCTGAATGAAACCATTGATACCGTTACCAAACTGGCCGAAAAATATGAA
TTTGGTGAAGCCGGTCGTACCCTGTATAACTTTATTTGGGATGATCTGTGCGATTGGTATATCGAAATGG
CAAAACTGCCGCTGTATGGTGATGATGAGGCAGCAAAAAAAACAACCCGTAGCGTTCTGGCATATGTGCT
GGATAATACCATGCGCCTGCTGCATCCGTTTATGCCGTTTATTACCGAAGAAATTTGGCAGAATCTGCCG
CATGAAGGTGAAAGCATTACCGTTGCACCGTGGCCTCAGGTTCGTCCGGAACTGAGCAATGAAGAGGCAG
CGGAAGAAATGCGTATGCTGGTTGATATTATTCGTGCCGTTCGTAATGTTCGTGCCGAAGTTAATACCCC
TCCGAGCAAACCGATTGCACTGTATATCAAAGTTAAAGACGAACAGGTTCGTGCAGCCCTGATGAAAAAT
CGTGCATATCTGGAACGTTTTTGCAATCCGAGCGAACTGCTGATTGATACCAATGTTCCTGCACCGGATA
AAGCAATGACCGCAGTGGTGACCGGTGCAGAACTGATTATGCCGCTGGAAGGCCTGATTAACATTGAAGA
AGAAATTAAACGCCTGGAAAAAGAACTTGATAAATGGAACAAAGAGGTGGAACGCGTCGAAAAAAAACTG
GCAAATGAAGGTTTTCTGGCCAAAGCACCAGCGCATGTTGTGGAAGAAGAACGTCGTAAACGTCAGGATT
ACATGGAAAAACGTGAAGCAGTTAAAGCACGTCTGGCCGAACTGAAACGTTAA
SEQ ID NO. 66
Amino Acid
ValRS-GsValRS-EcOpt
Geobacillus
MAQHEVSMPPKYDHRAVEAGRYEWWLKGKFFEATGDPNKRPFTIVIPPPNVTGKLHLGHAWDTTLQDIIT
RMKRMQGYDVLWLPGMDHAGIATQAKVEEKLRQQGLSRYDLGREKFLEETWKWKEEYAGHIRSQWAKLGL
GLDYTRERFTLDEGLSKAVREVFVSLYRKGLIYRGEYIINWDPVTKTALSDIEVVYKEVKGALYHLRYPL
ADGSGCIEVATTRPETMLGDTAVAVHPDDERYKHLIGKMVKLPIVGREIPIIADEYVDMEFGSGAVKITP
AHDPNDFEIGNRHNLPRILVMNEDGTMNENAMQYQGLDRFECRKQIVRDLQEQGVLFKIEEHVHSVGHSE
RSGAVIEPYLSTQWFVKMKPLAEAAIKLQQTDGKVQFVPERFEKTYLHWLENIRDWCISRQLWWGHRIPA
WYHKETGEIYVDHEPPKDIENWEQDPDVLDTWFSSALWPFSTMGWPDVESPDYKRYYPTDVLVTGYDIIF
FWVSRMIFQGLEFTGKRPFKDVLIHGLVRDAQGRKMSKSLGNGVDPMDVIDQYGADALRYFLATGSSPGQ
DLRFSTEKVEATWNFANKIWNASRFALMNMGGMTYEELDLSGEKTVADHWILTRLNETIDTVTKLAEKYE
FGEAGRTLYNFIWDDLCDWYIEMAKLPLYGDDEAAKKTTRSVLAYVLDNTMRLLHPFMPFITEEIWQNLP
HEGESITVAPWPQVRPELSNEEAAEEMRMLVDIIRAVRNVRAEVNTPPSKPIALYIKVKDEQVRAALMKN
RAYLERFCNPSELLIDTNVPAPDKAMTAVVTGAELIMPLEGLINIEEEIKRLEKELDKWNKEVERVEKKL
ANEGFLAKAPAHVVEEERRKRQDYMEKREAVKARLAELKR
SEQ ID NO. 67
DNA
MTF-GsMTF-EcOpt
Geobacillus stearothermophilus (codon-optimized for E. coli)
ATGACCAACATTGTGTTTATGGGCACACCGGATTTTGCAGTTCCGATTCTGCGTCAGCTGCTGCATGATG
GTTATCGTGTTGCAGCAGTTGTTACCCAGCCGGATAAACCGAAAGGTCGTAAACGTGAACCTGTTCCGCC
TCCGGTTAAAGTTGAAGCAGAACGTCGTGGTATTCCGGTTCTGCAGCCGACCAAAATTCGTGAACCGGAA
CAGTATGAACAGGTGCTGGCATTTGCACCGGATCTGATTGTTACCGCAGCATTTGGTCAGATTCTGCCGA
AAGCACTGCTGGATGCACCGAAATATGGTTGCATTAATGTTCATGCAAGCCTGCTGCCGGAACTGCGTGG
TGGTGCACCGATTCATTATGCAATTTGGCAGGGTAAAACCAAAACCGGTGTTACCATTATGTATATGGTT
GAACGTCTGGATGCCGGTGATATGCTGGCACAGGTTGAAGTGCCGATTGCAGAAACCGATACCGTTGGCA
CCCTGCATGATAAACTGAGCGCAGCGGGTGCAAAACTGCTGAGCGAAACCCTGCCGCTGCTGCTGGAAGG
CAATATTACACCGGTTCCGCAGGATGAAGAAAAAGCAACCTATGCACCTAATATTCGTCGTGAACAAGAA
CGTATTGATTGGACCCAGCCTGGTGAAGCCATTTATAACCATATTCGTGCCTTTCATCCGTGGCCTGTTA
CCTATACCACACAGGATGGTCATATTTGGAAAGTTTGGTGGGGTGAAAAAGTTCCTGCACCGCGTAGCGC
ACCGCCTGGCACCATTCTGGCACTGGAAGAAAATGGTATTGTTGTTGCAACCGGTAATGAAACCGCAATT
CGTATTACCGAACTGCAGCCTGCAGGTAAAAAACGTATGGCAGCCGGTGAATTTCTGCGTGGCGCAGGTA
GCCGTCTGGCAGTTGGTATGAAACTGGGTGAAGATCATGAACGTACCTAA
SEQ ID NO. 68
Amino Acid
MTF-GsMTF-EcOpt
Geobacillus stearothermophilus
MINIVFMGTPDFAVPILRQLLHDGYRVAAVVTQPDKPKGRKREPVPPPVKVEAERRGIPVLQPTKIREPE
QYEQVLAFAPDLIVTAAFGQILPKALLDAPKYGCINVHASLLPELRGGAPIHYAIWQGKTKTGVTIMYMV
ERLDAGDMLAQVEVPIAETDTVGTLHDKLSAAGAKLLSETLPLLLEGNITPVPQDEEKATYAPNIRREQE
RIDWTQPGEAIYNHIRAFHPWPVTYTTQDGHIWKVWWGEKVPAPRSAPPGTILALEENGIVVATGNETAI
RITELQPAGKKRMAAGEFLRGAGSRLAVGMKLGEDHERT
SEQ ID NO. 69
DNA
IF-1-GsuIF-1
Geobacillus subterraneus DSM 13552 (91A1)
ATGTTACTCATTCGAAGGAGGGAGAGCCGCTCGATGGCAAAAGACGATGTAATTGAAGTGGAAGGCACCG
TCATTGAAACATTGCCAAATGCGATGTTTCGTGTAGAATTAGAAAATGGGCACACAGTATTGGCCCATGT
GTCCGGCAAAATCCGTATGCACTTCATCCGCATTTTGCCTGGCGATAAAGTGACGGTGGAGTTGTCGCCG
TATGATTTAACGCGTGGACGGATTACGTATCGATATAAA
SEQ ID NO. 70
Amino Acid
IF-1-GsuIF-1
Geobacillus subterraneus DSM 13552 (91A1)
MLLIRRRESRSMAKDDVIEVEGTVIETLPNAMFRVELENGHTVLAHVSGKIRMHFlRILPGDKVTVELSP
YDLTRGRITYRYK
SEQ ID NO. 71
DNA
IF-2-GsuIF-2
Geobacillus subterraneus DSM 13552 (91A1)
ATGGTGTCCCGCTTTGCAAAGTGCCGGACCGGTATACGCTCGGCGGCGCGATCGGCAAAGACGCCCGCGT
CGTTGTCGCCGTCACCGACGAAGGGTTCGCGCGCCAATTGCAAACGATGCTCGACTGATCTTTATGGGGG
TGAATGTATGTCGAAAATGCGTGTGTACGAATACGCCAAAAAACATAATGTGCCAAGCAAGGACGTTATT
CATAAATTGAAAGAAATGAATATTGAAGTGAACAACCATATGACTATGCTCGAAGCCGATGTCGTCGAAA
AGCTCGATCATCAATACCGCGTGAACTCAGAGAAAAAAGCGGAAAAGAAAACGGAGAAACCGAAGCGGCC
GACGCCGGCGAAAGCCGCCGATTTTGCCGACGAGGAAATGTTTGAGGACAAGAAAGAAACGGCAAAGACG
AAGCCGGCGAAGAAAAAGGGAGCAGTGAAAGGAAAGGAAACGAAAAAAACAGAAGCACAGCAGCAAGAAA
AGAAACTGTTCCAAGCGGCGAAGAAAAAAGGAAAAGGACCGATGAAAGGCAAAAAACAAGCTGCCCCAGC
CTCAAAGCAGGCGCAGCAGCCGGCGAAAAAAGAAAAAGAGCTCCCGAAAAAAATTACGTTCGAAGGTTCG
CTCACGGTAGCCGAATTGGCGAAAAAACTTGGCCGCGAGCCGTCGGAAATCATTAAAAAACTGTTTATGC
TCGGCGTCATGGCGACGATTAACCAAGATTTAGACAAAGATGCGATCGAGCTCATTTGCTCTGATTACGG
AGTTGAAGTCGAAGAAAAAGTGACGATCGATGAAACGAATTTTGAAACGATCGAAATTGTCGATGCACCG
GAAGATTTGGTGGAACGGCCGCCGGTCGTCACGATTATGGGGCACGTTGACCACGGGAAAACAACGCTGC
TTGACGCAATCCGCCACTCGAAAGTGACCGAGCAAGAGGCGGGCGGTATTACACAGCATATCGGTGCTTA
TCAAGTCACGGTCAACGGCAAGAAAATTACGTTCCTCGATACGCCGGGGCATGAAGCGTTTACGACGATG
CGGGCGCGCGGTGCGCAAGTGACGGATATCGTCATCCTTGTTGTTGCTGCTGATGATGGGGTCATGCCGC
AGACGGTCGAGGCGATTAACCACGCCAAAGCGGCGAACGTACCGATTATCGTCGCCATTAACAAAATGGA
TAAGCCGGAAGCAAACCCGGATCGCGTTATGCAAGAGTTGATGGAGTACAACCTCGTTCCGGAAGAATGG
GGTGGCGATACGATTTTCTGCAAGCTGTCGGCGAAAACCCAAGACGGTATTGACCATCTGTTGGAAATGA
TTTTGCTTGTCAGCGAAATGGAAGAACTAAAAGCGAACCCGAACCGCCGCGCGCTCGGTACGGTGATCGA
AGCGAAGCTCGATAAAGGGCGCGGTCCGGTAGCGACGTTGCTCGTCCAAGCCGGTACGCTAAAAGTCGGT
GATCCGATTGTTGTCGGAACAACGTACGGACGCGTGCGCGCGATGGTCAATGACAGCGGTCGGCGTGTCA
AAGAAGCGGGTCCGTCGATGCCGGTCGAAATCACAGGGCTTCATGATGTGCCGCAAGCCGGGGACCGCTT
TATGGTATTTGAAGATGAGAAGAAAGCGCGACAAATCGGAGAAGCGCGGGCACAGCGGCAGCTGCAAGAG
CAGCGGAGCGTGAAAACGCGCGTCAGCTTGGACGATTTGTTTGAACAAATTAAGCAAGGTGAAATGAAAG
AGCTGAACTTGATCGTTAAGGCCGACGTCCAAGGATCGGTCGAAGCGCTTGTCGCCGCCTTGCAAAAAAT
CGATATCGAAGGCGTGCGTGTGAAAATTATCCACGCGGCGGTCGGCGCCATTACGGAGTCAGACATCTTG
TTGGCAACGACCTCGAACGCGATCGTCATCGGTTTTAACGTCCGTCCGGACACCAATGCGAAGCGGGCTG
CCGAATCAGAAAACGTCGACATCCGCCTCCACCGCATTATTTACAATGTCATCGAAGAAATTGAAGCGGC
GATGAAAGGGATGCTCGACCCAGAATATGAAGAAAAAGTGATCGGTCAGGCGGAAGTGCGGCAAACGTTC
AAAGTGTCGAAAGTCGGCACGATCGCCGGGTGCTACGTCACCGACGGCAAAATTACCCGCGACAGCAAAG
TGCGCCTTATCCGTCAAGGCATCGTCGTGTACGAAGGCGAAATCGACTCGCTCAAACGGTATAAAGATGA
TGTGCGTGAGGTGGCGCAAGGATACGAATGCGGCGTGACCATCAAAAACTTCAACGATATTAAAGAAGGG
GACGTCATCGAGGCGTACATCATGCAGGAAGTGGCTCGCGCA
SEQ ID NO. 72
Amino Acid
IF-2-GsuIF-2
Geobacillus subterraneus DSM 13552 (91A1)
MVSRFAKCRTGIRSAARSAKTPASLSPSPTKGSRANCKRCSTDLYGGECMSKMRVYEYAKKHNVPSKDVI
HKLKEMNIEVNNHMTMLEADVVEKLDHQYRVNSEKKAEKKTEKPKRPTPAKAADFADEEMFEDKKETAKT
KPAKKKGAVKGKETKKTEAQQQEKKLFQAAKKKGKGPMKGKKQAAPASKQAQQPAKKEKELPKKITFEGS
LTVAELAKKLGREPSEIIKKLFMLGVMATINQDLDKDAIELICSDYGVEVEEKVTIDETNFETIEIVDAP
EDLVERPPVVTIMGHVDHGKTTLLDAIRHSKVTEQEAGGITQHIGAYQVTVNGKKITFLDTPGHEAFTTM
RARGAQVTDIVILVVAADDGVMPQTVEAINHAKAANVPIIVAINKMDKPEANPDRVMQELMEYNLVPEEW
GGDTIFCKLSAKTQDGIDHLLEMILLVSEMEELKANPNRRALGTVIEAKLDKGRGPVAILLVQAGTLKVG
DPIVVGTTYGRVRAMVNDSGRRVKEAGPSMPVEITGLHDVPQAGDRFMVFEDEKKARQIGEARAQRQLQE
QRSVKTRVSLDDLFEQIKQGEMKELNLIVKADVQGSVEALVAALQKIDIEGVRVKIIHAAVGAITESDIL
LATTSNAIVIGFNVRPDTNAKRAAESENVDIRLHRIIYNVIEEIEAAMKGMLDPEYEEKVIGQAEVRQTF
KVSKVGTIAGCYVTDGKITRDSKVRLIRQGIVVYEGEIDSLKRYKDDVREVAQGYECGVTIKNFNDIKEG
DVIEAYIMQEVARA
SEQ ID NO. 73
DNA
IF-3-GsuIF-3
Geobacillus subterraneus DSM 13552 (91A1)
ATGGACTACGGCAAATTCCGCTTTGAGCAGCAAAAGAAAGAAAAAGAAGCGCGCAAAAAGCAAAAGGTGA
TCAACATTAAAGAGGTGCGCCTCAGCCCGACAATTGAGGAACACGACTTTAATACGAAACTACGCAATGC
GCGCAAGTTTTTAGAAAAAGGCGATAAAGTGAAGGCGACGATCCGCTTTAAAGGGCGGGCGATCACCCAT
AAAGAAATCGGGCAGCGCGTCCTTGACCGCTTCTCGGAAGCATGCGCTGATATCGCGGTCGTCGAAACGG
CGCCGAAATTGGAAGGGCGCAACATGTTTTTAGTGCTGGCACCGAAAAATGACAACAAG
SEQ ID NO. 74
Amino Acid
IF-3-GsuIF-3
Geobacillus subterraneus DSM 13552 (91A1)
MDYGKFRFEQQKKEKEARKKQKVINIKEVRLSPTIEEHDENTKLRNARKFLEKGDKVKATIRFKGRAITH
KEIGQRVLDRESEACADIAVVETAPKLEGRNMELVLAPKNDNK
SEQ ID NO. 75
DNA
EF-G-GsuEF-G
Geobacillus subterraneus DSM 13552 (91A1)
ATGGCAAGAGAGTTCTCCTTAGAAAACACTCGTAACATAGGAATCATGGCGCACATTGACGCCGGAAAAA
CGACGACGACGGAACGAATCCTGTTCTACACAGGCCGCGTTCATAAAATCGGGGAAACGCATGAAGGCTC
AGCTACGATGGACTGGATGGAACAAGAGCAAGAGCGCGGGATTACGATTACGTCGGCGGCGACAACGGCG
CAATGGAAAGGCCATCGCATCAACATCATCGACACGCCAGGGCACGTCGACTTCACGGTTGAGGTTGAAC
GTTCGTTGCGCGTGTTGGACGGAGCCATTACAGTTCTTGACGCCCAATCTGGTGTAGAACCGCAAACGGA
AACAGTTTGGCGTCAAGCGACTACATATGGTGTTCCGCGGATTGTATTCGTCAACAAAATGGACAAAATC
GGTGCGGACTTCTTGTATGCGGTAAAAACGCTCCATGACCGCTTACAAGCGAATGCCTACCCGGTGCAGT
TGCCGATCGGCGCTGAAGACCAATTCACCGGCATTATTGACCTCGTGGAAATGTGTGCATACCATTACCA
CGACGACCTTGGCAAAAACATCGAACGCATCGAAATTCCGGAAGACTACCGCGATTTAGCGGAAGAATAT
CATGGCAAGCTCATTGAGGCTGTTGCGGAACTCGATGAAGAGCTGATGATGAAATATTTAGAAGGAGAAG
AAATTACGAAAGAAGAGCTGAAAGCCGCAATCCGTAAGGCGACGATCAACGTTGAATTCTATCCAGTCTT
CTGCGGTTCAGCTTTTAAAAACAAAGGTGTTCAGCTGCTTCTTGACGGGGTTGTCGACTACTTGCCGTCT
CCGTTAGATATCCCGGCGATTCGCGGTATCATTCCGGATACGGAAGAAGAAGTGGCTCGCGAAGCACGCG
ATGACGCTCCGTTCTCCGCGTTGGCATTCAAAATTATGACTGACCCGTACGTTGGGAAGTTGACGTTCTT
CCGCGTCTACTCCGGAACGCTTGATTCCGGTTCTTACGTCATGAACTCAACGAAACGGAAGCGTGAACGG
ATCGGTCGCTTGCTGCAAATGCATGCGAACCACCGTCAAGAAATTTCGACAGTCTATGCCGGTGATATTG
CGGCAGCAGTAGGTTTAAAAGAAACAACGACCGGCGATACTCTATGTGATGAGAAAAATCTTGTCATCTT
AGAGTCGATGCAATTCCCAGAGCCGGTTATCTCGGTGGCGATCGAACCGAAATCGAAAGCCGACCAAGAT
AAGATGGGTCAAGCATTGCAAAAACTGCAAGAGGAAGACCCGACATTCCGTGCGCATACCGATCCGGAAA
CAGGACAAACGATCATTTCCGGGATGGGCGAGCTGCACTTGGACATTATCGTCGACCGGATGCGTCGCGA
ATTCAAAGTCGAGGCGAACGTTGGTGCACCGCAAGTTGCTTACCGTGAAACGTTCCGTCAATCGGCTCAA
GTCGAAGGGAAATTTATTCGCCAGTCCGGTGGTCGTGGTCAGTACGGTCACGTTTGGATCGAATTCACAC
CGAACGAACGCGGTAAAGGCTTTGAATTTGAAAATGCGATCGTCGGTGGGGTCGTTCCGAAAGAGTACGT
GCCGGCTGTTCAAGCTGGATTGGAAGAAGCGATGCAAAACGGTGTCTTAGCTGGCTACCCGGTTGTTGAC
ATCAAAGCGAAACTGTTTGATGGATCGTACCATGATGTCGACTCGAGTGAGATGGCGTTCAAAATTGCTG
CTTCGATGGCGTTGAAAAACGCGGCAGCGAAGTGTGAACCGGTTCTGCTTGAACCGATCATGAAAGTAGA
AGTCGTCATCCCTGAAGAATACCTCGGCGACATTATGGGTGACATCACATCCCGCCGCGGTCGCGTCGAA
GGGATGGAAGCGCGCGGAAACGCCCAAGTTGTTCGTGCAATGGTGCCGCTGGCCGAAATGTTCGGTTATG
CAACATCGCTCCGTTCGAACACGCAAGGGCGTGGAACGTTCTCGATGGTATTTGACCATTACGAAGAAGT
TCCGAAAAACATCGCCGATGAAATTATCTAAAGGCGAA
SEQ ID NO. 76
Amino Acid
EF-G-GsuEF-G
Geobacillus subterraneus DSM 13552 (91A1)
MAREFSLENTRNIGIMAHIDAGKITTTERILFYTGRVHKIGETHEGSATMDWMEQEQERGITITSAATTA
QWKGHRINIIDTPGHVDFTVEVERSLRVLDGAITVLDAQSGVEPQTETVWRQATTYGVPRIVFVNKMDKI
GADFLYAVKTLHDRLQANAYPVQLPIGAEDQFTGIIDLVEMCAYHYHDDLGKNIERIEIPEDYRDLAEEY
HGKLIEAVAELDEELMMKYLEGEEITKEELKAAIRKATINVEFYPVFCGSAFKNKGVQLLLDGVVDYLPS
PLDIPAIRGIIPDTEEEVAREARDDAPFSALAFKIMTDPYVGKLTFFRVYSGTLDSGSYVMNSTKRKRER
IGRLLQMHANHRQEISTVYAGDIAAAVGLKETTTGDTLCDEKNLVILESMQFPEPVISVAIEPKSKADQD
KMGQALQKLQEEDPTFRAHTDPETGQTIISGMGELHLDIIVDRMRREFKVEANVGAPQVAYRETFRQSAQ
VEGKFIRQSGGRGQYGHVWIEFTPNERGKGFEFENAIVGGVVPKEYVPAVQAGLEEAMQNGVLAGYPVVD
IKAKLFDGSYHDVDSSEMAFKIAASMALKNAAAKCEPVLLEPIMKVEVVIPEEYLGDIMGDITSRRGRVE
GMEARGNAQVVRAMVPLAEMFGYATSLRSNTQGRGTFSMVFDHYEEVPKNIADEIIKKNKGE
SEQ ID NO. 77
DNA
EF-Tu-GsuEF-Tu
Geobacillus subterraneus DSM 13552 (91A1)
ATGGCTAAAGCGAAATTTGAGCGTACGAAACCGCACGTCAACATTGGCACGATCGGCCACGTTGACCATG
GGAAAACGACGTTGACAGCTGCGATCACGACAGTTCTTGCGAAACAAGGTAAAGCAGAAGCGAGAGCGTA
CGACCAAATCGACGCTGCTCCGGAAGAGCGTGAACGCGGAATCACGATTTCGACGGCTCACGTTGAGTAT
GAAACAGAAAACCGTCACTATGCGCACGTTGACTGCCCGGGCCACGCTGACTACGTGAAAAACATGATCA
CGGGCGCAGCGCAAATGGACGGCGCGATCCTTGTTGTATCGGCTGCTGACGGTCCGATGCCGCAAACTCG
CGAACACATTCTTCTTTCCCGCCAAGTCGGTGTTCCGTACATCGTTGTTTTCTTGAACAAATGCGACATG
GTGGACGACGAAGAATTGCTTGAACTCGTTGAAATGGAAGTTCGCGATCTTCTTTCTGAATATGACTTCC
CGGGCGACGAAGTGCCGGTTATCAAAGGTTCGGCATTAAAAGCGCTCGAAGGCGATGCACAATGGGAAGA
AAAAATCGTTGAACTGATGAACGCGGTTGACGAGTACATCCCAACTCCGCAACGTGAAGTAGACAAACCG
TTCATGATGCCGGTTGAGGACGTCTTCTCGATCACGGGTCGTGGTACGGTTGCAACGGGCCGTGTTGAGC
GCGGTACGTTAAAAGTTGGTGACCCGGTTGAAATCATCGGTCTTTCGGACGAGCCGAAATCGACGACTGT
TACGGGTGTAGAAATGTTCCGTAAGCTTCTCGACCAAGCAGAAGCTGGTGACAACATCGGTGCGCTTCTC
CGCGGTGTATCGCGTGACGAAGTTGAGCGCGGTCAAGTATTGGCGAAACCGGGCTCGATCACGCCACACA
CGAAATTTAAAGCACAAGTTTACGTTCTGACGAAAGAAGAAGGCGGACGCCATACTCCGTTCTTCTCGAA
CTACCGTCCGCAATTCTACTTCCGTACAACGGACGTAACGGGCATCATCACGCTTCCAGAAGGCGTTGAA
ATGGTTATGCCTGGCGACAACGTTGAAATGACGGTTGAACTGATCGCTCCGATCGCGATCGAAGAAGGTA
CGAAATTCTCGATCCGTGAAGGCGGCCGCACGGTTGGTGCTGGTTCCGTATCGGAAATCATTGAG
SEQ ID NO. 78
Amino Acid
EF-Tu-GsuEF-Tu
Geobacillus subterraneus DSM 13552 (91A1)
MAKAKFERTKPHVNIGTIGHVDHGKTTLTAAITTVLAKQGKAEARAYDQIDAAPEERERGITISTAHVEY
ETENRHYAHVDCPGHADYVKNMITGAAQMDGAILVVSAADGPMPQTREHILLSRQVGVPYIVVFLNKCDM
VDDEELLELVEMEVRDLLSEYDFPGDEVPVIKGSALKALEGDAQWEEKIVELMNAVDEYIPTPQREVDKP
FMMPVEDVFSITGRGTVATGRVERGTLKVGDPVEIIGLSDEPKSTTVTGVEMFRKLLDQAEAGDNIGALL
RGVSRDEVERGQVLAKPGSITPHTKFKAQVYVLTKEEGGRHTPFFSNYRPQFYFRTTDVTGIITLPEGVE
MVMPGDNVEMTVELIAPIAIEEGTKFSIREGGRTVGAGSVSEIIE
SEQ ID NO. 79
DNA
EF-Ts-GsuEF-Ts
Geobacillus subterraneus DSM 13552 (91A1)
ATGGCGATTACAGCACAAATGGTAAAAGAGCTGCGCGAAAAAACGGGCGCAGGCATGATGGACTGCAAAA
AAGCGCTCACCGAAACGAACGGTGACATGGAAAAAGCGATCGACTGGCTGCGTGAAAAAGGAATTGCTAA
AGCAGCGAAAAAAGCAGATCGCATCGCAGCGGAAGGAATGACATACATCGCGACGGAAGGCAATGCGGCT
GTCATTTTGGAAGTAAACTCGGAAACGGACTTCGTTGCCAAAAACGAAGCGTTCCAAACGCTCGTTAAGG
AGCTGGCTGCACATCTGCTGAAACAAAAGCCAGCCACGCTTGATGAAGCGCTCGGACAAACGATGAGCAG
TGGTTCCACTGTTCAAGATTACATTAACGAAGCAGTTGCTAAAATCGGTGAAAAAATTACGCTCCGCCGC
TTTGCTGTTGTCAACAAAGCGGATGATGAAACGTTTGGCGCGTACTTGCACATGGGCGGGCGCATCGGCG
TATTAACATTATTAGCCGGCAACGCAACTGAAGAGGTCGCTAAAGATGTGGCGATGCATATTGCTGCGCT
CCATCCGAAATACGTTTCGCGCGATGAAGTGCCGCAAGAAGAGATTGCGCGCGAACGTGAAGTGTTGAAA
CAACAAGCGTTGAACGAAGGTAAGCCGGAAAACATCGTTGAAAAAATGGTTGAAGGCCGTCTGAAAAAGT
TTTACGAAGATGTTTGCCTGCTTGAGCAAGCGTTCGTGAAAAACCCGGATGTGACGGTACGCCAATACGT
CGAATCGAGCGGAGCAACCGTGAAGCAGTTCATCCGCTACGAAGTTGGTGAAGGGCTCGAAAAACGTCAA
GATAATTTCGCTGAAGAAGTCATGAGCCAAGTAAGAAAACAA
SEQ ID NO. 80
Amino Acid
EF-Ts-GsuEF-Ts
Geobacillus subterraneus DSM 13552 (91A1)
MAITAQMVKELREKTGAGMMDCKKALTETNGDMEKAIDWLREKGIAKAAKKADRIAAEGMTYIATEGNAA
VILEVNSETDFVAKNEAFQTLVKELAAHLLKQKPATLDEALGQTMSSGSTVQDYINEAVAKIGEKITLRR
FAVVNKADDETFGAYLHMGGRIGVLTLLAGNATEEVAKDVAMHIAALHPKYVSRDEVPQEEIAREREVLK
QQALNEGKPENIVEKMVEGRLKKFYEDVCLLEQAFVKNPDVTVRQYVESSGATVKQFIRYEVGEGLEKRQ
DNFAEEVMSQVRKQ
SEQ ID NO. 81
DNA
EF-4-GsuEF-4
Geobacillus subterraneus DSM 13552 (91A1)
ATGAACCGGGAAGAACGGTTGAAACGGCAGGAACGGATTCGCAACTTTTCGATTATCGCTCACATTGACC
ACGGAAAATCGACGCTTGCGGACCGCATTTTAGAAAAAACAGGTGCGCTGTCGGAGCGCGAGTTGCGCGA
GCAGACGCTCGATATGATGGAGCTCGAGCGCGAGCGCGGCATCACGATCAAATTGAATGCGGTCCAGTTG
ACATATAAAGCGAAAAACGGGGAAGAGTATATTTTCCATTTGATCGATACGCCGGGCCACGTCGATTTTA
CGTATGAAGTGTCGCGCAGCTTGGCTGCTTGCGAAGGAGCGATCTTAGTCGTCGATGCGGCGCAAGGCAT
TGAAGCGCAGACGCTCGCAAACGTGTATTTGGCCATTGACAACAATTTAGAAATTTTACCAGTCATTAAT
AAAATCGATTTGCCAAGCGCCGAGCCGGAGCGTGTCCGCCAAGAAATCGAAGACGTCATTGGCCTCGATG
CCTCTGAAGCGGTGCTCGCCTCCGCGAAAGTCGGCATCGGCGTCGAGGACATTTTAGAACAAATCGTGGA
AAAAATTCCTGCTCCGTCAGGCGATCCGGACGCGCCGTTGAAGGCGCTCATTTTTGATTCACTTTATGAC
CCGTACCGCGGCGTTGTCGCCTACGTCCGTATCGTCGATGGAACGGTTAAGCCGGGCCAGCGCATTAAAA
TGATGTCGACCGGCAAAGAGTTTGAAGTGACCGAAGTCGGCGTGTTTACACCAAAACCAAAAGTTGTCGA
CGAACTGATGGTCGGTGATGTCGGCTATTTAACTGCGTCGATCAAAAACGTACAAGATACGCGCGTCGGC
GATACGATTACCGATGCCGAACGGCCGGCTGCTGAGCCACTCCCTGGCTACCGGAAGCTCAATCCGATGG
TGTTTTGCGGCATGTACCCGATCGACACGGCGCGCTACAACGACTTGCGCGAAGCGTTAGAAAAGCTGCA
GCTCAACGATGCGGCGCTTCACTTTGAACCGGAAACGTCGCAGGCGCTCGGGTTTGGCTTTCGTTGCGGG
TTTCTCGGCTTGCTTCATATGGAGATTATCCAAGAGCGGATTGAACGTGAATTTCATATCGATTTAATTA
CAACGGCGCCGAGCGTTGTCTACAAAGTATATTTAACGGACGGAACGGAAGTCGATGTCGACAACCCGAC
GAACATGCCGGATCCGCAAAAAATCGACCGCATCGAAGAGCCGTATGTAAAAGCGACGATTATGGTGCCG
AACGACTACGTCGGACCGGTGATGGAGCTGTGCCAAGGAAAGCGTGGCACGTTCGTTGACATGCAATATT
TAGATGAAAAGCGGGTCATGTTGATTTACGATATTCCGCTGTCGGAAATCGTGTATGACTTTTTCGATGC
GTTAAAGTCGAACACGAAAGGGTATGCGTCGTTTGACTATGAATTGATCGGTTACCGGCCGTCCAATCTT
GTCAAAATGGATATTTTGTTGAATGGCGAAAAAATTGACGCTTTATCGTTTATTGTTCACCGCGATTCGG
CTTATGAGCGCGGCAAAGTGATCGTCGAGAAGCTGAAAGATTTAATTCCACGCCAACAGTTTGAAGTGCC
TGTGCAGGCGGCGATCGGCAATAAGATCATCGCCCGTTCGACGATCAAGGCGCTGCGTAAAAACGTGCTC
GCCAAATGTTACGGCGGCGACGTGTCGCGGAAACGGAAACTGCTTGAGAAACAAAAAGAAGGAAAGAAAC
GGATGAAACAAATCGGTTCGGTCGAAGTGCCGCAGGAAGCGTTTATGGCTGTCTTGAAAATCGACGACCA
GAAAAAA
SEQ ID NO. 82
Amino Acid
EF-4-GsuEF-4
Geobacillus subterraneus DSM 13552 (91A1)
MNREERLKRQERIRNFSIIAHIDHGKSTLADRILEKTGALSERELREQTLDMMELERERGITIKLNAVQL
TYKAKNGEEYIFHLIDTPGHVDFTYEVSRSLAACEGAILVVDAAQGIEAQTLANVYLAIDNNLEILPVIN
KIDLPSAEPERVRQEIEDVIGLDASEAVLASAKVGIGVEDILEQIVEKIPAPSGDPDAPLKALIFDSLYD
PYRGVVAYVRIVDGTVKPGQRIKMMSTGKEFEVTEVGVFTPKPKVVDELMVGDVGYLTASIKNVQDTRVG
DTITDAERPAAEPLPGYRKLNPMVFCGMYPIDTARYNDLREALEKLQLNDAALHFEPETSQALGFGFRCG
FLGLLHMEIIQERIEREFHIDLITTAPSVVYKVYLTDGTEVDVDNPTNMPDPQKIDRIEEPYVKATIMVP
NDYVGPVMELCQGKRGTFVDMQYLDEKRVMLIYDIPLSEIVYDFFDALKSNTKGYASFDYELIGYRPSNL
VKMDILLNGEKIDALSFIVHRDSAYERGKVIVEKLKDLIPRQQFEVPVQAAIGNKIIARSTIKALRKNVL
AKCYGGDVSRKRKLLEKQKEGKKRMKQIGSVEVPQEAFMAVLKIDDQKK
SEQ ID NO. 83
DNA
EF-P-GsuEF-P
Geobacillus subterraneus DSM 13552 (91A1)
ATGATTTCAGTGAACGATTTTCGCACAGGGCTTACGATTGAGGTCGACGGCGAGATTTGGCGCGTCCTTG
AGTTCCAGCATGTTAAGCCGGGCAAAGGGGCGGCGTTCGTCCGTTCGAAGCTGCGCAACTTGCGTACCGG
CGCCATTCAAGAGCGGACGTTCCGCGCTGGCGAAAAAGTAAACCGGGCACAAATTGATACGCGCAAAATG
CAATATTTATACGCTAACGGCGACTTGCATGTCTTTATGGATATGGAAACATACGAACAAATCGAGCTGC
CAGCGAAACAAATTGAGTATGAGCTGAAGTTCTTAAAAGAAAACATGGAAGTATTTATCATGATGTATCA
AGGCGAAACGATCGGTGTTGAGCTGCCGAACACCGTCGAGTTGAAAGTCGTTGAAACAGAGCCGGGCATC
AAAGGTGACACGGCTTCCGGCGGTTCGAAGCCGGCCAAGCTCGAAACCGGTCTTGTCGTTCAAGTGCCGT
TTTTCGTCAATGAAGGCGACACGCTCATCATTAACACGGCTGACGGTACGTACGTTTCGCGGGCA
SEQ ID NO. 84
Amino Acid
EF-P-GsuEF-P
Geobacillus subterraneus DSM 13552 (91A1)
MISVNDFRTGLTIEVDGEIWRVLEFQHVKPGKGAAFVRSKLRNLRTGAIQERTFRAGEKVNRAQIDTRKM
QYLYANGDLHVFMDMETYEQIELPAKQIEYELKFLKENMEVFIMMYQGETIGVELPNTVELKVVETEPGI
KGDTASGGSKPAKLETGLVVQVPFFVNEGDTLIINTADGTYVSRA
SEQ ID NO. 85
DNA
RF-1-GsuRF-1
Geobacillus subterraneus DSM 13552 (91A1)
ATGGATCCAGCCGTTATCAACGACCCGAAAAAGTTGCGCGATTATTCGAAAGAGCAGGCTGATTTGACTG
AAACGGTGCAAACGTACCGTGAATACAAGTCCGTTCGCAGTCAGCTCGCGGAAGCGAAGGCTATGCTGGA
AGAAAAACTTGAGCCAGAGCTGCGCGAGATGGTGAAAGAGGAAATTGATGAGCTCGAAGAACGGGAAGAA
GCGCTCGTTGAGAAGTTGAAAGTGTTGCTTTTGCCGAAAGATCCGAATGATGAGAAAAACGTCATTATGG
AAATTCGTGCCGCCGCCGGTGGCGAGGAAGCCGCGCTGTTTGCCGGCGACTTGTACCGGATGTATACGCG
CTATGCGGAGTCGCAAGGGTGGAAAACGGAAGTGATCGAAGCAAGCCCAACAGGTCTTGGCGGCTATAAA
GAAATCATCTTTATGGTCAATGGGAAAGGGGCGTATTCGAAGCTGAAGTTTGAAAACGGCGCTCATCGCG
TCCAACGCGTCCCGGAAACGGAATCAGGCGGACGCATCCATACATCGACGGCAACGGTCGCCTGCTTGCC
GGAAATGGAAGAAGTCGAAGTCGAAATTCATGAAAAAGACATTCGCGTCGATACGTACGCCTCGAGCGGG
CCAGGGGGACAAAGCGTGAACACGACGATGTCAGCCGTACGCCTCACCCATATTCCGACCGGCATTGTCG
TTACTTGCCAAGACGAAAAATCGCAAATTAAAAACAAAGAAAAAGCGATGAAAGTGTTGCGCGCCCGCAT
TTACGACAAATACCAGCAAGAAGCGCGCGCCGAGTATGACCAAACGCGTAAGCAAGCAGTCGGCACCGGC
GATCGCTCAGAGCGCATCCGCACGTACAACTTCCCGCAAAACCGCGTCACTGACCACCGTATCGGGTTGA
CGATTCAAAAGCTTGACCTCGTGTTAGACGGGCAGCTCGATGAAATTATCGAGGCGCTCATTTTAGACGA
CCAGTCGAAAAAACTGGAGCAAGCGAACGATGCGTCG
SEQ ID NO. 86
Amino Acid
RF-1-GsuRF-1
Geobacillus subterraneus DSM 13552 (91A1)
MDPAVINDPKKLRDYSKEQADLTETVQTYREYKSVRSQLAEAKAMLEEKLEPELREMVKEEIDELEEREE
ALVEKLKVLLLPKDPNDEKNVIMEIRAAAGGEEAALFAGDLYRMYTRYAESQGWKTEVIEASPTGLGGYK
EIIFMVNGKGAYSKLKFENGAHRVQRVPETESGGRIHTSTATVACLPEMEEVEVEIHEKDIRVDTYASSG
PGGQSVNTTMSAVRLTHIPTGIVVTCQDEKSQIKNKEKAMKVLRARIYDKYQQEARAEYDQTRKQAVGTG
DRSERIRTYNFPQNRVTDHRIGLTIQKLDLVLDGQLDEIIEALILDDQSKKLEQANDAS
SEQ ID NO. 87
DNA
RF-2-Gsu-RF2
Geobacillus subterraneus DSM 13552 (91A1)
ATGGCCGCGCCCGGCTTTTGGGATGACCAGAAAGCGGCGCAGGCGATCATTTCCGAAGCGAATGCGCTCA
AGGAATTAGTCGGCGAGTTTGAATCGCTCGCGGAACGGTTCGACAACTTGGAAGTGACGTATGAGTTGTT
GAAAGAGGAGCCGGATGACGAGCTGCAGGCTGAACTTGTGGAAGAAGCGAAAAAATTGACGAAAGACTTC
AGCCAGTTTGAGCTGCAGCTGTTGCTCAACGAGCCGTACGACCAAAATAACGCGATTTTGGAGCTTCATC
CGGGTGCGGGCGGCACGGAATCGCAAGACTGGGCGTCGATGCTGTTGCGCATGTACACGCGCTGGGCGGA
GAAAAAAGGATTTAAAGTCGAAACACTGGATTATCTCCCAGGCGAGGAAGCCGGGGTGAAAAGCGTCACC
TTGCTTATCAAGGGACATAATGCATACGGCTACTTAAAGGCGGAAAAAGGGGTACACCGGCTTGTGCGCA
TCTCCCCGTTTGACGCCTCAGGCCGCCGCCATACGTCGTTCGTGTCATGCGAAGTCGTGCCGGAGATGGA
CGATAACATTGAGATTGAGATCCGTCCGGAAGAGCTGAAAATCGACACGTACCGCTCAAGCGGTGCGGGC
GGGCAGCACGTCAACACGACCGACTCCGCGGTGCGCATCACCCACTTGCCGACCGGCATTGTCGTTACGT
GCCAATCGGAGCGGTCGCAAATTAAAAACCGCGAAAAAGCGATGAATATGTTAAAAGCGAAGCTGTATCA
AAAGAAAATGGAGGAACAGCAAGCTGAACTCGCCGAGCTGCGCGGCGAGCAAAAAGAAATCGGCTGGGGC
AGCCAAATCCGCTCCTACGTCTTCCATCCGTATTCGCTTGTCAAAGACCATCGGACGAATGTGGAGGTCG
GCAACGTGCAAGCGGTGATGGATGGGGAAATCGATGTGTTCATTGACGCGTATTTGCGCGCGAAATTGAA
G
SEQ ID NO. 88
Amino Acid
RF-2-GsuRF-2
Geobacillus subterraneus DSM 13552 (91A1)
MAAPGFWDDQKAAQAIISEANALKELVGEFESLAERFDNLEVTYELLKEEPDDELQAELVEEAKKLTKDF
SQFELQLLLNEPYDQNNAILELHPGAGGTESQDWASMLLRMYTRWAEKKGFKVETLDYLPGEEAGVKSVT
LLIKGHNAYGYLKAEKGVHRLVRISPFDASGRRHTSFVSCEVVPEMDDNIEIEIRPEELKIDTYRSSGAG
GQHVNTTDSAVRITHLPTGIVVTCQSERSQIKNREKAMNMLKAKLYQKKMEEQQAELAELRGEQKEIGWG
SQIRSYVFHPYSLVKDHRTNVEVGNVQAVMDGEIDVFIDAYLRAKLK
SEQ ID NO. 89
DNA
RRF-GsuRRF
Geobacillus subterraneus DSM 13552 (91A1)
ATGGCAAAGCAAGTGATCCAACAGGCGAAAGAAAAAATGGATAAAGCTGTGCAAGCGTTCAGCCGCGAGT
TGGCGACCGTCCGTGCCGGTCGGGCGAACGCGGGGTTGCTTGAGAAAGTAACCGTTGACTATTACGGTGT
CGCAACGCCGATCAACCAGCTCGCTACGATCAGCGTGCCGGAAGCGCGTATGCTTGTCATTCAGCCGTAT
GACAAATCGGTCATTAAAGAAATGGAAAAAGCGATTTTAGCGTCGGACTTAGGAGTGACGCCGTCGAATG
ACGGATCGGTTATCCGCCTTGTCATTCCGCCGCTTACTGAAGAACGTCGCCGTGAACTGGCGAAGCTCGT
CAAAAAATATTCGGAAGAAGCGAAAGTTGCGGTGCGCAACATCCGTCGCGATGCAAACGATGAGCTGAAA
AAACTCGAGAAAAATAGCGAGATTACGGAAGATGAGCTGCGCAGCTATACCGACGAAGTGCAAAAGCTGA
CCGACAGCCATATCGCCAAAATTGACGCCATCACAAAAGAGAAAGAAAAAGAAGTGATGGAAGTA
SEQ ID NO. 90
Amino Acid
RRF-GsuRRF
Geobacillus subterraneus DSM 13552 (91A1)
MAKQVIQQAKEKMDKAVQAFSRELATVRAGRANAGLLEKVTVDYYGVATPINQLATISVPEARMLVIQPY
DKSVIKEMEKAILASDLGVTPSNDGSVIRLVIPPLTEERRRELAKLVKKYSEEAKVAVRNIRRDANDELK
KLEKNSEITEDELRSYTDEVQKLTDSHIAKIDAITKEKEKEVMEV
SEQ ID NO. 91
DNA
AlaRS-GsuAlaRS
Geobacillus subterraneus DSM 13552 (91A1)
ATGAGAGTTTTTTTATATAAAAGACCAAAGGGGAGGATTGTTATGAAAAAGTTAACATCTGCCGAAGTGC
GGCGTATGTTTTTGCAGTTTTTCCAAGAAAAAGGCCATGCGGTCGAGCCGAGCGCTTCGCTCATTCCTGT
CGATGACCCGTCGTTATTATGGATCAACAGCGGTGTCGCGACGCTGAAAAAATATTTTGATGGCCGTATC
ATCCCGGACAACCCGCGCATTTGCAATGCGCAAAAATCGATCCGCACAAACGACATCGAAAATGTCGGGA
AAACGGCTCGCCACCATACGTTTTTTGAAATGCTCGGCAACTTTTCGATCGGCGATTATTTCAAGCGTGA
AGCGATTCATTGGGCATGGGAGTTTTTAACAAGTGAAAAGTGGATTGGTTTTGATCCAGAGCGGTTGTCA
GTCACTGTTCATCCGGAAGACGAAGAGGCGTATAACATTTGGCGCAACGAGATCGGTCTTCCTGAAGAGC
GGATTATTCGTTTAGAAGGAAACTTCTGGGATATCGGTGAAGGCCCGAGCGGTCCGAACACGGAAATTTT
TTATGACCGCGGTGAAGCGTTCGGCAACGATCCAAACGATCCAGAACTGTATCCAGGCGGGGAAAATGAC
CGCTACTTAGAAGTATGGAATCTCGTCTTTTCACAGTTCAACCATAACCCGGACGGCACGTACACGCCGC
TGCCGAAGAAAAACATCGATACCGGCATGGGCTTAGAGCGGATGTGCTCGATTTTGCAAGATGTACCGAC
GAACTTTGAAACTGATTTGTTCATGCCGATCATCCGCGCGACTGAGCAGATCGCGGGTGAGCAATACGGC
AAAGATCCGAATAAAGACGTTGCTTTTAAGGTCATCGCTGACCATATTCGTGCCGTGACGTTTGCGGTCG
GCGACGGGGCGCTGCCGTCGAACGAAGGACGAGGCTATGTATTGCGCCGCCTGCTTCGCCGCGCTGTGCG
CTATGCGAAACAAATCGGCATTGACCGTCCATTTATGTATGAGCTTGTTCCGGTTGTCGGTGAAATTATG
CAAGACTATTATCCGGAAGTGAAAGAAAAAGCCGATTTCATCGCCCGCGTCATTCGGACGGAAGAAGAGC
GGTTCCACGAAACGCTTCATGAAGGGCTCGCCATTTTGGCAGAAGTGATGGAAAAGGCGAAAAAACAAGG
AAGCACCGTCATTCCAGGAGAAGAGGCGTTCCGCTTGTACGATACGTACGGCTTCCCGCTCGAGCTGACG
GAAGAATATGCTGCTGAAGCGGGCATGTCGGTCGATCACGCCGGTTTTGAGCGCGAGATGGAGCGCCAGC
GCGAACGGGCCCGTGCCGCTCGCCAAGATGTCGATTCGATGCAAGTGCAAGGCGGGGTGCTCGGCGACAT
TAAAGACGAAAGCCGTTTTGTCGGCTACGATGAGCTCGTCGTTTCTTCGACGGTCATTGCCATCATTAAA
GACGGACAGCTCGTGGAGGAAGTCGGGACTGGCGAGGAAGCACAAATCATCGTTGATGTGACGCCGTTTT
ACGCCGAAAGCGGCGGACAAATCGCTGACCAAGGTGTGTTTGAAGGCGAAACGGGAACAGCGGTCGTCAA
AGATGTGCAAAAAGCACCGAACGGTCAGCACCTCCATTCGATTGTCGTCGAACGCGGTGCGGTGAAAAAA
GGCGATCGCTATACGGCGCGCGTCGATGAAGTGAAGCGGTCGCAAATCGTGAAAAACCATACGGCGACCC
ACTTGCTTCATCAAGCGTTAAAAGACGTTCTTGGCCGCCATGTCAACCAGGCCGGATCACTCGTTGCCCC
GGATCGGCTTCGCTTTGACTTTACTCATTTCGGGCAAGTGAAGCCTGATGAGCTCGAGCGCATTGAGGCG
ATCGTCAATGAACAAATTTGGAAGAGTATTCCGGTCGACATTTTTTACAAACCGCTCGAGGAAGCAAAAG
CGATGGGGGCGATGGCGCTGTTTGGTGAAAAATACGGCGATATCGTCCGCGTTGTTAAAGTTGGCGACTA
CAGCTTAGAGTTGTGCGGCGGCTGCCATGTGCCGAATACAGCGGCCATTGGGTTGTTTAAAATCGTCTCC
GAGTCCGGCATCGGTGCCGGCACGCGCCGGATTGAAGCGGTGACTGGGGAAGCGGCATACCGCTTTATGA
GCGAACAGCTTGCTCTGTTGCAAGAAGCGGCGCAAAAGCTGAAAACGAGCCCGAGAGAGCTGAATGCCCG
CCTTGATGGGCTGTTTGCCGAACTGCGCCAACTGCAGCGCGAAAATGAGTCGCTTGCTGCCCGTCTCGCC
CATATGGAGGCGGAACACCTCACCCGTCAAGTGAAAGAGGTGGGCGGTGTGCCGGTATTAGCCGCAAAAG
TGCAGGCGAACGACATGAACCAATTGCGGGCGATGGCTGATGACTTGAAGCAAAAACTAGGGACGGCGGT
CATCGTGTTAGCGGCCGTGCAAGGTGGCAAAGTCCAATTGATTGCTGCGGTGACTGATGACTTAGTGAAA
AAAGGATACCACGCCGGCAAACTCGTCAAAGAAGTGGCTTCACGTTGCGGCGGCGGAGGCGGCGGACGTC
CTGATATGGCGCAGGCCGGTGGGAAGGACGCGAACAAAGTCGGCGAAGCGCTCGATTATGTCGAAACATG
GGTCAAATCCATTTCC
SEQ ID NO. 92
Amino Acid
AlaRS-GsuAlaRS
Geobacillus subterraneus DSM 13552 (91A1)
MRVFLYKRPKGRIVMKKLTSAEVRRMFLQFFQEKGHAVEPSASLIPVDDPSLLWINSGVATLKKYFDGRI
IPDNPRICNAQKSIRINDIENVGKTARHHTFFEMLGNFSIGDYFKREAIHWAWEFLTSEKWIGFDPERLS
VTVHPEDEEAYNIWRNEIGLPEERIIRLEGNFWDIGEGPSGPNTEIFYDRGEAFGNDPNDPELYPGGEND
RYLEVWNLVFSQFNHNPDGTYTPLPKKNIDTGMGLERMCSILQDVPTNFETDLFMPIIRATEQIAGEQYG
KDPNKDVAFKVIADHIRAVIFAVGDGALPSNEGRGYVLRRLLRRAVRYAKQIGIDRPFMYELVPVVGEIM
QDYYPEVKEKADFIARVIRTEEERFHETLHEGLAILAEVMEKAKKQGSTVIPGEEAFRLYDTYGFPLELT
EEYAAEAGMSVDHAGFEREMERQRERARAARQDVDSMQVQGGVLGDIKDESRFVGYDELVVSSTVIAIIK
DGQLVEEVGTGEEAQIIVDVTPFYAESGGQIADQGVFEGETGTAVVKDVQKAPNGQHLHSIVVERGAVKK
GDRYTARVDEVKRSQIVKNHTATHLLHQALKDVLGRHVNQAGSLVAPDRLRFDFTHFGQVKPDELERIEA
IVNEQIWKSIPVDIFYKPLEEAKAMGAMALFGEKYGDIVRVVKVGDYSLELCGGCHVPNTAAIGLFKIVS
ESGIGAGTRRIEAVTGEAAYRFMSEQLALLQEAAQKLKTSPRELNARLDGLFAELRQLQRENESLAARLA
HMEAEHLTRQVKEVGGVPVLAAKVQANDMNQLRAMADDLKQKLGTAVIVLAAVQGGKVQLIAAVTDDLVK
KGYHAGKLVKEVASRCGGGGGGRPDMAQAGGKDANKVGEALDYVETWVKSIS
SEQ ID NO. 93
DNA
ArgRS-GsuArgRS
Geobacillus subterraneus DSM 13552 (91A1)
ATGAACATTGTCGGACAAATGAAAGAACAGCTGAAAGAGGAAATTCGCCAGGCGGTGGGAAAAGCCGGGC
TGGTGGCGGCTGAGGAGCTGCCAGAAGTATTGCTTGAGGTGCCGCGCGAAAAGGCTCATGGCGATTATTC
GACGAATATCGCCATGCAGCTCGCCCGCATCGCGAAAAAGCCACCGCGGGCAATCGCCGAAGCCATCGTT
GAAAAGTTTGACGCCGAGCGTGTTTCGGTGGCGCGCATCGAGGTAGCCGGCCCAGGGTTTATTAACTTTT
ACATGGACAATCGCTATTTGACAGCGGTTGTGCCGGCGATTTTGCAAGCGGGCCAAGCGTATGGCGAGTC
GAATGTCGGCAAAGGGGAAAAAGTGCAAGTCGAGTTCGTCTCGGCTAACCCGACCGGCAACTTGCATTTA
GGTCATGCTCGCGGTGCGGCGGTTGGCGATTCACTTAGCAATATTTTGGCGAAAGCCGGATTCGATGTGA
CGCGTGAATATTACATTAATGATGCCGGCAAACAAATTTATAACTTGGCGAAATCAGTCGAAGCCCGCTA
TTTCCAAGCGCTCGGTACCGATATGCCGCTGCCGGAGGACGGCTATTACGGTGACGACATCGTGGAAATC
GGCAAAAAGCTCGCCGATGAATATGGCGATCGGTTCGTCCATGTGGACGAAGAAGAACGACTCGCCTTTT
TCCGCGAATACGGCCTCCGTTATGAGCTCGACAAAATTAAAAACGATTTGGCTGCCTTCCGCGTTCCATT
TGACGTTTGGTATTCGGAAACATCGCTTTATGAGAGCGGCAAAATCGATGAGGCGCTCTCAACGCTGCGT
GAGCGCGGTTACATTTACGAACAGGACGGAGCCACATGGTTTCGTTCGACGGCGTTTGGCGATGACAAAG
ACCGTGTGTTAATCAAGCAAGACGGAACGTATACGTATTTGCTTCCGGACATCGCTTACCATCAAGATAA
GCTGCGGCGTGGGTTCACGAAGCTAATCAACGTCTGGGGAGCGGATCATCATGGCTACATCCCGCGCATG
AAAGCGGCGATCGCTGCGCTCGGCTACGATCCAGAAGCGCTCGAGGTCGAAATTATCCAAATGGTGAACT
TATACCAAAACGGCGAGCGCGTCAAAATGAGCAAACGTACTGGCAAAGCGGTGACGATGCGCGAGCTGAT
GGAAGAAGTCGGCGTCGATGCTGTCCGCTACTTCTTCGCTATGCGTTCGGGCGATACGCATCTCGATTTT
GATATGGACTTGGCTGTTGCCCAGTCGAATGAAAACCCGGTCTACTATGTCCAATATGCACATGCCCGCG
TCTCAAGCATTCTCCGTCAAGCAAAAGAGCATCAACTGTCGTATGAAGGCGACGTCGATCTTCATCATCT
CGTGGAAACAGAAAAAGAAATCGAGCTGCTCAAAGCGCTTGGCGACTTCCCGGACGTTGTCGCTGAGGCG
GCCTTGAAACGGATGCCACATCGCGTCACCGCCTATGCGTTTGATTTGGCGTCGGCGCTCCACAGCTTTT
ACAATGCGGAAAAAGTGCTTGACCTAGACCAGATCGAAAAAACGAAAGCTCGTCTCGCGCTTGTCAAGGC
GGTGCAAATCACGCTGCAAAACGCTCTAGCGTTAATCGGCGTCTCAGCGCCGGAACAAATG
SEQ ID NO. 94
Amino Acid
ArgRS-GsuArgRS
Geobacillus subterraneus DSM 13552 (91A1)
MNIVGQMKEQLKEEIRQAVGKAGLVAAEELPEVLLEVPREKAHGDYSTNIAMQLARIAKKPPRAIAEAIV
EKFDAERVSVARIEVAGPGFINFYMDNRYLTAVVPAILQAGQAYGESNVGKGEKVQVEFVSANPTGNLHL
GHARGAAVGDSLSNILAKAGFDVTREYYINDAGKQIYNLAKSVEARYFQALGTDMPLPEDGYYGDDIVEI
GKKLADEYGDRFVHVDEEERLAFFREYGLRYELDKIKNDLAAFRVPFDVWYSETSLYESGKIDEALSTLR
ERGYIYEQDGATWFRSTAFGDDKDRVLIKQDGTYTYLLPDIAYHQDKLRRGFTKLINVWGADHHGYIPRM
KAAIAALGYDPEALEVEIIQMVNLYQNGERVKMSKRTGKAVTMRELMEEVGVDAVRYFFAMRSGDTHLDF
DMDLAVAQSNENPVYYVQYAHARVSSILRQAKEHQLSYEGDVDLHHLVETEKEIELLKALGDFPDVVAEA
ALKRMPHRVTAYAFDLASALHSFYNAEKVLDLDQIEKTKARLALVKAVQITLQNALALIGVSAPEQM
SEQ ID NO. 95
DNA
AsnRS-GsuAsnRS
Geobacillus subterraneus DSM 13552 (91A1)
ATGGACGTGTCGATTATTGGAGGGAATGTGTACGTGAAAACGACGATTGCTGAAGTGAACCAATATGTAG
GTCAAGAAGTCACGATCGGCGCTTGGTTGGCGAACAAGCGCTCGAGCGGAAAAATCGCCTTTTTACAGCT
GCGTGATGGGACTGGCTTTATTCAAGGTGTAGTTGAAAAAGCGAACGTCTCAGAAGAGGTATTTCAACGT
GCGAAAACGCTGACGCAAGAAACGTCGCTCTATGTGACCGGCACGGTGCGCGTCGACGAGCGTTCACCGT
TCGGTTATGAGCTTTCGGTGACGAACATACAGGTCATCAATGAAGCGGTCGATTATCCGATTACGCCAAA
AGAACACGGTGTCGAGTTTTTAATGGATCATCGTCACCTTTGGCTTCGTTCGCGGCGCCAACATGCGATC
ATGAAAATCCGCAACGAATTGATCCGTGCGACGTATGAGTTTTTTAACGAACGTGGCTTCGTCAAAGTCG
ATGCGCCGATTTTGACTGGCAGCGCACCGGAAGGAACGACCGAGCTGTTCCATACGAAGTATTTTGACGA
GGATGCCTATTTATCGCAAAGCGGCCAGCTATATATGGAAGCAGCAGCCATGGCGCTCGGTAAAGTGTTT
TCGTTCGGTCCGACATTCCGTGCCGAAAAGTCGAAAACGCGCCGCCATTTGATCGAATTTTGGATGATCG
AGCCTGAAATGGCGTTTTACGAATTTGAAGACAATTTGCGGCTGCAAGAAGAGTATGTCTCTTATCTCGT
ACAGTCGGTGCTTAGCCGTTGCCAACTTGAGCTCGGGCGCCTTGGACGCGACGTCACCAAGCTTGAGCTT
GTCAAGCCGCCGTTTCCGCGTCTAACGTATGACGAAGCGATCAAGCTGCTGCATGACAAAGGGTTTACCG
ATATCGAATGGGGCGATGACTTCGGTGCGCCGCATGAGACAGCCATCGCTGAAAGCTTCGACAAGCCGGT
GTTTATCACTCACTACCCGACGTCGTTAAAGCCGTTTTATATGCAGCCAGATCCGAACCGTCCGGACGTC
GTGCTATGTGCTGATTTAATCGCGCCGGAGGGATACGGGGAGATTATCGGCGGTTCCGAGCGCATTCATG
ATTATGAGCTGCTCAAGCAGCGTCTCGAGGAGCATCATTTGCCGCTTGAAGCATATGAATGGTATTTAGA
TTTGCGCAAATACGGTTCCGTGCCGCACTCCGGATTCGGGCTCGGCCTCGAGCGAACGGTTGCTTGGATT
TGCGGCGTTGAGCATGTACGCGAGACGATCCCGTTTCCGCGGTTGCTCAACCGTCTATACCCG
SEQ ID NO. 96
Amino Acid
AsnRS-GsuAsnRS
Geobacillus subterraneus DSM 13552 (91A1)
MDVSIIGGNVYVKTTIAEVNQYVGQEVTIGAWLANKRSSGKIAFLQLRDGTGFIQGVVEKANVSEEVFQR
AKTLTQETSLYVTGTVRVDERSPFGYELSVTNIQVINEAVDYPITPKEHGVEFLMDHRHLWLRSRRQHAI
MKIRNELIRATYEFFNERGFVKVDAPILTGSAPEGTTELFHTKYFDEDAYLSQSGQLYMEAAAMALGKVF
SFGPTFRAEKSKTRRHLIEFWMIEPEMAFYEFEDNLRLQEEYVSYLVQSVLSRCQLELGRLGRDVTKLEL
VKPPFPRLTYDEAIKLLHDKGFTDIEWGDDFGAPHETAIAESFDKPVFITHYPTSLKPFYMQPDPNRPDV
VLCADLIAPEGYGEIIGGSERIHDYELLKQRLEEHHLPLEAYEWYLDLRKYGSVPHSGFGLGLERTVAWI
CGVEHVRETIPFPRLLNRLYP
SEQ ID NO. 97
DNA
AspRS-GsuAspRS
Geobacillus subterraneus DSM 13552 (91A1)
ATGTTTCAAACACTTGAGCTTCGTCATAAAGTGGCGAAGGCGGTGCGCAACTTTTTAGACGGCGAACGCT
TTTTAGAAGTGGAGACGCCAATGTTGACGAAAAGCACACCGGAAGGGGCGCGCGATTATTTAGTGCCAAG
CCGCGTTCATCCGGGGGAATTTTACGCCTTGCCGCAGTCGCCGCAAATTTTTAAGCAGCTTTTGATGGTC
GGCGGTTTTGAACGCTATTACCAAATCACTCGTTGCTTCCGCGATGAAGATTTGCGCGCTGACCGCCAGC
CAGAGTTTACGCAAATTGACATTGAAATGTCGTTTGTCGACCAAGAAGACATCATCGATTTAACCGAACG
GATGATGGCGGCGGTCGTCAAAGCAACTAAAGGGATTGACATTCCGCGCCCATTTCCACGCATCACGTAT
GACGAAGCGATGAGCCGTTACGGTTCCGATAAGCCGGACGTACGTTTTGGCCTTGAGCTTGTCGATGTGT
CGGAAGCGGTCCGCGGCTCCGCGTTTCAAGTGTTCGCCCGCGCCGTTGAGCAAGGTGGTCAAGTGAAGGC
AATCAACGTAAAAGGAGCGGCGAGCCGTTATTCGCGTAAAGACATTGACGCGTTAGCGGAGTTTGCCGGC
CGCTACGGAGCGAAAGGGCTCGCTTGGTTAAAAGTTGAAGGCGGGGAGCTGAAAGGGCCGATCGCCAAGT
TTTTCGTCGATGATGAGCAAACAGCGCTGCGCCAGCTGCTTGCTGCCGAAGATGGGGATTTGCTGTTGTT
TGTTGCTGACGAGAAGGCGATTGTCGCGGCGGCTCTTGGTGCGTTGCGGTTAAAGCTCGGCAAAGAGCTT
GGCTTGATCGATGAAACGAAGCTCGCTTTTTTATGGGTAACAGATTGGCCGCTTTTAGAGTACGACGAAG
AAGAAGGCCGCTATTACGCCGCCCACCATCCGTTTACGATGCCGGTGCGTGACGATATCCCGCTGCTTGA
GACAAACCCAGGCGCTGTTCGGGCGCAGGCGTATGATTTAGTGTTAAACGGCTATGAGCTTGGCGGCGGT
TCGCTCCGTATTTTTGAGCGCGATGTACAAGAAAAAATGTTCCGCGCTCTAGGATTTGACCAGGAAGAGG
CGCGCCGCCAGTTTGGCTTCCTGCTTGAGGCGTTTGAATATGGCACTCCGCCGCATGGCGGTATCGCCCT
CGGCCTCGATCGACTTGTGATGCTCTTAGCTGGGCGCACAAACTTGCGCGATACGATCGCCTTCCCGAAA
ACTGCGAGCGCCAGCTGCCTGCTTACTGAAGCGCCGGGACCGGTCAGTGAAAAACAACTGAAAGAGTTGC
ATTTGGCTGTGGTGCTTCCCGACCAGCAA
SEQ ID NO. 98
Amino Acid
AspRS-GsuAspRS
Geobacillus subterraneus DSM 13552 (91A1)
MFQTLELRHKVAKAVRNFLDGERFLEVETPMLTKSTPEGARDYLVPSRVHPGEFYALPQSPQIFKQLLMV
GGFERYYQITRCFRDEDLRADRQPEFTQIDIEMSFVDQEDIIDLTERMMAAVVKATKGIDIPRPFPRITY
DEAMSRYGSDKPDVRFGLELVDVSEAVRGSAFQVFARAVEQGGQVKAINVKGAASRYSRKDIDALAEFAG
RYGAKGLAWLKVEGGELKGPIAKFFVDDEQTALRQLLAAEDGDLLLFVADEKAIVAAALGALRLKLGKEL
GLIDETKLAFLWVTDWPLLEYDEEEGRYYAAHHPFTMPVRDDIPLLETNPGAVRAQAYDLVLNGYELGGG
SLRIFERDVQEKMFRALGFDQEEARRQFGFLLEAFEYGTPPHGGIALGLDRLVMLLAGRTNLRDTIAFPK
TASASCLLTEAPGPVSEKQLKELHLAVVLPDQQ
SEQ ID NO. 99
DNA
CysRS-GsuCysRS
Geobacillus subterraneus DSM 13552 (91A1)
ATGAAAGGAAGAGCGAATATGAGCAGTATCCGACTTTATAATACGTTGACGCGAAAAAAGGAAACGTTTG
AGCCGCTCGAACCGAACAAAGTGAAAATGTATGTATGTGGCCCGACGGTCTATAATTATATTCATATCGG
CAATGCTCGCGCCGCTATCGTCTTTGATACGATCCGCCGTTATTTAGAGTTCCGCGGTTATGATGTGACG
TATGTATCCAACTTTACTGATGTCGACGACAAGCTAATCAGGGCGGCCCGCGAGCTTGGTGAGAGCGTGC
CGGCGATCGCCGAGCGGTTTATTGAGGCGTATTTTGAGGACATTGAGGCGCTCGGCTGCAAAAAAGCAGA
TATCCATCCGCGCGTGACGGAAAATATCGAAACGATTATCGAATTCATTCAAGCGCTCATTGACAAAGGC
TATGCGTACGAAGTCGATGGTGACGTATACTATCGGACGCGCAAGTTTGATGGCTACGGCAAATTGTCGC
ATCAGTCGATCGATGAGCTACAAGCGGGGGCGCGCATCGAAGTTGGGGAAAAGAAAGATGATCCACTCGA
TTTTGCTCTTTGGAAAGCAGCGAAAGAAGGAGAGATTTCTTGGGACAGCCCATGGGGGAAAGGGCGGCCC
GGCTGGCATATCGAATGTTCAGCGATGGCGCGCAAATATTTAGGAGATACGATCGACATTCATGCTGGCG
GCCAAGACTTAACGTTTCCACACCATGAAAACGAAATTGCCCAATCGGAAGCACTGACCGGCAAACCGTT
TGCGAAATATTGGCTGCACAATGGGTATTTAAATATTAACAATGAAAAAATGTCCAAGTCGCTTGGCAAC
TTTGTACTTGTTCACGATATCATCCGGCAGATTGACCCACAAGTGTTGCGTTTCTTTATGCTGTCGGTGC
ACTATCGCCACCCGATCAACTATAGCGAGGAGCTGCTTGAGAGCGCTCGGCGTGGTCTCGAACGCTTGAG
GACAGCATACGGTAATTTGCAGCACCGGCTTGGGGCGAGCACGAACTTAACCGATAACGACGGCGAGTGG
CTTTCGCGCCTCGCGGATATCCGCGCCTCGTTCATTCGTGAAATGGACGATGATTTCAACACAGCAAACG
GCATTGCGGTCTTGTTCGAGCTCGCCAAACAAGCGAACTTGTATTTGCAGGAGAAAACGACATCCGAGAA
TGTCATTCACGCGTTTTTGCGCGAATTTGAGCAGCTGATGGATGTACTCGGCCTTACTTTGAAACAAGAG
GAGTTGCTTGACGAAGAAATTGAGGCGCTGATCCGCCAGCGCAATGAAGCGCGGAAAAATCGTGACTTTG
CCTTAGCCGACCGCATCCGCGACGAGTTGAAAGCAAAAAATATCATTTTGGAAGATACGCCGCAAGGGAC
GAGATGGAAACGGGGATCG
SEQ ID NO. 100
Amino Acid
CysRS-GsuCysRS
Geobacillus subterraneus DSM 13552 (91A1)
MKGRANMSSIRLYNTLTRKKETFEPLEPNKVKMYVCGPTVYNYIHIGNARAAIVFDTIRRYLEFRGYDVT
YVSNFTDVDDKLIRAARELGESVPAIAERFIEAYFEDIEALGCKKADIHPRVTENIETIIEFIQALIDKG
YAYEVDGDVYYRTRKFDGYGKLSHQSIDELQAGARIEVGEKKDDPLDFALWKAAKEGEISWDSPWGKGRP
GWHIECSAMARKYLGDTIDIHAGGQDLTFPHHENEIAQSEALTGKPFAKYWLHNGYLNINNEKMSKSLGN
FVLVHDIIRQIDPQVLRFFMLSVHYRHPINYSEELLESARRGLERLRTAYGNLQHRLGASTNLIDNDGEW
LSRLADIRASFIREMDDDFNTANGIAVLFELAKQANLYLQEKTTSENVIHAFLREFEQLMDVLGLTLKQE
ELLDEEIEALIRQRNEARKNRDFALADRIRDELKAKNIILEDTPQGTRWKRGS
SEQ ID NO. 101
DNA
GluRS-GsuGluRS
Geobacillus subterraneus DSM 13552 (91A1)
ATGGAATTGGAGGTTTGGACGATGGCAAAAAACGTGCGCGTGCGCTATGCGCCGAGCCCGACTGGCCATT
TGCATATCGGTGGGGCACGGACAGCGCTGTTTAACTATTTGTTTGCCCGCCATTACGGCGGAAAAATGAT
CGTCCGCATCGAAGATACGGATATTGAACGGAACGTTGAAGGCGGCGAAGAGTCGCAGCTTGAAAACTTA
AAATGGCTTGGCATCGATTATGACGAATCGATTGATAAGGACGGCGGATATGGGCCGTATCGTCAGACGG
AACGGCTCGATATCTATCGGAAGTATGTGAACGAGCTGCTTGAACAAGGGCATGCGTATAAATGTTTTTG
TACACCGGAAGAGCTCGAGCGGGAACGTGAGGAGCAACGGGCGGCAGGTATTGCTGCTCCGCAATACAGC
GGCAAATGCCGCCATTTAACGCCGGAGCAAGTTGCCGAGCTTGAAGCACAAGGAAAACCGTATACGATCC
GCTTGAAAGTGCCGGAAGGGAAAACGTATGAAGTAGATGATTTAGTGCGCGGTAAAGTGACGTTTGAATC
GAAAGACATCGGCGATTGGGTCATTGTGAAGGCGAACGGTATTCCGACGTACAACTTTGCCGTTGTCATT
GATGACCATTTGATGGAAATCAGCCATGTGTTCCGCGGTGAGGAGCATTTATCCAACACGCCGAAACAGC
TAATGGTGTACGAATATTTCGGTTGGGAGCCACCGCAATTCGCCCATATGACATTGATTGTCAACGAGCA
GCGGAAAAAGCTATCCAAGCGCGATGAATCGATTATCCAGTTCGTGTCGCAATATAAAGAGCTCGGCTAT
TTGCCGGAGGCGATGTTCAACTTTTTCGCCCTTCTTGGCTGGTCGCCGGAAGGAGAAGAAGAAATTTTTA
CGAAGGACGAGCTCATCCGCATTTTTGATGTCGCCCGGCTGTCGAAATCGCCGTCGATGTTTGATACGAA
AAAGCTGACATGGATGAACAACCAATATATCAAAAAGCTGGATCTCGACAGGCTTGTCGAGCTGGCGTTG
CCGCATTTAGTGAAAGCCGGACGCCTGCCGGCAGATATGAGTGATGAGCAGCGGCAATGGGCACGCGATT
TGATTGCCTTGTACCAAGAGCAAATGAGCTACGGTGCGGAGATCGTTTCGCTGTCCGAGCTGTTCTTTAA
AGAAGAAGTCGAATACGAAGACGAAGCCCGCCAAGTGCTCGCCGAAGAACAAGTACCGGATGTGCTCTCC
GCCTTTTTGGCGAATGTGCGTGAGCTTGAGCCGTTTACGGCGGATGAGATTAAAGCAGCGATCAAAGCAG
TGCAAAAATCGACAGGGCAAAAAGGCAAGAAGCTGTTTATGCCGATTCGCGCCGCAGTGACTGGGCAAAC
ACACGGACCGGAACTGCCGTTTGCCATCCAACTGCTTGGCAAACAAAAGGTGATTGAACGGCTCGAACGG
GCACTGCATGAAAAATTT
SEQ ID NO. 102
Amino Acid
GluRS-GsuGluRS
Geobacillus subterraneus DSM 13552 (91A1)
MELEVWTMAKNVRVRYAPSPTGHLHIGGARTALFNYLFARHYGGKMIVRIEDTDIERNVEGGEESQLENL
KWLGIDYDESIDKDGGYGPYRQTERLDIYRKYVNELLEQGHAYKCFCTPEELEREREEQRAAGIAAPQYS
GKCRHLTPEQVAELEAQGKPYTIRLKVPEGKTYEVDDLVRGKVTFESKDIGDWVIVKANGIPTYNFAVVI
DDHLMEISHVFRGEEHLSNTPKQLMVYEYFGWEPPQFAHMTLIVNEQRKKLSKRDESIIQFVSQYKELGY
LPEAMFNFFALLGWSPEGEEEIFTKDELIRIFDVARLSKSPSMFDTKKLTWMNNQYIKKLDLDRLVELAL
PHLVKAGRLPADMSDEQRQWARDLIALYQEQMSYGAEIVSLSELFFKEEVEYEDEARQVLAEEQVPDVLS
AFLANVRELEPFTADEIKAAIKAVQKSTGQKGKKLFMPIRAAVTGQTHGPELPFAIQLLGKQKVIERLER
ALHEKF
SEQ ID NO. 103
DNA
GlyRS-GsuGlyRS
Geobacillus subterraneus DSM 13552 (91A1)
ATGGAGGAGGATGATGACATGGCTGCAACAATGGAAGAAATCGTTGCCCACGCCAAGCATCGCGGCTTCG
TGTTTCCGGGGTCGGAAATTTACGGTGGGCTGGCGAACACATGGGATTACGGTCCGCTCGGTGTCGAGCT
GAAAAATAACATTAAACGGGCGTGGTGGAAAAAGTTCGTCCAAGAATCGCCACACAATGTCGGTTTGGAC
GCTGCCATTTTAATGAACCCAAAAACGTGGGAAGCATCCGGCCATTTAGGCAACTTCAACGATCCGATGG
TCGACTGCAAACAGTGTAAAGCGCGTCATCGCGCCGACAAGCTGATTGAGCAGGCACTTGAAGAAAAAGG
AATTGAGATGGTCGTTGACGGTTTGCCGCTTGCCAAGATGGAAGAGCTTATCCGTGAATACGACATCGCT
TGTCCAGAATGCGGCAGTCGTGACTTTACGAACGTGCGTCAGTTTAATTTAATGTTCAAAACATACCAAG
GTGTCACCGAATCAAGCGCTAACGAAATTTATTTGCGCCCGGAGACGGCCCAAGGTATTTTTGTCAACTT
TAAAAACGTCCAGCGCACGATGCGCAAAAAATTACCGTTTGGCATCGCGCAAATCGGAAAAAGTTTCCGC
AACGAAATTACGCCAGGGAACTTTACGTTCCGCACACGTGAATTTGAACAAATGGAGCTTGAGTTTTTCT
GCAAACCGGGCGAAGAGCTGAAATGGTTCGACTACTGGAAACAATTTTGCAAGGAATGGCTGTTGTCGCT
CGGCATGAACGAAGAACATATCCGCCTGCGCGACCATACGAAAGAAGAATTATCCCACTATAGTAATGCG
ACGACTGATATCGAGTATCAGTTCCCGTTCGGCTGGGGCGAGCTCTGGGGTATTGCGTCGCGCACCGATT
ACGACTTAAAACAGCATATGGAACACTCCGGTGAGGATTTCCATTATCTTGACCAAGAAACGAATGAGCG
CTACATCCCGTACTGCATTGAGCCGTCGCTCGGTGCCGACCGTGTCACGCTCGCGTTTATGATTGACGCC
TATGACGAGGAAGAGCTCGAAGACGGCACGACCCGGACAGTTATGCATTTGCATCCAGCGCTTGCGCCGT
ACAAAGCAGCTGTCTTGCCGTTATCGAAAAAGCTGGGTGACGGAGCGCGCCGAATTTATGAAGAGCTCGC
GAAGCATTTCATGGTCGACTACGATGAAACAGGTTCGATTGGCAAGCGGTATCGTCGTCAAGATGAAATC
GGCACGCCGTTTTGTATCACGTACGACTTTGAGTCCGAGCAAGATGGCCAAGTAACCGTTCGTGACCGTG
ACACGATGGAACAAGTGCGGTTGCCGATTGGGGAGCTCAAAGCCTTTTTGGATAAAAAAATTGCCTTT
SEQ ID NO. 104
Amino Acid
GlyRS-GsuGlyRS
Geobacillus subterraneus DSM 13552 (91A1)
MEEDDDMAATMEEIVAHAKHRGFVFPGSEIYGGLANTWDYGPLGVELKNNIKRAWWKKFVQESPHNVGLD
AAILMNPKTWEASGHLGNFNDPMVDCKQCKARHRADKLIEQALEEKGIEMVVDGLPLAKMEELIREYDIA
CPECGSRDFTNVRQFNLMFKTYQGVTESSANEIYLRPETAQGIFVNFKNVQRTMRKKLPFGIAQIGKSFR
NEITPGNFTFRTREFEQMELEFFCKPGEELKWFDYWKQFCKEWLLSLGMNEEHIRLRDHTKEELSHYSNA
TTDIEYQFPFGWGELWGIASRTDYDLKQHMEHSGEDFHYLDQETNERYIPYCIEPSLGADRVTLAFMIDA
YDEEELEDGTTRTVMHLHPALAPYKAAVLPLSKKLGDGARRIYEELAKHFMVDYDETGSIGKRYRRQDEI
GTPFCITYDFESEQDGQVTVRDRDTMEQVRLPIGELKAFLDKKIAF
SEQ ID NO. 105
DNA
HisRS-GsuHisRS
Geobacillus subterraneus DSM 13552 (91A1)
ATGGCTTTTCAAATTCCAAGAGGGACACAAGATTTATTACCGGGTGAAACGGAAAAATGGCAATATGTCG
AACAAGTGGCCCGCGACCTGTGTAGACGGTACGGCTATGAAGAAATACGGACGCCGATTTTTGAACATAC
GGAGCTGTTTTTACGTGGCGTTGGTGATACGACCGATATCGTCCAAAAAGAGATGTACACGTTTGAAGAC
AAAGGGGGCCGTGCGTTGACGCTCCGTCCGGAAGGAACCGCACCGGTCGTGCGGGCGTTCGTCGAGCATA
AGCTGTACGGCAGCCCGAATCAGCCGGTCAAGTTGTATTATGCGGGACCAATGTTCCGTTATGAGCGGCC
GGAAGCCGGACGGTTCCGCCAATTCGTCCAGTTTGGTGTTGAGGCAATTGGCAGCAGTGATCCGGCGATT
GACGCCGAGGTGATGGCGTTAGCGATGCATATTTATAAGGCGCTTGGTTTAAAACACATCCGGCTCGTAA
TCAACAGTTTAGGCGATGTAGACAGCCGCCGGGCGCATCGCGAAGCGCTTGTCCGCCATTTTTCTGACCG
CATTCATGAACTGTGCCCGGACTGTCAGGCGCGGCTTGAGACGAATCCGCTCCGCATTCTCGATTGTAAA
AAGGACCGCGATCATGAACTGATGGCGTCAGCACCGTCGATTTTAGACTATTTGAATGACGAATCGCGCG
CGTATTTTGAGAAGGTGAAGCAATATTTAACGATGCTTGACATCCCGTTTGTCATTGACTCGCGGCTCGT
GCGCGGCCTCGATTATTACAACCATACGACGTTTGAAATTATGAGCGAGGCTGAAGGATTCGGCGCAGCG
GCGACTCTTTGCGGCGGCGGACGCTATAACGGGCTTGTGCAAGAAATTGGCGGCCCGGAAACGCCTGGCA
TCGGCTTTGCGTTAAGCATTGAACGGCTGCTGGCGGCGCTTGAAGCGGAAGGGATTGAACTGCCGATCCA
TCGAGGAATCGATTGCTATGTTGTCGCTGTCGGTGAGCGGGCAAAAGATGAAACTGTCCGCCTCGTTTAC
GAATTGCGCCGTGCCGGCCTGCGTGTGGAGCAAGACTATTTAGGTCGAAAAATGAAGGCACAGCTGAAGG
CAGCTGACCGTCTTGGCGCATCATTCGTTGCCATCATCGGCGACGAGGAGCTGGAAAAACAGACAGCAGC
TGTGAAACACATGGCGAGCGGCGAGCAAACTGATGTGCCGCTTGGAGAGTTGGCGTCCTTTTTAATAGAA
CGAACAAAACGGGAGGAG
SEQ ID NO. 106
Amino Acid
HisRS-GsuHisRS
Geobacillus subterraneus DSM 13552 (91A1)
MAFQIPRGTQDLLPGETEKWQYVEQVARDLCRRYGYEEIRTPIFEHTELFLRGVGDTTDIVQKEMYTFED
KGGRALTLRPEGTAPVVRAFVEHKLYGSPNQPVKLYYAGPMFRYERPEAGRFRQFVQFGVEAIGSSDPAI
DAEVMALAMHIYKALGLKHIRLVINSLGDVDSRRAHREALVRHFSDRIHELCPDCQARLETNPLRILDCK
KDRDHELMASAPSILDYLNDESRAYFEKVKQYLTMLDIPFVIDSRLVRGLDYYNHTTFEIMSEAEGFGAA
ATLCGGGRYNGLVQEIGGPETPGIGFALSIERLLAALEAEGIELPIHRGIDCYVVAVGERAKDETVRLVY
ELRRAGLRVEQDYLGRKMKAQLKAADRLGASFVAIIGDEELEKQTAAVKHMASGEQTDVPLGELASFLIE
RTKREE
SEQ ID NO. 107
DNA
IleRS-GsuIleRS
Geobacillus subterraneus DSM 13552 (91A1)
ATGGACTACAAAGAGACGCTGCTCATGCCGCAAACGGAGTTCCCGATGCGTGGCAACTTGCCGAAGCGGG
AGCCGGAAATGCAAAAAAAATGGGAGGAAATGGACATTTACCGGAAAGTGCAGGAGCGGACGAAAGGACG
GCCGCTGTTTGTGCTGCACGACGGCCCGCCATACGCCAACGGTGATATTCATATGGGCCATGCATTAAAT
AAAATTTTAAAAGATATTATCGTCCGCTACAAGTCGATGAGCGGCTTTTGTGCGCCGTATGTGCCTGGCT
GGGATACACATGGCTTACCGATTGAAACGGCACTGACGAAGCAAGGTGTCGACCGCAAATCGATGAGTGT
CGCTGAGTTCCGCAAGCTGTGCGAACAATACGCGTATGAGCAAATCGACAACCAGCGCCAACAGTTTAAA
CGGCTCGGGGTGCGGGGCGATTGGGACAACCCGTACATTACGCTCAAGCCGGAATACGAAGCCCAGCAAA
TTAAAGTGTTCGGTGAAATGGCGAAAAAAGGGCTCATTTATAAAGGGCTGAAGCCGGTGTATTGGTCGCC
GTCGAGCGAATCGGCGCTCGCCGAAGCGGAAATCGAATATAAAGACAAACGGTCGCCGTCGATTTATGTC
GCGTTCCCAGTTAAAGATGGTAAAGGTGTGCTTCAAGGGGATGAACGAATCGTCATTTGGACGACGACAC
CGTGGACGATTCCAGCGAACTTGGCGATCGCCGTTCACCCGGATTTGGACTACTATATTGTCGAAGCAAA
CGGGCAAAAATACGTTGTTGCTGCGGCCTTGGCGGAATCGGTAGCGAAAGAAGTCGGCTGGGAGGCATGG
TCCGTCGTCAAAACGGTAAAAGGAAAAGAACTTGAGTACGTAGTCGCCAAACATCCGTTTTACGAGCGCG
ACTCGCTTGTCGTCTGCGGCGAGCACGTCACGACCGACGCCGGTACCGGCTGCGTTCATACGGCACCAGG
ACACGGGGAAGACGACTTTATCGTCGGACAAAAATACGGGCTTCCGGTTCTTTGCCCGGTTGATGAGCGC
GGCTATATGACAGAAGAAGCGCCTGGATTTGCAGGGATGTTTTACGACGAGGCGAACAAAGCGATTACAC
AAAAGCTCGAGGAAGTTGGAGCGCTCCTTAAGCTCAGCTTCATTACCCACTCGTATCCGCATGATTGGCG
GACGAAGCAACCGACAATTTTCCGAGCGACGACACAATGGTTTGCCTCCATTGATAAAATTCGTGATCAA
CTTCTTGATGCCATCAAGGAAACGAAATGGGTGCCAGAATGGGGAGAAATCCGCATCCATAACATGGTGC
GCGACCGCGGTGACTGGTGCATCTCCCGCCAACGCGCTTGGGGCGTGCCAATTCCGGTCTTTTACGGCGA
AAACGGCGAGCCGATCATCACAGATGAGACGATCGAGCACGTGTCAAACCTATTCCGCCAGTACGGCTCG
AATGTTTGGTTTGAGCGTGAGGCGAAAGACTTATTGCCGGAAGGATTCACCCATCCGTCCAGCCCGAACG
GCCTCTTTACGAAAGAGACGGATATTATGGACGTCTGGTTTGACTCCGGTTCGTCGCATCAAGCCGTGCT
TGTTGAACGCGATGACCTAGAGCGTCCGGCTGATTTATACTTAGAAGGATCTGACCAATATCGCGGCTGG
TTTAACTCGTCGCTGTCTACAGCCGTTGCCGTCACCGGAAAAGCACCGTATAAAGGGGTGTTAAGCCATG
GCTTCGTTTTAGACGGCGAAGGGCGAAAAATGAGCAAATCGCTCGGCAACGTCGTCGTGCCGGCCAAAGT
CATGGAACAGCTCGGTGCCGACATTTTACGCCTTTGGGTCGCCTCGGTTGACTATCAGGCGGATGTACGC
ATTTCCGATAACATTTTAAAACAAGTGTCCGAAGTGTATCGGAAAATCCGCAATACGTTCCGCTTTATGC
TCGGCAACTTGTTTGATTTTGACCCGAATCAAAACGCTGTGCCGGTTGGGGAGCTTGGCGAAGTCGATCG
CTACATGTTAGCGAAATTAAATAAACTCATCGCTAAAGTGAAAAAGGCGTATGACAGCTATGATTTTGCT
GCTGTTTATCATGAGATGAACCATTTCTGCACCGTCGAGTTAAGCGCATTTTATTTGGATATGGCGAAAG
ACATTTTGTACATCGAAGCGGCCGATTGTCGTGCCCGCCGTGCGGTGCAGACGGTGCTGTATGAAACGGT
TGTCGCCTTGGCGAAGCTCATTGCGCCGATTTTGCCGCACACGGCCGATGAAGTGTGGGAGCATATCCCG
AACCGGAAAGAGCAAGTGGAAAGCGTCCAGCTCACCGACATGCCGGAGTCAATGGCCATCGATGGTGAAG
AAGCGCTGCTTGCGAAATGGGATGCGTTTATGGATGTACGAGATGACATTTTAAAAGCGCTCGAGAATGC
GCGTAATGAAAAAGTGATCGGTAAGTCGCTCACGGCGAGCGTCACTGTTTACCCGAAAGACGAAGTGCGG
GCGCTTTTGGCTTCGATCAACGAGGACTTGCGCCAACTTCTCATCGTTTCCGCGTTTTCGGTCGCCGATG
AATCGTATGACGCCGCGCCAGCCGAAGCAGAACGGCTCAACCATGTGGCCGTCATCGTTCGCCCGGCGGA
AGGTGAGACGTGCGAACGTTGCTGGACGGTGACACCGGACGTCGGACGCGATGAGTCCCACCCGACGCTT
TGTCCGCGCTGCGCACATATTGTGAACGAACATTATTCGGCA
SEQ ID NO. 108
Amino Acid
IleRS-GsuIleRS
Geobacillus subterraneus DSM 13552 (91A1)
MDYKETLLMPQTEFPMRGNLPKREPEMQKKWEEMDIYRKVQERTKGRPLFVLHDGPPYANGDIHMGHALN
KILKDIIVRYKSMSGFCAPYVPGWDTHGLPIETALTKQGVDRKSMSVAEFRKLCEQYAYEQIDNQRQQFK
RLGVRGDWDNPYITLKPEYEAQQIKVFGEMAKKGLIYKGLKPVYWSPSSESALAEAEIEYKDKRSPSIYV
AFPVKDGKGVLQGDERIVIWTTTPWTIPANLAIAVHPDLDYYIVEANGQKYVVAAALAESVAKEVGWEAW
SVVKTVKGKELEYVVAKHPFYERDSLVVCGEHVTTDAGTGCVHTAPGHGEDDFIVGQKYGLPVLCPVDER
GYMTEEAPGFAGMFYDEANKAITQKLEEVGALLKLSFITHSYPHDWRTKQPTIFRATTQWFASIDKIRDQ
LLDAIKETKWVPEWGEIRIHNMVRDRGDWCISRQRAWGVPIPVFYGENGEPIITDETIEHVSNLFRQYGS
NVWFEREAKDLLPEGFTHPSSPNGLFTKETDIMDVWFDSGSSHQAVLVERDDLERPADLYLEGSDQYRGW
FNSSLSTAVAVTGKAPYKGVLSHGFVLDGEGRKMSKSLGNVVVPAKVMEQLGADILRLWVASVDYQADVR
ISDNILKQVSEVYRKIRNTFRFMLGNLFDFDPNQNAVPVGELGEVDRYMLAKLNKLIAKVKKAYDSYDFA
AVYHEMNHFCTVELSAFYLDMAKDILYIEAADCRARRAVQTVLYETVVALAKLIAPILPHTADEVWEHIP
NRKEQVESVQLTDMPESMAIDGEEALLAKWDAFMDVRDDILKALENARNEKVIGKSLTASVTVYPKDEVR
ALLASINEDLRQLLIVSAFSVADESYDAAPAEAERLNHVAVIVRPAEGETCERCWTVTPDVGRDESHPTL
CPRCAHIVNEHYSA
SEQ ID NO. 109
DNA
LeuRS-GsuLeuRS
Geobacillus subterraneus DSM 13552 (91A1)
ATGAGGAGGAGTGCGACGATGAGTTTCAACCATCGCGAAATTGAGAAAAAGTGGCAGGATTATTGGGAAC
AGCATAAAACGTTCCGCACCCCGGATGAAAGCGATAAACCGAAGTTTTACGTGTTGGATATGTTTCCGTA
TCCGTCTGGCGCTGGCTTGCACGTCGGCCATCCGGAAGGGTATACGGCGACTGATATTTTGGCGCGCATG
AAGCGGATGCAAGGGTACAATGTCCTTCACCCGATGGGGTGGGACGCGTTCGGATTGCCGGCAGAACAAT
ATGCGCTCGATACCGGCAACGACCCGGCCGAATTTACGCAAAAAAACATCGACAACTTCCGCCGGCAAAT
TAAGTCGCTTGGTTTTTCGTATGACTGGGATCGGGAAATTAACACGACTGATCCGAACTATTACAAATGG
ACGCAATGGATTTTCTTGAAGCTGTATGAAAAAGGGCTCGCCTACATGGACGAAGTACCGGTCAACTGGT
GTCCGGCGCTTGGCACCGTGCTGGCGAACGAAGAAGTCATCAACGGCCGGAGCGAGCGCGGTGGGCATCC
GGTCATCCGCAAGCCAATGCGGCAATGGATGCTGAAAATTACCGCCTATGCCGACCGGCTGCTCGAAGAT
TTGGAGGAGCTTGACTGGCCGGAAAGCATTAAAGAAATGCAACGCAACTGGATCGGCCGTTCGGAAGGAG
CGGAAATTGAGTTTGCTGTCGACGGCCATGACGAGTCGTTCACGGTATTTACGACGCGGCCAGATACGCT
GTTTGGCGCCACGTACGCAGTGTTGGCTCCGGAACATCCGCTTGTTGAGAAAATTACAACGCCGGAGCAA
AAACCAGCCGTTGATGCTTACTTAAAAGAAGTGCAAAGCAAAAGCGACCTCGAGCGCACCGACTTGGCGA
AAGAAAAAACAGGCGTGTTCACTGGTGCGTACGCCATCCATCCAGTTACCGGCGACAAGCTGCCGATTTG
GATCGCCGATTACGTGTTGATGGGCTACGGCACTGGGGCGATCATGGCTGTACCGGCGCATGATGAGCGC
GACTACGAGTTTGCGAAAACATTCAACTTGCCGATCAAAGAAGTCGTTGCCGGCGGGAATGTCGAAAACG
AGCCGTACACTGGCGACGGGGAGCACATCAACTCTGAGTTTTTGAACGGCTTGAACAAACAAGAAGCGAT
CGAAAAAATGATCGCCTGGCTTGAAGAAAACGGAAAAGGACAAAAGAAAGTGTCGTACCGGCTGCGCGAC
TGGTTGTTTAGCCGCCAACGCTACTGGGGTGAGCCGATTCCGGTCATCCATTGGGAAGATGGGACGATGA
CGACGGTGCCGGAAGAAGAATTGCCGCTTGTCTTGCCGAAAACGGATGAAATTAAACCGTCGGGAACGGG
TGAATCGCCGCTCGCCAACATCGAAGAATGGGTCAATGTTGTCGATCCGAAAACCGGGAAAAAAGGGCGG
CGTGAAACAAACACGATGCCGCAATGGGCGGGAAGCTGCTGGTATTATTTGCGCTACATCGACCCGCATA
ACGACAAACAGCTCGCCGATCCGGAAAAGTTGAAACAATGGCTGCCGGTTGACGTCTACATCGGCGGGGC
GGAGCATGCGGTCTTGCACTTGCTGTACGCTCGCTTCTGGCATAAAGTGTTGTACGACCTTGGCATCGTG
CCGACGAAAGAGCCGTTCCAAAAGCTGTTTAACCAAGGGATGATCTTAGGCGAAAACAATGAAAAAATGA
GCAAATCGAAAGGCAATGTCGTCAACCCGGATGATATCGTCGAGAGCCATGGCGCGGATACGTTGCGGCT
GTATGAAATGTTTATGGGGCCGCTTGAAGCGTCGATCGCCTGGTCGACGAAAGGGCTTGACGGAGCGCGC
CGTTTCTTAGAGCGCGTCTGGCGTCTGTTTGTCACCGAAGATGGTCAACTGAACCCGAACATCGTTGACG
AGCCAGCGAACGATACGCTCGAGCGCGTCTACCATCAAACGGTGAAAAAAGTGACGGAAGACTACGAAGC
GCTGCGCTTCAACACCGCCATTTCGCAGCTGATGGTGTTCATTAACGAAGCGTATAAAGCGGAGCAGATG
AAAAAAGAATATATGGAAGGGTTCGTCAAGCTCTTATCGCCGGTTTGCCCGCATATTGGCGAAGAGCTCT
GGCAAAAGCTCGGCCATACTGACACCATCGCCTATGAACCATGGCCGACATATGACGAAGCGAAACTCGT
CGAAGATGTCGTTGAAATCGTGATCCAAATCAACGGCAAAGTGCGGGCGAAACTGAACGTGCCGGCGGAC
TTATCGAAAGAGGCGCTAGAAGAACGGGCGCTCGCCGATGAAAAAATTAAAGAGCAGCTTGCAGGGAAAA
CGGTGCGTAAGGTGATCACTGTCCCTGGTAAGCTCGTCAATATCGTCGCCAAC
SEQ ID NO. 110
Amino Acid
LeuRS-GsuLeuRS
Geobacillus subterraneus DSM 13552 (91A1)
MRRSATMSFNHREIEKKWQDYWEQHKTFRTPDESDKPKFYVLDMFPYPSGAGLHVGHPEGYTATDILARM
KRMQGYNVLHPMGWDAFGLPAEQYALDTGNDPAEFTQKNIDNFRRQIKSLGFSYDWDREINTTDPNYYKW
TQWIFLKLYEKGLAYMDEVPVNWCPALGTVLANEEVINGRSERGGHPVIRKPMRQWMLKITAYADRLLED
LEELDWPESIKEMQRNWIGRSEGAEIEFAVDGHDESFTVFTTRPDTLFGATYAVLAPEHPLVEKITTPEQ
KPAVDAYLKEVQSKSDLERTDLAKEKTGVFTGAYAIHPVTGDKLPIWIADYVLMGYGTGAIMAVPAHDER
DYEFAKTFNLPIKEVVAGGNVENEPYTGDGEHINSEFLNGLNKQEAIEKMIAWLEENGKGQKKVSYRLRD
WLFSRQRYWGEPIPVIHWEDGTMTTVPEEELPLVLPKTDEIKPSGTGESPLANIEEWVNVVDPKTGKKGR
RETNTMPQWAGSCWYYLRYIDPHNDKQLADPEKLKQWLPVDVYIGGAEHAVLHLLYARFWHKVLYDLGIV
PIKEPFQKLFNQGMILGENNEKMSKSKGNVVNPDDIVESHGADTLRLYEMFMGPLEASIAWSTKGLDGAR
RFLERVWRLFVTEDGQLNPNIVDEPANDTLERVYHQTVKKVTEDYEALRFNTAISQLMVFINEAYKAEQM
KKEYMEGFVKLLSPVCPHIGEELWQKLGHTDTIAYEPWPTYDEAKLVEDVVEIVIQINGKVRAKLNVPAD
LSKEALEERALADEKIKEQLAGKTVRKVITVPGKLVNIVAN
SEQ ID NO. 111
DNA
LysRS-GsuLysRS
Geobacillus subterraneus DSM 13552 (91A1)
ATGAGCCATGAAGAATTGAACGACCAATTGCGTGTCCGCCGGGAAAAGTTAAAAAAAATCGAAGAGCTAG
GTGTCGACCCGTTTGGCAAACGGTTCGAGCGCACGCATAAAGCAGAAGAGCTGTTTAAACTGTACGGCGA
TTTGTCCAAAGAAGAACTTGAAGATCAGCAAATTGAAGTCGCTGTCGCCGGCCGCATTATGACGAAACGC
GGTAAAGGAAAAGCAGGATTTGCTCACATTCAAGACGTCACAGGGCAAATTCAAATTTATGTCCGCCAAG
ACGATGTCGGTGAACAGCAATATGAGCTGTTTAAAATCTCTGACCTTGGTGATATCGTCGGTGTGCGCGG
CACTATGTTCAAAACAAAAGTCGGCGAGCTTTCCATCAAAGTGTCATCATATGAATTTTTAACAAAAGCA
TTGCGTCCATTGCCGGAAAAATACCATGGTTTAAAGGACGTCGAACAACGTTACCGCCAACGTTATCTCG
ACTTAACTATGAATCCGCAAAGTAAGCAGACGTTTATCACCCGTAGTCTCATTATTCAATCGATGCGGCG
TTATCTCGACAGCCAAGGTTATTTGGAAGTCGAAACACCGATGATGCACGCCATAGCAGGTGGTGCGGCT
GCACGTCCGTTTATTACGCACCATAATGCCCTTGATATGACACTTTATATGCGAATCGCCATCGAACTCC
ATTTAAAACGGCTCATCGTCGGCGGTTTGGAAAAAGTGTATGAAATCGGACGCGTCTTCCGGAATGAGGG
GATTTCCACCCGTCACAATCCGGAGTTTACGATGCTTGAACTGTACGAGGCATATGCCGACTTCCGTGAC
ATCATGAAATTGACAGAAAACTTAATTGCTCACATTGCCACGGAAGTGCTTGGCACGACGAAAATTCAAT
ACGGCGAACATACCGTCGATTTAACGCCTGAATGGCGGCGACTTCATATGGTCGATGCGATTAAAGAATA
CGTCGGCGTTGATTTCTGGCGGCACATGGACGACGAGGAAGCGCGGGCGTTGGCGAAAGAACATGGGGTC
GAAATCGCCCCGCACATGACGTTTGGTCATATCGTCAATGAATTTTTTGAACAAAAAGTCGAGTCGCAAC
TCATCCAACCGACGTTCATTTATGGCCACCCTGTCGAAATTTCGCCGTTAGCTAAGAAAAACCCGGACGA
TCCACGCTTTACCGATCGATTTGAGCTATTTATCGTTGGACGTGAACATGCGAACGCGTTTACGGAACTA
AACGATCCGATCGACCAGCGCCAACGTTTCGAAGCACAGTTGAAAGAACGTGAACAAGGGAACGATGAAG
CGCACGAAATGGACGAAGATTTCCTCGAAGCGCTCGAGTACGGTATGCCTCCAACAGGCGGACTCGGCAT
CGGCGTTGACCGTCTAGTCATGCTCTTGACTAACTCTCCGTCCATTCGGGATGTGTTACTCTTCCCGCAA
ATGCGTCATAAA
SEQ ID NO. 112
Amino Acid
LysRS-GsuLysRS
Geobacillus subterraneus DSM 13552 (91A1)
MSHEELNDQLRVRREKLKKIEELGVDPFGKRFERTHKAEELFKLYGDLSKEELEDQQIEVAVAGRIMTKR
GKGKAGFAHIQDVTGQIQIYVRQDDVGEQQYELFKISDLGDIVGVRGTMFKTKVGELSIKVSSYEFLTKA
LRPLPEKYHGLKDVEQRYRQRYLDLTMNPQSKQTFITRSLIIQSMRRYLDSQGYLEVETPMMHAIAGGAA
ARPFITHHNALDMTLYMRIAIELHLKRLIVGGLEKVYEIGRVFRNEGISTRHNPEFTMLELYEAYADFRD
IMKLTENLIAHIATEVLGTTKIQYGEHTVDLTPEWRRLHMVDAIKEYVGVDFWRHMDDEEARALAKEHGV
EIAPHMTFGHIVNEFFEQKVESQLIQPTFIYGHPVEISPLAKKNPDDPRFTDRFELFIVGREHANAFTEL
NDPIDQRQRFEAQLKEREQGNDEAHEMDEDFLEALEYGMPPTGGLGIGVDRLVMLLTNSPSIRDVLLFPQ
MRHK
SEQ ID NO. 113
DNA
MetRS-GsuMetRS
Geobacillus subterraneus DSM 13552 (91A1)
ATGGAGAAAAAGACGTTTTATTTGACGACGCCGATTTATTATCCGAGCGACAAATTGCACATCGGCCATG
CTTATACAACAGTGGCGGGGGATACGCTAGCGCGCTATAAACGGATGCGCGGTTACGATGTTATGTATTT
GACGGGAACCGATGAGCACGGGCAAAAAATTCAACGCAAGGCGGAGGAAAAAGGAGTAACGCCGCAGCAA
TATGTCGATGAGATCGTCGCTGGCATTCAGGAGCTATGGAAAAAGCTCGACATTTCTTATGACGATTTCA
TCCGTACAACGCAGGAGCGGCATAAAAAAGTAGTCGAAAAGATTTTCGCGCGTCTTGTCGAACAAGGGGA
TATTTATTTAGGTGAATATGAAGGATGGTATTGCACGCCATGCGAATCGTTTTACACTGAGCGACAGCTT
GTCGACGGCAACTGCCCGGACTGTGGTCGTCCGGTTGAAAAAGTGAAAGAGCAGTCGTACTTTTTCCGAA
TGAGCAAATACGTCGACCGTTTGCTTCAATATTATGAGGAAAATCCAGATTTCATCCAGCCGGAATCGCG
GAAAAACGAAATGATTAACAATTTTATTAAGCCGGGGCTTGAAGATTTAGCTGTGTCGCGGACGACGTTT
GACTGGGGCATTAAAGTGCCGGGCGATCCGAAACATGTCATTTACGTCTGGATTGACGCGCTTGCCAACT
ATATTACAGCGCTCGGTTACGGCACGGACAATGATGAAAAGTTCCGCAAATATTGGCCGGCCGATGTCCA
TTTAGTCGGCAAGGAAATCATCCGCTTTCATACGATTTATTGGCCGATTATGCTCATGGCGCTTGACTTG
CCGCTGCCGAAAAAAGTATTCGGTCATGGCTGGCTGCTCATGAAAGACGGGAAAATGTCGAAATCGAAAG
GCAATGTCGTTGACCCGGTGACGTTGATCGATCGATACGGACTCGATGCGCTTCGTTATTATTTACTCAG
GGAAGTGCCGTTCGGTTCTGACGGCGTATTCACGCCGGAAGGATTTATTGAGCGCATCAACTACGATTTA
GCCAATGACCTAGGCAATTTATTGAATCGTACAGTAGCGATGATTAAGAAATATTTTGATGGGGTGATTC
CGCCGTACCGCGGTCCGAAAACGCCGTTTGACGAAGAGCTGGTACAAACGGCGCGTGAGGTGGTCCGTCA
GTATGAGGAAGCGATGGAACGGATGGAGTTTTCCGTTGCCCTTGCTTCGGTTTGGCAACTGATTGGCCGG
ACGAACAAATACATTGATGAGACGCAGCCATGGGTATTGGCCAAAGATGAAAGCAAACGGGAAGAGCTTG
CTTCTGTCATGACCCACCTAGCCGAGTCGCTCCGCCATACGGCAGTGCTGTTGCAGCCGTTTTTGACACG
CACGCCAGAGCGCATTTTTGCCCAGCTCGGCATTGCCGACCGTTCATTAAAAGAGTGGGATAGCTTGTAC
GAGTTCGGGCTCATTCCGGAAGGAACAAACGTGCAAAAAGGAGAACCACTGTTCCCGCGCCTTGATATTG
AAGCGGAAGTCGAGTACATTAAGGCGCATATGCAAGGCGGCAAGCCGGCGGTGGAACCCGTTAAAGAGGA
GAAGCAAGCGGCTGAGACGGCCGAAATCTCAATTGATGAGTTTGCCAAAGTTGACTTGCGCGTTGCTGAA
GTCGTGCATGCTGAACGGATGAAAAACGCCAATAAGCTGTTGAAGCTCCAACTTGATCTTGGCGGCGAGA
AACGGCAAGTCATCTCTGGTATCGCTGAATTTTACAAACCAGAGGAACTCATCGGCAAAAAGGTCATTTG
CGTCGCCAATTTAAAACCGGCCAAACTGCGCGGTGAGTGGTCGGAAGGAATGATTTTGGCCGGCGGTAAC
GGCGGAGAGTTTTCACTGGCGACCGTCGATCAACATGTGCCAAACGGAACAAAAATTAAA
SEQ ID NO. 114
Amino Acid
MetRS-GsuMetRS
Geobacillus subterraneus DSM 13552 (91A1)
MEKKTFYLTTPIYYPSDKLHIGHAYTTVAGDTLARYKRMRGYDVMYLTGTDEHGQKIQRKAEEKGVTPQQ
YVDEIVAGIQELWKKLDISYDDFIRTTQERHKKVVEKIFARLVEQGDIYLGEYEGWYCTPCESFYTERQL
VDGNCPDCGRPVEKVKEQSYFFRMSKYVDRLLQYYEENPDFIQPESRKNEMINNFIKPGLEDLAVSRTTF
DWGIKVPGDPKHVIYVWIDALANYITALGYGTDNDEKFRKYWPADVHLVGKEIIRFHTIYWPIMLMALDL
PLPKKVFGHGWLLMKDGKMSKSKGNVVDPVTLIDRYGLDALRYYLLREVPFGSDGVFTPEGFIERINYDL
ANDLGNLLNRTVAMIKKYFDGVIPPYRGPKTPFDEELVQTAREVVRQYEEAMERMEFSVALASVWQLIGR
INKYIDETQPWVLAKDESKREELASVMTHLAESLRHTAVLLQPFLTRTPERIFAQLGIADRSLKEWDSLY
EFGLIPEGTNVQKGEPLFPRLDIEAEVEYIKAHMQGGKPAVEPVKEEKQAAETAEISIDEFAKVDLRVAE
VVHAERMKNANKLLKLQLDLGGEKRQVISGIAEFYKPEELIGKKVICVANLKPAKLRGEWSEGMILAGGN
GGEFSLATVDQHVPNGTKIK
SEQ ID NO. 115
DNA
Phe-aRS-GsuPhe-aRS
Geobacillus subterraneus DSM 13552 (91A1)
ATGAGGGACGGGTTTTTTTATTTTGTTAGAGGAGGGATTGGCGTGAAAGAACGGTTGCATGAGCTTGAAC
GAGAAGCGCTTGAAAAAATTGAACAAGCTGGCGATTTAAAAGCGCTCAACGATGTGCGTGTCGCCTATTT
AGGCAAAAAAGGGCCGATTACCGAAGTGCTGCGCGGCATGGGAGCATTGCCGTCAGAAGAGCGTCCGAAA
ATTGGTGCGCTTGCCAATGAGGTAAGAGAGGCGATCCAAAAGGCGCTCGAAGCAAAACAAACGAAACTGG
AAGAAGAAGAAGTCGAGCGGAAGTTGGCGGCTGAAGCGATCGATGTGACGCTTCCGGGCCGTCCGGTGAA
ACTGGGGAATCCTCATCCGCTGACGCGCGTCATCGAGGAAATTGAAGATTTGTTTATCGGCATGGGCTAT
ACGGTCGCCGAAGGTCCGGAAGTCGAGACCGATTATTACAATTTTGAGGCGCTCAATTTGCCGAAAGGAC
ACCCGGCCCGCGATATGCAAGATTCGTTTTATATTACGGAAGAAATTCTGCTTCGCACCCACACGTCGCC
GATGCAGGCACGGACGATGGAAAAACATCGCGGGCGCGGTCCGGTAAAAATCATTTGCCCGGGGAAAGTG
TATCGCCGCGATACCGATGATGCGACCCATTCACATCAGTTTACGCAAATTGAAGGATTGGTTGTTGACC
GCAACATCCGGATGAGCGATTTAAAAGGGACGCTGCGCGAATTTGCCCGCAAGCTGTTCGGTGAAGGGCG
CGACATCCGTTTTCGTCCGAGCTTTTTCCCGTTTACCGAGCCTTCAGTCGAGGTCGATGTGTCCTGCTTC
CGCTGCGAAGGGCACGGCTGCAGCGTTTGCAAAGGTACGGGCTGGATTGAAATTTTAGGCGCTGGCATGG
TGCACCCGAACGTGCTTGAGATGGCCGGCTTTGATTCGAAAACGTATACCGGATTTGCGTTCGGCATGGG
GCCGGAGCGGATCGCGATGTTGAAATACGGCATTGATGACATCCGCCATTTCTATCAGAACGATCTTCGT
TTCTTGCAACAATTTTTGCGTGTC
SEQ ID NO. 116
Amino Acid
Phe-aRS-GsuPhe-aRS
Geobacillus subterraneus DSM 13552 (91A1)
MRDGFFYFVRGGIGVKERLHELEREALEKIEQAGDLKALNDVRVAYLGKKGPITEVLRGMGALPSEERPK
IGALANEVREAIQKALEAKQTKLEEEEVERKLAAEAIDVTLPGRPVKLGNPHPLTRVIEEIEDLFIGMGY
TVAEGPEVETDYYNFEALNLPKGHPARDMQDSFYITEEILLRTHTSPMQARTMEKHRGRGPVKIICPGKV
YRRDTDDATHSHQFTQIEGLVVDRNIRMSDLKGTLREFARKLFGEGRDIRFRPSFFPFTEPSVEVDVSCF
RCEGHGCSVCKGTGWIEILGAGMVHPNVLEMAGFDSKTYTGFAFGMGPERIAMLKYGIDDIRHFYQNDLR
FLQQFLRV
SEQ ID NO. 117
DNA
Phe-bRS-GsuPhe-bRS
Geobacillus subterraneus DSM 13552 (91A1)
ATGCTCGTTTCTTATCGTTGGCTAGGCGAATACGTCGATTTGACGGGCGTGACGGCGGAACAACTCGCTG
ATCGCATTACAAAAAGCGGCATTGAAGTCGAGCGGGTTGAAGCGCTTGAGCGGGGAATGAAAGGAGTCGT
CATCGGCCATGTGCTCGAATGCGAGCCACACCCAAACGCCGATAAACTGCGGAAATGTCTTGTTGATCTT
GGCGAAGGAGAGCCGGTGCAAATCATTTGCGGTGCCCCGAACGTCGCCAAGGGGCAAAAAGTTGCTGTAG
CGAAAGTTGGAGCGAGACTGCCGGGCAATTTTAAAATCAAACGGGCGAAGCTGCGCGGCGAAGAGTCGAA
CGGCATGATTTGCTCGCTCCAAGAACTCGGTGTTGAAACAAAAGTCGTGCCGAAAGAATACGCCGAAGGC
ATTTTCGTCTTCCCAAGCGACGCGCCGGTCGGCGCTGATGCGCTTGAATGGCTCGGCTTGCACGATGAAG
TGCTCGAACTCGCCTTGACGCCGAATCGCGCCGATTGCTTAAGCATGCTTGGCGTTGCCTACGAAGTCGC
TGCGATTCTCGGCCGCGATGTGAAGTTGCCGGAAACGGCGGTGAACGAAAATGAAGAAAGCGTCCATGAC
TACATTTCTGTCCGTGTCGAGGCGCCGGAAGACAATCCGCTGTACGCCGGACGGATCGTGAAAAACGTCC
AAATCGGCCCGTCGCCGCTTTGGATGCAAGCGCGCTTGATGGCGGCCGGCATTCGTCCACACAACAATGT
TGTCGATATCACCAACTACATTTTGCTTGAGTACGGCCAGCCGCTTCACGCGTTTGACTACGACCGTCTC
GGTTCGAAGGAGATCGTCGTTCGTCGTGCCAAGGCGGGAGAAATGATCGTGACGCTTGACGATGTCGAGC
GGAAGCTGACTGAAGATCATCTCGTCATCACAAACGGCCGTGAGCCGGTCGCCTTAGCCGGTGTGATGGG
CGGAGCGAACTCGGAAGTGCAGGATGACACGAAAACAGTGTTCATCGAAGCCGCGTATTTTACGAGCCCG
GTCATCCGCCAGGCGGTGAAAGACCACGGGTTGCGCAGCGAAGCGAGCACCCGGTTTGAAAAAGGGATTG
ATCCGGCGCGGACGAAAGAAGCGCTCGAGCGCGCTGCTGCTTTGATGGCAGAATACGCCGGCGGCGAGGT
CGTCAGCGGTATCGTGGAAGCTAATACATGGAAAGAAGAGCCGGTTGTCGTAACGGTGGCGCTGGAACGC
ATCAACGGCGTCCTCGGCACAGCGATGACGAAAGAGGAAGTAGCTGGCATTCTTTCAAACTTGCAATTCT
CGTTTACGGAAGATAATGGAACGTTTACAATCCATGTTCCATCGCGCCGCCGCGATATTACGATCGAAGA
AGATATTATCGAGGAAGTCGCCCGTTTGTATGGCTACGACCATTTGCCAGCGACTTTGCCGGTGGCCGAA
GCAAAACCGGGCGAGTTGACACCGTACCAAGCGAAACGCCGCCGTGTCCGCCGCTATTTCGAAGGCGCGG
GCTTGTTCCAGGCGATCACGTATTCGCTTACCAGTCCGGACAAAGCGACGCGGTTTGCTTTGGAGACAAC
CGAACCAGTCCGCTTGGCGTTGCCGATGAGTGAGGAGCGGAGCGTTCTCCGGCAAAGCTTGGTGCCGCAT
TTGCTCGAAGCGGCGAGCTACAACCGTGCCCGCCAAGTTGAGAACGTCGCGCTATATGAAATCGGCTCTG
TCTATTTGTCCAAGGGGGAAAATGTCCAACCGGCGGAAAAAGAACGGCTCGCCGGCGTCATCACCGGTTT
ATGGCATGCCCACCTTTGGCAAGGAGAGAAAAAAGCAGCTGATTTCTATGTTGCAAAAGGCGTGCTTGAC
GGCTTGTTCGCCCTGCTTGGGCTGTCTGATCGCATCAGCTACCGTCCGGCGAAGCGTGCTGATTTGCATC
TGGGGCGGACAGCGGAGATTGTGCTTGACGGCAAAGAGATCGGCTTTGTCGGCCAGCTCCATCCGGCTGT
ACAAAAAGAGTACGATTTGAAAGAAACGTATGTCTTTGAACTCGCCTTCGCTGAGCTACTGAATACAGAA
GGCGAAACGATCCGTTACGAGTCGATTCCGCGCTTCCCGTCAGTCGTGCGCGACATCGCTTTAGTCGTCG
ACGACAATGTCGAAGCAGGTGCTCTCAAGCAGGCGATCGCCGAAGCGGGGAACCCGCTATTAAAAGACGT
GGCCCTCTTTGACGTCTATAAAGGCGACCGTCTGCCGGCCGGGAAAAAATCGCTCGCCTTCTCGCTCCGC
TACTACGATCCGGAACGGACGCTCACTGATGAGGAAGTTACTGCCGTCCATGAACGGGTTTTGGCAGCGG
TCGAGGAGCAGTTTGGCGCGGTGTTGCGCGGG
SEQ ID NO. 118
Amino Acid
Phe-bRS-GsuPhe-bRS
Geobacillus subterraneus DSM 13552 (91A1)
MLVSYRWLGEYVDLTGVTAEQLADRITKSGIEVERVEALERGMKGVVIGHVLECEPHPNADKLRKCLVDL
GEGEPVQIICGAPNVAKGQKVAVAKVGARLPGNFKIKRAKLRGEESNGMICSLQELGVETKVVPKEYAEG
IFVFPSDAPVGADALEWLGLHDEVLELALTPNRADCLSMLGVAYEVAAILGRDVKLPETAVNENEESVHD
YISVRVEAPEDNPLYAGRIVKNVQIGPSPLWMQARLMAAGIRPHNNVVDITNYILLEYGQPLHAFDYDRL
GSKEIVVRRAKAGEMIVTLDDVERKLTEDHLVITNGREPVALAGVMGGANSEVQDDTKTVFIEAAYFTSP
VIRQAVKDHGLRSEASTRFEKGIDPARTKEALERAAALMAEYAGGEVVSGIVEANTWKEEPVVVTVALER
INGVLGTAMTKEEVAGILSNLQFSFTEDNGTFTIHVPSRRRDITIEEDIIEEVARLYGYDHLPAILPVAE
AKPGELTPYQAKRRRVRRYFEGAGLFQAITYSLTSPDKATRFALETTEPVRLALPMSEERSVLRQSLVPH
LLEAASYNRARQVENVALYEIGSVYLSKGENVQPAEKERLAGVITGLWHAHLWQGEKKAADFYVAKGVLD
GLFALLGLSDRISYRPAKRADLHLGRTAEIVLDGKEIGFVGQLHPAVQKEYDLKETYVFELAFAELLNTE
GETIRYESIPRFPSVVRDIALVVDDNVEAGALKQAIAEAGNPLLKDVALFDVYKGDRLPAGKKSLAFSLR
YYDPERTLTDEEVTAVHERVLAAVEEQFGAVLRG
SEQ ID NO. 119
DNA
ProRS-GsuProRS
Geobacillus subterraneus DSM 13552 (91A1)
ATGACATTCAAAAATTCTTCCTATAATGAAAGAGAGAAAACGAGGTGGCTATTGATGAGACAAAGTCAAG
GGTTTATTCCGACATTGCGCGAAGTGCCGGCGGACGCGGAAGTGAAAAGCCATCAGCTCCTGTTGCGGGC
CGGCTTCGTCCGCCAAAGCGCAAGCGGCGTCTACACGTTTTTGCCGCTCGGGCAACGTGTTTTGCAAAAA
GTGGAAGCGATTATTCGTGAGGAGATGAATCGCGCCGGAGCATTGGAGCTTCTCATGCCTGCTTTGCAGC
CGGCTGAGCTTTGGCAGCAGTCCGGGCGCTGGTATTCGTATGGACCGGAGCTCATGCGCCTGAAAGACCG
TCACGAGCGCGATTTCGTTCTCGGACCGACACACGAAGAGATGATTACTACGATCGTTCGCGATGAAGTG
AAAACGTATAAGCGGCTGCCGCTTATCTTGTATCAAATTCAAACGAAATTCCGTGATGAAAAACGTCCGC
GTTTCGGGCTGTTGCGCGGTCGCGAGTTCATCATGAAAGATGCGTATTCATTCCACACATCGCAGGAAAG
TTTGGACGAAACGTACAATAAAATGTATGAAGCGTACGCGAACATTITCCGCCGCTGCGGCTTAAATTIC
CGCGCTGTCATTGCTGACTCCGGAGCGATGGGCGGCAAAGATACGCACGAGTTTATGGTGCTGTCTGATA
TTGGCGAGGATACGATCGCTTATTCCGATGCGTCCGACTATGCGGCCAACATTGAAATGGCACCGGTCGT
CACTACGTATGAAAAAAGCAGTGAGCCGCTGGTGGAACTGAAAAAAGTGGCGACCCCGGAGCAAAAAACG
ATTGCTGAAGTTGCTTCGTATTTGCAAGTAGCACCGGAACGTTGCATTAAATCGCTTTTATTTAACGTTG
ATGGCCGCTACGTGCTCGTTCTGGTGCGCGGCGATCATGAAGCGAATGATGTGAAAGTGAAAAATGTGCT
TGATGCGACTGTCGTGGAGCTGGCGACACCGGAAGAAACAGCACGAGTGATGAACTGCCCGGTTGGTTCG
CTCGGCCCGATTGGCGTCAGCGAAGAGGTGACGATTATCGCCGATCATGCTGTCGCGGCGATCGTAAACG
GCGTCTGCGGCGCCAATGAGGAAGGATACCATTATACGGGTGTCAATCCAGACCGCGATTTTGCCGTCAG
TCAATATGCGGATTTGCGTTTCGTCCAAGAAGGCGACCCTTCTCCGGATGGCAACGGGACGATCCGCTTC
GCTCGTGGCATTGAAGTTGGACATGTGTTTAAGCTCGGTACGAAATATAGCGAGGCGATGAACGCCGTTT
ACCTCGACGAAAATGGTCGGACACAGACGATGATTATGGGTTGCTACGGCATTGGCGTCTCTAGGCTCGT
TGCGGCGATCGCCGAGCAGTTCGCCGATGAGAACGGGCTTGTATGGCCGGTTTCGGTCGCACCGTTTCAC
GTTCATTTGCTGACGGCGAACGCGAAAAGCGATGAACAGCGCATGCTGGCTGAAGAGTGGTACGAAAAAC
TCGGACAGGCCGGATTTGACGTGTTGTATGATGACCGTCCGGAACGGGCCGGGGTGAAGTTTGCCGACAG
CGATTTGATCGGCATCCCGCTCCGCGTCACCGTTGGCAAGCGGGCAAGTGAAGGTGTGGTCGAAGTAAAA
GTTCGGAAAACAGGCGAGACGTTTGACGTGCCGGTCGGTGAGCTGATCGAAACAGTGCGCCGTCTTTTGC
AAGGA
SEQ ID NO. 120
Amino Acid
ProRS-GsuProRSt
Geobacillus subterraneus DSM 13552 (91A1)
MTFKNSSYNEREKTRWLLMRQSQGFIPTLREVPADAEVKSHQLLLRAGFVRQSASGVYTFLPLGQRVLQK
VEAIIREEMNRAGALELLMPALQPAELWQQSGRWYSYGPELMRLKDRHERDFVLGPTHEEMITTIVRDEV
KTYKRLPLILYQIQTKFRDEKRPRFGLLRGREFIMKDAYSFHTSQESLDETYNKMYEAYANIFRRCGLNF
RAVIADSGAMGGKDTHEFMVLSDIGEDTIAYSDASDYAANIEMAPVVTTYEKSSEPLVELKKVATPEQKT
IAEVASYLQVAPERCIKSLLFNVDGRYVLVLVRGDHEANDVKVKNVLDATVVELATPEETARVMNCPVGS
LGPIGVSEEVTIIADHAVAAIVNGVCGANEEGYHYTGVNPDRDFAVSQYADLRFVQEGDPSPDGNGTIRF
ARGIEVGHVFKLGTKYSEAMNAVYLDENGRTQTMIMGCYGIGVSRLVAAIAEQFADENGLVWPVSVAPFH
VHLLTANAKSDEQRMLAEEWYEKLGQAGFDVLYDDRPERAGVKFADSDLIGIPLRVTVGKRASEGVVEVK
VRKTGETFDVPVGELIETVRRLLQG
SEQ ID NO. 121
DNA
SerRS-GsuSerRS
Geobacillus subterraneus DSM 13552 (91A1)
ATGGTGGATAAGGAGGTAAAGCGAATGCTGGATGTGAAATTACTACGCACCCAATTTCAAGAGGTGAAAG
AAAAACTGCTGCAGCGCGGCGACGACTTGGCCAACATCGACCGGTTTGAGCAGCTTGATAAAGAGCGTCG
TCGTTTGATCGCTCAGGTGGAGGAGTTAAAAAGCAAGCGCAATGAGGTGTCGCAACAAATTGCTGTCTTA
AAGCGTGAAAAAAAGGACGCCGAGTCGTTGATCGTCGAAATGCGCGAAGTCGGCGACCGCATTAAACAAA
TGGACGAGCAAATTCGCCAACTTGAAGAAGAGCTCGACAGCCTTCTGTTATCGATTCCGAATGTACCGCA
TGAGTCAGTGCCAGTCGGTCAGTCGGAAGAAGATAATGTCGAAGTGCGAAGATGGGGGGAACCGCGTTCG
TTCTCGTTCGAACCGAAGCCACATTGGGACATTGCTGACCAACTCGGTTTGCTCGATTTTGAGCGGGCTG
CCAAAGTGGCAGGAAGTCGGTTTGTGTTTTACAAAGGACTAGGGGCTCGTCTTGAGCGGGCATTAATCAA
CTTTATGCTCGACATCCATCTCGATGAATTTGGCTATCAAGAGGTGTTGCCGCCATACTTAGTGAACCGG
GCGAGCATGATCGGAACAGGGCAATTGCCAAAATTTGCGGAAGACGCGTTCCACTTGGACAATGAAGACT
ATTTTCTCATTCCAACAGCGGAAGTGCCTGTGACGAATTTGCATCGCGATGAAATTTTAACGGCTGATGA
CTTGCCGCTTTACTATGCGGCTTACAGCGCGTGCTTCCGCGCCGAAGCTGGCTCGGCTGGCCGTGACACG
CGGGGGCTCATCCGCCAGCACCAATTCAATAAAGTGGAGCTCGTCAAGTTCGTCAAGCCGGAGGATTCAT
ATGACGAGTTGGAAAAATTGACGCACCAAGCCGAAACGATCCTGCAACGGCTCGGACTTCCGTATCGCGT
CGTAGCCTTGTGTACAGGGGATCTGGGATTTTCAGCGGCGAAGACGTATGATATTGAGGTGTGGCTGCCA
AGCTATGGAACGTATCGGGAAATTTCGTCGTGCAGCAACTTTGAGGCGTTCCAGGCGCGCCGAGCTAATA
TCCGCTTCCGTCGCGAGCCGAAAGCAAAGCCAGAATATGTGCATACGCTAAACGGTTCGGGGCTAGCCAT
CGGCCGCACGGTTGCTGCCATTTTGGAAAACTACCAACAAGAAGACGGATCGGTCGTCATCCCGGAAGCG
CTCCGTCCATATATGGGGAATCGGGATGTCATTCGC
SEQ ID NO. 122
Amino Acid
SerRS-GsuSerRS
Geobacillus subterraneus DSM 13552 (91A1)
MVDKEVKRMLDVKLLRTQFQEVKEKLLQRGDDLANIDRFEQLDKERRRLIAQVEELKSKRNEVSQQIAVL
KREKKDAESLIVEMREVGDRIKQMDEQIRQLEEELDSLLLSIPNVPHESVPVGQSEEDNVEVRRWGEPRS
FSFEPKPHWDIADQLGLLDFERAAKVAGSRFVFYKGLGARLERALINFMLDIHLDEFGYQEVLPPYLVNR
ASMIGTGQLPKFAEDAFHLDNEDYFLIPTAEVPVTNLHRDEILTADDLPLYYAAYSACFRAEAGSAGRDT
RGLIRQHQFNKVELVKFVKPEDSYDELEKLTHQAETILQRLGLPYRVVALCTGDLGFSAAKTYDIEVWLP
SYGTYREISSCSNFEAFQARRANIRFRREPKAKPEYVHTLNGSGLAIGRTVAAILENYQQEDGSVVIPEA
LRPYMGNRDVIR
SEQ ID NO. 123
DNA
ThrRS-GsuThrRS
Geobacillus subterraneus DSM 13552 (91A1)
ATGCCAGACGTTATTCGCATTACGTTCCCGGACGGGGCGAAAAAGGAGTTTCCGAGCGGAACGTCAACTG
AGGACATCGCTGCCTCGATCAGTCCGGGATTGAAGAAAAAAGCGATTGCCGGGAAACTGAACGGCCGGTT
TGTTGATTTACGCACGCCGCTTCAAGAAGACGGCGAGCTTGTCATTATTACCCAGGACATGCCTGAGGCA
CTTGATATTTTGCGTCATAGCACCGCCCATTTAATGGCGCAAGCGATCAAGCGGCTGTATGACAACGTCA
AGCTTGGCGTCGGCCCGGTCATTGAAAACGGCTTCTACTATGATATTGATATGGAACATAAGCTGACGCC
GGATGATTTGCCGAAAATTGAGGCGGAAATGCGCAAAATCGTAAAGGAAAATCTTGACGTTGTTCGCAAA
GAGGTGAGCCGTGACGAGGCGATTCGCCTGTATGAAAAAATTGGTGATCACTTGAAACTGGAGCTCATCA
ACGATATTCCGGAAGGCGAGACGATTTCCATTTACGAGCAAGGCGAGTTTTTCGATCTTTGTCGGGGTGT
GCACGTGCCGTCGACCGGGAAAATCAAAGAGTTCAAGCTGCTCAGCATCTCGGGGGCCTACTGGCGCGGT
GACAGCAACAACAAAATGCTGCAGCGTATTTACGGTACGGCGTTTTTCAAAAAAGAAGATCTGGACCATT
ATTTGCAGTTGCTCGAAGAGGCGAAAGAGCGCGATCATCGCAAATTGGGCAAAGAGCTTGAGCTATTTAC
GACATCACAAAAAGTCGGACAAGGACTGCCGCTTTGGTTGCCGAAAGGGGCGACGATCCGTCGCTTGATT
GAACGGTACATTGTCGATAAAGAAATCGCCCTTGGTTATGATCATGTATATACGCCGGTGCTCGGCAGTG
TGGAGCTGTATAAAACCTCAGGACACTGGGACCATTATAAAGAAAACATGTTCCCACCGATGGAAATGGA
TAACGAAGAGCTCGTGCTGCGGCCGATGAACTGCCCGCACCATATGATGATTTATAAAAGCAAGCTTCAT
AGCTACCGTGAGCTGCCGATCCGCATCGCCGAGCTCGGCACGATGCATCGCTACGAAATGTCCGGGGCGC
TTACTGGACTGCAGCGTGTCCGCGGCATGACGCTCAACGACGCCCATATTTTCGTGCGCCCGGATCAAAT
TAAAGACGAGTTTAAGCGCGTCGTTAATTTGATTTTGGAAGTATACAAAGACTTTGGGCTGGACGAATAT
TCGTTCCGCCTGTCGTACCGCGACCCACAAGATAAAGAAAAATATTACGACGACGACGAGATGTGGGAAA
AGGCGCAACGCATGCTGCGCGAGGCGATGGATGAACTTGGCCTCGATTACTACGAAGCGGAAGGGGAAGC
AGCGTTTTACGGACCGAAGCTCGATGTGCAAGTGCGCACGGCACTCGGCAAAGATGAGACGCTGTCGACT
GTACAGCTTGACTTCCTCTTGCCGGAGCGGTTTGACTTAACATATATCGGCGAAGATGGAAAACCGCACC
GCCCGGTCGTCATCCACCGCGGCGTTGTTTCCACGATGGAACGGTTTGTCGCCTTCTTGATCGAAGAATA
CAAAGGGGCATTTCCAACGTGGCTCGCCCCGGTGCAAGTGGAAGTCATCCCGGTATCGTCGGAAGCCCAT
CTCGATTATGCGTATGAAGTGAAACAAGCGCTGCAAGTAAACGGCTTCCGCGTCGAAGTCGACGAACGGG
ATGAAAAAATCGGCTATAAAATCCGCGAAGCGCAAATGCAAAAAATTCCTTATATGCTCGTTGTCGGCGA
CAAAGAAGCGGCCGAGCGAGCGGTCAACGTCCGCCGCTACGGTGAAAAAGAAAGCGAGACTGTGGCGCTT
GACAAGTTTATCGCGATGCTAGAAGAAGATGTGCGGCAAAAACGAGTGAAAAAACGA
SEQ ID NO. 124
Amino Acid
ThrRS-GsuThrRS
Geobacillus subterraneus DSM 13552 (91A1)
MPDVIRITFPDGAKKEFPSGTSTEDIAASISPGLKKKAIAGKLNGRFVDLRTPLQEDGELVIITQDMPEA
LDILRHSTAHLMAQAIKRLYDNVKLGVGPVIENGFYYDIDMEHKLTPDDLPKIEAEMRKIVKENLDVVRK
EVSRDEAIRLYEKIGDHLKLELINDIPEGETISIYEQGEFFDLCRGVHVPSTGKIKEFKLLSISGAYWRG
DSNNKMLQRIYGTAFFKKEDLDHYLQLLEEAKERDHRKLGKELELFTTSQKVGQGLPLWLPKGATIRRLI
ERYIVDKEIALGYDHVYTPVLGSVELYKTSGHWDHYKENMFPPMEMDNEELVLRPMNCPHHMMIYKSKLH
SYRELPIRIAELGTMHRYEMSGALTGLQRVRGMTLNDAHIFVRPDQIKDEFKRVVNLILEVYKDFGLDEY
SFRLSYRDPQDKEKYYDDDEMWEKAQRMLREAMDELGLDYYEAEGEAAFYGPKLDVQVRTALGKDETLST
VQLDFLLPERFDLTYIGEDGKPHRPVVIHRGVVSTMERFVAFLIEEYKGAFPTWLAPVQVEVIPVSSEAH
LDYAYEVKQALQVNGFRVEVDERDEKIGYKIREAQMQKIPYMLVVGDKEAAERAVNVRRYGEKESETVAL
DKFIAMLEEDVRQKRVKKR
SEQ ID NO. 125
DNA
TrpRS-GsuTrpRS
Geobacillus subterraneus DSM 13552 (91A1)
ATGAAAACCATTTTTTCTGGCATTCAGCCAAGCGGCGTCATTACCCTTGGCAACTACATTGGTGCGATGC
GACAATTTGTCGAACTGCAGCATGAGTACAACTGCTATTTTTGCATTGTCGACCAACATGCCATTACTGT
TCCGCAAAATCCGAACGAACTGCAACAAAACATTCGCCGTCTCGCTGCCTTATATTTGGCAGTCGGCATC
GATCCTAAACAGGCGACGCTGTTCGTTCAATCGGAGGTGCCGGCGCACGCCCAAGCGGCTTGGATGCTGC
AATGCATCGTCTATATCGGCGAACTGGAGCGGATGACGCAGTTTAAAGACAAATCAGCCGGTAAAGAGGC
GGTCAGTGCCGGGTTGCTCACGTATCCACCGCTTATGGCAGCCGACATTTTGCTTTACAACACGGACATT
GTCCCAGTCGGCGAAGACCAAAAGCAGCACATCGAGCTGACGCGCGATTTAGCTGAGCGCTTCAACAAAC
GGTACGGCGAGCTGTTCACTATCCCGGAAGCGCGCATCCCGAAAATCGGCGCCCGCATTATGTCGCTTAC
CGATCCGACGAAAAAAATGAGCAAATCTGACCCAAACCCGAAATCGTTTATTACGCTGCTTGACGACGCC
AAAACGATTGAAAAGAAAATTAAAAGTGCTGTGACCGATTCAGAAGGAACGATTCGCTATGACAAGGAAG
CGAAACCGGGCATTTCGAACTTGCTCAACATTTATTCGATTTTATCGGGTCAGCCGATTGACGAACTTGA
GCGGCAATACGAAGGAAAAGGATACGGGGTCTTTAAATCCGATTTGGCCCAAGTGGTCATTGAAACGCTC
CAACCGATCCAAGAGCGGTATTATCATTGGCTCGAAAGTGAAGAGCTCGACCGCGTCCTAGACGAAGGGG
CGGAAAAAGCGAACCGTGTCGCCTCGGAAATGGTGCGCAAAATGGAACAAGCCATGGGGCTTGGGCGGCG
TCGG
SEQ ID NO. 126
Amino Acid
TrpRS-GsTrpRS
Geobacillus subterraneus DSM 13552 (91A1)
MKTIFSGIQPSGVITLGNYIGAMRQFVELQHEYNCYFCIVDQHAITVPQNPNELQQNIRRLAALYLAVGI
DPKQATLFVQSEVPAHAQAAWMLQCIVYIGELERMTQFKDKSAGKEAVSAGLLTYPPLMAADILLYNTDI
VPVGEDQKQHIELTRDLAERFNKRYGELFTIPEARIPKIGARIMSLTDPTKKMSKSDPNPKSFIILLDDA
KTIEKKIKSAVTDSEGTIRYDKEAKPGISNLLNIYSILSGQPIDELERQYEGKGYGVFKSDLAQVVIETL
QPIQERYYHWLESEELDRVLDEGAEKANRVASEMVRKMEQAMGLGRRR
SEQ ID NO. 127
DNA
TyrRS-GsuTyrRS
Geobacillus subterraneus DSM 13552 (91A1)
ATGAACCTGCTTGAAGAACTGCAATGGCGCGGACTTGTCAATCAAACGACGGATGAGGATGGGCTTCGAA
AGCTCCTGAATGAGGAGAAGGTGACGCTTTATTGCGGGTTTGACCCGACAGCAGACAGCTTGCATATCGG
CCATTTGGTCACGATCATGACCTTGCGTCGTTTCCAACAGGCGGGGCATCAACCGATCGCCTTAGTCGGC
GGCGCCACCGGGTTGATCGGCGATCCGAGTGGCAGAAAAAGCGAGCGCACGCTCAACGCCAAGGAGACGG
TCGAGACGTGGAGCGCCCGAATCAAAGCGCAACTCGAGCGGTTTCTTGATTTTGAGGCTGAGAGCAATCC
AGCGAAAATCAAAAACAACTACGACTGGATCGGGCCGCTTGATGTCATCTCGTTTTTGCGTGACATCGGC
AAGCATTTCAGCGTCAATTACATGCTTGCGAAAGAATCGGTGCAGTCGCGCATTGAAATGGGCATTTCGT
TTACCGAGTTCAGCTATATGATGCTGCAGGCGTACGACTTCCTCAACTTGTACGAAACGGAAGGTTGCCG
ACTACAAATCGGTGGCAGCGACCAATGGGGCAACATCACGGCGGGGCTTGAGCTCATCCGCAGAACGAAA
GGTGAGGCGAAAGCATTTGGTTTGACGGTTCCGCTCGTGACGAAAGCCGATGGGACGAAGTTCGGAAAAA
CGGAAAGCGGCGCGGTTTGGCTCGATCCGGAAAAAACGTCGCCGTATGAGTTTTACCAGTTCTGGATCAA
CACCGATGACCGCGATGTGATCCGTTACTTAAAATATTTCACGTTCTTGACAAAAGAAGAGATCGACGCG
CTTGAACAAGAGCTGCGCGAAGCGCCGGAGAAGCGGGTGGCGCAAAAAACGCTTGCTTCCGAAGTGACGA
AGCTCGTGCATGGCGAAGAGGCGCTCAATCAAGCGATTCGTATTTCAGAAGCACTCTTTAGCGGCGACAT
TGCCGAACTGACGGCTGCGGAAATCGAGCAAGGGTTTAAAAACGTGCCGTCGTTTGTCCATGAAGGAGGC
GACGTCCCGCTCGTCGAGCTGCTCGTAGCTGCCGGCATCTCGCCATCGAAGCGGCAGGCGCGCGAAGATG
TTCAAAACGGTGCGATTTATGTCAACGGCGAGCGCATCCAAGATGTCGGCGCTGTCTTAACGGCCGAACA
CCGTTTGGAAGGGCGGTTTACCGTGATCCGCCGCGGCAAGAAGAAGTATTATTTAATCCGCTACGCT
SEQ ID NO. 128
Amino Acid
TyrRS-GsuTyrRS
Geobacillus subterraneus DSM 13552 (91A1)
MNLLEELQWRGLVNQTTDEDGLRKLLNEEKVTLYCGFDPTADSLHIGHLVTIMTLRRFQQAGHQPIALVG
GATGLIGDPSGRKSERTLNAKETVETWSARIKAQLERFLDFEAESNPAKIKNNYDWIGPLDVISFLRDIG
KHFSVNYMLAKESVQSRIEMGISFTEFSYMMLQAYDFLNLYETEGCRLQIGGSDQWGNITAGLELIRRTK
GEAKAFGLTVPLVTKADGTKFGKTESGAVWLDPEKTSPYEFYQFWINTDDRDVIRYLKYFTFLTKEEIDA
LEQELREAPEKRVAQKTLASEVTKLVHGEEALNQAIRISEALFSGDIAELTAAEIEQGFKNVPSFVHEGG
DVPLVELLVAAGISPSKRQAREDVQNGAIYVNGERIQDVGAVLTAEHRLEGRFTVIRRGKKKYYLIRYA
SEQ ID NO. 129
DNA
ValRS-GsuValRS
Geobacillus subterraneus DSM 13552 (91A1)
ATGAAAGGGGCTTTTTTGCTTGCCTATCGGACGGTTGATCCTGTAGGCAACACAGCCATTGTTTATCACA
TGAAGGAGGGAATAAAAGTGGCACAGCATGAAGTGTCGATGCCGCCAAAATACGATCACCGCGCTGTTGA
AGCGGGGCGCTATGACTGGTGGCTGAAAGGCAAGTTTTTTGAAACGACCGGCGATCCGGACAAACAACCG
TTTACGATCGTTATCCCACCGCCGAACGTCACAGGCAAACTGCATTTGGGCCATGCGTGGGATACGACGC
TGCAAGACATCATTACGCGCATGAAGCGGATGCAAGGGTATGATGTCCTATGGCTTCCGGGTATGGACCA
TGCCGGCATCGCCACCCAGGCGAAAGTGGAAGAAAAATTGCGCCAACAAGGACTGTCCCGCTACGATTTA
GGACGGGAAAAATTTTTGGAAGAAACGTGGAAATGGAAAGAAGAATATGCCGGCCATATCCGCAGCCAAT
GGGCAAAATTAGGGCTCGGCCTCGATTACACGCGCGAGCGGTTTACGCTTGATGAAGGGCTGTCAAAAGC
CGTACGCGAAGTGTTCGTCTCGCTTTACCGGAAAGGGCTCATTTACCGCGGTGAATACATTATCAACTGG
GATCCGGCGACCAAAACCGCCTTGTCCGACATCGAGGTCATTTACAAGGAAGTGAAAGGTGCGCTTTATC
ATTTGCGCTATCCGCTCGCTGACGGCTCGGGCTACATTGAAGTAGCGACAACCCGTCCAGAAACGATGCT
CGGTGACACGGCCGTCGCGGTTCATCCGGATGACGAGCGGTATAAACACTTGATCGGCAAGATGGTGAAA
TTGCCAATCGTTGGCCGGGAAATTCCGATCATCGCTGATGAGTATGTCGATATGGAATTCGGTTCCGGCG
CGGTAAAAATTACACCGGCACACGATCCGAACGACTTTGAAGTTGGCAACCGCCACAACTTGCCGCGCAT
TCTCGTCATGAACGAAGACGGTACAATGAACGAAAACGCATTGCAATATCAAGGGCTTGACCGGTTTGAA
TGCCGGAAGCAAATCGTCCGTGATTTACAAGAGCAAGGCGTCCTCTTTAAAATTGAGGAACACGTCCACT
CGGTCGGGCACAGTGAACGGAGCGGCGCCGTTGTTGAACCGTATTTGTCGACACAATGGTTCGTAAAAAT
GAAGCCGCTCGCGGAAGCTGCCATCAAGATGCAGCAAACAGAAGGAAAAGTGCAATTTGTGCCGGAGCGG
TTTGAAAAAACGTACTTGCACTGGCTTGAGAACATTCGCGACTGGTGCATTTCGCGTCAGCTTTGGTGGG
GGCACCGCATTCCGGCGTGGTACCATAAAGAAACGGGTGAAATTTACGTCGACCACGAGCCGCCGGCAGA
CATTGAAAATTGGGAGCAAGACCCGGATGTGCTTGATACATGGTTCAGCTCGGCACTCTGGCCGTTCTCC
ACAATGGGGTGGCCGGATACGGAAGCGCCGGACTACAAGCGCTATTACCCGACCGATGTGCTTGTCACCG
GCTATGACATCATTTTCTTCTGGGTGTCGCGCATGATTTTCCAAGGGCTTGAGTTCACTGGGAAGAGACC
GTTTAAAGATGTGTTGATCCACGGCCTCGTCCGCGACGCTCAAGGAAGAAAAATGAGCAAGTCGCTCGGC
AACGGTGTCGACCCGATGGATGTCATTGACCAATACGGCGCCGATGCGCTCCGCTACTTCCTAGCGACCG
GTAGCTCGCCAGGACAAGATTTGCGCTTTAGCACGGAAAAAGTTGAGGCGACGTGGAATTTTGCTAACAA
AATTTGGAACGCTTCACGTTTCGCCTTAATGAACATGGGCGGCATGACATATGAGGAGCTCGATTTGAGC
GGCGAAAAAACGGTCGCCGACCATTGGATTTTAACGCGCTTAAATGAAACGATCGACACGGTGACGAAGC
TCGCCGACAAATACGAGTTTGGTGAAGTCGGTCGCACGTTGTACAACTTTATTTGGGACGATTTGTGCGA
CTGGTACATTGAAATGGCGAAGCTGCCGCTTTACGGCGATGATGAGACAGCGAAAAAGACGACGCGTTCA
GTTTTAGCGTATGTGCTTGACAATACGATGCGCTTGTTGCATCCATTCATGCCGTTCATTACCGAGGAAA
TTTGGCAAAACTTGCCGCATGACGGCGAATCGATTACCGTTGCCTCGTGGCCGCAAGTGCGTCCGGAGCT
GTCAAACGAAGAAGCGGCGGAAGAAATGCGGATGCTCGTTGACATTATCCGCGCGGTCCGAAACGTTCGT
GCCGAAGTCAATACGCCGCCGAGCAAACCGATTGCGCTCTACATTAAGACAAAAGACGAACAAGTGCGCG
CAGCGCTTATGAAAAACCGCGCTTATCTCGAACGGTTCTGCAATCCGAGCGAATTGATCATTGACACGGA
TGTTCCGGCGCCAGAAAAAGCGATGACTGCTGTCGTCACAGGGGCAGAGCTCATTTTGCCGCTTGAAGGA
CTCATCAATATCGAAGAAGAAATCAAGCGGCTTGAGAAAGAGCTCGACAAATGGAACAAAGAAGTCGAGC
GTGTCGAAAAGAAACTGGCGAACGAAGGCTTTTTGGCAAAAGCGCCGGCTCATGTCGTCGAGGAAGAGCG
GCGCAAGCGGCAAGATTACATCGAAAAACGCGAAGCAGTGAAAGCGCGTCTTGCCGAGTTGAAACGG
SEQ ID NO. 130
Amino Acid
ValRS-GsuValRS
Geobacillus subterraneus DSM 13552 (91A1)
MKGAFLLAYRTVDPVGNTAIVYHMKEGIKVAQHEVSMPPKYDHRAVEAGRYDWWLKGKFFETTGDPDKQP
FTIVIPPPNVTGKLHLGHAWDTTLQDIITRMKRMQGYDVLWLPGMDHAGIATQAKVEEKLRQQGLSRYDL
GREKFLEETWKWKEEYAGHIRSQWAKLGLGLDYTRERFTLDEGLSKAVREVFVSLYRKGLIYRGEYIINW
DPATKTALSDIEVIYKEVKGALYHLRYPLADGSGYIEVATTRPETMLGDTAVAVHPDDERYKHLIGKMVK
LPIVGREIPIIADEYVDMEFGSGAVKITPAHDPNDFEVGNRHNLPRILVMNEDGTMNENALQYQGLDRFE
CRKQIVRDLQEQGVLFKIEEHVHSVGHSERSGAVVEPYLSTQWFVKMKPLAEAAIKMQQTEGKVQFVPER
FEKTYLHWLENIRDWCISRQLWWGHRIPAWYHKETGEIYVDHEPPADIENWEQDPDVLDTWFSSALWPFS
TMGWPDTEAPDYKRYYPTDVLVTGYDIIFFWVSRMIFQGLEFTGKRPFKDVLIHGLVRDAQGRKMSKSLG
NGVDPMDVIDQYGADALRYFLATGSSPGQDLRFSTEKVEATWNFANKIWNASRFALMNMGGMTYEELDLS
GEKTVADHWILTRLNETIDTVTKLADKYEFGEVGRTLYNFIWDDLCDWYIEMAKLPLYGDDETAKKTTRS
VLAYVLDNTMRLLHPFMPFITEEIWQNLPHDGESITVASWPQVRPELSNEEAAEEMRMLVDIIRAVRNVR
AEVNTPPSKPIALYIKTKDEQVRAALMKNRAYLERFCNPSELIIDTDVPAPEKAMTAVVTGAELILPLEG
LINIEEEIKRLEKELDKWNKEVERVEKKLANEGFLAKAPAHVVEEERRKRQDYIEKREAVKARLAELKR
SEQ ID NO. 131
DNA
MTF-GsuMTF
Geobacillus subterraneus DSM 13552 (91A1)
ATGCTGATGACGAACATTGTCTTTATGGGAACGCCTGATTTTGCGGTGCCGGTTTTACGGCAGCTGCTTG
ATGACGGGTATCGGGTTGTTGCCGTTGTTACGCAGCCGGACAAGCCGAAAGGGCGAAAGCGCGAGCTTGT
TCCGCCCCCCGTTAAGGTCGAGGCGCAAAAACACGGCATCCCGGTATTGCAACCGACGAAAATTCGTGAA
CCGGAACAATACGAACAAGTGCTGGCGTTTGCGCCTGACTTGATCGTGACCGCGGCATTTGGACAAATTT
TGCCTAAGGCTCTGCTTGACGCTCCCAAATATGGCTGCATTAATGTTCACGCCTCGCTTCTTCCCGAGCT
GCGCGGCGGTGCGCCGATCCATTATGCCATTTGGCAAGGGAAAACGAAAACAGGTGTCACGATTATGTAT
ATGGCGGAAAAGTTGGATGCCGGCGACATGTTGACGCAAGTCGAAGTGCCGATTGAAGAAACCGATACCG
TCGGCACACTGCATGATAAATTGAGCGCTGCCGGGGCTAAACTATTATCAGAAACGCTCCCGCTTTTATT
GGAAGGTAACCTTGCGCCTATTCCGCAAGAGGAAGAGAAAGCGACATATGCTCCGAATATCCGGCGTGAA
CAAGAGCGGATTGACTGGGCGCAGCCTGGTGAGGCGATTTACAACCATATCCGTGCTTTTCATCCGTGGC
CGGTTACGTATACGACATACGACGGGAACGTTTGGAAAATCTGGTGGGGCGAAAAAGTGCCGGCGCCAAG
CTTAGCGTCGCCAGGCACGATTTTATCGCTTGAGGAAGACGGCATCGTCGTCGCCACCGGCAGTGAGACG
GCCATTAAAATTACTGAATTGCAGCCGGCCGGCAAAAAGCGAATGGCGGCCAGCGAGTTTTTGCGCGGTG
CTGGCAGCCGGCTTGCGGTCGGCACGAAGCTAGGAGAGAACAATGAACGTACG
SEQ ID NO. 132
Amino Acid
MTF-GsuMTF
Geobacillus subterraneus DSM 13552 (91A1)
MLMTNIVFMGTPDFAVPVLRQLLDDGYRVVAVVTQPDKPKGRKRELVPPPVKVEAQKHGIPVLQPTKIRE
PEQYEQVLAFAPDLIVTAAFGQILPKALLDAPKYGCINVHASLLPELRGGAPIHYAIWQGKTKTGVTIMY
MAEKLDAGDMLTQVEVPIEETDTVGTLHDKLSAAGAKLLSETLPLLLEGNLAPIPQEEEKATYAPNIRRE
QERIDWAQPGEAIYNHIRAFHPWPVTYTTYDGNVWKIWWGEKVPAPSLASPGTILSLEEDGIVVATGSET
AIKITELQPAGKKRMAASEFLRGAGSRLAVGTKLGENNERT
SEQ ID NO. 133
Amino Acid
RF-1-Mut-GsRF-1-EcOpt
Geobacillus stearothermophilus
MFDRLEAVEQRYEKLNELLMEPDVINDPKKLRDYSKEQADLGETVQTYREYKSVREQLAEAKAMLEEKLE
PELREMVKEEIGELEEREEALVEKLKVLLLPKDPNDEKNVIMEIRAAAGGEEAALFAGDLYRMYTRYAES
QGWKTEVIEASPTGLGGYKEIIFMINGKGAYSKLKFENGAHRVQRVPETESGGRIHTSTATVACLPEMEE
IEVEINEKDIRVDTFASSGPGGQSVNTTMSAVRLTHIPTGIVVICQDEKSQIKNKEKAMKVLRARIYDKY
QQEARAEYDQTRKQAVGTGDRSERIRTYNFPQNRVIDHRIGLTIQKLDQVPDGHLDEIIEALILDDQAKK
LEQANDAS
SEQ ID NO. 134
Amino Acid
muGFP + His6 tag + C-tag
Aequorea victoria
MRGSHHHHHHGSSKGEELFTGVVPILVELDGDVNGHKFSVRGEGEGDATNGKLTLKFICTIGKLPVPWPT
LVTTLTYGVLCFSRYPDHMKRHDFFKSAMPEGYVQERTISFKDDGTYKTRAEVKFEGDTLVNRIELKGID
FKEDGNILGHKLEYNFNSHNVYITADKQKNGIKAYFKIRHNVEDGSVQLADHYQQNTPIGDGPVLLPDNH
YLSTQSVLSKDPNEKRDHMVLLEDVTAAGITHGMDELYKGSEPEA
SEQ ID NO. 135
Amino Acid
deGFP
Aequorea victoria
MELFTGVVPILVELDGDVNGHKFSVSGEGEGDATYGKLTLKFICTTGKLPVPWPTLVTTLTYGVQCFSRY
PDHMKQHDFFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSALSKDPNEK
RDHMVLLEFVTAAGI
SEQ ID NO. 136
Amino Acid
T7 RNA Polymerase
T7 Bacteriophage
MNTINIAKNDFSDIELAAIPFNTLADHYGERLAREQLALEHESYEMGEARFRKMFERQLKAGEVADNAAA
KPLITILLPKMIARINDWFEEVKAKRGKRPTAFQFLQEIKPEAVAYITIKTTLACLTSADNITVQAVASA
IGRAIEDEARFGRIRDLEAKHFKKNVEEQLNKRVGHVYKKAFMQVVEADMLSKGLLGGEAWSSWHKEDSI
HVGVRCIEMLIESTGMVSLHRQNAGVVGQDSETIELAPEYAEAIATRAGALAGISPMFQPCVVPPKPWTG
ITGGGYWANGRRPLALVRTHSKKALMRYEDVYMPEVYKAINIAQNTAWKINKKVLAVANVITKWKHCPVE
DIPAIEREELPMKPEDIDMNPEALTAWKRAAAAVYRKDKARKSRRISLEFMLEQANKFANHKAIWFPYNM
DWRGRVYAVSMFNPQGNDMTKGLLTLAKGKPIGKEGYYWLKIHGANCAGVDKVPFPERIKFIEENHENIM
ACAKSPLENTWWAEQDSPFCFLAFCFEYAGVQHHGLSYNCSLPLAFDGSCSGIQHFSAMLRDEVGGRAVN
LLPSETVQDIYGIVAKKVNEILQADAINGTDNEVVTVTDENTGEISEKVKLGTKALAGQWLAYGVTRSVT
KRSVMTLAYGSKEFGFRQQVLEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLMFLGQFRLQPTINTNKDSEID
AHKQESGIAPNFVHSQDGSHLRKTVVWAHEKYGIESFALIHDSFGTIPADAANLFKAVRETMVDTYESCD
VLADFYDQFADQLHESQLDKMPALPAKGNLNLRDILESDFAFA

What is claimed is:

1. A system for recombinant cell-free expression comprising:
a core recombinant protein mixture having at least the following components:
a plurality of initiation factors (IFs);
a plurality of elongation factors (EFs);
a plurality of peptide release factors (RFs);
at least one ribosome recycling factor (RRF);
a plurality of aminoacyl-tRNA-synthetases (RSs); and
at least one methionyl-tRNA transformylase (MTF);
at least one nucleic acid synthesis template;
a reaction mixture having cell-free reaction components necessary for in vitro macromolecule synthesis; and
wherein the above components are situated in a bioreactor configured for cell-free expression of macromolecules.

2. The system of claim 1, wherein the components of said core recombinant protein mixture comprises a core recombinant protein mixture derived from a bacteria.

3. The system of claim 2, wherein said core recombinant protein mixture derived from bacteria comprises a core recombinant protein mixture wherein at least one components is derived from a thermophilic bacteria.

4. The system of any one of claims 2, and 3, wherein said thermophilic bacteria comprises a thermophilic Bacillaceae bacteria, or Geobacillus thermophilic bacteria.

5. The system of claim 4, wherein said Geobacillus thermophilic bacteria is selected from the group consisting of: Geobacillus subterraneus, and Geobacillus stearothermophilus.

6. The system of claim 1, wherein said core recombinant protein mixture derived from bacteria comprises a core recombinant protein mixture wherein at least one components is derived from a non-thermophilic bacteria, or a combination of non-thermophilic and thermophilic bacteria.

7. The system of claim 6, wherein said non-thermophilic bacteria comprise Escherichia coli.

8. The system of claim 1, wherein said plurality of initiation factors (IFs) comprises a plurality of initiation factors derived from thermophilic bacteria.

9. The system of any one of claims 1, and 8, wherein said plurality of initiation factors derived from thermophilic bacteria comprise IF1, IF2, IF3, or a fragment or variant of any of the same.

10. The system of any one of claims 1, 8, and 9, wherein the plurality of initiation factors are selected from the group of amino acid sequences consisting of: SEQ ID NOs. 2, 4, 6, 70, 72, and 74, or a sequence having at least 90% sequence identity.

11. The system of claim 1, wherein said plurality of elongation factors (EFs) comprises a plurality of elongation factors derived from thermophilic bacteria.

12. The system of any one of claims 1, and 11, wherein said plurality of elongation factors derived from thermophilic bacteria comprise EF-G; EF-Tu; EF-Ts; EF-4; EF-P, or a fragment or variant of any of the same.

13. The system of any one of claims 1, 11, and 12, wherein the plurality of elongation factors are selected from the group of amino acid sequences consisting of: SEQ ID NOs. 8, 10, 12, 14, 16, 76, 78, 80, 82, and 84, or a sequence having at least 90% sequence identity.

14. The system of claim 1, wherein said plurality of peptide release factors (RFs) comprises a plurality of peptide release factors is derived from thermophilic bacteria, or a Bacillus bacteria.

15. The system of any one of claims 1, and 14, wherein said plurality of peptide release factors derived from a thermophilic bacteria comprise RF1, RF2, and RF3, or a fragment or variant of any of the same.

16. The system of any one of claims 1, 14, and 15, wherein the plurality of peptide release factors are selected from the group of amino acid sequences consisting of: SEQ ID NOs. 18, 20, 22, 86, 88, or a sequence having at least 90% sequence identity.

17. The system of claim 1, wherein said ribosome recycling factor (RRF) comprises a ribosome recycling factor derived from thermophilic bacteria.

18. The system of any one of claims 1, and 17, wherein said ribosome recycling factor is derived from Geobacillus.

19. The system of any one of claims 1, 17, and 18, wherein the ribosome recycling factor comprises a ribosome recycling factor according to amino acid sequences SEQ ID NOs. 14, and 90, or a sequence having at least 90% sequence identity.

20. The system of claim 1, wherein said plurality of aminoacyl-tRNA-synthetases (RSs) comprises a plurality of aminoacyl-tRNA-synthetases derived from thermophilic bacteria, or E. Coli.

21. The system of any one of claims 1, and 20, wherein the plurality of aminoacyl-tRNA-synthetases comprises AlaRS; ArgRS; AsnRS; AspRS; CysRS; GlnRS; GluRS; GlyRS; HisRS; IleRS; LeuRS; LysRS; MetRS; PheRS (a); PheRS (b); ProRS; SerRS; ThrRS; TrpRS; TyrRS; and ValRS, or a fragment or variant of any of the same.

22. The system of any one of claims 1, 20, and 21, wherein said plurality of aminoacyl-tRNA-synthetases are selected from the group of amino acid sequences consisting of: SEQ ID NOs. 26, 28. 32, 34, 36, 38, 40, 42, 44, 46, 48, 50, 52, 54, 56, 58, 60, 62, 64, 66, 94, 96, 98, 100, 102, 104, 106, 108, 110, 112, 114, 116, 118, 120, 122, 124, 126, 128, and 130, or a sequence having at least 90% sequence identity

23. The system of claim 1, wherein said methionyl-tRNA transformylase (MTF) comprises a methionyl-tRNA transformylase derived from thermophilic bacteria.

24. The system of claims 1, and 23, wherein said methionyl-tRNA transformylase is derived from Geobacillus.

25. The system of any one of claims 1, 23, and 24, wherein the methionyl-tRNA transformylase comprises a methionyl-tRNA transformylase according to amino acid sequences SEQ ID NOs. 68, and 132, or a sequence having at least 90% sequence identity.

26. The system of claim 1, wherein said nucleic acid synthesis template comprises a DNA template.

27. The system of claim 26, wherein said DNA template comprises a linear DNA template having:
at least one target sequence operably linked to a promoter, and wherein said target sequence may optionally be codon optimized;
at least one ribosome binding site (RBS);
at least one expression product cleavage site; and
at least one tag.

28. The system of claim 1, wherein said nucleic acid synthesis template comprises an RNA template.

29. The system of claim 1, wherein said reaction mixture comprises one or more of the following components:
a quantity of ribosomes, and optionally a quantity of ribosomes derived from thermophilic bacteria;
a quantity of RNase inhibitor;
a quantity of RNA polymerase;
a quantity of tRNAs, and optionally a quantity of tRNAs derived from thermophilic bacteria;
a buffer; and
a quantity of amino acids.

30. The system of claim 29, wherein said reaction mixture further comprises one or more of the following components:
Tris-Acetate;
Mg(OAc)2;
K⁺-glutamate;
amino-acetate;
NaCl;
KCl;
MgCl₂;
DTT;
octyl-b-glycoside;
NAD;
NADP;
sorbitol;
FADH;
CoA;
PLP; and
SAM.

31. The system of any of claims 1, and 29, and further comprising an energy source.

32. The system of claim 32, wherein said energy source comprises a quantity of nucleotide tri-phosphates (NTPs).

33. The system of claim 32, wherein said nucleotide tri-phosphates comprise one or more of the nucleotide tri-phosphates selected from the group consisting of: adenine triphosphate (ATP);
Guanosine triphosphate (GTP), Uridine triphosphate UTP, and Cytidine triphosphate (CTP).

34. The system of any of claims 31, 32, and 33, wherein said energy source comprises an inorganic polyphosphate-based energy regeneration system.

35. The system of claim 34, wherein said inorganic polyphosphate-based energy regeneration system comprises:
a cellular adenosine triphosphate (ATP) energy regeneration system comprising:
a quantity of Adenosyl Kinase (Gst AdK) enzyme;
a quantity of Polyphosphate Kinase (Taq PPK) enzyme;
a quantity of inorganic polyphosphate (PPi); and
a quantity of adenosine monophosphate (AMP);
wherein said AdK and PPK enzymes work synergistically to regenerate cellular ATP energy from PPi and AMP.

36. The system of claim 1, wherein said bioreactor comprises a continuous flow bioreactor.

37. A recombinant cell-free expression reaction mixture comprising:
a plurality of initiation factors (IFs);
a plurality of elongation factors (EF);
a plurality of release factors (RF)
at least one ribosome recycling factor (RRF);
a plurality of aminoacyl-tRNA-synthetases (RSs); and
at least one methionyl-tRNA transformylase (MTF);

38. The system of claim 37, wherein said plurality of initiation factors (IFs) comprise a plurality of initiation factors derived from thermophilic bacteria.

39. The system of any one of claims 37, and 38, wherein said plurality of initiation factors derived from thermophilic bacteria comprise IF1, IF2, IF3, or a fragment or variant of any of the same.

40. The system of any one of claims 37, 38, and 39, wherein the plurality of initiation factors are selected from the group of amino acid sequences consisting of: SEQ ID NOs. 2, 4, 6, 70, 72, and 74, or a sequence having at least 90% sequence identity.

41. The system of claim 37, wherein said plurality of elongation factors (EFs) comprise a plurality of elongation factors derived from thermophilic bacteria.

42. The system of any one of claims 37, and 41, wherein said plurality of elongation factors derived from a thermophilic bacteria comprises EF-G, EF-Tu, EF-Ts, EF-4, EF-P, or a fragment or variant of any of the same.

43. The system of any one of claims 37, 41, and 42, wherein the plurality of elongation factors are selected from the group of amino acid sequences consisting of: SEQ ID NOs. 8, 10, 12, 14, 16, 76, 78, 80, 82, and 84, or a sequence having at least 90% sequence identity.

44. The system of claim 37, wherein said plurality of peptide release factors (RFs) comprise a plurality of release factors derived from thermophilic bacteria, or a Bacillus sp. bacteria.

45. The system of any one of claims 37, and 44, wherein the plurality of peptide release factors comprises RF1, RF2, and RF3, or a fragment or variant of any of the same.

46. The system of any one of claims 37, 44, and 45, wherein the plurality of peptide release factors are selected from the group of amino acid sequences consisting of: SEQ ID NOs. 18, 20, 22, 86, 88, or a sequence having at least 90% sequence identity.

47. The system of claim 37, wherein said ribosome recycling factor (RRF) comprise a ribosome recycling factor derived from thermophilic bacteria.

48. The system of any one of claims 37, and 47, wherein said ribosome recycling factor derived from Geobacillus.

49. The system of any one of claims 37, 47, and 48, wherein the ribosome recycling factor comprise a ribosome recycling factor according to amino acid sequence SEQ ID NOs. 14, and 90, or a sequence having at least 90% sequence identity.

50. The system of claim 37, wherein said plurality of aminoacyl-tRNA-synthetases (RSs) comprise a plurality of aminoacyl-tRNA-synthetases wherein at least one is derived from thermophilic bacteria.

51. The system of any one of claims 37, and 50, wherein the plurality of aminoacyl-tRNA-synthetases comprise AlaRS; ArgRS; AsnRS; AspRS; CysRS; GlnRS; GluRS; GlyRS; HisRS; IleRS; LeuRS; LysRS; MetRS; PheRS (a); PheRS (b); ProRS; SerRS; ThrRS; TrpRS; TyrRS; and ValRS, or a fragment or variant of any of the same.

52. The system of any one of claims 37, 50, and 51, wherein said plurality of aminoacyl-tRNA-synthetases are selected from the group of amino acid sequences consisting of: SEQ ID NOs. 26, 28. 32, 34, 36, 38, 40, 42, 44, 46, 48, 50, 52, 54, 56, 58, 60, 62, 64, 66, 94, 96, 98, 100, 102, 104, 106, 108, 110, 112, 114, 116, 118, 120, 122, 124, 126, 128, and 130, or a sequence having at least 90% sequence identity

53. The system of any one of claims 37, wherein said methionyl-tRNA transformylase (MTF) comprises a methionyl-tRNA transformylase derived from thermophilic bacteria.

54. The system of any one of claims 37, and 53, wherein said methionyl-tRNA transformylase derived from Geobacillus.

55. The system of any one of claims 37, 53, and 54, wherein the methionyl-tRNA transformylase comprises a methionyl-tRNA transformylase according to amino acid sequence SEQ ID NOs. 68, and 132, or a sequence having at least 90% sequence identity.

56. An isolated nucleotide comprising a nucleotide selected from the group consisting of:
SEQ ID NOs. 1, 3, 5 69, 71, and 73;
SEQ ID NOs. 7, 9, 11, 13, 15, 75, 77, 79, 81, and 83;
SEQ ID NOs. 17, 19, 21, 85, and 87;
SEQ ID NOs. 23, and 89; and
SEQ ID NO. 25, 27, 29, 31, 33, 35,37, 39, 41, 43, 45, 47, 49, 51, 53, 55, 57, 59, 61, 63, 65, 67, 91, 93, 95, 97, 99, 101, 103, 105, 107, 109, 111, 113, 115, 117, 119, 121, 123, 125, 127, 129 and 131.

57. An expression vector comprising at least one of the nucleotide sequences of claim 56, operably linked to a promoter.

58. A bacteria transformed by one of the expression vectors of claim 57.

59. The transformed bacteria of claim 58, wherein said bacteria comprises E. coli.

60. A peptide comprising an amino acid sequence selected from the group consisting of:
SEQ ID NOs. 2, 4, 6, 70, 72 and 74;
SEQ ID NOs. 8, 10, 12, 14, 16, 76, 78, 80, 82, and 84;
SEQ ID NOs. 18, 20, 22, 86, 88;
SEQ ID NOs. 14, and 90;
SEQ ID NOs. 26, 28. 32, 34, 36, 38, 40, 42, 44, 46, 48, 50, 52, 54, 56, 58, 60, 62, 64, 66, 94, 96, SEQ ID NOs. 98, 100, 102, 104, 106, 108, 110, 112, 114, 116, 118, 120, 122, 124, 126, 128, and 130; and
SEQ ID NOs. 68, and 132, or a fragment or variant of any of the same.

61. A cell-free expression system using at least one of the peptides of claim 60.


SEQUENCE LISTING (136 sequences)

SEQ ID NO: 1 - DNA, 219 bp, Geobacillus
atggccaaagatgatgtgattgaagttgaaggcaccgttattgaaaccctgccgaatgcaatgtttcgtgttgaactggaaaatggtcataccgttctggcacatgttagcggtaaaattcgcatgcactttattcgtattctgcctggtgatcgtgttaccgttgaactgagcccgtacgatctgacccgtggtcgtattacctatcgttataaatga

SEQ ID NO: 2 - protein, 72 aa, Geobacillus
MAKDDVIEVEGTVIETLPNAMFRVELENGHTVLAHVSGKIRMHFIRILPGDRVTVELSPYDLTRGRITYRYK

SEQ ID NO: 3 - DNA, 2220 bp, Geobacillus stearothermophilus
atgagcaaaatgcgcgtttatgagtacgccaaaaaacagaatgttccgagcaaagatgtgatccacaaactgaaagaaatgaacatcgaagtgaacaaccatatggcaatgctggaagcagatgttgttgaaaaactggatcatcagtatcgtccgaataccggcaaaaaagaagaaaaaaaagccgagaagaaaaccgagaaaccgaaacgtccgacaccagcaaaagcagcagattttgcagatgaagaaatcttcgatgatagcaaagaagcagccaaaatgaaaccggcaaagaaaaaaggtgcaccgaaaggtaaagaaaccaaaaaaaccgaagcacagcagcaagagaaaaaactgctgcaggcagcgaaaaagaaaggcaaaggtccggcaaaagggaaaaaacaggcagcaccggcagccaaacaggcaccgcagcctgcgaaaaaagaaaaagaactgccgaaaaaaatcacctttgaaggtagcctgaccgttgcagaactggcaaaaaaactgggtcgtgaaccgagcgaaattatcaaaaaactgtttatgctgggtgtgatggccaccattaatcaggatctggataaagatgccattgaactgatttgcagcgattatggtgttgaggttgaagaaaaagtgaccatcgatgaaaccaactttgaagccattgaaattgttgatgcaccggaagatctggttgaacgtccgcctgttgttaccattatgggtcatgttgatcatggtaaaaccacactgctggatgcaattcgtcatagcaaagttaccgaacaagaagcaggcggtattacacagcatattggtgcatatcaggttaccgtgaacgataagaaaatcacgtttctggatacaccgggtcatgaagcatttaccaccatgcgtgcacgtggtgcacaggtgaccgatattgttattctggttgttgcagcagatgatggcgttatgccgcagaccgttgaagcaattaatcatgcaaaagccgcaaacgttccgattattgttgccatcaacaaaatcgataaaccggaagcaaatccggatcgtgttatgcaagaactgatggaatataatctggttccggaagaatggggtggtgataccattttttgtaaactgagcgccaaaaccaaagaaggtctggaccatctgctggaaatgattctgctggttagcgaaatggaagaactgaaagccaatccgaatcgtcgtgcagttggcaccgttattgaagccaaactggacaaaggtcgtggtccggttgcgaccctgctgattcaggcaggcaccctgcgtgttggtgatccgattgttgtgggcaccacctatggtcgtgttcgtgcaatggttaatgatagcggtcgtcgtgttaaagaagcaaccccgagcatgccggttgaaattaccggtctgcatgaagttccgcaggcaggcgatcgttttatggtttttgaagatgagaaaaaggcacgccagattgccgaagcacgtgcacagcgtcagctgcaagaacagcgtagcgttaaaacccgtgttagcctggatgacctgtttgagcagattaaacagggtgaaatgaaagagctgaacctgattgttaaagccgatgttcagggtagcgttgaagccctggttgcagcactgcagaaaattgatgttgaaggtgttcgcgtgaaaattatccatgcagccgttggtgcaattaccgaaagcgatattagcctggcaaccgcaagcaatgcaattgtgattggttttaatgttcgtccggatgcaaatgcaaaacgtgcagcagaaagtgaaaaagtggatattcgtctgcaccgcattatctataacgtgatcgaagaaattgaggcagccatgaaaggtatgctggatccggaatatgaagagaaagttattggtcaggcagaagttcgtcagacctttaaagttagcaaagtgggtacaattgccggttgttatgttaccgatggtaaaattacccgtgatagtaaagttcgtctgattcgtcagggtattgttgtgtatgaaggtgaaattgatagcctgaaacgctataaagatgatgttcgtgaagttgcccagggttatgaatgtggtctgaccattaaaaacttcaacgacattaaagagggcgacgttatcgaagcctatatcatgcaagaagttgcacgcgcataa

SEQ ID NO: 4 - protein, 736 aa, Geobacillus stearothermophilus
MSKMRVYEYAKKQNVPSKDVIHKLKEMNIEVNNHMAMLEADVVEKLDHQYRPNTGKKEEKKAEKKTEKPKRPTPAKAADFADEEIFDDSKEAAKMKPAKKKGAPKGKETKKTEAQQQEKKLLQAAKKKGKGPAKGKKQAAPAAKQAPQPAKKEKELPKKITFEGSLTVAELAKKLGREPSEIIKKLFMLGVMATINQDLDKDAIELICSDYGVEVEEKVTIDETNFEAIEIVDAPEDLVERPPVVTIMGHVDHGKTTLLDAIRHSKVTEQEAGGITQHIGAYQVTVNDKKITFLDTPGHEAFTTMRARGAQVTDIVILWAADDGVMPQTVEAINHAKAANVPIIVAINKIDKPEANPDRVMQELMEYNLVPEEWGGDTIFCKLSAKTKEGLDHLLEMILLVSEMEELKANPNRRAVGTVIEAKLDKGRGPVATLLIQAGTLRVGDPIWGTTYGRVRAMVNDSGRRVKEATPSMPVEITGLHEVPQAGDRFMVFEDEKKARQIAEARAQRQLQEQRSVKTRVSLDDLFEQIKQGEMKELNLIVKADVQGSVEALVAALQKIDVEGVRVKIIHAAVGAITESDISLATASNAIVIGFNVRPDANAKRAAESEKVDIRLHRIIYNVIEEIEAAMKGMLDPEYEEKVIGQAEVRQTFKVSKVGTIAGCYVTDGKITRDSKVRLIRQGIWYEGEIDSLKRYKDDVREVAQGYECGLTIKNFNDIKEGDVIEAYIMQEVARA

SEQ ID NO: 5 - DNA, 522 bp, Geobacillus
atgatcagcaaggactttatcatcaatgagcagattcgtgcacgtgaagttcgtctgattgatcagaatggtgaacagctgggtatcaaaagcaaacaagaagcactggaaattgcagcacgtcgtaatctggatctggttctggtggcaccgaatgcaaaaccgcctgtttgtcgtattatggattatggcaaatttcgcttcgagcagcagaaaaaagaaaaagaggcacgcaaaaagcagaaagtgatcaatgttaaagaagtgcgtctgagcccgaccattgaagaacatgattttaacaccaaactgcgcaacgcacgcaaatttctggaaaaaggtgataaagtgaaagccaccattcgttttaaaggtcgtgcaatcacccataaagaaattggtcagcgtgttctggatcgttttagcgaagcatgtgcagatattgcagttgttgaaaccgcaccgaaaatggatggtcgtaatatgtttctggtgctggctccgaaaaacgacaacaaataa

SEQ ID NO: 6 - protein, 173 aa, Geobacillus
MISKDFIINEQIRAREVRLIDQNGEQLGIKSKQEALEIAARRNLDLVLVAPNAKPPVCRIMDYGKFRFEQQKKEKEARKKQKVINVKEVRLSPTIEEHDFNTKLRNARKFLEKGDKVKATIRFKGRAITHKEIGQRVLDRFSEACADIAVVETAPKMDGRNMFLVLAPKNDNK

SEQ ID NO: 7 - DNA, 2079 bp, Geobacillus
atggcacgtgaattcagcctggaaaaaacccgtaatattggtattatggcccatatcgatgcaggtaaaaccaccaccaccgaacgtattctgttttataccggtcgtgtgcataaaattggtgaagttcatgaaggtgcagcaaccatggattggatggaacaagaacaagagcgtggtattaccattaccagcgcagccaccaccgcacagtggaaaggtcatcgtattaacattattgatacaccgggtcacgttgattttaccgttgaagttgaacgtagcctgcgtgttctggatggtgcaattaccgtgctggatgcacagagcggtgttgaaccgcagaccgaaaccgtttggcgtcaggcaaccacctatggtgttccgcgtattgtttttgtgaacaagatggataaaatcggtgccgatttcctgtatagcgttaaaaccctgcatgatcgtctgcaggcaaatgcacatccggttcagctgccgattggtgcagaagatcagtttagcggtattattgatctggttgaaatgtgcgcctatcactatcatgatgaactgggcaaaaacatcgaacgcattgatattccggaagaatatcgtgatatggccgaagagtatcacaacaaactgattgaagcagttgcagaactggatgaagaactgatgatgaaatatctggaaggcgaagaaattaccgcagaggaactgaaagcagcaattcgtaaagcaaccattagcgtggaattttttccggttttttgtggtagcgccttcaaaaacaaaggtgtgcagctgctgctggatggcgttgttgattatctgccgagtccggtggatattcctgcaattcgtggtgttgttccggataccgaagaagaagttacacgcgaagcaagtgatgatgcaccgtttgcagcactggcctttaaaatcatgaccgatccgtatgttggtaagctgacctttattcgtgtttatagcggcaccctggatagcggtagctatgttatgaataccaccaaaggtaaacgtgaacgtattggtcgtctgctgcagatgcatgcaaatcatcgtcaagaaatcagcaaagtttatgccggtgatattgcagcagcagttggtctgaaagataccacaaccggtgataccctgtgtgatgaaaaacatccggtgattctggaaagcatgcagtttccggaaccggttattagcgttgcaattgaaccgaaaagcaaagccgatcaggataaaatgagccaggcactgcagaaactgcaagaagaggatccgacctttcgtgcacataccgatccggaaaccggtcagaccattattagtggtatgggtgaactgcatctggatatcattgttgatcgtatgcgtcgcgaatttaaagttgaagcaaatgttggtgcaccgcaggttgcatatcgtgaaacctttcgtaaaagcgcacaggttgaaggcaaatttatccgtcagagtggtggtcgtggtcagtatggtcatgtttggattgaattttcaccgaacgaacgcggtaaaggctttgaatttgaaaatgcaattgttggtggtgtggtgccgaaagaatatgttccggcagttcaggcaggtctggaagaggcaatgcagaatggtgttctggcaggttatccggttgttgatattaaagccaaactgttcgatggcagctatcacgatgttgatagcagcgaaatggcattcaaaattgcagcaagcctggcactgaaaaatgccgcaaccaaatgtgatcctgttctgctggaaccgattatgaaagtggaagttgttatccctgaggaatatctgggtgatattatgggcgatattaccagccgtcgtggtcgcattgaaggtatggaagcacgtggtaatgcccaggttgttcgtgcaatggttccgctggcagaaatgtttggttatgcaaccagcctgcgtagcaatacccaaggtcgtggcacctttagcatggtttttgatcattatgaagaggtgcccaaaaacattgccgatgagatcatcaaaaaaaacaagggcgaataa

SEQ ID NO: 8 - protein, 692 aa, Geobacillus
MAREFSLEKTRNIGIMAHIDAGKTTTTERILFYTGRVHKIGEVHEGAATMDWMEQEQERGITITSAATTAQWKGHRINIIDTPGHVDFTVEVERSLRVLDGAITVLDAQSGVEPQTETVWRQATTYGVPRIVFVNKMDKIGADFLYSVKTLHDRLQANAHPVQLPIGAEDQFSGIIDLVEMCAYHYHDELGKNIERIDIPEEYRDMAEEYHNKLIEAVAELDEELMMKYLEGEEITAEELKAAIRKATISVEFFPVFCGSAFKNKGVQLLLDGVVDYLPSPVDIPAIRGVVPDTEEEVTREASDDAPFAALAFKIMTDPYVGKLTFIRVYSGTLDSGSYVMNTTKGKRERIGRLLQMHANHRQEISKVYAGDIAAAVGLKDTTTGDTLCDEKHPVILESMQFPEPVISVAIEPKSKADQDKMSQALQKLQEEDPTFRAHTDPETGQTIISGMGELHLDIIVDRMRREFKVEANVGAPQVAYRETFRKSAQVEGKFIRQSGGRGQYGHVWIEFSPNERGKGFEFENAIVGGVVPKEYVPAVQAGLEEAMQNGVLAGYPVVDIKAKLFDGSYHDVDSSEMAFKIAASLALKNAATKCDPVLLEPIMKVEVVIPEEYLGDIMGDITSRRGRIEGMEARGNAQVVRAMVPLAEMFGYATSLRSNTQGRGTFSMVFDHYEEVPKNIADEIIKKNKGE

SEQ ID NO: 9 - DNA, 1188 bp, Geobacillus
atggccaaagccaaatttgaacgtaccaaaccgcatgttaatattggcaccattggtcatgttgatcatggtaaaaccacactgaccgcagcaattaccaccgttctggcaaaacagggtaaagccgaagcaaaagcatatgatcagattgatgcagcaccggaagaacgtgaacgtggtattaccattagcaccgcacatgttgaatatgaaaccgatgcacgtcattatgcccatgttgattgtccgggtcatgcagattatgtgaaaaatatgattaccggtgcagcacagatggatggtgcaattctggttgttagcgcagcagatggtccgatgccgcagacacgtgaacatattctgctgagccgtcaggttggtgttccgtatattgttgtgtttctgaacaaatgcgatatggtggatgatgaagaactgctggaactggttgaaatggaagttcgtgatctgctgtccgaatatgattttccgggtgatgaagttccggttattaaaggtagcgcactgaaagcactggaaggtgatccgcagtgggaagaaaaaatcattgaactgatgaatgccgtggatgagtatattccgacaccgcagcgtgaagttgataaaccgtttatgatgccgatcgaagatgtgtttagcattaccggtcgtggcaccgttgcaaccggtcgcgttgaacgtggcaccctgaaagttggtgatccggttgaaattattggtctgagtgatgaaccgaaaaccaccaccgttaccggtgttgaaatgtttcgtaaactgttagatcaggccgaagccggtgataatattggtgcactgctgcgtggtgtttcacgtgatgaggtggaacgtggtcaggttctggcgaaacctggtagcattacaccgcataccaaattcaaagcacaggtttatgttctgaccaaagaagaaggcggtcgtcataccccgttttttagcaattatcgtccgcagttttatttccgtaccaccgatgttaccggtattattaccctgccggaaggtgtggaaatggttatgcctggtgataacgttgaaatgaccgtggaactgattgcaccgattgcaattgaagaaggcaccaaatttagcattcgtgaaggtggtcgtaccgttggtgcaggtagcgttagcgaaattatcgaataa

SEQ ID NO: 10 - protein, 395 aa, Geobacillus
MAKAKFERTKPHVNIGTIGHVDHGKTTLTAAITTVLAKQGKAEAKAYDQIDAAPEERERGITISTAHVEYETDARHYAHVDCPGHADYVKNMITGAAQMDGAILVVSAADGPMPQTREHILLSRQVGVPYIVVFLNKCDMVDDEELLELVEMEVRDLLSEYDFPGDEVPVIKGSALKALEGDPQWEEKIIELMNAVDEYIPTPQREVDKPFMMPIEDVFSITGRGTVATGRVERGTLKVGDPVEIIGLSDEPKTTTVTGVEMFRKLLDQAEAGDNIGALLRGVSRDEVERGQVLAKPGSITPHTKFKAQVYVLTKEEGGRHTPFFSNYRPQFYFRTTDVTGIITLPEGVEMVMPGDNVEMTVELIAPIAIEEGTKFSIREGGRTVGAGSVSEIIE

SEQ ID NO: 11 - DNA, 885 bp, Geobacillus
atggcaattaccgcacagatggttaaagaactgcgtgaaaaaaccggtgcaggtatgatggattgtaaaaaagcactgaccgaaaccaatggcgatatggaaaaagcaattgattggctgcgcgaaaaaggtattgcaaaagcagcaaaaaaagccgatcgtattgcagcagaaggtatggcatatattgcagttgaaggtaataccgcagttatcctggaagttaatagcgaaaccgattttgtggcaaaaaacgaagcatttcagaccctggtgaaagagctggcagcacatctgctgaaacagaaaccggcaagcctggatgaagcactgggtcagaccatggataatggtagcaccgttcaggattatatcaatgaagccattgccaaaatcggcgaaaaaatcaccctgcgtcgttttgcagttgttaataaagcagatggtgaaacctttggtgcctatctgcatatgggtggtcgtattggtgttctgaccctgctggcaggtaatgcaagcgaagatgttgcaaaagatgtggcaatgcatattgcagccctgcatccgaaatatgttagccgtgatgatgttccgcaagaagaaattgcacacgaacgtgaagttctgaaacagcaggcactgaatgaaggcaaaccggaaaaaattgtggaaaagatggttgaaggtcgcctgaacaaattctatgaagatgtttgtctgctggaacaggcctttgttaaaaatccggatgttaccgttcgtcagtatgttgaaagcaatggtgccaccgttaaacagtttattcgttatgaagttggtgagggcttagaaaaacgccaggataattttgccgaagaagttatgagccaggttcgcaaacagtaa

SEQ ID NO: 12 - protein, 294 aa, Geobacillus
MAITAQMVKELREKTGAGMMDCKKALTETNGDMEKAIDWLREKGIAKAAKKADRIAAEGMAYIAVEGNTAVILEVNSETDFVAKNEAFQTLVKELAAHLLKQKPASLDEALGQTMDNGSTVQDYINEAIAKIGEKITLRRFAVVNKADGETFGAYLHMGGRIGVLTLLAGNASEDVAKDVAMHIAALHPKYVSRDDVPQEEIAHEREVLKQQALNEGKPEKIVEKMVEGRLNKFYEDVCLLEQAFVKNPDVTVRQYVESNGATVKQFIRYEVGEGLEKRQDNFAEEVMSQVRKQ

SEQ ID NO: 13 - DNA, 1827 bp, Geobacillus
atgaaccgtgaggaacgtctgaaacgtcaggagcgtattcgtaacttcagcatcattgcgcacatcgaccacggtaaaagcaccctggcggatcgtatcctggagaaaaccggtgcgctgagcgagcgtgaactgcgtgaacagaccctggacatgatggatctggagcgtgaacgtggtatcaccattaagctgaacgcggtgcaactgacctataaggcgaaaaacggcgaggaatacatcttccacctgattgacaccccgggccacgtggattttacctatgaagttagccgtagcctggcggcgtgcgaaggtgcgattctggtggttgatgcggcgcagggtattgaggcgcaaaccctggcgaacgtgtacctggcgattgacaacaacctggaaatcctgccggttatcaacaaaattgatctgccgagcgcggagccggaacgtgtgcgtcaggagatcgaagacgttattggtctggatgcgagcgaggcggtgctggcgagcgcgaaggttggtatcggcattgaggaaatcctggagcaaattgtggaaaaaattccggcgccgagcggtgacccggatgcgccgctgaaggcgctgatctttgacagcctgtacgatccgtatcgtggcgtggttgcgtacgtgcgtattgttgacggtaccgttaagccgggccagcgtatcaaaatgatgagcaccggcaaggagttcgaagtgaccgaggtgggcgtttttaccccgaagcaaaaaatcgttgacgaactgaccgtgggtgatgttggctatctgaccgcgagcattaagaacgtgaaagatacccgtgttggtgacaccattaccgatgcggagcgtccggcggcggaaccgctgccgggttaccgtaaactgaacccgatggttttctgcggcatgtatccgatcgacaccgcgcgttacaacgatctgcgtgaggcgctggaaaagctgcagctgaacgacgcggcgctgcacttcgagccggaaaccagccaagcgctgggtttcggctttcgttgcggttttctgggcctgctgcacatggagatcattcaggaacgtatcgagcgtgaatttcacatcgatctgattaccaccgcgccgagcgtggtttataaagtgcacctgaccgacggtaccgaggtgagcgttgataacccgaccaacatgccggacccgcaaaaaatcgatcgtattgaggaaccgtatgtgaaggcgaccattatggttccgaacgactacgtgggcccggttatggaactgtgccagggtaaacgtggcaccttcgtggacatgcaatacctggatgagaagcgtgttatgctgatctatgacattccgctgagcgaaatcgtttacgacttctttgatgcgctgaagagcaacaccaaaggttacgcgagctttgattatgagctgattggctaccgtccgagcaacctggtgaaaatggacatcctgctgaacggtgaaaagattgatgcgctgagcttcatcgttcaccgtgaggcggcgtatgaacgtggcaaagtgattgttgagaagctgaaagacctgatcccgcgtcagcaatttgaagtgccggttcaggcggcgattggtaacaaaatcattgcgcgtagcaccatcaaggcgctgcgtaaaaacgtgctggcgaagtgctacggtggcgatgttagccgtaagcgtaaactgctggagaagcagaaagaaggtaagaaacgtatgaaacagattggtagcgttgaggtgccgcaagaagcgttcatggcggtgctgaagatcgacgatcaaaagaaa

SEQ ID NO: 14 - protein, 609 aa, Geobacillus
MNREERLKRQERIRNFSIIAHIDHGKSTLADRILEKTGALSERELREQTLDMMDLERERGITIKLNAVQLTYKAKNGEEYIFHLIDTPGHVDFTYEVSRSLAACEGAILVVDAAQGIEAQTLANVYLAIDNNLEILPVINKIDLPSAEPERVRQEIEDVIGLDASEAVLASAKVGIGIEEILEQIVEKIPAPSGDPDAPLKALIFDSLYDPYRGVVAYVRIVDGTVKPGQRIKMMSTGKEFEVTEVGVFTPKQKIVDELTVGDVGYLTASIKNVKDTRVGDTITDAERPAAEPLPGYRKLNPMVFCGMYPIDTARYNDLREALEKLQLNDAALHFEPETSQALGFGFRCGFLGLLHMEIIQERIEREFHIDLITTAPSVVYKVHLTDGTEVSVDNPTNMPDPQKIDRIEEPYVKATIMVPNDYVGPVMELCQGKRGTFVDMQYLDEKRVMLIYDIPLSEIVYDFFDALKSNTKGYASFDYELIGYRPSNLVKMDILLNGEKIDALSFIVHREAAYERGKVIVEKLKDLIPRQQFEVPVQAAIGNKIIARSTIKALRKNVLAKCYGGDVSRKRKLLEKQKEGKKRMKQIGSVEVPQEAFMAVLKIDDQKK

SEQ ID NO: 15 - DNA, 555 bp, Geobacillus
atgatcagcgtgaacgacttccgtaccggtctgaccatcgaagttgatggcgagatttggcgtgtgctggaattccagcacgttaagccgggtaaaggcgcggcgtttgtgcgtagcaagctgcgtaacctgcgtaccggtgcgatccaagaacgtaccttccgtgcgggcgagaaggtgaaccgtgcgcagattgacacccgtaaaatgcaatacctgtatgcgaacggtgaccagcacgtttttatggatatggagacctacgaacagatcgagctgccggcgaaacaaattgagtatgaactgaagttcctgaaagaaaacatggaagtgtttatcatgatgtaccaaggtgaaaccatcggcattgagctgccgaacaccgttgagctgaaggtggttgagaccgaaccgggtattaaaggtgataccgcgagcggtggcagcaagccggcgaaactggaaaccggcctggtggttcaggtgccgttctttgttaacgagggtgacaccctgatcattaacaccgcggatggcacctatgttagccgtgcg

SEQ ID NO: 16 - protein, 185 aa, Geobacillus
MISVNDFRTGLTIEVDGEIWRVLEFQHVKPGKGAAFVRSKLRNLRTGAIQERTFRAGEKVNRAQIDTRKMQYLYANGDQHVFMDMETYEQIELPAKQIEYELKFLKENMEVFIMMYQGETIGIELPNTVELKVVETEPGIKGDTASGGSKPAKLETGLVVQVPFFVNEGDTLIINTADGTYVSRA

SEQ ID NO: 17 - DNA, 1077 bp, Geobacillus stearothermophilus
atgtttgatcgtctggaagcagttgaacagcgttatgaaaaactgaatgaactgctgatggaaccggatgttattaacgatccgaaaaaactgcgcgattatagcaaagaacaggcagatctggaagaaaccgttcagacctatcgtgagtataaaagcgttcgtgaacagctggccgaagcaaaagcaatgctggaagagaaactggaacctgaactgcgtgaaatggtgaaagaagaaattggcgaactggaagaacgtgaagaagcactggttgagaaactgaaagttctgctgctgccgaaagatccgaatgatgaaaaaaacgtgatcatggaaattcgtgcagcagccggtggcgaagaagcagcactgtttgccggtgatctgtatcgtatgtatacccgttatgcagaaagccaaggttggaaaaccgaagttattgaagcaagcccgaccggtttaggtggttataaagaaatcatcttcatgatcaatggcaagggtgcatacagcaaactgaaatttgaaaatggtgcacatcgtgttcagcgtgttccggaaaccgaaagcggtggtcgtattcataccagcaccgcaaccgttgcatgtctgccggaaatggaagaaatcgaagtggaaatcaacgagaaagatattcgcgttgatacctttgcaagcagcggtcctggtggtcagagcgttaataccaccatgagcgcagttcgtctgacccatattccgaccggtattgttgttacctgtcaggatgaaaaatcccagatcaaaaacaaagaaaaagccatgaaagtgctgcgtgcccgtatctatgataaatatcagcaagaggcacgtgcggaatatgatcagacccgtaaacaggcagttggcaccggtgatcgtagcgaacgtattcgtacctataactttccgcagaatcgtgttaccgatcatcgtattggtctgaccattcaaaaactggatcaggttctggatggtcatctggatgaaattatcgaagcactgattctggatgaccaggcaaaaaagctggaacaggcaaatgatgcaagctaa

SEQ ID NO: 18 - protein, 358 aa, Geobacillus stearothermophilus
MFDRLEAVEQRYEKLNELLMEPDVINDPKKLRDYSKEQADLEETVQTYREYKSVREQLAEAKAMLEEKLEPELREMVKEEIGELEEREEALVEKLKVLLLPKDPNDEKNVIMEIRAAAGGEEAALFAGDLYRMYTRYAESQGWKTEVIEASPTGLGGYKEIIFMINGKGAYSKLKFENGAHRVQRVPETESGGRIHTSTATVACLPEMEEIEVEINEKDIRVDTFASSGPGGQSVNTTMSAVRLTHIPTGIVVTCQDEKSQIKNKEKAMKVLRARIYDKYQQEARAEYDQTRKQAVGTGDRSERIRTYNFPQNRVTDHRIGLTIQKLDQVLDGHLDEIIEALILDDQAKKLEQANDAS

SEQ ID NO: 19 - DNA, 984 bp, Geobacillus stearothermophilus
atggcagcaccgaatttttgggatgatcagaaagcagcacaggcagttattagcgaagcaaatgcactgaaagatctggtggaagaatttagcagcctggaagaacgttttgataatctggaagttacctacgaactgctgaaagaagaaccggacgacgaactgcaggcagaactggttgaagaggcaaaaaaactgatgaaagattttagcgaatttgaactgcagctgctgctgaatgaaccgtatgatcaaaataatgccatcctggaactgcatcctggtgccggtggcaccgaaagccaggattgggcaagcatgctgctgcgtatgtatacccgttgggcagaaaaaaaaggctttaaagttgaaaccctggattatctgcctggtgaagaagcaggtattaaaagcgttaccctgctgattaaaggccataatgcatatggttatctgaaagccgaaaaaggtgttcatcgtctggttcgtattagcccgtttgatgcaagcggtcgtcgtcataccagctttgttagctgtgaagttgtgccggaactggatgataacattgaaattgaaattcgccctgaagaactgaagattgatacctatcgtagcagcggtgcaggcggtcagcatgttaataccaccgatagcgcagtgcgtattacccatctgccgaccggtattgttgttacctgtcagagcgaacgtagccagattaaaaaccgtgaaaaagccatgaatatgctgaaagccaaactgtaccagaagaaattagaagaacagcaggccgagctggccgaactgcgtggtgaacagaaagaaattggttggggtaatcagattcgcagctatgtttttcatccgtacagcctggttaaagatcatcgtaccaatgttgaagttggtaatgttcaggccgttatggatggtgaaattgatgtttttatcgatgcatacctgcgtgccaaactgaaataa

SEQ ID NO: 20 - protein, 327 aa, Geobacillus stearothermophilus
MAAPNFWDDQKAAQAVISEANALKDLVEEFSSLEERFDNLEVTYELLKEEPDDELQAELVEEAKKLMKDFSEFELQLLLNEPYDQNNAILELHPGAGGTESQDWASMLLRMYTRWAEKKGFKVETLDYLPGEEAGIKSVTLLIKGHNAYGYLKAEKGVHRLVRISPFDASGRRHTSFVSCEVVPELDDNIEIEIRPEELKIDTYRSSGAGGQHVNTTDSAVRITHLPTGIVVTCQSERSQIKNREKAMNMLKAKLYQKKLEEQQAELAELRGEQKEIGWGNQIRSYVFHPYSLVKDHRTNVEVGNVQAVMDGEIDVFIDAYLRAKLK

SEQ ID NO: 21 - DNA, 1578 bp, Bacillus sp. X1
atgggtaacgatttcaagaaagaagtgctgagccgtcgtacctttgcgatcattagccatccggatgcgggcaagaccaccctgaccgagaaactgctgctgttcggtggcgcgatccgtgatgcgggtaccgttaaggcgaagaaaaccggcaaatacgcgaccagcgactggatggaaatcgagaaacagcgtggtattagcgtgaccagcagcgttatgcaattcgattacaacggttatcgtgtgaacattctggacaccccgggccaccaggactttagcgaagatacctatcgtaccctgatggcggtggacagcgcggttatgatcattgatagcgcgaagggcatcgaggaccaaaccattaagctgttcaaagtgtgccgtatgcgtggtatcccgattttcacctttatcaacaagctggaccgtcagggcaaacaaccgctggagctgctggcggaactggaggaagttctgggtatcgagagctacccgatgaactggccgattggtatgggcaaagaatttctgggcatctatgatcgttactataaccgtattgagcagttccgtgtgaacgaggaagagcgttttatcccgctgaacgaagacggtgaaattgagggcaaccacaagctggttagcagcggtctgtacgagcagaccctggaagagatcatgctgctgaacgaggcgggtaacgaatttagcgcggagcgtgtggcggcgggtcaactgaccccggttttctttggtagcgcgctgaccaacttcggcgtgcagacctttctggaaacctatctgcaatttgctccgccgccgaaggcgcgtaacagcagcatcggcgagattgatccgctgagcgaagagtttagcggcttcgtttttaaaattcaggcgaacatgaacccggcgcaccgtgaccgtatcgcgttcgtgcgtatttgcagcggcaagtttgagcgtggcatgagcgttaacctgccgcgtctgggcaagcagctgaaactgacccaaagcaccagcttcatggcggaagagcgtaacaccgtggaagaggcggttagcggtgacatcattggcctgtacgataccggtacctatcagatcggcgataccctgaccgtgggcaaaaacgacttccagtttgagcgtctgccgcaattcaccccggaactgtttgtgcgtgttagcgcgaagaacgttatgcgtcagaagagcttttacaaaggtctgcaccagctggtgcaagaaggcgcgattcaactgtacaagaccgttaaaaccgatgagtatctgctgggtgcggtgggccagctgcaattcgaagtttttgagcaccgtatgaagaacgaatataacgcggaagtgctgatggaacgtctgggtagcaaaatcgcgcgttggattgaaaacgacgaggttgatgaaaacctgagcagcagccgtagcctgctggtgaaagaccgttacgatcactatgttttcctgtttgagaacgacttcgcgctgcgttggtttcaggaaaagaacccgaccatcaaactgtacaacccgatggaccaacacgat

SEQ ID NO: 22 - protein, 526 aa, Bacillus sp. X1
MGNDFKKEVLSRRTFAIISHPDAGKTTLTEKLLLFGGAIRDAGTVKAKKTGKYATSDWMEIEKQRGISVTSSVMQFDYNGYRVNILDTPGHQDFSEDTYRTLMAVDSAVMIIDSAKGIEDQTIKLFKVCRMRGIPIFTFINKLDRQGKQPLELLAELEEVLGIESYPMNWPIGMGKEFLGIYDRYYNRIEQFRVNEEERFIPLNEDGEIEGNHKLVSSGLYEQTLEEIMLLNEAGNEFSAERVAAGQLTPVFFGSALTNFGVQTFLETYLQFAPPPKARNSSIGEIDPLSEEFSGFVFKIQANMNPAHRDRIAFVRICSGKFERGMSVNLPRLGKQLKLTQSTSFMAEERNTVEEAVSGDIIGLYDTGTYQIGDTLTVGKNDFQFERLPQFTPELFVRVSAKNVMRQKSFYKGLHQLVQEGAIQLYKTVKTDEYLLGAVGQLQFEVFEHRMKNEYNAEVLMERLGSKIARWIENDEVDENLSSSRSLLVKDRYDHYVFLFENDFALRWFQEKNPTIKLYNPMDQHD

SEQ ID NO: 23 - DNA, 558 bp, Geobacillus
atggccaaacaggttattcagcaggccaaagaaaaaatggataaagccgttcaggcatttacccgtgaactggcaagcattcgtgcaggtcgtgcaaatgcaggtctgctggaaaaagttaccgttgattattatggtgttccgacgccgattaatcagctggcgagcattagcgttccggaagcacgtctgctggtgattcagccgtatgataaaagcgcaatcaaagagatggaaaaagcaattctggcaagcgatctgggtctgaccccgagcaatgatggtagcgttattcgtctggttattccgcctctgaccgaagaacgtcgtcgcgaactggcgaaactggtgaaaaaatacagcgaagatgcaaaagttgccgtgcgtaatattcgtcgtgatgcaaatgatgagctgaaaaagctggaaaagaatggcgaaattaccgaagatgaactgcgtagctataccgatgaagttcagaaactgaccgatgatcatatcgcaaaaattgacgccatcaccaaagagaaagaaaaagaagtcatggaagtttaa

SEQ ID NO: 24 - protein, 185 aa, Geobacillus
MAKQVIQQAKEKMDKAVQAFTRELASIRAGRANAGLLEKVTVDYYGVPTPINQLASISVPEARLLVIQPYDKSAIKEMEKAILASDLGLTPSNDGSVIRLVIPPLTEERRRELAKLVKKYSEDAKVAVRNIRRDANDELKKLEKNGEITEDELRSYTDEVQKLTDDHIAKIDAITKEKEKEVMEV

SEQ ID NO: 25 - DNA, 2637 bp, Geobacillus stearothermophilus
atgaaaaaactgaccagcgcacaggttcgtcgcatgtttctggaattttttcaagaaaaaggtcatgccgttgaaccgagcgcaagcctgattccggttgatgatccgagcctgctgtggattaatagcggtgttgcaaccctgaaaaaatactttgatggtcgtattgttccggaaaatccgcgtatttgtaatgcccagaaaagcattcgtaccaacgatattgaaaatgtgggtaaaaccgcacgccatcacaccttttttgaaatgctgggcaattttagcatcggcgattatttcaaacgtgaagcaattcattgggcctgggaatttctgaccagtgataaatggattggttttgatccggaacgtctgagcgttaccgttcatccggaagatgaagaagcatataacatttggcgcaatgaaattggtctgccggaagaacgtattattcgtctggaaggtaacttttgggatattggtgaaggtccgagcggtccgaataccgaaatcttttatgatcgtggtgaagcctttggtaatgatccgaatgatcctgaactgtatccaggtggtgaaaatgatcgttatctggaagtttggaatctggtgtttagccagtttaatcataatccggatggcacctatacaccgctgccgaaaaaaaacattgataccggcatgggtttagaacgtatgtgtagcattctgcaggatgttccgaccaattttgaaaccgacctgtttctgccgattattcgtgcaaccgagcagattgccggtgaacgttatggtgaagatccggataaagatgttgcctttaaagtgattgccgatcatattcgcgcagttacctttgcaattggtgatggtgcactgccgagcaatgaaggtcgtggttatgttctgcgtcgtctgctgcgtcgtgcagttcgttatgcaaaacatattggtattgaacgtccgttcatgtatgaactggttccggttgttggtgaaatcatgcacgattattatcccgaggttaaagagaaagccgattttattgcacgtgtgattcgtaccgaagaagaacgttttcacgaaaccctgcatgaaggtctggcaattctggcagaagttattgaaaaagcaaaagaacagggttccgatgttattccgggtgaagaggcatttcgtctgtatgatacctatggttttccgattgaactgaccgaagaatatgcagccgaagcaggtatgaccgttgatcatgcaggttttgaacgtgaaatggaacgtcagcgtgaacgtgcccgtgcagcacgtcaggatgttgatagtatgcaggttcaaggtggtgttctgggtgatattaaagatgaaagtcgctttgtgggctatgatgagctggttgcagcaagcaccgttattgcaattgttaaagatggtcgtctggtggaagaagttaaagcaggcgaagaagcacagattattgttgatgttaccccgttttatgcagaaagcggtggtcagattgcagatcagggtgtttttgaaagcgaaaccggcaccgcagttgtgaaagatgttcagaaagcaccgaatggtcagcatctgcatgcaattattgtggaacatggcaccgttaaaaaaggtagccgttataccgcacgtgttgatgaagcaaaacgtatgcgtattgtgaaaaatcataccgcaacacatctgctgcatcaggcactgaaagacgttctgggtcgtcatgttaatcaggcaggtagcctggttgcaccggatcgtctgcgttttgactttacccattttggtcaggttaaacccgaagaactggaacgtattgaagcgattgttaatgagcagatttggaaaagcctgccggtggatattttctataaaccgctggaagaggcaaaagcaatgggtgcaatggcactgtttggtgaaaaatatggtgatattgtgcgtgtggttaaagtgggtgattatagcctggaactgtgtggtggttgtcatgtgccgaataccagcgccattggtctgtttaaaatcgttagcgaaagcggtattggtgcaggcacccgtcgcattgaagcagttaccggtgaagcagcatatcgttttatgagcgaacagctggccattctgcaagaagcagcacagaaactgaaaaccagtccgaaagaactgaatgcacgtctggatggcctgtttgcagaactgaaagaattagaacgcgaaaatgaaagcctggcagcccgtctggcacatatggaagcagaacatctgacccgtcaggtaaaagatgttaatggtgttccggttctggcagcaaaagttcaggcaaatgatatgaatcagctgcgtgccatggccgatgatctgaaacaaaaactgggtacagcagttattgttctggcaagcgcacaaggtggtaaagttcagctgattgcagccgttacagatgacctggtaaaaaaaggttttcatgcgggtaaactggttaaagaagttgcaagccgttgcggtggtggtggcggtggtcgtccggatctggcacaggcaggcggtaaagatccgagcaaagttggtgaagcactgggttatgttgaaacctgggttaaaagcgtgagctaa

SEQ ID NO: 26 - protein, 878 aa, Geobacillus stearothermophilus
MKKLTSAQVRRMFLEFFQEKGHAVEPSASLIPVDDPSLLWINSGVATLKKYFDGRIVPENPRICNAQKSIRTNDIENVGKTARHHTFFEMLGNFSIGDYFKREAIHWAWEFLTSDKWIGFDPERLSVTVHPEDEEAYNIWRNEIGLPEERIIRLEGNFWDIGEGPSGPNTEIFYDRGEAFGNDPNDPELYPGGENDRYLEVWNLVFSQFNHNPDGTYTPLPKKNIDTGMGLERMCSILQDVPTNFETDLFLPIIRATEQIAGERYGEDPDKDVAFKVIADHIRAVTFAIGDGALPSNEGRGYVLRRLLRRAVRYAKHIGIERPFMYELVPVVGEIMHDYYPEVKEKADFIARVIRTEEERFHETLHEGLAILAEVIEKAKEQGSDVIPGEEAFRLYDTYGFPIELTEEYAAEAGMTVDHAGFEREMERQRERARAARQDVDSMQVQGGVLGDIKDESRFVGYDELVAASTVIAIVKDGRLVEEVKAGEEAQIIVDVTPFYAESGGQIADQGVFESETGTAVVKDVQKAPNGQHLHAIIVEHGTVKKGSRYTARVDEAKRMRIVKNHTATHLLHQALKDVLGRHVNQAGSLVAPDRLRFDFTHFGQVKPEELERIEAIVNEQIWKSLPVDIFYKPLEEAKAMGAMALFGEKYGDIVRVVKVGDYSLELCGGCHVPNTSAIGLFKIVSESGIGAGTRRIEAVTGEAAYRFMSEQLAILQEAAQKLKTSPKELNARLDGLFAELKELERENESLAARLAHMEAEHLTRQVKDVNGVPVLAAKVQANDMNQLRAMADDLKQKLGTAVIVLASAQGGKVQLIAAVTDDLVKKGFHAGKLVKEVASRCGGGGGGRPDLAQAGGKDPSKVGEALGYVETWVKSVS

SEQ ID NO: 27 - DNA, 1674 bp, Geobacillus
atgaatattgtgggccagatcaaagaaaaaatgaaagaagaaattcgtcaggcagcagttcgtgcaggtctggcaagcgcagatgaactgccggatgttctgctggaagttccgcgtgataaagcacatggtgattatagcaccaatattgcaatgcagctggcacgtattgcaaaaaaaccgcctcgtgcaattgccgaagcaattgttggtcagctggatcgtgaacgtatgagcgttgcccgtattgaaattgcaggtccgggttttatcaacttctatatggataatcgttacctgaccgcagttgttccggcaattctgcaggcaggtcaggcatatggtgaaagtaatgttggtaatggtgagaaagtccaggttgaatttgttagcgcaaatccgaccggtgatctgcatctgggtcatgcacgtggtgcagcagttggtgatagcctgtgtaatattctggcaaaagcaggttttgatgtgacccgtgaatactatattaatgatgcaggcaagcagatctacaatctggccaaaagcgttgaagcacgttattttcaggcactgggtgttgatatgccgctgccggaagatggttattatggtgatgatattgtggaaatcggcaaaaaactggccgaagaatatggtgatcgtttcgttgaaatggaagaagaggaacgtctggcattttttcgtgattatggtctgcgttatgagctggaaaaaatcaaaaaagatctggccgattttcgcgttccgtttgatgtttggtatagcgaaaccagcctgtatgaaagcggtaaaattgatgaagcactgagcaccctgcgtgaacgtggttatatctatgaacaggatggtgcaacctggtttcgtagcaccgcatttggagatgataaagatcgtgttctgattaaacaggacggcacctatacctatctgctgccggatattgcatatcatcaggataaactgcgtcgcggttttaagaaactgattaacatttggggtgccgatcatcatggttatattcctcgcatgaaagcagcaattgcagcactgggttatgatccggaagcactggaagttgaaattattcagatggtgaatctgtatcagaatggcgaacgtgtgaaaatgagcaaacgtaccggtaaagcagttaccatgcgtgaactgatggaagaggttggtgttgatgcagttcgttatttctttgcaatgcgtagcggtgatacccatctggattttgatatggatctggcagttagccagagcaatgaaaatccggtttattatgttcagtatgcccatgcgcgtgttagcagcattctgcgtcaggcggaagaacagcatattagctatgatggtgatctggcactgcatcatctggttgaaaccgaaaaagaaattgagctgctgaaagtgctgggtgattttccggatgttgttgcagaagcagcactgaaacgtatgccgcatcgtgttaccgcatatgcatttgacctggccagcgcactgcatagcttttataacgccgaaaaagttctggatctggacaacatcgaaaaaaccaaagcacgtctggccctggttaaagccgttcagattacactgcagaatgcactggccctgattggtgtgagcgcaccggaacaaatgtaa

SEQ ID NO: 28 - protein, 557 aa, Geobacillus
MNIVGQIKEKMKEEIRQAAVRAGLASADELPDVLLEVPRDKAHGDYSTNIAMQLARIAKKPPRAIAEAIVGQLDRERMSVARIEIAGPGFINFYMDNRYLTAVVPAILQAGQAYGESNVGNGEKVQVEFVSANPTGDLHLGHARGAAVGDSLCNILAKAGFDVTREYYINDAGKQIYNLAKSVEARYFQALGVDMPLPEDGYYGDDIVEIGKKLAEEYGDRFVEMEEEERLAFFRDYGLRYELEKIKKDLADFRVPFDVWYSETSLYESGKIDEALSTLRERGYIYEQDGATWFRSTAFGDDKDRVLIKQDGTYTYLLPDIAYHQDKLRRGFKKLINIWGADHHGYIPRMKAAIAALGYDPEALEVEIIQMVNLYQNGERVKMSKRTGKAVTMRELMEEVGVDAVRYFFAMRSGDTHLDFDMDLAVSQSNENPVYYVQYAHARVSSILRQAEEQHISYDGDLALHHLVETEKEIELLKVLGDFPDVVAEAALKRMPHRVTAYAFDLASALHSFYNAEKVLDLDNIEKTKARLALVKAVQITLQNALALIGVSAPEQM

SEQ ID NO: 29 - DNA, 1329 bp, Geobacillus
atggatgtgagcattattggtggtaatcagtgtgttaaaaccaccaccattgccgaagttaatcagtatgttggtcagcaggttaccattggtgcatggctggcaaataaacgtagcagcggtaaaattgtttttctgcagctgcgtgatggcaccggttttattcagggtgttgttgaaaaagccaatgttagcgaagaggtttttcagcgtgcaaaaaccctgacacaagaaaccagcctgtatgtgaccggcaccgttcgtattgatgaacgtagcccgtttggttatgaactgagcgttgccgatctgcaggttattcaagaagcagttgattatccgattacgccgaaagaacatggtgttgaatttctgatggatcatcgtcatctgtggctgcgtagccgtcgtcagcatgcaattatgaaaattcgcaacgaaattatccgtgccacctatgaatttttcaacgatcgtggttttgtgaaagtggatgcaccgattctgaccggtagcgcaccggaaggcaccaccgaactgtttcataccaaatatttcgatgaggatgcatatctgagccagagcggtcagctgtatatggaagcagcagcaatggcactgggtaaagtttttagctttggtccgacctttcgtgccgaaaaaagcaaaacccgtcgccatctgattgaattttggatggttgaaccggaaatggccttttatgaatttgaagataatctgcgcctgcaagaggaatatgttagctatctggttcagagcgttctggaacgttgtcgtctggaactgggtcgcctgggtcgtgatgttagcaaactggaattagttaaaccgccttttccgcgtctgacctatgatgaagcaattaaactgctgcatgaaaaaggcctgaccgatattgaatggggtgatgattttggtgcaccgcatgaaaccgcaattgcagaaagctttgataaaccggtgtttatcacccattatccgaccagcctgaaaccgttttatatgcagccggatccgaatcgtccggatgttgttctgtgtgcagatctgattgctccggaaggttatggtgaaattattggcggtagcgaacgcatccatgattatgagctgctgaaacgtcgcctggaagaacatcatctgccgctggaagcatatgaatggtatctggatctgcgtaaatatggtagcgttccgcatagcggttttggtctgggtttagaacgtaccgttgcatggatttgcggtgttgaacatgtgcgtgaaaccattccgtttccacgtctgctgaatcgtctgtatccgtaa

SEQ ID NO: 30 - protein, 442 aa, Geobacillus
MDVSIIGGNQCVKTTTIAEVNQYVGQQVTIGAWLANKRSSGKIVFLQLRDGTGFIQGVVEKANVSEEVFQRAKTLTQETSLYVTGTVRIDERSPFGYELSVADLQVIQEAVDYPITPKEHGVEFLMDHRHLWLRSRRQHAIMKIRNEIIRATYEFFNDRGFVKVDAPILTGSAPEGTTELFHTKYFDEDAYLSQSGQLYMEAAAMALGKVFSFGPTFRAEKSKTRRHLIEFWMVEPEMAFYEFEDNLRLQEEYVSYLVQSVLERCRLELGRLGRDVSKLELVKPPFPRLTYDEAIKLLHEKGLTDIEWGDDFGAPHETAIAESFDKPVFITHYPTSLKPFYMQPDPNRPDVVLCADLIAPEGYGEIIGGSERIHDYELLKRRLEEHHLPLEAYEWYLDLRKYGSVPHSGFGLGLERTVAWICGVEHVRETIPFPRLLNRLYP

SEQ ID NO: 31 - DNA, 1773 bp, Geobacillus
atggaacgcacctattattgtggtgaagttccggaaaccgcagttggtgaacgtgttgttctgaaaggttgggttcagaaacgtcgtgatttaggtggtctgatttttatcgatctgcgtgatcgtaccggtattgttcaggttgttgcaagtccggatgttagcgcagaagcactggcagcagcagaacgtgttcgtagcgaatatgttctgagcgttgaaggcaccgttgttgcccgtgcaccggaaacagttaatccgaatattgcaaccggtcgcattgaaattcaggcagaacgtattgaaattatcaacgaagcaaaaacccctccgtttagcattagtgatgataccgatgcagccgaagatgttcgtctgaaatatcgttatctggatctgcgtcgtccggttatgtttcagaccctggcactgcgtcataaaatcaccaaaaccgttcgtgattttctggatagcgaacgctttctggaaattgaaaccccgatgctgaccaaaagcacaccggaaggtgcacgtgattatctggttccgagccgtgttcatccgggtgaattttatgcactgccgcagagtccgcagatctttaaacagctgctgatggttggtggtgtggaacgttattatcagattgcacgttgttttcgtgatgaggacctgcgtgcagatcgtcagccggaatttacccagattgatattgaaatgagcttcatcgagcaagaggatatcattgatctgaccgaacgtatgatggcagcagttgttaaagcagcaaaaggtattgatattccgcgtccgtttccgcgtattacctatgatgaagcaatgagctgttatggtagcgataaaccggatattcgttttggtctggaactggttgatgtgagcgaaattgttcgtgatagcgcatttcaggtttttgcgcgtgcagttaaagaaggtggtcaggttaaagcaattaatgcaaaaggtgcagcaccgcgttatagccgtaaagatattgatgcactgggcgaatttgcaggtcgttatggtgccaaaggtctggcatggctgaaagcagaaggtgaagaactgaaaggtccgattgcaaaattctttaccgatgaagaacaggcagccctgcgtcgtgcactggccgttgaagatggtgacctgctgctgtttgttgcagatgaaaaagcaattgttgcagcagcactgggtgcgctgcgtctgaaactgggtaaagaactgggtctgattgatgaagccaaactggcatttctgtgggttaccgattggcctctgctggaatacgatgaagaggaaggtcgctattacgcagcacatcatccgtttaccatgccggtgcgtgatgatatcccgctgctggaaaccaatccgagcgcagttcgtgcacaggcatatgatctggttctgaatggttatgaattaggtggtggtagcctgcgtatttttgaacgtgatgtgcaagaaaaaatgtttcgtgccctgggttttagcgaagaagaagcacgtcgtcagtttggttttctgttagaagcatttgaatatggcacccctccgcatggtggtattgcactgggtttagatcgtctggttatgctgctggcaggtcgtaccaatctgcgcgataccattgcatttccgaaaaccgccagcgcaagctgtctgctgaccgaagcaccgggtcctgttagcgacaaacagctggaagaactgcatctggcagttgttctgccggaaaatgaataa

SEQ ID NO: 32 - protein, 590 aa, Geobacillus
MERTYYCGEVPETAVGERVVLKGWVQKRRDLGGLIFIDLRDRTGIVQVVASPDVSAEALAAAERVRSEYVLSVEGTVVARAPETVNPNIATGRIEIQAERIEIINEAKTPPFSISDDTDAAEDVRLKYRYLDLRRPVMFQTLALRHKITKTVRDFLDSERFLEIETPMLTKSTPEGARDYLVPSRVHPGEFYALPQSPQIFKQLLMVGGVERYYQIARCFRDEDLRADRQPEFTQIDIEMSFIEQEDIIDLTERMMAAVVKAAKGIDIPRPFPRITYDEAMSCYGSDKPDIRFGLELVDVSEIVRDSAFQVFARAVKEGGQVKAINAKGAAPRYSRKDIDALGEFAGRYGAKGLAWLKAEGEELKGPIAKFFTDEEQAALRRALAVEDGDLLLFVADEKAIVAAALGALRLKLGKELGLIDEAKLAFLWVTDWPLLEYDEEEGRYYAAHHPFTMPVRDDIPLLETNPSAVRAQAYDLVLNGYELGGGSLRIFERDVQEKMFRALGFSEEEARRQFGFLLEAFEYGTPPHGGIALGLDRLVMLLAGRTNLRDTIAFPKTASASCLLTEAPGPVSDKQLEELHLAVVLPENE

SEQ ID NO: 33 - DNA, 1401 bp, Geobacillus
atgagcagcattcgtctgtataataccctgacgcgtaaaaaagaaccgtttgaaccgctggaaccgaacaaagttaaaatgtatgtttgtggtccgaccgtgtataactatattcatattggtaatgcccgtgcagccattgtgtttgataccattcgtcgttatctggaatttcgcggttatgatgttacctatgtgagcaattttaccgacgtggatgacaaactgattaaagcagcacgtgaactgggtgaaagcgttccggcaattgcagaacgttttattgaagcctatttcgaagatattcaggccctgggttgtaaaaaagcagatattcatccgcgtgtgaccgaaaatatcgataccattattgaatttatccaggcgctgatcgataaaggctatgcatatgaagttgatggcgacgtttattatcgtacccgtaaatttcgcgaatatggcaaactgagccatcagagcattgatgaactgcaggcaggcgcacgtattgaaattggtgaaaaaaaagatgatccgctggattttgcactgtggaaagcagcaaaagaaggtgaaatttgttgggatagcccgtggggtaaaggtcgtcctggttggcatattgaatgtagcgcaatggcacgtaaatatctgggtgatacgattgatattcatgccggtggtcaggatctgacctttccgcatcatgaaaatgaaattgcacagagcgaagcactgaccggtaaaccgtttgccaaatattggctgcataatggctatctgaacatcaacaacgagaaaatgagcaaaagcctgggtaattttgttctggtgcatgatattattcgcgagattgatccgcaggttctgcgcttttttatgctgagcgttcattatcgtcatccgatcaattatagcgaagaactgctggaaagcgcacgtcgtggtctggaacgtctgaaaaccgcatatagcaatctgcagcaccgtctgcaggcaagcaccaatctgaccgataatgatgaagaatgggttagccgtattgccgatattcgtgcaagctttattcgtgaaatggatgatgattttaacaccgccaatggtattgccgttctgtttgaactggcaaaacaggcaaatctgtatctgcaagaaaaaaccacctccgaaaaagtgattcatgcatttctgcgtgaatttgaacagctggcagatgttctgggtctgaccctgaaacaggatgagctgctggatgaagaaattgaagccctgattcagaaacgtaatgaagcccgtaaaaatcgtgattttgccctggcagatcgtattcgtgatgaattacgtgcgaaaaacatcatcctggaagatacaccgcagggcacccgttggaaacgtggttaa

SEQ ID NO: 34 - protein, 466 aa, Geobacillus
MSSIRLYNTLTRKKEPFEPLEPNKVKMYVCGPTVYNYIHIGNARAAIVFDTIRRYLEFRGYDVTYVSNFTDVDDKLIKAARELGESVPAIAERFIEAYFEDIQALGCKKADIHPRVTENIDTIIEFIQALIDKGYAYEVDGDVYYRTRKFREYGKLSHQSIDELQAGARIEIGEKKDDPLDFALWKAAKEGEICWDSPWGKGRPGWHIECSAMARKYLGDTIDIHAGGQDLTFPHHENEIAQSEALTGKPFAKYWLHNGYLNINNEKMSKSLGNFVLVHDIIREIDPQVLRFFMLSVHYRHPINYSEELLESARRGLERLKTAYSNLQHRLQASTNLTDNDEEWVSRIADIRASFIREMDDDFNTANGIAVLFELAKQANLYLQEKTTSEKVIHAFLREFEQLADVLGLTLKQDELLDEEIEALIQKRNEARKNRDFALADRIRDELRAKNIILEDTPQGTRWKRG

SEQ ID NO: 35 - DNA, 1665 bp, E. coli
atgagcgaagcagaagcacgtccgaccaactttattcgtcagattattgatgaagatctggccagcggtaaacataccaccgttcatacccgttttccgcctgaaccgaatggttatctgcatattggtcatgccaaaagcatttgcctgaattttggtattgcccaggattataaaggtcagtgcaatctgcgtttcgatgataccaatccggtgaaagaagatatcgaatacgtcgagagcatcaaaaatgatgttgaatggctgggttttcattggagcggtaatgttcgttatagcagcgattattttgatcagctgcatgcctatgcaatcgaactgattaacaaaggtctggcctatgttgatgaactgacaccggaacaaattcgtgaatatcgtggtacactgacccagcctggtaaaaatagcccgtatcgtgatcgtagcgttgaagaaaatctggccctgtttgaaaaaatgcgtgccggtggttttgaagaaggtaaagcctgtctgcgtgcaaaaattgatatggcaagcccgtttattgttatgcgtgatccggttctgtatcgcatcaaatttgcagaacatcatcagaccggtaacaaatggtgtatctatccgatgtatgatttcacccattgcattagtgatgccctggaaggtattacccatagcctgtgtaccctggaatttcaggataatcgtcgtctgtatgattgggtgttagacaatatcaccattccggtgcatccgcgtcagtatgaatttagccgtctgaatctggaatacaccgttatgagcaaacgtaaactgaatctgctggtgaccgataaacatgttgaaggttgggatgatccgcgtatgccgaccattagcggtctgcgtcgtcgtggttataccgcagcaagcatccgtgaattttgtaaacgtattggtgtgaccaaacaggataacaccattgaaatggccagcctggaaagctgtattcgcgaagatctgaatgaaaatgcaccgcgtgcaatggcagttatcgatccggttaaactggtgatcgaaaattatcaaggtgaaggtgaaatggtgaccatgccgaatcatccgaataaaccggaaatgggtagccgtcaggttccgtttagcggtgaaatttggattgatcgtgcagattttcgtgaagaagccaacaaacagtataaacgtctggttctgggtaaagaagttcgtctgcgtaacgcctatgttattaaagcagaacgtgttgaaaaagatgccgaaggcaatattaccaccattttttgtacctatgacgcagataccctgagcaaagatccggcagatggtcgtaaagttaaaggtgttattcattgggttagcgcagcacatgcactgccggttgaaattcgcctgtatgatcgtctgtttagcgttccgaatccgggtgcagcagatgattttctgagcgttattaatccggaaagcctggttattaaacagggttttgccgaaccgagcctgaaagatgcagttgcaggtaaagcatttcagtttgaacgcgaaggttatttttgtctggatagccgtcatagcaccgcagaaaaaccggtgtttaatcgtaccgttggtctgcgtgatacctgggcaaaagttggtgaataa

SEQ ID NO: 36 - protein, 554 aa, E. coli
MSEAEARPTNFIRQIIDEDLASGKHTTVHTRFPPEPNGYLHIGHAKSICLNFGIAQDYKGQCNLRFDDTNPVKEDIEYVESIKNDVEWLGFHWSGNVRYSSDYFDQLHAYAIELINKGLAYVDELTPEQIREYRGTLTQPGKNSPYRDRSVEENLALFEKMRAGGFEEGKACLRAKIDMASPFIVMRDPVLYRIKFAEHHQTGNKWCIYPMYDFTHCISDALEGITHSLCTLEFQDNRRLYDWVLDNITIPVHPRQYEFSRLNLEYTVMSKRKLNLLVTDKHVEGWDDPRMPTISGLRRRGYTAASIREFCKRIGVTKQDNTIEMASLESCIREDLNENAPRAMAVIDPVKLVIENYQGEGEMVTMPNHPNKPEMGSRQVPFSGEIWIDRADFREEANKQYKRLVLGKEVRLRNAYVIKAERVEKDAEGNITTIFCTYDADTLSKDPADGRKVKGVIHWVSAAHALPVEIRLYDRLFSVPNPGAADDFLSVINPESLVIKQGFAEPSLKDAVAGKAFQFEREGYFCLDSRHSTAEKPVFNRTVGLRDTWAKVGE

SEQ ID NO: 37 - DNA, 1473 bp, Geobacillus
atggccaaagaagttcgcgttcgttacgcaccgagtccgaccggtcatctgcatattggtggtgcacgtaccgcactgtttaattacctgtttgcacgtcatcatggtggcaaaatgattgtgcgtattgaagataccgatatcgaacgtaatgttgaaggtggtgaaaaaagccagctggaaaatctgaaatggctgggcattgattatgatgaaagcattgatcaggatggtggttatggtccgtatcgtcagaccgaacgtctggatatttatcgcaaatatgtgaacgaactgctggaacagggtcatgcctataaatgtttttgtacaccggaagaactggaacgtgaacgtgaagcacagcgtgcagcaggtattgcagcaccgcagtatagcggtaaatgtcgtcatctgacaccggaacaggttgccgaactggaagcacagggtaaaccgtataccattcgtctgaaagttccggaaggtaaaacctatgaattctatgatctggtgcgtggcaaagttgtgtttgaaagcaaagatgttggtggcgattgggttattgttaaagcaaatggtattccgacctataactttgccgttgtgattgatgatcacctgatggaaatttcacatgtgtttcgtggtgaagaacatctgagcaataccccgaaacagctgatggtgtatgaatattttggttgggaaccgcctcagtttgcacatctgaccctgattgttaatgaacagcgtaaaaaactgagcaaacgcgacgaaagcattattcagtttgtgagccagtataaagaactgggttatctgccggaagccatgtttaacttttttgcactgttaggttggtcaccggaaggtgaagaagaaatctttaccaaagatgaactgatccgcatgtttgatgttagccgtctgagcaaaagcccgagtatgtttgataccaaaaagctgacctggatgaacaaccagtacatcaaaaaactggatctggatcgtctggttgaactggcactgccgcatctggttaaagcaggtcgtctgcctgcagatatgaccgatgagcagcgtcagtgggcacgtgatctgattgcactgtatcaagagcagatgagctatggtgcagaaattgttccgctgagcgaactgtttttcaaagaagagattgattacgaggatgaagcacgtcaggttctggcagaagaacaggttccggcagttctgagcacctttctggaaagcgttcgtgagctggaaccgtttaccgcagatgaaattaaagcagcaattaaagccgttcagaaagcaaccggtcagaaagggaaaaaactgtttatgccgattcgtgcagccgttacaggtcagacccatggtccggaactgccgtttgcaattcagctgctgggtaaagaaaaagtgattgaacgcctggaacgcgcactgcaagaaaaattctaa

SEQ ID NO: 38 - protein, 490 aa, Geobacillus
MAKEVRVRYAPSPTGHLHIGGARTALFNYLFARHHGGKMIVRIEDTDIERNVEGGEKSQLENLKWLGIDYDESIDQDGGYGPYRQTERLDIYRKYVNELLEQGHAYKCFCTPEELEREREAQRAAGIAAPQYSGKCRHLTPEQVAELEAQGKPYTIRLKVPEGKTYEFYDLVRGKVVFESKDVGGDWVIVKANGIPTYNFAVVIDDHLMEISHVFRGEEHLSNTPKQLMVYEYFGWEPPQFAHLTLIVNEQRKKLSKRDESIIQFVSQYKELGYLPEAMFNFFALLGWSPEGEEEIFTKDELIRMFDVSRLSKSPSMFDTKKLTWMNNQYIKKLDLDRLVELALPHLVKAGRLPADMTDEQRQWARDLIALYQEQMSYGAEIVPLSELFFKEEIDYEDEARQVLAEEQVPAVLSTFLESVRELEPFTADEIKAAIKAVQKATGQKGKKLFMPIRAAVTGQTHGPELPFAIQLLGKEKVIERLERALQEKF

SEQ ID NO: 39 - DNA, 1383 bp, Geobacillus
atggcagttaccatggaagaaattgttgcacatgcaaaacatcgtggttttgtttttccgggtagcgaaatttatggtggtctggcaaatacctgggattatggtccgctgggtgttgaactgaaaaataacattaaacgtgcctggtggaaaaaattcgttcaagaaagcccgtataatgttggtctggatgcagcaattctgatgaatccgcgtacctgggaagcaagcggtcatctgggtaactttaatgatccgatggttgattgcaaacagtgtaaagcacgtcatcgtgcagataaactgattgaaaaagccctggaagaaaaaggcattgagatgattgttgatggtctgccgctggcaaaaatggatgaactgattaaagaatatgatatcgcctgtccggaatgtggtagccgtgattttaccaatgttcgtcagtttaacctgatgttcaaaacctatcagggtgttaccgaaagcagcgccaatgaaatttatctgcgtccggaaaccgcacagggtatttttgttaatttcaaaaatgtgcagcgcaccatgcgtaaaaaactgccgtttggtattgcacagattggcaaaagctttcgcaacgaaattacccctggtaattttacctttcgcacccgtgaatttgagcagatggaactggaatttttctgtaaaccgggtgaagaactgcagtggctggaatattggaaacagttttgtaaagaatggctgctgagcctgggtatgaaagaagataatattcgtctgcgtgatcatgccaaagaagaactgagccattatagcaatgcaaccaccgatatcgaatatcattttccgtttggttggggtgaactgtggggtattgcaagccgtaccgattatgatctgaaacgccatatggaatatagcggtgaagatttccattacctggatcaagaaaccaacgaacgttatattccgtattgtattgaaccgagtctgggtgcagatcgtgttaccctggcatttatgattgatgcctatgatgaagaggaacttgaagatggtacaacccgtaccgtgatgcatctgcatccggcactggcaccgtataaagcagcagtgctgccgttaagcaaaaaactggcagatggtgcacgtcgtatttatgaggaactggcaaaacacttcatggtggattatgatgaaaccggtagtattggtaaacgttatcgtcgtcaggatgaaattggcaccccgttttgtattacctatgattttgaaagcgaacaggatggtcaggttaccgttcgtgatcgtgatacaatggaacaggttcgtctgccgattggcgaactgaaagcatttctggaagagaaaatcgccttctaa

SEQ ID NO: 40 - protein, 460 aa, Geobacillus
MAVTMEEIVAHAKHRGFVFPGSEIYGGLANTWDYGPLGVELKNNIKRAWWKKFVQESPYNVGLDAAILMNPRTWEASGHLGNFNDPMVDCKQCKARHRADKLIEKALEEKGIEMIVDGLPLAKMDELIKEYDIACPECGSRDFTNVRQFNLMFKTYQGVTESSANEIYLRPETAQGIFVNFKNVQRTMRKKLPFGIAQIGKSFRNEITPGNFTFRTREFEQMELEFFCKPGEELQWLEYWKQFCKEWLLSLGMKEDNIRLRDHAKEELSHYSNATTDIEYHFPFGWGELWGIASRTDYDLKRHMEYSGEDFHYLDQETNERYIPYCIEPSLGADRVTLAFMIDAYDEEELEDGTTRTVMHLHPALAPYKAAVLPLSKKLADGARRIYEELAKHFMVDYDETGSIGKRYRRQDEIGTPFCITYDFESEQDGQVTVRDRDTMEQVRLPIGELKAFLEEKIAF

SEQ ID NO: 41 - DNA, 1281 bp, Geobacillus
atggcatttcagattccgcgtggcacccaggatgttctgcctggtgataccgaaaaatggcagtatgttgaacatgttgcacgtaatctgtgtagccgttatggttatcgtgaaattcgtaccccgatttttgaacacaccgaactgtttctgcgtggtgtgggtgataccaccgatattgttcagaaagaaatgtataccttcgaggataaaggtggtcgtgcactgaccctgcgtccggaaggcaccgcaccggttgttcgtgcatttgtggaacataaactgtatggtagtccgcatcagccgctgaaactgtattattcaggtccgatgtttcgttatgaacgtcctgaagcaggtcgttttcgtcagtttgttcagtttggtgttgaagcactgggtagcagcgatccggcaattgatgcagaagttatggcactggcaatgcatatttatgaagccctgggtctgaaacgtattcgtctggtgattaatagcctgggtgatctggatagccgtcgtgcacatcgtgaagcgctggttcgtcattttagcagccgtattcatgaactgtgtccggattgtcagacccgtctgcataccaatccgctgcgtattctggattgtaaaaaagatcgtgatcatgagctgatggcaaccgcaccgagcatcctggattatctgaatgaagatagccgtgcctatttcgagaaagtgaaacagtatctgaccaatctgggtattccgtttgttattgatagtcgtctggttcgtggtctggattattacaatcataccacctttgaaatcatgagcgaagccgaaggttttggtgcagcagcaaccctgtgtggtggtggtcgttataatggtctggttcaagaaattggtggtccggaaacacctggtattggttttgcactgagcattgaacgtctgctggcagcactggatgccgaaggtgttgaactgccggttgaaagtggcctggattgttatgttgttgcagttggtgaacgtgcaaaagatgaagcagtgcgtctggtttatgccctgcgtcgtagcggtctgcgtgttgatcaggattacctgggtcgtaaactgaaagcacagctgaaagcagcagatcgtctgggtgcaagctttgttgcaattattggtgatgaggaactggaacgtcaagaagcagcagttaaacatatggcaagcggtgaacagaccaatgttccgctgggtgaactggcacattttctgcatgaacgtattggcaaagaagaataa

SEQ ID NO: 42 - protein, 426 aa, Geobacillus
MAFQIPRGTQDVLPGDTEKWQYVEHVARNLCSRYGYREIRTPIFEHTELFLRGVGDTTDIVQKEMYTFEDKGGRALTLRPEGTAPVVRAFVEHKLYGSPHQPLKLYYSGPMFRYERPEAGRFRQFVQFGVEALGSSDPAIDAEVMALAMHIYEALGLKRIRLVINSLGDLDSRRAHREALVRHFSSRIHELCPDCQTRLHTNPLRILDCKKDRDHELMATAPSILDYLNEDSRAYFEKVKQYLTNLGIPFVIDSRLVRGLDYYNHTTFEIMSEAEGFGAAATLCGGGRYNGLVQEIGGPETPGIGFALSIERLLAALDAEGVELPVESGLDCYVVAVGERAKDEAVRLVYALRRSGLRVDQDYLGRKLKAQLKAADRLGASFVAIIGDEELERQEAAVKHMASGEQTNVPLGELAHFLHERIGKEE

SEQ ID NO: 43 - DNA, 2775 bp, Geobacillus stearothermophilus
atggactacaaagaaaccctgctgatgccgcagaccgaatttccgatgcgtggtaatctgccgaaacgtgaaccggaaatgcagaaaaaatgggaagagatggatatctaccgcaaagttcaagaacgtaccaaaggtcgtccgctgtttgttctgcatgatggtccgccttatgcaaatggtgatattcatatgggtcatgccctgaacaaaatcctgaaagatattatcgtgcgctataagagcatgaatggttattgtgcaccgtatgttccaggttgggatacccatggtctgccgattgaaaccgcactggcaaaacagggtgttgatcgtaaaagcatgagcgttgcagaatttcgtaaacgttgtgaacagtatgcctatgagcagattgataatcagcgtcgtcagtttaaacgtctgggtgttcgtggtgattgggataatccgtatattaccctgaaaccggaatatgaagcacagcagattaaagtgtttggcgagatggcaaaaaaaggcctgatctataaaggtctgaaacctgtttattggagcccgagcagcgaaagtgcactggcagaagcagaaattgagtataaagataaacgctccccgagcatttatgttgcctttccggttaaagatggtaaaggtgttctggaaggtgatgaacgtattgtgatttggaccaccacaccgtggaccattccggcaaatctggcaattgcagttcatccggatctggattatcatgttgttgatgttagcggtaaacgttatgttgttgcagcagcactggccgaaagcgttgcaaaagaaattggttgggatgcatggtcagttgtgaaaaccgttaaaggtaaagaactggaatatgtggttgcgaaacacccgttttatgaacgtgatagcctggttgtttgtggtgaacatgtgaccaccgatgcaggcaccggttgtgttcataccgcacctggtcatggtgaagatgattttctggttggtcagaaatatggcctgccggttctgtgtccggtggatgaacgtggttatatgaccgaagaagcaccgggttttgaaggtatgttttatgaggatgccaacaaagcgattacgcagaaactggaagaagttggcgcactgctgaaactgggttttattacccatagctatccgcatgattggcgtaccaaacagccgaccatttttcgtgcaaccacacagtggtttgcaagcattgataaaattcgcaatgaactgctgcaggccatcaaagaaacaaaatggatcccggaatggggtgaaattcgcattcataacatggttcgtgatcgcggtgattggtgtattagccgtcagcgtgcatggggtgttccgattccggtgttttatggtgaaaatggtgaaccgattatcaccgatgaaaccattgaacatgttagcaacctgtttcgtcagtatggtagcaatgtttggtttgaacgtgaagcaaaagatctgctgccggaaggttttacccatccgagcagcccgaatggtatttttacaaaagaaaccgatatcatggacgtgtggtttgatagcggtagcagccatcaggcagttctggtggaacgtgatgatctgatgcgtccggcagatctgtatctggaaggcagcgatcagtatcgtggttggtttaatagcagcctgagcaccgcagttgcagtgaccggtaaagcaccgtataaaggtgtgctgagccatggttttgtgctggatggtgaaggtcgtaaaatgagcaaaagcctgggtaatgttgttgttcctgcaaaagttatggaacagtttggtgcagatattctgcgtctgtgggttgccagcgttgattatcaggcagatgttcgtattagcgatcatattctgaaacaggtgagcgaagtgtatcgcaaaattcgtaatacctttcgctttatgctgggtaacctgtttgattttgatccgaatcagaatgcagttccgattggtgaactgggtgaagttgatcgttatatgctggccaaactgaataaactgatcgccaaagtgaaaaaagcctatgatagctacgatttcgcagccgtttatcatgaaatgaaccatttttgtaccgttgaactgagcgccttttatctggatatggcaaaagatatcctgtatatcgaagcagcagatagccgtgcacgtcgtgcagttcagaccgttctgtatgaaaccgttgttgcactggcgaaactgattgcaccgattctgccgcataccgcagatgaagtttgggaacatattccgaatcgtcgtgaaaatgtggaaagcgttcagctgaccgatatgccggaaccgattgcaattgatggcgaagaggcactgctggcaaaatgggatgcctttatggatgttcgtgatgatatgctgaaagcactggaaaatgcccgtaacgaaaaagtgattggtaaaagcctgaccgcaagcgttattgtttatccgaaagatgaagcacgtaaactgctggcgagcctggatgccgatctgcgtcagctgctgattgttagcgcatttagcattgcagatgaaccgtatgatgctgcccctgcagaagccgaacgtctggatcatgttgccgttctggttcgtcctgccgaaggtgaaacctgcgaacgttgttggaccgttacaccggcagttggtcaggatccgagccatccgaccttttgtccgcgttgtgcacatattgttaacgaacattatagcgcctaa

SEQ ID NO: 44 - protein, 924 aa, Geobacillus stearothermophilus
MDYKETLLMPQTEFPMRGNLPKREPEMQKKWEEMDIYRKVQERTKGRPLFVLHDGPPYANGDIHMGHALNKILKDIIVRYKSMNGYCAPYVPGWDTHGLPIETALAKQGVDRKSMSVAEFRKRCEQYAYEQIDNQRRQFKRLGVRGDWDNPYITLKPEYEAQQIKVFGEMAKKGLIYKGLKPVYWSPSSESALAEAEIEYKDKRSPSIYVAFPVKDGKGVLEGDERIVIWTTTPWTIPANLAIAVHPDLDYHVVDVSGKRYVVAAALAESVAKEIGWDAWSVVKTVKGKELEYVVAKHPFYERDSLVVCGEHVTTDAGTGCVHTAPGHGEDDFLVGQKYGLPVLCPVDERGYMTEEAPGFEGMFYEDANKAITQKLEEVGALLKLGFITHSYPHDWRTKQPTIFRATTQWFASIDKIRNELLQAIKETKWIPEWGEIRIHNMVRDRGDWCISRQRAWGVPIPVFYGENGEPIITDETIEHVSNLFRQYGSNVWFEREAKDLLPEGFTHPSSPNGIFTKETDIMDVWFDSGSSHQAVLVERDDLMRPADLYLEGSDQYRGWFNSSLSTAVAVTGKAPYKGVLSHGFVLDGEGRKMSKSLGNVVVPAKVMEQFGADILRLWVASVDYQADVRISDHILKQVSEVYRKIRNTFRFMLGNLFDFDPNQNAVPIGELGEVDRYMLAKLNKLIAKVKKAYDSYDFAAVYHEMNHFCTVELSAFYLDMAKDILYIEAADSRARRAVQTVLYETVVALAKLIAPILPHTADEVWEHIPNRRENVESVQLTDMPEPIAIDGEEALLAKWDAFMDVRDDMLKALENARNEKVIGKSLTASVIVYPKDEARKLLASLDADLRQLLIVSAFSIADEPYDAAPAEAERLDHVAVLVRPAEGETCERCWTVTPAVGQDPSHPTFCPRCAHIVNEHYSA

SEQ ID NO: 45 - DNA, 2418 bp, Geobacillus stearothermophilus
atgagctttaaccaccgtgaaatcgaacagaaatggcaggattattgggagaagaataaaacctttcgtacaccggatgatgatgacaaaccgaaattctatgtgctggatatgtttccgtatccgagcggtgcaggtctgcatgttggtcatccggaaggttataccgcaaccgatattctggcacgtatgaaacgtatgcagggttataatgttctgcatccgatgggttgggatgcatttggtctgcctgcagaacagtatgcactggataccggtaatgatccggcagaatttacccagaaaaacatcgataactttcgtcgccagattaaaagcctgggttttagctatgattgggatcgtgaaatcaataccaccgatccgaattattacaaatggacccagtggatcttcctgaaactgtatgaaaaaggtctggcctatatggatgaagttccggttaattggtgtccggcactgggcaccgttctggcaaatgaagaagttattaacggtcgtagcgaacgtggtggccatccggttattcgtaaaccgatgcgtcagtggatgctgaaaattaccgcatatgcagatcgtctgctggaagatctggaagaattagattggcctgaaagcatcaaagaaatgcagcgtaattggattggtcgtagtgaaggtgcagaaattgaatttgcagttgatggtcacgatgaaacctttaccgtttttaccacacgtccggatacactgtttggtgcaacctataccgtgctggcaccggaacatccgctggttgaaaaaatcaccactccggaacagaaacctgccgttgatgcatatctgaaagaaattcagagcaaaagcgatctggaacgtaccgatctggccaaagaaaaaaccggtgtgtttaccggtgcatatgccattcatcctgttaccggtgatcgcctgccgatttggattgcagattatgttctgatgagctatggtacaggtgcaattatggcagttccggcacatgatgaacgtgattatgaattcgccaaaaaattccatctgccgatgaaagaagttgttgcaggcggtaatattgagaaagaagcatatacaggcgacggcgaacatattaacagcgaatttctgaatggcctgaataaacaagaggccatcgataaaatgattgcctggctggaagaacatggtaaaggtcgtaaaaaagttagctatcgtctgcgtgattggctgtttagccgtcagcgttattggggtgaaccgattccgattattcattgggaagatggcaccatgacaccggttccggaagaagaactgccgctggttctgccgaaaaccgatgaaattcgtccgagcggcaccggtgaaagtccgctggcaaatattgaagaatgggttaatgttgtggatccgaaaacgggtaaaaaaggtcgtcgcgaaaccaataccatgccgcagtgggcaggtagctgttggtattatctgcgttatattgatccgcacaacgataaacagctggcagatccggaaaaactgaaaaaatggctgccggttgatgtgtatattggtggtgccgaacatgcagtgctgcatctgctgtatgcacgtttttggcataaatttctgtatgacctgggtattgttccgaccaaagaaccgtttcagaaactgtttaatcagggtatgattctgggcgagaacaacgaaaaaatgagcaaaagtaaaggcaatgtggtgaacccggatgatattattgaaagccatggtgcagataccctgcgtctgtatgagatgtttatgggtccgctggaagcaagcattgcatggtcaaccaaaggcctggatggtgcacgtcgttttctggatcgtgtttggcgtctgtttgttaccgaaaatggtgaactgaatccgaacattgttgatgaaccggcaaatgataccctggaacgcatttatcatcagaccgttaaaaaagtgaccgaggattatgaagccctgcgttttaataccgcaattagccagctgatggtgtttattaacgaagcctataaagccgagcagatgaaaaaagaatatatggaaggcttcgtgaaactgctgagtccggtttgtccgcatattggtgaagaactgtggcagaaactgggtcataccgataccattgcatatgaaccgtggccgacctatgatgaaaccaaactggttgaagatgtggtggaaattgttgtgcagattaatggtaaagtgcgtagtcgcctgcatgtgcctgttgatctgcctaaagaagccttagaagaacgcgcactggcggatgaaaagattaaagaacagctggaaggtaaaaccgtgcgtaaagttattgccgttccgggtaaactggttaatattgttgccaactaa

SEQ ID NO: 46 - protein, 805 aa, Geobacillus stearothermophilus
MSFNHREIEQKWQDYWEKNKTFRTPDDDDKPKFYVLDMFPYPSGAGLHVGHPEGYTATDILARMKRMQGYNVLHPMGWDAFGLPAEQYALDTGNDPAEFTQKNIDNFRRQIKSLGFSYDWDREINTTDPNYYKWTQWIFLKLYEKGLAYMDEVPVNWCPALGTVLANEEVINGRSERGGHPVIRKPMRQWMLKITAYADRLLEDLEELDWPESIKEMQRNWIGRSEGAEIEFAVDGHDETFTVFTTRPDTLFGATYTVLAPEHPLVEKITTPEQKPAVDAYLKEIQSKSDLERTDLAKEKTGVFTGAYAIHPVTGDRLPIWIADYVLMSYGTGAIMAVPAHDERDYEFAKKFHLPMKEVVAGGNIEKEAYTGDGEHINSEFLNGLNKQEAIDKMIAWLEEHGKGRKKVSYRLRDWLFSRQRYWGEPIPIIHWEDGTMTPVPEEELPLVLPKTDEIRPSGTGESPLANIEEWVNVVDPKTGKKGRRETNTMPQWAGSCWYYLRYIDPHNDKQLADPEKLKKWLPVDVYIGGAEHAVLHLLYARFWHKFLYDLGIVPTKEPFQKLFNQGMILGENNEKMSKSKGNVVNPDDIIESHGADTLRLYEMFMGPLEASIAWSTKGLDGARRFLDRVWRLFVTENGELNPNIVDEPANDTLERIYHQTVKKVTEDYEALRFNTAISQLMVFINEAYKAEQMKKEYMEGFVKLLSPVCPHIGEELWQKLGHTDTIAYEPWPTYDETKLVEDVVEIVVQINGKVRSRLHVPVDLPKEALEERALADEKIKEQLEGKTVRKVIAVPGKLVNIVAN

SEQ ID NO: 47 - DNA, 1485 bp, Geobacillus stearothermophilus
atgagccatgaagaactgaatgatcagctgcgtgttcgtcgtgaaaaactgaaaaaaatcgaagaactgggcgttgatccgtttggtaaacgttttgaacgtacccataaagcccaagaactgtttgaactgtatggtgatctgagcaaagaggaactggaagaaaaacaaattgaagttgcagttgccggtcgcattatgaccaaacgtggtaaaggtaaagcaggctttgcacatattcaggatgttaccggtcagattcagatttatgtgcgtcaggatgatgttggtgaacagcagtatgaactgttcaaaattagcgatctgggtgatattgttggtgttcgtggcaccatgtttaaaaccaaagtgggtgaactgagcattaaagtgagcagctatgaatttctgaccaaagcactgcgtccgctgccggaaaaatatcatggtctgaaagatattgaacagcgttatcgtcagcgctatctggatctgattatgaatccggaaagcaaaaaaacctttattacccgctcactgattatccagagcatgcgtcgttatctggatagccgtggatatctggaagttgaaaccccgatgatgcatgccgttgccggtggtgcagcagcacgtccgtttattacacatcataatgcactggatatgaccctgtatatgcgtattgcaattgaactgcatctgaaacgtctgattgttggcggtctggaaaaagtgtatgaaattggtcgtgtgtttcgcaatgaaggtattagcacccgtcataatccggaatttaccatgctggaactgtacgaagcatatgccgattttcacgatattatggaactgaccgaaaacctgattgcccatattgcaaccgaagttctgggcaccaccaaaattcagtatgatgaacatgttgttgacctgacaccggaatggcgtcgtctgcatatggttgatgcaattaaagaatatgtcggcgtggatttttggcgtcagatgagtgatgaagaagcacgcgaactggcaaaagaacatggtgtggaagttgcaccgcatatgacctttggccatattgtgaacgaattctttgagcagaaagtggaaagccatctgattcagccgacctttatctatggtcatccggttgaaattagtccgctggccaaaaaaaacccggatgatcctcgttttaccgatcgttttgagctgtttattgtgggtcgtgaacatgcaaatgcctttaccgaactgaacgatccgattgatcagcgtcagcgttttgaagcacagctgaaagaacgtgaacagggtaatgatgaagcacacgaaatggatgaagattttctggaagcactggaatatggtatgcctccgaccggtggtttaggtattggtgttgatcgtctggttatgctgctgaccaatagtccgagcattcgtgatgttctgctgtttccgcagatgcgtcataaataa

SEQ ID NO: 48 - protein, 494 aa, Geobacillus stearothermophilus
MSHEELNDQLRVRREKLKKIEELGVDPFGKRFERTHKAQELFELYGDLSKEELEEKQIEVAVAGRIMTKRGKGKAGFAHIQDVTGQIQIYVRQDDVGEQQYELFKISDLGDIVGVRGTMFKTKVGELSIKVSSYEFLTKALRPLPEKYHGLKDIEQRYRQRYLDLIMNPESKKTFITRSLIIQSMRRYLDSRGYLEVETPMMHAVAGGAAARPFITHHNALDMTLYMRIAIELHLKRLIVGGLEKVYEIGRVFRNEGISTRHNPEFTMLELYEAYADFHDIMELTENLIAHIATEVLGTTKIQYDEHVVDLTPEWRRLHMVDAIKEYVGVDFWRQMSDEEARELAKEHGVEVAPHMTFGHIVNEFFEQKVESHLIQPTFIYGHPVEISPLAKKNPDDPRFTDRFELFIVGREHANAFTELNDPIDQRQRFEAQLKEREQGNDEAHEMDEDFLEALEYGMPPTGGLGIGVDRLVMLLTNSPSIRDVLLFPQMRHK

SEQ ID NO: 49 - DNA, 1953 bp, Geobacillus stearothermophilus
atggaaaaaaagaccttctatctgaccacgccgatctattatccgagcgatcgtctgcatattggtcatgcatataccaccgttgccggtgatgcaatggcacgttataaacgtatgcgtggttatgatgttatgtatctgaccggcaccgatgaacatggtcagaaaattcagcgtaaagccgaagaaaaaggtgttacaccgcagcagtatgttgatgaaattgttgcaggtattcaagaactgtggaaaaaactggatatcagctatgatgatttcatccgtaccacacaagaacgccataaaaaagttgttgagcagatttttacccgtctggttgaacagggtgatatttatctgggtgaatatgaaggttggtattgtaccccgtgtgaaagcttttataccgaacgtcagctggttgatggtaattgtccggattgtggtcgtccggttgaaaaagttaaagaggaaagctattttttccgcatgagcaaatatgttgatcgcctgctgcagtattatgaagaaaacccggatttcattcagccggaaagccgtaaaaatgagatgattaacaactttatcaaacctggcctggaagatctggcagttagccgtaccacctttgattggggtattaaagttccgggtaatccgaaacatgtgatctatgtttggattgatgcactggccaactatattaccgcattaggttatggcaccgataacgatgaaaaattccgtaaatattggcctgccgatgttcatctggttggtaaagaaattgttcgcttccataccatttattggccgattatgctgatggcactgggtctgccgctgccgaaaaaagtttttggtcatggttggctgctgatgaaagatggtaaaatgagcaaaagcaaaggcaatgttgttgatccggttacactgattgatcgttatggtctggatgcactgcgttattatctgctgcgtgaagttccgtttggtgcagatggtgtttttacaccggaaggttttattgagcgcatcaattatgatctggcaaatgatctgggtaatctgctgcatcgtaccgttgcaatgatcgaaaaatactttgatggtgtgattccgccttatcgtggtccgaaaacaccgtttgatcaagagctggttcagaccgcacgtgaagttgttcgtcagtatgaagaggcaatggaaggtatggaatttagcgttgcactggcagcagtttggcagctgattagtcgtaccaataaatacattgatgaaacccagccgtgggtgttagcaaaagatgaacagaaacgtgatgaactggcagccgttatgacccatctggcagaaagcctgcgtcataccgcagttctgctgcagccgtttctgacccgcacaccggaacgtatgctggcacagctgggtattaccgatcatagcctgaaagaatgggatagcctgtatgattttggtctgattccggaaggcaccaaagttcagaaaggtgaaccgctgtttccgcgtctggatattgaagcagaagtggaatatatcaaagcccatatgcaaggtggtaaaccggcagccgaaccggttaaagaagaaaaaaaagcagccgaagcagcggaaattagcatcgatgaatttgcaaaagttgatctgcgtgttgccgaagttattcatgcagaacgtatgaaaaacgccgataaactgctgaaactgcagctggatttaggtggtgaaaaacgtcaggttattagcggtattgccgaattctataaaccggaagaactggtgggtaaaaaagtgatttgtgtggcaaatctgaaaccggcaaaactgcgtggtgaatggtctgaaggcatgattctggcaggcggtagcggtggtgaatttagcctggcaaccgttgatcagcatgttccgaatggtacgaaaatcaaataa

SEQ ID NO: 50 - protein, 650 aa, Geobacillus stearothermophilus
MEKKTFYLTTPIYYPSDRLHIGHAYTTVAGDAMARYKRMRGYDVMYLTGTDEHGQKIQRKAEEKGVTPQQYVDEIVAGIQELWKKLDISYDDFIRTTQERHKKVVEQIFTRLVEQGDIYLGEYEGWYCTPCESFYTERQLVDGNCPDCGRPVEKVKEESYFFRMSKYVDRLLQYYEENPDFIQPESRKNEMINNFIKPGLEDLAVSRTTFDWGIKVPGNPKHVIYVWIDALANYITALGYGTDNDEKFRKYWPADVHLVGKEIVRFHTIYWPIMLMALGLPLPKKVFGHGWLLMKDGKMSKSKGNVVDPVTLIDRYGLDALRYYLLREVPFGADGVFTPEGFIERINYDLANDLGNLLHRTVAMIEKYFDGVIPPYRGPKTPFDQELVQTAREVVRQYEEAMEGMEFSVALAAVWQLISRTNKYIDETQPWVLAKDEQKRDELAAVMTHLAESLRHTAVLLQPFLTRTPERMLAQLGITDHSLKEWDSLYDFGLIPEGTKVQKGEPLFPRLDIEAEVEYIKAHMQGGKPAAEPVKEEKKAAEAAEISIDEFAKVDLRVAEVIHAERMKNADKLLKLQLDLGGEKRQVISGIAEFYKPEELVGKKVICVANLKPAKLRGEWSEGMILAGGSGGEFSLATVDQHVPNGTKIK

SEQ ID NO: 51 - DNA, 1035 bp, Geobacillus
atgaaagaacgcctgtatgaactgaaacgtcaggcactggaacaaattggtcaggcacgtgatctgcgtatgctgaatgatgttcgtgttgcatatctgggtaaaaaaggtccgattaccgaagttctgcgtggtatgggtgcactgcctccggaagaacgtccgaaaattggtgcactggcaaatgaagttcgtgaagcaattcagcaggccctggaagcaaaacaggcaaaacttgaacaagaagaagtggaacgtaaactggcagccgaagcaattgatgttaccctgcctggtcgtccggttagcctgggtaatccgcatccgctgacacgtgttattgaagaaattgaggacctgtttattggcatgggttataccgttgcagaaggtccggaagttgaaaccgattattacaattttgaagccctgaatctgccgaaaggtcatccggcacgcgatatgcaggatagcttttatatcaccgaagaaattctgctgcgtacccatacctcaccgatgcaggcacgtaccatggaaaaacatcgtggtcgtggtccggttaaaatcatttgtccgggtaaagtttatcgtcgcgataccgatgatgcaacccatagccatcagtttacacagattgaaggtctggttgtggatcgtaatattcgtatgagcgatctgaaaggcaccctgcgtgaatttgcccgtaaactgtttggtgaaggtcgtgatattcgttttcgtccgagcttttttccgtttaccgaaccgagcgttgaagttgatgttagctgttttcgttgtgaaggccgtggttgcggtgtttgtaaaggcaccggttggattgaaattttaggtgcaggtatggttcatccgaatgttctggaaatggcaggttttgatagtaaaacctataccggttttgcattcggtatgggtcctgaacgtattgcaatgctgaaatatggcattgatgatatccgccacttctatcagaatgatctgcgctttctgcgtcagtttctgcgtgtttaa

SEQ ID NO: 52 - protein, 344 aa, Geobacillus
MKERLYELKRQALEQIGQARDLRMLNDVRVAYLGKKGPITEVLRGMGALPPEERPKIGALANEVREAIQQALEAKQAKLEQEEVERKLAAEAIDVTLPGRPVSLGNPHPLTRVIEEIEDLFIGMGYTVAEGPEVETDYYNFEALNLPKGHPARDMQDSFYITEEILLRTHTSPMQARTMEKHRGRGPVKIICPGKVYRRDTDDATHSHQFTQIEGLVVDRNIRMSDLKGTLREFARKLFGEGRDIRFRPSFFPFTEPSVEVDVSCFRCEGRGCGVCKGTGWIEILGAGMVHPNVLEMAGFDSKTYTGFAFGMGPERIAMLKYGIDDIRHFYQNDLRFLRQFLRV

SEQ ID NO: 53 - DNA, 2415 bp, Geobacillus stearothermophilus
atgctggttagctatcgttggctgggtgaatatgttgatctgaccggtattaccgcaaaagaactggcagaacgtattaccaaaagcggtattgaagttgaacgtgttgaagcactggatcgtggtatgaatggtgttgttattggtcatgttctggaatgtgaaccgcatccgaatgcagataaactgcgtaaatgtctggttgatttaggtgaaggtgaaccggtgcgtattatttgtggtgcaccgaatgttgcaaaaggtcagaaagttgcagttgccaaagttggtgcagttctgcctggtaactttaaaatcaaacgtgcaaaactgcgtggcgaagaaagcaatggtatgatttgtagcctgcaagaactgggtgttgaaaccaaagttgttccgaaagaatatgccgatggcatttttgtttttccgagtgatgcaccggttggtgccgatgcactggaatggctgggtctgcatgatgaagttctggaactggcactgaccccgaatcgtgcagattgtctgagcatgattggtgttgcctatgaagttgcagcaattctgggtcgtgatgttaaactgccggaagcagcagttaaagaaaatagcgaacatgtgcacgaatatatcagcgttcgtgtggaagcaccggaagataatccgctgtatgcaggtcgtattgttaaaaatgttcgtattggtccgagtccgctgtggatgcaggcacgtctgatggcagcaggtattcgtccgcataataatgttgttgacatcaccaactatatcctgctggaatatggtcagccgctgcatgcatttgattatgatcgtctgggtagcaaagaaattgttgttcgtcgtgcaaaagccggtgaaaccattattaccctggatgatgttgaacgtaaactgaccgaaaatcatctggtgattaccaatggtcgcgaaccggttgcactggcaggcgttatgggtggtgccaatagcgaagttcgtgatgataccaccaccgtttttattgaagcagcctatttcaccagtccggttattcgtcaggccgttaaagatcatggtctgcgtagcgaagcgagcacccgttttgaaaaaggtattgatccggcacgtaccaaagaggccctggatcgcgcagcagcactgatgagcgaatatgcaggcggtgaagttgttggtggtattgttgaagccagcgtttggcgtcaggatccggttgttgttaccgttacactggaacgcattaatggtgttctgggcaccgcaatgaccaaagaagaagtggctgccattctgagcaatctgcagtttccgtttaccgaagataatggcacctttaccattcatgttccgagccgtcgtcgtgatattgcaattgaagaagatattattgaagaggcagcccgtctgtatggttatgatcgcctgcctgcaacactgccggttgccgaagcaaaacctggtggtctgacaccgcatcaggcaaaacgtcgtcgcgttcgtcgttatctggaaggcaccggtctgtttcaggcaattacctatagcctgacctcaccggataaagcaacccgctttgccctggaaaccgcagaaccgattcgtctggcactgccgatgagtgaagaacgtagcgttctgcgtcagagcctgattccgcatctgctggaagccgcaagctataatcgtgcacgtcaggttgaagatgttgccctgtatgaaattggtagcgtttatctgagcaaaggtgaacatgtacagcctgcagaaaaagaacgtttagccggtgtgctgacaggtctgtggcatgcacatctgtggcagggtgaaaaaaaagccgttgatttttatgtggccaaaggtattctggatggtctgtttgatctgctgggtttagcagcacgtattgaatataaaccggcaaaacgcgctgatctgcatccgggtcgtaccgcagatattgtgctggatggccgtgtgattggttttgttggtcagctgcatcctgcagttcagaaagagtatgatctgaaagaaacctatgtgtttgagctggccctgaccgatctgctgaatgcagaaagcgaagcaattcgttatgaacctattccgcgttttccgagcgttgtgcgcgacattgcactggttgttgatgaaaatgttgaagcgggtgcactgaaacaggcaatcgaagaagcaggtaaaccgctggttaaagatgttagcctgttcgatgtttataaaggcgatcgtctgccggatggtaaaaaaagtctggcatttagcctgcgttattatgatccggaacgcaccctgacagatgaagaggttgcagcagtgcatgaacgtgtgctggcagcagttgaaaaacagtttggtgccgtgctgcgtggttaa

SEQ ID NO: 54 - protein, 804 aa, Geobacillus stearothermophilus
MLVSYRWLGEYVDLTGITAKELAERITKSGIEVERVEALDRGMNGVVIGHVLECEPHPNADKLRKCLVDLGEGEPVRIICGAPNVAKGQKVAVAKVGAVLPGNFKIKRAKLRGEESNGMICSLQELGVETKVVPKEYADGIFVFPSDAPVGADALEWLGLHDEVLELALTPNRADCLSMIGVAYEVAAILGRDVKLPEAAVKENSEHVHEYISVRVEAPEDNPLYAGRIVKNVRIGPSPLWMQARLMAAGIRPHNNVVDITNYILLEYGQPLHAFDYDRLGSKEIVVRRAKAGETIITLDDVERKLTENHLVITNGREPVALAGVMGGANSEVRDDTTTVFIEAAYFTSPVIRQAVKDHGLRSEASTRFEKGIDPARTKEALDRAAALMSEYAGGEVVGGIVEASVWRQDPVVVTVTLERINGVLGTAMTKEEVAAILSNLQFPFTEDNGTFTIHVPSRRRDIAIEEDIIEEAARLYGYDRLPATLPVAEAKPGGLTPHQAKRRRVRRYLEGTGLFQAITYSLTSPDKATRFALETAEPIRLALPMSEERSVLRQSLIPHLLEAASYNRARQVEDVALYEIGSVYLSKGEHVQPAEKERLAGVLTGLWHAHLWQGEKKAVDFYVAKGILDGLFDLLGLAARIEYKPAKRADLHPGRTADIVLDGRVIGFVGQLHPAVQKEYDLKETYVFELALTDLLNAESEAIRYEPIPRFPSVVRDIALVVDENVEAGALKQAIEEAGKPLVKDVSLFDVYKGDRLPDGKKSLAFSLRYYDPERTLTDEEVAAVHERVLAAVEKQFGAVLRG

SEQ ID NO: 55 - DNA, 1704 bp, Geobacillus stearothermophilus
atgcgtcagagccaggcatttattccgacactgcgtgaagttccggcagatgcagaagttaaaagccatcagctgctgctgcgtgcaggttttattcgtcagagcgcaagcggtgtttatacctttctgccgctgggtcagcgtgtgctgcagaaagttgaagcaattattcgcgaagaaatgaatcgtattggtgccatggaactgtttatgcctgcactgcagcctgcagaactgtggcagcagagcggtcgttggtatagctatggtccggaactgatgcgtctgaaagatcgtcatgaacgtgattttgcactgggtccgacacatgaagagatgattaccgcaattgttcgtgatgaggtgaaaacctataaacgtctgcctctggttctgtatcagatccagaccaaattccgtgatgaaaaacgtccgcgttttggtctgttacgtggtcgtgaatttatgatgaaagatgcctatagcttccataccagcaaagaaagcctggatgaaacctacaacaatatgtatgaagcctacgccaacatttttcgtcgttgcggtctgaattttcgtgcagttattgcagatagcggtgcaattggtggtaaagatacccacgaattcatggttctgagcgatattggtgaagataccattgcatatagtgatgcaagcgattatgcagccaatattgaaatggcaccggttgttgcaacctatgaaaaaagtgatgaacctccggcagaactgaagaaagttgccacaccgggtcagaaaaccattgccgaagttgcaagccatctgcaaattagtccggaacgttgtattaaaagcctgctgtttaatgtggatggtcgttatgttctggtgctggttcgtggtgatcatgaagcaaatgaagtgaaagtgaaaaatgtgctggatgccaccgttgttgaactggcaaaaccggaagaaaccgaacgtgttatgaatgcaccgattggtagcctgggtcctattggtgttagcgaagatgttaccgttattgccgatcatgcagttgcagcaattgttaatggtgtttgtggtgccaatgaagagggctatcattacattggtgtgaatccgggtcgcgattttgcagttagccagtatgccgatctgcgttttgttaaagaaggtgatccgagtccggatggtaaaggcaccattcgttttgcacgtggtattgaagttggccatgtttttaaactgggcaccaaatatagcgaagccatgaatgcagtttatctggatgagaatggtcagacccagacaatgattatgggttgttatggtattggcgttagccgtctggttgcagccattgcagaacagtttgccgatgaacatggtctggtttggcctgcaagcgttgcaccgtttcatattcatctgctgaccgcaaatgccaaatcagatgaacagcgtgcactggccgaagaatggtatgaaaaactgggtcaagcaggttttgaagtgctgtatgatgatcgtccagaacgtgccggtgttaaatttgccgatagcgatctgattggtattccgctgcgtgttaccgtgggtaaacgtgcaggcgaaggtgttgttgaagttaaagttcgtaaaaccggtgaaacctttgatgttccggttagcgaactggttgataccgcacgtcgtctgctgcagagctaa

SEQ ID NO: 56 - protein, 567 aa, Geobacillus stearothermophilus
MRQSQAFIPTLREVPADAEVKSHQLLLRAGFIRQSASGVYTFLPLGQRVLQKVEAIIREEMNRIGAMELFMPALQPAELWQQSGRWYSYGPELMRLKDRHERDFALGPTHEEMITAIVRDEVKTYKRLPLVLYQIQTKFRDEKRPRFGLLRGREFMMKDAYSFHTSKESLDETYNNMYEAYANIFRRCGLNFRAVIADSGAIGGKDTHEFMVLSDIGEDTIAYSDASDYAANIEMAPVVATYEKSDEPPAELKKVATPGQKTIAEVASHLQISPERCIKSLLFNVDGRYVLVLVRGDHEANEVKVKNVLDATVVELAKPEETERVMNAPIGSLGPIGVSEDVTVIADHAVAAIVNGVCGANEEGYHYIGVNPGRDFAVSQYADLRFVKEGDPSPDGKGTIRFARGIEVGHVFKLGTKYSEAMNAVYLDENGQTQTMIMGCYGIGVSRLVAAIAEQFADEHGLVWPASVAPFHIHLLTANAKSDEQRALAEEWYEKLGQAGFEVLYDDRPERAGVKFADSDLIGIPLRVTVGKRAGEGVVEVKVRKTGETFDVPVSELVDTARRLLQS

SEQ ID NO: 57 - DNA, 1275 bp, Geobacillus stearothermophilus
atgctggatgtgaaaattctgcgtacccagtttgaagaggtgaaagaaaaactgatgcagcgtggtggtgatctgaccaatattgatcgttttgaacagctggataaagatcgtcgtcgtctgattgcagaagttgaagaactgaaaagcaaacgcaatgatgttagccagcagattgcagttctgaaacgcgaaaaaaaagatgcagaaccgctgattgcacagatgcgtgaagttggtgatcgtattaaacgtatggatgagcagattcgtcagctggaagcagaactggatgatctgctgctgagcattccgaatgttccgcatgaaagcgttccgattggccagagcgaagaagataacgttgaagttcgtcgttggggtgaaccgcgtagctttagctttgaaccgaaaccgcattgggaaattgcagatcgtctgggtctgctggattttgaacgtgcagcaaaagttgcaggtagccgttttgttttctataaaggtctgggtgcacgtctggaacgtgcactgattaactttatgctggatattcacctggatgagtttggctatgaagaagttctgcctccgtatctggttaatcgtgcaagcatgattggcaccggtcagctgccgaaatttgcagaagatgcatttcatctggatagcgaggattattttctgattccgaccgcagaagttccggttaccaatctgcatcgtgatgaaattctggcagcagatgacctgccgatctattatgcagcatatagcgcatgttttcgtgcagaagcaggtagcgcaggtcgtgatacccgtggtctgattcgccagcatcagttcaataaagttgaactggtgaaattcgtgaagccggaagatagctatgatgaactggaaaagctgacccgtcaggcagaaaccattctgcagcgtctgggcctgccgtatcgtgttgttgcactgtgtaccggtgatctgggttttagcgttgcaaaaacctatgatattgaagtttggctgccgagctatggcacctatcgtgaaattagcagctgtagcaattttgaagcatttcaggcacgtcgtgccaatattcgttttcgtcgtgatccgaaagcaaaaccggaatatgttcataccctgaatggtagcggtctggcaattggtcgtaccgttgcagcaattctggaaaattatcagcaagaagatggcagcgttattgttccggaagcactgcgtccgtatatgggcaatcgtgatgttattcgttaa

SEQ ID NO: 58 - protein, 424 aa, Geobacillus stearothermophilus
MLDVKILRTQFEEVKEKLMQRGGDLTNIDRFEQLDKDRRRLIAEVEELKSKRNDVSQQIAVLKREKKDAEPLIAQMREVGDRIKRMDEQIRQLEAELDDLLLSIPNVPHESVPIGQSEEDNVEVRRWGEPRSFSFEPKPHWEIADRLGLLDFERAAKVAGSRFVFYKGLGARLERALINFMLDIHLDEFGYEEVLPPYLVNRASMIGTGQLPKFAEDAFHLDSEDYFLIPTAEVPVTNLHRDEILAADDLPIYYAAYSACFRAEAGSAGRDTRGLIRQHQFNKVELVKFVKPEDSYDELEKLTRQAETILQRLGLPYRVVALCTGDLGFSVAKTYDIEVWLPSYGTYREISSCSNFEAFQARRANIRFRRDPKAKPEYVHTLNGSGLAIGRTVAAILENYQQEDGSVIVPEALRPYMGNRDVIR

SEQ ID NO: 59 - DNA, 1959 bp, Geobacillus
atgccggatgttattcgtattacctttccggatggtgccgaaaaagaatttccgaaaggcaccaccaccgaagatgttgcagcaagcattagtccgggtctgaaaaaaaaggcaattgcgggtaaactgaatggtcgttttgttgatctgcgtacaccgctgcatgaagatggtgaactggtgattattacccaggatatgccggaagcactggatattctgcgtcatagcaccgcacatctgatggcacaggcaattaaacgtctgtatggcaatgtgaaattaggtgttggtccggtgattgaaaacggcttctattatgatatcgacatggaacataaactgacaccggatgatctgccgaaaattgaagcagaaatgcgcaaaatcgtgaaagagaacctggatattgttcgcaaagaagttagtcgcgaagaggcaattcgcctgtatgaagaaattggtgatgaactgaaactggaactgattgcagatattccggaaggtgaaccgattagcatttatgaacagggcgaattttttgatctgtgccgtggtgttcatgttccgagcaccggtaaaatcaaagaatttaaactgctgagcatcagcggtgcatattggcgtggtgatagcaataacaaaatgctgcagcgtatttatggcaccgcgtttttcaaaaaagaagatctggatcgttatctgcgtctgctggaagaagcaaaagaacgcgatcatcgtaaactgggtaaagagctggaactgtttaccaccagtcagcaggttggtcagggtctgccgctgtggctgccgaaaggtgcaaccattcgtcgtattattgaacgctatatcgtggataaagaagttgcactgggttacgatcatgtttatacaccggttctgggtagcgttgaactgtataaaaccagcggtcattgggatcactacaaagaaaatatgtttccgcctatggaaatggacaatgaagaactggttctgcgtccgatgaattgtccgcatcacatgatgatctataaaagcaaactgcacagctatcgtgaactgccgattcgtattgcagaactgggcaccatgcatcgttatgaaatgagcggtgcactgaccggtctgcagcgtgttcgtggtatgaccctgaatgatgcacatatctttgttcgtccggatcagatcaaagatgaattcaaacgtgtggtgaacctgatcctggaagtgtataaagattttggcatcgaagaatacagcttccgtctgagttatcgtgatccgcatgataaagaaaaatactatgatgacgatgaaatgtgggaaaaagcacagcgtatgctgcgtgaagcaatggatgaattaggtctggattattatgaagccgaaggtgaagcagccttttatggtccgaaactggatgttcaggttcgtaccgcactgggaaaagatgaaaccctgagcaccgttcagctggattttctgctgccggaacgtttcgatctgacctatattggtgaagatggcaaaccgcatcgtccggttgttattcatcgtggtgttgttagcaccatggaacgttttgtggcatttctgatcgaagagtataaaggtgcatttccgacctggctggcaccggttcaggttaaagttattccggttagtccggaagcgcacctggattatgcatatgatgttcagcgtaccctgaaagaacgtggttttcgtgttgaagttgatgaacgcgacgaaaaaatcggctataaaatccgtgaagcacagatgcagaaaatcccgtatatgctggttgttggtgataaagaggttagcgaacgcgcagttaatgttcgtcgttatggtgaaaaagaaagccgtaccatgggccttgatgaatttatggccctgctggcagatgatgttcgtgaaaaacgtacccgtctgggcaaagcacagtaa

SEQ ID NO: 60 - protein, 651 aa, Geobacillus
MPDVIRITFPDGAEKEFPKGTTTEDVAASISPGLKKKAIAGKLNGRFVDLRTPLHEDGELVIITQDMPEALDILRHSTAHLMAQAIKRLYGNVKLGVGPVIENGFYYDIDMEHKLTPDDLPKIEAEMRKIVKENLDIVRKEVSREEAIRLYEEIGDELKLELIADIPEGEPISIYEQGEFFDLCRGVHVPSTGKIKEFKLLSISGAYWRGDSNNKMLQRIYGTAFFKKEDLDRYLRLLEEAKERDHRKLGKELELFTTSQQVGQGLPLWLPKGATIRRIIERYIVDKEVALGYDHVYTPVLGSVELYKTSGHWDHYKENMFPPMEMDNEELVLRPMNCPHHMMIYKSKLHSYRELPIRIAELGTMHRYEMSGALTGLQRVRGMTLNDAHIFVRPDQIKDEFKRVVNLILEVYKDFGIEEYSFRLSYRDPHDKEKYYDDDEMWEKAQRMLREAMDELGLDYYEAEGEAAFYGPKLDVQVRALGKDETLSTVQLDFLLPERFDLTYIGEDGKPHRPVVIHRGVVSTMERFVAFLIEEYKGAFPTWLAPVQVKVIPVSPEAHLDYAYDVQRTLKERGFRVEVDERDEKIGYKIREAQMQKIPYMLVVGDKEVSERAVNVRRYGEKESRTMGLDEFMALLADDVREKRTRLGKAQ

SEQ ID NO: 61 - DNA, 987 bp, Geobacillus stearothermophilus
atgaaaaccatctttagcggtattcagccgagcggtgttattaccctgggtaactatattggtgcactgcgtcagtttattgaactgcagcatgaatataactgctatttctgcattgttgatcagcatgcaattaccgtttggcaggatccgcatgaactgcgccagaatattcgtcgtctggcagcactgtatctggcagttggtattgatccgacacaggcaaccctgtttattcagagcgaagttccggcacatgcacaggcagcatggatgctgcaatgtattgtttatattggcgaactggaacgcatgacccagtttaaagaaaaaagcgcaggtaaagaagcagttagcgcaggtctgctgacctatccgcctctgatggcagccgatattctgctgtataacaccgatattgttccggttggtgatgatcagaaacagcatatcgaactgacccgtgatctggcagaacgttttaacaaacgttatggtgagctgtttaccattccggaagcacgtattccgaaagttggtgcacgtattatgagcctggtggatccgaccaaaaaaatgagcaaaagcgatccgaatccgaaagcctatattacactgctggatgatgcaaaaaccatcgagaaaaaaatcaaaagtgccgtgaccgatagcgaaggcaccattcgttatgataaagaagccaaaccgggtattagcaacctgctgaacatttatagcaccctgagcggtcagagcattgaagaattagaacgtaaatatgaaggcaaaggctacggtgtttttaaagcagatctggcacaggttgttattgaaaccctgcgtccgattcaagaacgttatcatcattggatggaaagcgaagaactggatcgtgttctggatgaaggtgcagaaaaagcaaatcgtgttgcaagcgaaatggtgcgtaaaatggaacaggcaatgggtctgggtcgtcgtcgttaa

SEQ ID NO: 62 - protein, 328 aa, Geobacillus stearothermophilus
MKTIFSGIQPSGVITLGNYIGALRQFIELQHEYNCYFCIVDQHAITVWQDPHELRQNIRRLAALYLAVGIDPTQATLFIQSEVPAHAQAAWMLQCIVYIGELERMTQFKEKSAGKEAVSAGLLTYPPLMAADILLYNTDIVPVGDDQKQHIELTRDLAERFNKRYGELFTIPEARIPKVGARIMSLVDPTKKMSKSDPNPKAYITLLDDAKTIEKKIKSAVTDSEGTIRYDKEAKPGISNLLNIYSTLSGQSIEELERKYEGKGYGVFKADLAQVVIETLRPIQERYHHWMESEELDRVLDEGAEKANRVASEMVRKMEQAMGLGRRR

SEQ ID NO: 63 - DNA, 1260 bp, Geobacillus stearothermophilus
atggatctgctggcagaactgcagtggcgtggtctggtgaatcagaccaccgatgaagatggtctgcgtgaactgctgaaagaagaacgcgttaccctgtattgtggttttgatccgaccgcagatagcctgcatattggtaatctggcagcaattctgaccctgcgtcgttttcagcaggcaggtcatcagccgattgcactggttggtggtgcaaccggtctgattggtgatccgagcggtaaaaaaagcgaacgtaccctgaatgcaaaagaaaccgttgaagcatggtcagcacgtattcaagaacagctgagccgttttctggattttgaagcacatggtaatccggcaaaaatcaagaacaactatgattggattggtccgctggatgttattacctttctgcgtgatgttggcaaacatttcagcgtgaattatatgatggccaaagaaagcgttcagagccgtattgaaaccggtattagctttaccgaattcagctatatgatgctgcaggcctatgattttctgcgtctgtatgaaaccgaaggttgtcgtctgcagattggtggtagcgatcagtggggcaatattaccgcaggtctggaactgattcgtaaaaccaaaggtgaagcacgtgcatttggtctgaccattccgctggttaccaaagcagatggtacaaaatttggtaaaaccgaaagcggcaccatttggctggataaagaaaaaaccagtccgtatgagttctaccagttttggattaataccgatgatcgtgatgtgatccgctacctgaaatactttacatttctgagcaaagaagagatcgaagcctttgaacaagaactgcgtgaagcaccggaaaaacgtgcagcacagaaagcactggcagaagaagttaccaaactggttcatggtgaagaagcactgcgtcaggcagttcgtattagcgaagcactgtttagcggtgatattggcaacctgaccgcagcagaaattgaacagggttttaaagatgttccgagctttgttcatgaaggtggtgatgtgccgctggtcgaactgctggttagcgcaggtattagcccgagcaaacgtcaggcacgtgaagatattcagaatggtgccatttatgtgaatggtgaacgtctgcaggatgttggtgcgattctgacagcagaacatcgtctggaaggtcgttttaccgttattcgtcgtggcaaaaaaaagtattacctgattcgctatgcctaa

SEQ ID NO: 64 - protein, 419 aa, Geobacillus stearothermophilus
MDLLAELQWRGLVNQTTDEDGLRELLKEERVTLYCGFDPTADSLHIGNLAAILTLRRFQQAGHQPIALVGGATGLIGDPSGKKSERTLNAKETVEAWSARIQEQLSRFLDFEAHGNPAKIKNNYDWIGPLDVITFLRDVGKHFSVNYMMAKESVQSRIETGISFTEFSYMMLQAYDFLRLYETEGCRLQIGGSDQWGNITAGLELIRKTKGEARAFGLTIPLVTKADGTKFGKTESGTIWLDKEKTSPYEFYQFWINTDDRDVIRYLKYFTFLSKEEIEAFEQELREAPEKRAAQKALAEEVTKLVHGEEALRQAVRISEALFSGDIGNLTAAEIEQGFKDVPSFVHEGGDVPLVELLVSAGISPSKRQAREDIQNGAIYVNGERLQDVGAILTAEHRLEGRFTVIRRGKKKYYLIRYA

SEQ ID NO: 65 - DNA, 2643 bp, Geobacillus
atggcacagcatgaagttagcatgcctccgaaatatgatcatcgtgcagttgaagcaggtcgttatgaatggtggctgaaaggtaaattctttgaagcaaccggtgatccgaataaacgtccgtttaccattgttattccgcctccgaatgtgaccggtaaactgcatctgggtcatgcatgggataccacactgcaggatattatcacccgtatgaaacgtatgcagggttatgatgttctgtggctgcctggtatggatcatgcaggtattgcaacccaggcaaaagttgaagaaaaactgcgtcagcagggtctgagccgttatgatctgggtcgtgaaaaatttctggaagaaacctggaaatggaaagaagaatacgcaggtcatattcgtagccagtgggcaaaattaggtctgggtttagattatacccgtgaacgttttaccctggatgaaggtctgagcaaagcagttcgtgaagtttttgttagcctgtatcgtaaaggtctgatttatcgcggtgagtatatcattaattgggaccctgttaccaaaaccgcactgagcgatattgaagtggtttacaaagaagttaaaggcgcactgtatcatctgcgttatccgctggcagatggtagcggttgtattgaagttgcaaccacacgtccggaaaccatgctgggtgataccgcagttgcagttcatcctgatgatgaacgttataaacatctgatcggcaaaatggtgaaactgccgattgttggtcgcgaaattccgattattgcagatgaatatgtggacatggaatttggtagtggtgccgtgaaaattacaccggcacatgatccgaacgattttgaaattggtaatcgccataatctgcctcgtattctggtgatgaatgaagatggcaccatgaatgaaaatgccatgcagtatcaaggtctggatcgttttgaatgccgtaaacaaattgttcgcgatctgcaagaacagggtgttctgtttaaaatcgaagaacatgtgcatagcgttggtcatagcgaacgtagcggtgcagttattgaaccgtatctgagcacccagtggtttgttaaaatgaaaccgctggccgaagcagcaattaaactgcagcagaccgatggtaaagttcagtttgtgccggaacgctttgaaaaaacctatctgcattggctggaaaacattcgtgattggtgtattagccgtcagctgtggtggggtcatcgtattccggcatggtatcataaagaaaccggtgaaatttatgtggatcacgaaccgcctaaagatatcgaaaattgggaacaagatccggatgttctggatacctggtttagcagcgcactgtggccgtttagcaccatgggttggcctgatgttgaaagtccggattataaacgttattatccgaccgatgtgctggttaccggttatgatattatctttttttgggtgagccgcatgatttttcaaggcctggaatttaccggcaaacgcccttttaaagatgttctgattcatggtctggtgcgtgatgcacagggtcgtaaaatgagcaaaagcttaggtaatggtgttgatccgatggatgtgattgatcagtatggtgcagatgcactgcgttattttctggcaaccggtagcagccctggtcaggatctgcgttttagcaccgaaaaagtggaagcaacgtggaattttgccaacaaaatttggaatgcaagccgttttgcactgatgaacatgggtggtatgacctatgaagaactggatctgagcggtgaaaaaacagttgcggatcattggattctgacccgtctgaatgaaaccattgataccgttaccaaactggccgaaaaatatgaatttggtgaagccggtcgtaccctgtataactttatttgggatgatctgtgcgattggtatatcgaaatggcaaaactgccgctgtatggtgatgatgaggcagcaaaaaaaacaacccgtagcgttctggcatatgtgctggataataccatgcgcctgctgcatccgtttatgccgtttattaccgaagaaatttggcagaatctgccgcatgaaggtgaaagcattaccgttgcaccgtggcctcaggttcgtccggaactgagcaatgaagaggcagcggaagaaatgcgtatgctggttgatattattcgtgccgttcgtaatgttcgtgccgaagttaatacccctccgagcaaaccgattgcactgtatatcaaagttaaagacgaacaggttcgtgcagccctgatgaaaaatcgtgcatatctggaacgtttttgcaatccgagcgaactgctgattgataccaatgttcctgcaccggataaagcaatgaccgcagtggtgaccggtgcagaactgattatgccgctggaaggcctgattaacattgaagaagaaattaaacgcctggaaaaagaacttgataaatggaacaaagaggtggaacgcgtcgaaaaaaaactggcaaatgaaggttttctggccaaagcaccagcgcatgttgtggaagaagaacgtcgtaaacgtcaggattacatggaaaaacgtgaagcagttaaagcacgtctggccgaactgaaacgttaa

SEQ ID NO: 66 - protein, 880 aa, Geobacillus
MAQHEVSMPPKYDHRAVEAGRYEWWLKGKFFEATGDPNKRPFTIVIPPPNVTGKLHLGHAWDTTLQDIITRMKRMQGYDVLWLPGMDHAGIATQAKVEEKLRQQGLSRYDLGREKFLEETWKWKEEYAGHIRSQWAKLGLGLDYTRERFTLDEGLSKAVREVFVSLYRKGLIYRGEYIINWDPVTKTALSDIEVVYKEVKGALYHLRYPLADGSGCIEVATTRPETMLGDTAVAVHPDDERYKHLIGKMVKLPIVGREIPIIADEYVDMEFGSGAVKITPAHDPNDFEIGNRHNLPRILVMNEDGTMNENAMQYQGLDRFECRKQIVRDLQEQGVLFKIEEHVHSVGHSERSGAVIEPYLSTQWFVKMKPLAEAAIKLQQTDGKVQFVPERFEKTYLHWLENIRDWCISRQLWWGHRIPAWYHKETGEIYVDHEPPKDIENWEQDPDVLDTWFSSALWPFSTMGWPDVESPDYKRYYPTDVLVTGYDIIFFWVSRMIFQGLEFTGKRPFKDVLIHGLVRDAQGRKMSKSLGNGVDPMDVIDQYGADALRYFLATGSSPGQDLRFSTEKVEATWNFANKIWNASRFALMNMGGMTYEELDLSGEKTVADHWILTRLNETIDTVTKLAEKYEFGEAGRTLYNFIWDDLCDWYIEMAKLPLYGDDEAAKKTTRSVLAYVLDNTMRLLHPFMPFITEEIWQNLPHEGESITVAPWPQVRPELSNEEAAEEMRMLVDIIRAVRNVRAEVNTPPSKPIALYIKVKDEQVRAALMKNRAYLERFCNPSELLIDTNVPAPDKAMTAVVTGAELIMPLEGLINIEEEIKRLEKELDKWNKEVERVEKKLANEGFLAKAPAHVVEEERRKRQDYMEKREAVKARLAELKR

SEQ ID NO: 67 - DNA, 960 bp, Geobacillus stearothermophilus
atgaccaacattgtgtttatgggcacaccggattttgcagttccgattctgcgtcagctgctgcatgatggttatcgtgttgcagcagttgttacccagccggataaaccgaaaggtcgtaaacgtgaacctgttccgcctccggttaaagttgaagcagaacgtcgtggtattccggttctgcagccgaccaaaattcgtgaaccggaacagtatgaacaggtgctggcatttgcaccggatctgattgttaccgcagcatttggtcagattctgccgaaagcactgctggatgcaccgaaatatggttgcattaatgttcatgcaagcctgctgccggaactgcgtggtggtgcaccgattcattatgcaatttggcagggtaaaaccaaaaccggtgttaccattatgtatatggttgaacgtctggatgccggtgatatgctggcacaggttgaagtgccgattgcagaaaccgataccgttggcaccctgcatgataaactgagcgcagcgggtgcaaaactgctgagcgaaaccctgccgctgctgctggaaggcaatattacaccggttccgcaggatgaagaaaaagcaacctatgcacctaatattcgtcgtgaacaagaacgtattgattggacccagcctggtgaagccatttataaccatattcgtgcctttcatccgtggcctgttacctataccacacaggatggtcatatttggaaagtttggtggggtgaaaaagttcctgcaccgcgtagcgcaccgcctggcaccattctggcactggaagaaaatggtattgttgttgcaaccggtaatgaaaccgcaattcgtattaccgaactgcagcctgcaggtaaaaaacgtatggcagccggtgaatttctgcgtggcgcaggtagccgtctggcagttggtatgaaactgggtgaagatcatgaacgtacctaa

SEQ ID NO: 68 - protein, 319 aa, Geobacillus stearothermophilus
MTNIVFMGTPDFAVPILRQLLHDGYRVAAVVTQPDKPKGRKREPVPPPVKVEAERRGIPVLQPTKIREPEQYEQVLAFAPDLIVTAAFGQILPKALLDAPKYGCINVHASLLPELRGGAPIHYAIWQGKTKTGVTIMYMVERLDAGDMLAQVEVPIAETDTVGTLHDKLSAAGAKLLSETLPLLLEGNITPVPQDEEKATYAPNIRREQERIDWTQPGEAIYNHIRAFHPWPVTYTTQDGHIWKVWWGEKVPAPRSAPPGTILALEENGIVVATGNETAIRITELQPAGKKRMAAGEFLRGAGSRLAVGMKLGEDHERT

SEQ ID NO: 69 - DNA, 249 bp, Geobacillus subterraneus
atgttactcattcgaaggagggagagccgctcgatggcaaaagacgatgtaattgaagtggaaggcaccgtcattgaaacattgccaaatgcgatgtttcgtgtagaattagaaaatgggcacacagtattggcccatgtgtccggcaaaatccgtatgcacttcatccgcattttgcctggcgataaagtgacggtggagttgtcgccgtatgatttaacgcgtggacggattacgtatcgatataaa

SEQ ID NO: 70 - protein, 83 aa, Geobacillus subterraneus
MLLIRRRESRSMAKDDVIEVEGTVIETLPNAMFRVELENGHTVLAHVSGKIRMHFIRILPGDKVTVELSPYDLTRGRITYRYK

SEQ ID NO: 71 - DNA, 2352 bp, Geobacillus subterraneus
atggtgtcccgctttgcaaagtgccggaccggtatacgctcggcggcgcgatcggcaaagacgcccgcgtcgttgtcgccgtcaccgacgaagggttcgcgcgccaattgcaaacgatgctcgactgatctttatgggggtgaatgtatgtcgaaaatgcgtgtgtacgaatacgccaaaaaacataatgtgccaagcaaggacgttattcataaattgaaagaaatgaatattgaagtgaacaaccatatgactatgctcgaagccgatgtcgtcgaaaagctcgatcatcaataccgcgtgaactcagagaaaaaagcggaaaagaaaacggagaaaccgaagcggccgacgccggcgaaagccgccgattttgccgacgaggaaatgtttgaggacaagaaagaaacggcaaagacgaagccggcgaagaaaaagggagcagtgaaaggaaaggaaacgaaaaaaacagaagcacagcagcaagaaaagaaactgttccaagcggcgaagaaaaaaggaaaaggaccgatgaaaggcaaaaaacaagctgccccagcctcaaagcaggcgcagcagccggcgaaaaaagaaaaagagctcccgaaaaaaattacgttcgaaggttcgctcacggtagccgaattggcgaaaaaacttggccgcgagccgtcggaaatcattaaaaaactgtttatgctcggcgtcatggcgacgattaaccaagatttagacaaagatgcgatcgagctcatttgctctgattacggagttgaagtcgaagaaaaagtgacgatcgatgaaacgaattttgaaacgatcgaaattgtcgatgcaccggaagatttggtggaacggccgccggtcgtcacgattatggggcacgttgaccacgggaaaacaacgctgcttgacgcaatccgccactcgaaagtgaccgagcaagaggcgggcggtattacacagcatatcggtgcttatcaagtcacggtcaacggcaagaaaattacgttcctcgatacgccggggcatgaagcgtttacgacgatgcgggcgcgcggtgcgcaagtgacggatatcgtcatccttgttgttgctgctgatgatggggtcatgccgcagacggtcgaggcgattaaccacgccaaagcggcgaacgtaccgattatcgtcgccattaacaaaatggataagccggaagcaaacccggatcgcgttatgcaagagttgatggagtacaacctcgttccggaagaatggggtggcgatacgattttctgcaagctgtcggcgaaaacccaagacggtattgaccatctgttggaaatgattttgcttgtcagcgaaatggaagaactaaaagcgaacccgaaccgccgcgcgctcggtacggtgatcgaagcgaagctcgataaagggcgcggtccggtagcgacgttgctcgtccaagccggtacgctaaaagtcggtgatccgattgttgtcggaacaacgtacggacgcgtgcgcgcgatggtcaatgacagcggtcggcgtgtcaaagaagcgggtccgtcgatgccggtcgaaatcacagggcttcatgatgtgccgcaagccggggaccgctttatggtatttgaagatgagaagaaagcgcgacaaatcggagaagcgcgggcacagcggcagctgcaagagcagcggagcgtgaaaacgcgcgtcagcttggacgatttgtttgaacaaattaagcaaggtgaaatgaaagagctgaacttgatcgttaaggccgacgtccaaggatcggtcgaagcgcttgtcgccgccttgcaaaaaatcgatatcgaaggcgtgcgtgtgaaaattatccacgcggcggtcggcgccattacggagtcagacatcttgttggcaacgacctcgaacgcgatcgtcatcggttttaacgtccgtccggacaccaatgcgaagcgggctgccgaatcagaaaacgtcgacatccgcctccaccgcattatttacaatgtcatcgaagaaattgaagcggcgatgaaagggatgctcgacccagaatatgaagaaaaagtgatcggtcaggcggaagtgcggcaaacgttcaaagtgtcgaaagtcggcacgatcgccgggtgctacgtcaccgacggcaaaattacccgcgacagcaaagtgcgccttatccgtcaaggcatcgtcgtgtacgaaggcgaaatcgactcgctcaaacggtataaagatgatgtgcgtgaggtggcgcaaggatacgaatgcggcgtgaccatcaaaaacttcaacgatattaaagaaggggacgtcatcgaggcgtacatcatgcaggaagtggctcgcgca

SEQ ID NO: 72 - protein, 784 aa, Geobacillus subterraneus
MVSRFAKCRTGIRSAARSAKTPASLSPSPTKGSRANCKRCSTDLYGGECMSKMRVYEYAKKHNVPSKDVIHKLKEMNIEVNNHMTMLEADVVEKLDHQYRVNSEKKAEKKTEKPKRPTPAKAADFADEEMFEDKKETAKTKPAKKKGAVKGKETKKTEAQQQEKKLFQAAKKKGKGPMKGKKQAAPASKQAQQPAKKEKELPKKITFEGSLTVAELAKKLGREPSEIIKKLFMLGVMATINQDLDKDAIELICSDYGVEVEEKVTIDETNFETIEIVDAPEDLVERPPVVTIMGHVDHGKTTLLDAIRHSKVTEQEAGGITQHIGAYQVTVNGKKITFLDTPGHEAFTTMRARGAQVTDIVILVVAADDGVMPQTVEAINHAKAANVPIIVAINKMDKPEANPDRVMQELMEYNLVPEEWGGDTIFCKLSAKTQDGIDHLLEMILLVSEMEELKANPNRRALGTVIEAKLDKGRGPVATLLVQAGTLKVGDPIVVGTTYGRVRAMVNDSGRRVKEAGPSMPVEITGLHDVPQAGDRFMVFEDEKKARQIGEARAQRQLQEQRSVKTRVSLDDLFEQIKQGEMKELNLIVKADVQGSVEALVAALQKIDIEGVRVKIIHAAVGAITESDILLATTSNAIVIGFNVRPDTNAKRAAESENVDIRLHRIIYNVIEEIEAAMKGMLDPEYEEKVIGQAEVRQTFKVSKVGTIAGCYVTDGKITRDSKVRLIRQGIVVYEGEIDSLKRYKDDVREVAQGYECGVTIKNFNDIKEGDVIEAYIMQEVARA

SEQ ID NO: 73 - DNA, 339 bp, Geobacillus subterraneus
atggactacggcaaattccgctttgagcagcaaaagaaagaaaaagaagcgcgcaaaaagcaaaaggtgatcaacattaaagaggtgcgcctcagcccgacaattgaggaacacgactttaatacgaaactacgcaatgcgcgcaagtttttagaaaaaggcgataaagtgaaggcgacgatccgctttaaagggcgggcgatcacccataaagaaatcgggcagcgcgtccttgaccgcttctcggaagcatgcgctgatatcgcggtcgtcgaaacggcgccgaaattggaagggcgcaacatgtttttagtgctggcaccgaaaaatgacaacaag

SEQ ID NO: 74 - protein, 113 aa, Geobacillus subterraneus
MDYGKFRFEQQKKEKEARKKQKVINIKEVRLSPTIEEHDFNTKLRNARKFLEKGDKVKATIRFKGRAITHKEIGQRVLDRFSEACADIAVVETAPKLEGRNMFLVLAPKNDNK

SEQ ID NO: 75 - DNA, 2076 bp, Geobacillus subterraneus
atggcaagagagttctccttagaaaacactcgtaacataggaatcatggcgcacattgacgccggaaaaacgacgacgacggaacgaatcctgttctacacaggccgcgttcataaaatcggggaaacgcatgaaggctcagctacgatggactggatggaacaagagcaagagcgcgggattacgattacgtcggcggcgacaacggcgcaatggaaaggccatcgcatcaacatcatcgacacgccagggcacgtcgacttcacggttgaggttgaacgttcgttgcgcgtgttggacggagccattacagttcttgacgcccaatctggtgtagaaccgcaaacggaaacagtttggcgtcaagcgactacatatggtgttccgcggattgtattcgtcaacaaaatggacaaaatcggtgcggacttcttgtatgcggtaaaaacgctccatgaccgcttacaagcgaatgcctacccggtgcagttgccgatcggcgctgaagaccaattcaccggcattattgacctcgtggaaatgtgtgcataccattaccacgacgaccttggcaaaaacatcgaacgcatcgaaattccggaagactaccgcgatttagcggaagaatatcatggcaagctcattgaggctgttgcggaactcgatgaagagctgatgatgaaatatttagaaggagaagaaattacgaaagaagagctgaaagccgcaatccgtaaggcgacgatcaacgttgaattctatccagtcttctgcggttcagcttttaaaaacaaaggtgttcagctgcttcttgacggggttgtcgactacttgccgtctccgttagatatcccggcgattcgcggtatcattccggatacggaagaagaagtggctcgcgaagcacgcgatgacgctccgttctccgcgttggcattcaaaattatgactgacccgtacgttgggaagttgacgttcttccgcgtctactccggaacgcttgattccggttcttacgtcatgaactcaacgaaacggaagcgtgaacggatcggtcgcttgctgcaaatgcatgcgaaccaccgtcaagaaatttcgacagtctatgccggtgatattgcggcagcagtaggtttaaaagaaacaacgaccggcgatactctatgtgatgagaaaaatcttgtcatcttagagtcgatgcaattcccagagccggttatctcggtggcgatcgaaccgaaatcgaaagccgaccaagataagatgggtcaagcattgcaaaaactgcaagaggaagacccgacattccgtgcgcataccgatccggaaacaggacaaacgatcatttccgggatgggcgagctgcacttggacattatcgtcgaccggatgcgtcgcgaattcaaagtcgaggcgaacgttggtgcaccgcaagttgcttaccgtgaaacgttccgtcaatcggctcaagtcgaagggaaatttattcgccagtccggtggtcgtggtcagtacggtcacgtttggatcgaattcacaccgaacgaacgcggtaaaggctttgaatttgaaaatgcgatcgtcggtggggtcgttccgaaagagtacgtgccggctgttcaagctggattggaagaagcgatgcaaaacggtgtcttagctggctacccggttgttgacatcaaagcgaaactgtttgatggatcgtaccatgatgtcgactcgagtgagatggcgttcaaaattgctgcttcgatggcgttgaaaaacgcggcagcgaagtgtgaaccggttctgcttgaaccgatcatgaaagtagaagtcgtcatccctgaagaatacctcggcgacattatgggtgacatcacatcccgccgcggtcgcgtcgaagggatggaagcgcgcggaaacgcccaagttgttcgtgcaatggtgccgctggccgaaatgttcggttatgcaacatcgctccgttcgaacacgcaagggcgtggaacgttctcgatggtatttgaccattacgaagaagttccgaaaaacatcgccgatgaaattatcaaaaaaaataaaggcgaa

SEQ ID NO: 76 - protein, 692 aa, Geobacillus subterraneus
MAREFSLENTRNIGIMAHIDAGKTTTTERILFYTGRVHKIGETHEGSATMDWMEQEQERGITITSAATTAQWKGHRINIIDTPGHVDFTVEVERSLRVLDGAITVLDAQSGVEPQTETVWRQATTYGVPRIVFVNKMDKIGADFLYAVKTLHDRLQANAYPVQLPIGAEDQFTGIIDLVEMCAYHYHDDLGKNIERIEIPEDYRDLAEEYHGKLIEAVAELDEELMMKYLEGEEITKEELKAAIRKATINVEFYPVFCGSAFKNKGVQLLLDGVVDYLPSPLDIPAIRGIIPDTEEEVAREARDDAPFSALAFKIMTDPYVGKLTFFRVYSGTLDSGSYVMNSTKRKRERIGRLLQMHANHRQEISTVYAGDIAAAVGLKETTTGDTLCDEKNLVILESMQFPEPVISVAIEPKSKADQDKMGQALQKLQEEDPTFRAHTDPETGQTIISGMGELHLDIIVDRMRREFKVEANVGAPQVAYRETFRQSAQVEGKFIRQSGGRGQYGHVWIEFTPNERGKGFEFENAIVGGVVPKEYVPAVQAGLEEAMQNGVLAGYPVVDIKAKLFDGSYHDVDSSEMAFKIAASMALKNAAAKCEPVLLEPIMKVEVVIPEEYLGDIMGDITSRRGRVEGMEARGNAQVVRAMVPLAEMFGYATSLRSNTQGRGTFSMVFDHYEEVPKNIADEIIKKNKGE

SEQ ID NO: 77 - DNA, 1185 bp, Geobacillus subterraneus
atggctaaagcgaaatttgagcgtacgaaaccgcacgtcaacattggcacgatcggccacgttgaccatgggaaaacgacgttgacagctgcgatcacgacagttcttgcgaaacaaggtaaagcagaagcgagagcgtacgaccaaatcgacgctgctccggaagagcgtgaacgcggaatcacgatttcgacggctcacgttgagtatgaaacagaaaaccgtcactatgcgcacgttgactgcccgggccacgctgactacgtgaaaaacatgatcacgggcgcagcgcaaatggacggcgcgatccttgttgtatcggctgctgacggtccgatgccgcaaactcgcgaacacattcttctttcccgccaagtcggtgttccgtacatcgttgttttcttgaacaaatgcgacatggtggacgacgaagaattgcttgaactcgttgaaatggaagttcgcgatcttctttctgaatatgacttcccgggcgacgaagtgccggttatcaaaggttcggcattaaaagcgctcgaaggcgatgcacaatgggaagaaaaaatcgttgaactgatgaacgcggttgacgagtacatcccaactccgcaacgtgaagtagacaaaccgttcatgatgccggttgaggacgtcttctcgatcacgggtcgtggtacggttgcaacgggccgtgttgagcgcggtacgttaaaagttggtgacccggttgaaatcatcggtctttcggacgagccgaaatcgacgactgttacgggtgtagaaatgttccgtaagcttctcgaccaagcagaagctggtgacaacatcggtgcgcttctccgcggtgtatcgcgtgacgaagttgagcgcggtcaagtattggcgaaaccgggctcgatcacgccacacacgaaatttaaagcacaagtttacgttctgacgaaagaagaaggcggacgccatactccgttcttctcgaactaccgtccgcaattctacttccgtacaacggacgtaacgggcatcatcacgcttccagaaggcgttgaaatggttatgcctggcgacaacgttgaaatgacggttgaactgatcgctccgatcgcgatcgaagaaggtacgaaattctcgatccgtgaaggcggccgcacggttggtgctggttccgtatcggaaatcattgag

SEQ ID NO: 78 - protein, 395 aa, Geobacillus subterraneus
MAKAKFERTKPHVNIGTIGHVDHGKTTLTAAITTVLAKQGKAEARAYDQIDAAPEERERGITISTAHVEYETENRHYAHVDCPGHADYVKNMITGAAQMDGAILVVSAADGPMPQTREHILLSRQVGVPYIVVFLNKCDMVDDEELLELVEMEVRDLLSEYDFPGDEVPVIKGSALKALEGDAQWEEKIVELMNAVDEYIPTPQREVDKPFMMPVEDVFSITGRGTVATGRVERGTLKVGDPVEIIGLSDEPKSTTVTGVEMFRKLLDQAEAGDNIGALLRGVSRDEVERGQVLAKPGSITPHTKFKAQVYVLTKEEGGRHTPFFSNYRPQFYFRTTDVTGIITLPEGVEMVMPGDNVEMTVELIAPIAIEEGTKFSIREGGRTVGAGSVSEIIE

SEQ ID NO: 79 - DNA, 882 bp, Geobacillus subterraneus
atggcgattacagcacaaatggtaaaagagctgcgcgaaaaaacgggcgcaggcatgatggactgcaaaaaagcgctcaccgaaacgaacggtgacatggaaaaagcgatcgactggctgcgtgaaaaaggaattgctaaagcagcgaaaaaagcagatcgcatcgcagcggaaggaatgacatacatcgcgacggaaggcaatgcggctgtcattttggaagtaaactcggaaacggacttcgttgccaaaaacgaagcgttccaaacgctcgttaaggagctggctgcacatctgctgaaacaaaagccagccacgcttgatgaagcgctcggacaaacgatgagcagtggttccactgttcaagattacattaacgaagcagttgctaaaatcggtgaaaaaattacgctccgccgctttgctgttgtcaacaaagcggatgatgaaacgtttggcgcgtacttgcacatgggcgggcgcatcggcgtattaacattattagccggcaacgcaactgaagaggtcgctaaagatgtggcgatgcatattgctgcgctccatccgaaatacgtttcgcgcgatgaagtgccgcaagaagagattgcgcgcgaacgtgaagtgttgaaacaacaagcgttgaacgaaggtaagccggaaaacatcgttgaaaaaatggttgaaggccgtctgaaaaagttttacgaagatgtttgcctgcttgagcaagcgttcgtgaaaaacccggatgtgacggtacgccaatacgtcgaatcgagcggagcaaccgtgaagcagttcatccgctacgaagttggtgaagggctcgaaaaacgtcaagataatttcgctgaagaagtcatgagccaagtaagaaaacaa

SEQ ID NO: 80 - protein, 294 aa, Geobacillus subterraneus
MAITAQMVKELREKTGAGMMDCKKALTETNGDMEKAIDWLREKGIAKAAKKADRIAAEGMTYIATEGNAAVILEVNSETDFVAKNEAFQTLVKELAAHLLKQKPATLDEALGQTMSSGSTVQDYINEAVAKIGEKITLRRFAVVNKADDETFGAYLHMGGRIGVLTLLAGNATEEVAKDVAMHIAALHPKYVSRDEVPQEEIAREREVLKQQALNEGKPENIVEKMVEGRLKKFYEDVCLLEQAFVKNPDVTVRQYVESSGATVKQFIRYEVGEGLEKRQDNFAEEVMSQVRKQ

SEQ ID NO: 81 - DNA, 1827 bp, Geobacillus subterraneus
atgaaccgggaagaacggttgaaacggcaggaacggattcgcaacttttcgattatcgctcacattgaccacggaaaatcgacgcttgcggaccgcattttagaaaaaacaggtgcgctgtcggagcgcgagttgcgcgagcagacgctcgatatgatggagctcgagcgcgagcgcggcatcacgatcaaattgaatgcggtccagttgacatataaagcgaaaaacggggaagagtatattttccatttgatcgatacgccgggccacgtcgattttacgtatgaagtgtcgcgcagcttggctgcttgcgaaggagcgatcttagtcgtcgatgcggcgcaaggcattgaagcgcagacgctcgcaaacgtgtatttggccattgacaacaatttagaaattttaccagtcattaataaaatcgatttgccaagcgccgagccggagcgtgtccgccaagaaatcgaagacgtcattggcctcgatgcctctgaagcggtgctcgcctccgcgaaagtcggcatcggcgtcgaggacattttagaacaaatcgtggaaaaaattcctgctccgtcaggcgatccggacgcgccgttgaaggcgctcatttttgattcactttatgacccgtaccgcggcgttgtcgcctacgtccgtatcgtcgatggaacggttaagccgggccagcgcattaaaatgatgtcgaccggcaaagagtttgaagtgaccgaagtcggcgtgtttacaccaaaaccaaaagttgtcgacgaactgatggtcggtgatgtcggctatttaactgcgtcgatcaaaaacgtacaagatacgcgcgtcggcgatacgattaccgatgccgaacggccggctgctgagccactccctggctaccggaagctcaatccgatggtgttttgcggcatgtacccgatcgacacggcgcgctacaacgacttgcgcgaagcgttagaaaagctgcagctcaacgatgcggcgcttcactttgaaccggaaacgtcgcaggcgctcgggtttggctttcgttgcgggtttctcggcttgcttcatatggagattatccaagagcggattgaacgtgaatttcatatcgatttaattacaacggcgccgagcgttgtctacaaagtatatttaacggacggaacggaagtcgatgtcgacaacccgacgaacatgccggatccgcaaaaaatcgaccgcatcgaagagccgtatgtaaaagcgacgattatggtgccgaacgactacgtcggaccggtgatggagctgtgccaaggaaagcgtggcacgttcgttgacatgcaatatttagatgaaaagcgggtcatgttgatttacgatattccgctgtcggaaatcgtgtatgactttttcgatgcgttaaagtcgaacacgaaagggtatgcgtcgtttgactatgaattgatcggttaccggccgtccaatcttgtcaaaatggatattttgttgaatggcgaaaaaattgacgctttatcgtttattgttcaccgcgattcggcttatgagcgcggcaaagtgatcgtcgagaagctgaaagatttaattccacgccaacagtttgaagtgcctgtgcaggcggcgatcggcaataagatcatcgcccgttcgacgatcaaggcgctgcgtaaaaacgtgctcgccaaatgttacggcggcgacgtgtcgcggaaacggaaactgcttgagaaacaaaaagaaggaaagaaacggatgaaacaaatcggttcggtcgaagtgccgcaggaagcgtttatggctgtcttgaaaatcgacgaccagaaaaaa

SEQ ID NO: 82 - protein, 609 aa, Geobacillus subterraneus
MNREERLKRQERIRNFSIIAHIDHGKSTLADRILEKTGALSERELREQTLDMMELERERGITIKLNAVQLTYKAKNGEEYIFHLIDTPGHVDFTYEVSRSLAACEGAILVVDAAQGIEAQTLANVYLAIDNNLEILPVINKIDLPSAEPERVRQEIEDVIGLDASEAVLASAKVGIGVEDILEQIVEKIPAPSGDPDAPLKALIFDSLYDPYRGVVAYVRIVDGTVKPGQRIKMMSTGKEFEVTEVGVFTPKPKVVDELMVGDVGYLTASIKNVQDTRVGDTITDAERPAAEPLPGYRKLNPMVFCGMYPIDTARYNDLREALEKLQLNDAALHFEPETSQALGFGFRCGFLGLLHMEIIQERIEREFHIDLITTAPSVVYKVYLTDGTEVDVDNPTNMPDPQKIDRIEEPYVKATIMVPNDYVGPVMELCQGKRGTFVDMQYLDEKRVMLIYDIPLSEIVYDFFDALKSNTKGYASFDYELIGYRPSNLVKMDILLNGEKIDALSFIVHRDSAYERGKVIVEKLKDLIPRQQFEVPVQAAIGNKIIARSTIKALRKNVLAKCYGGDVSRKRKLLEKQKEGKKRMKQIGSVEVPQEAFMAVLKIDDQKK

SEQ ID NO: 83 - DNA, 555 bp, Geobacillus subterraneus
atgatttcagtgaacgattttcgcacagggcttacgattgaggtcgacggcgagatttggcgcgtccttgagttccagcatgttaagccgggcaaaggggcggcgttcgtccgttcgaagctgcgcaacttgcgtaccggcgccattcaagagcggacgttccgcgctggcgaaaaagtaaaccgggcacaaattgatacgcgcaaaatgcaatatttatacgctaacggcgacttgcatgtctttatggatatggaaacatacgaacaaatcgagctgccagcgaaacaaattgagtatgagctgaagttcttaaaagaaaacatggaagtatttatcatgatgtatcaaggcgaaacgatcggtgttgagctgccgaacaccgtcgagttgaaagtcgttgaaacagagccgggcatcaaaggtgacacggcttccggcggttcgaagccggccaagctcgaaaccggtcttgtcgttcaagtgccgtttttcgtcaatgaaggcgacacgctcatcattaacacggctgacggtacgtacgtttcgcgggca

SEQ ID NO: 84 - protein, 185 aa, Geobacillus subterraneus
MISVNDFRTGLTIEVDGEIWRVLEFQHVKPGKGAAFVRSKLRNLRTGAIQERTFRAGEKVNRAQIDTRKMQYLYANGDLHVFMDMETYEQIELPAKQIEYELKFLKENMEVFIMMYQGETIGVELPNTVELKVVETEPGIKGDTASGGSKPAKLETGLVVQVPFFVNEGDTLIINTADGTYVSRA

SEQ ID NO: 85 - DNA, 1017 bp, Geobacillus subterraneus
atggatccagccgttatcaacgacccgaaaaagttgcgcgattattcgaaagagcaggctgatttgactgaaacggtgcaaacgtaccgtgaatacaagtccgttcgcagtcagctcgcggaagcgaaggctatgctggaagaaaaacttgagccagagctgcgcgagatggtgaaagaggaaattgatgagctcgaagaacgggaagaagcgctcgttgagaagttgaaagtgttgcttttgccgaaagatccgaatgatgagaaaaacgtcattatggaaattcgtgccgccgccggtggcgaggaagccgcgctgtttgccggcgacttgtaccggatgtatacgcgctatgcggagtcgcaagggtggaaaacggaagtgatcgaagcaagcccaacaggtcttggcggctataaagaaatcatctttatggtcaatgggaaaggggcgtattcgaagctgaagtttgaaaacggcgctcatcgcgtccaacgcgtcccggaaacggaatcaggcggacgcatccatacatcgacggcaacggtcgcctgcttgccggaaatggaagaagtcgaagtcgaaattcatgaaaaagacattcgcgtcgatacgtacgcctcgagcgggccagggggacaaagcgtgaacacgacgatgtcagccgtacgcctcacccatattccgaccggcattgtcgttacttgccaagacgaaaaatcgcaaattaaaaacaaagaaaaagcgatgaaagtgttgcgcgcccgcatttacgacaaataccagcaagaagcgcgcgccgagtatgaccaaacgcgtaagcaagcagtcggcaccggcgatcgctcagagcgcatccgcacgtacaacttcccgcaaaaccgcgtcactgaccaccgtatcgggttgacgattcaaaagcttgacctcgtgttagacgggcagctcgatgaaattatcgaggcgctcattttagacgaccagtcgaaaaaactggagcaagcgaacgatgcgtcg

SEQ ID NO: 86 - protein, 339 aa, Geobacillus subterraneus
MDPAVINDPKKLRDYSKEQADLTETVQTYREYKSVRSQLAEAKAMLEEKLEPELREMVKEEIDELEEREEALVEKLKVLLLPKDPNDEKNVIMEIRAAAGGEEAALFAGDLYRMYTRYAESQGWKTEVIEASPTGLGGYKEIIFMVNGKGAYSKLKFENGAHRVQRVPETESGGRIHTSTATVACLPEMEEVEVEIHEKDIRVDTYASSGPGGQSVNTTMSAVRLTHIPTGIVVTCQDEKSQIKNKEKAMKVLRARIYDKYQQEARAEYDQTRKQAVGTGDRSERIRTYNFPQNRVTDHRIGLTIQKLDLVLDGQLDEIIEALILDDQSKKLEQANDAS

SEQ ID NO: 87 - DNA, 981 bp, Geobacillus subterraneus
atggccgcgcccggcttttgggatgaccagaaagcggcgcaggcgatcatttccgaagcgaatgcgctcaaggaattagtcggcgagtttgaatcgctcgcggaacggttcgacaacttggaagtgacgtatgagttgttgaaagaggagccggatgacgagctgcaggctgaacttgtggaagaagcgaaaaaattgacgaaagacttcagccagtttgagctgcagctgttgctcaacgagccgtacgaccaaaataacgcgattttggagcttcatccgggtgcgggcggcacggaatcgcaagactgggcgtcgatgctgttgcgcatgtacacgcgctgggcggagaaaaaaggatttaaagtcgaaacactggattatctcccaggcgaggaagccggggtgaaaagcgtcaccttgcttatcaagggacataatgcatacggctacttaaaggcggaaaaaggggtacaccggcttgtgcgcatctccccgtttgacgcctcaggccgccgccatacgtcgttcgtgtcatgcgaagtcgtgccggagatggacgataacattgagattgagatccgtccggaagagctgaaaatcgacacgtaccgctcaagcggtgcgggcgggcagcacgtcaacacgaccgactccgcggtgcgcatcacccacttgccgaccggcattgtcgttacgtgccaatcggagcggtcgcaaattaaaaaccgcgaaaaagcgatgaatatgttaaaagcgaagctgtatcaaaagaaaatggaggaacagcaagctgaactcgccgagctgcgcggcgagcaaaaagaaatcggctggggcagccaaatccgctcctacgtcttccatccgtattcgcttgtcaaagaccatcggacgaatgtggaggtcggcaacgtgcaagcggtgatggatggggaaatcgatgtgttcattgacgcgtatttgcgcgcgaaattgaag

SEQ ID NO: 88 - protein, 327 aa, Geobacillus subterraneus
MAAPGFWDDQKAAQAIISEANALKELVGEFESLAERFDNLEVTYELLKEEPDDELQAELVEEAKKLTKDFSQFELQLLLNEPYDQNNAILELHPGAGGTESQDWASMLLRMYTRWAEKKGFKVETLDYLPGEEAGVKSVTLLIKGHNAYGYLKAEKGVHRLVRISPFDASGRRHTSFVSCEVVPEMDDNIEIEIRPEELKIDTYRSSGAGGQHVNTTDSAVRITHLPTGIVVTCQSERSQIKNREKAMNMLKAKLYQKKMEEQQAELAELRGEQKEIGWGSQIRSYVFHPYSLVKDHRTNVEVGNVQAVMDGEIDVFIDAYLRAKLK

SEQ ID NO: 89 - DNA, 555 bp, Geobacillus subterraneus
atggcaaagcaagtgatccaacaggcgaaagaaaaaatggataaagctgtgcaagcgttcagccgcgagttggcgaccgtccgtgccggtcgggcgaacgcggggttgcttgagaaagtaaccgttgactattacggtgtcgcaacgccgatcaaccagctcgctacgatcagcgtgccggaagcgcgtatgcttgtcattcagccgtatgacaaatcggtcattaaagaaatggaaaaagcgattttagcgtcggacttaggagtgacgccgtcgaatgacggatcggttatccgccttgtcattccgccgcttactgaagaacgtcgccgtgaactggcgaagctcgtcaaaaaatattcggaagaagcgaaagttgcggtgcgcaacatccgtcgcgatgcaaacgatgagctgaaaaaactcgagaaaaatagcgagattacggaagatgagctgcgcagctataccgacgaagtgcaaaagctgaccgacagccatatcgccaaaattgacgccatcacaaaagagaaagaaaaagaagtgatggaagta

SEQ ID NO: 90 - protein, 185 aa, Geobacillus subterraneus
MAKQVIQQAKEKMDKAVQAFSRELATVRAGRANAGLLEKVTVDYYGVATPINQLATISVPEARMLVIQPYDKSVIKEMEKAILASDLGVTPSNDGSVIRLVIPPLTEERRRELAKLVKKYSEEAKVAVRNIRRDANDELKKLEKNSEITEDELRSYTDEVQKLTDSHIAKIDAITKEKEKEVMEV

SEQ ID NO: 91 - DNA, 2676 bp, Geobacillus subterraneus
atgagagtttttttatataaaagaccaaaggggaggattgttatgaaaaagttaacatctgccgaagtgcggcgtatgtttttgcagtttttccaagaaaaaggccatgcggtcgagccgagcgcttcgctcattcctgtcgatgacccgtcgttattatggatcaacagcggtgtcgcgacgctgaaaaaatattttgatggccgtatcatcccggacaacccgcgcatttgcaatgcgcaaaaatcgatccgcacaaacgacatcgaaaatgtcgggaaaacggctcgccaccatacgttttttgaaatgctcggcaacttttcgatcggcgattatttcaagcgtgaagcgattcattgggcatgggagtttttaacaagtgaaaagtggattggttttgatccagagcggttgtcagtcactgttcatccggaagacgaagaggcgtataacatttggcgcaacgagatcggtcttcctgaagagcggattattcgtttagaaggaaacttctgggatatcggtgaaggcccgagcggtccgaacacggaaattttttatgaccgcggtgaagcgttcggcaacgatccaaacgatccagaactgtatccaggcggggaaaatgaccgctacttagaagtatggaatctcgtcttttcacagttcaaccataacccggacggcacgtacacgccgctgccgaagaaaaacatcgataccggcatgggcttagagcggatgtgctcgattttgcaagatgtaccgacgaactttgaaactgatttgttcatgccgatcatccgcgcgactgagcagatcgcgggtgagcaatacggcaaagatccgaataaagacgttgcttttaaggtcatcgctgaccatattcgtgccgtgacgtttgcggtcggcgacggggcgctgccgtcgaacgaaggacgaggctatgtattgcgccgcctgcttcgccgcgctgtgcgctatgcgaaacaaatcggcattgaccgtccatttatgtatgagcttgttccggttgtcggtgaaattatgcaagactattatccggaagtgaaagaaaaagccgatttcatcgcccgcgtcattcggacggaagaagagcggttccacgaaacgcttcatgaagggctcgccattttggcagaagtgatggaaaaggcgaaaaaacaaggaagcaccgtcattccaggagaagaggcgttccgcttgtacgatacgtacggcttcccgctcgagctgacggaagaatatgctgctgaagcgggcatgtcggtcgatcacgccggttttgagcgcgagatggagcgccagcgcgaacgggcccgtgccgctcgccaagatgtcgattcgatgcaagtgcaaggcggggtgctcggcgacattaaagacgaaagccgttttgtcggctacgatgagctcgtcgtttcttcgacggtcattgccatcattaaagacggacagctcgtggaggaagtcgggactggcgaggaagcacaaatcatcgttgatgtgacgccgttttacgccgaaagcggcggacaaatcgctgaccaaggtgtgtttgaaggcgaaacgggaacagcggtcgtcaaagatgtgcaaaaagcaccgaacggtcagcacctccattcgattgtcgtcgaacgcggtgcggtgaaaaaaggcgatcgctatacggcgcgcgtcgatgaagtgaagcggtcgcaaatcgtgaaaaaccatacggcgacccacttgcttcatcaagcgttaaaagacgttcttggccgccatgtcaaccaggccggatcactcgttgccccggatcggcttcgctttgactttactcatttcgggcaagtgaagcctgatgagctcgagcgcattgaggcgatcgtcaatgaacaaatttggaagagtattccggtcgacattttttacaaaccgctcgaggaagcaaaagcgatgggggcgatggcgctgtttggtgaaaaatacggcgatatcgtccgcgttgttaaagttggcgactacagcttagagttgtgcggcggctgccatgtgccgaatacagcggccattgggttgtttaaaatcgtctccgagtccggcatcggtgccggcacgcgccggattgaagcggtgactggggaagcggcataccgctttatgagcgaacagcttgctctgttgcaagaagcggcgcaaaagctgaaaacgagcccgagagagctgaatgcccgccttgatgggctgtttgccgaactgcgccaactgcagcgcgaaaatgagtcgcttgctgcccgtctcgcccatatggaggcggaacacctcacccgtcaagtgaaagaggtgggcggtgtgccggtattagccgcaaaagtgcaggcgaacgacatgaaccaattgcgggcgatggctgatgacttgaagcaaaaactagggacggcggtcatcgtgttagcggccgtgcaaggtggcaaagtccaattgattgctgcggtgactgatgacttagtgaaaaaaggataccacgccggcaaactcgtcaaagaagtggcttcacgttgcggcggcggaggcggcggacgtcctgatatggcgcaggccggtgggaaggacgcgaacaaagtcggcgaagcgctcgattatgtcgaaacatgggtcaaatccatttcc

SEQ ID NO: 92 - protein, 892 aa, Geobacillus subterraneus
MRVFLYKRPKGRIVMKKLTSAEVRRMFLQFFQEKGHAVEPSASLIPVDDPSLLWINSGVATLKKYFDGRIIPDNPRICNAQKSIRTNDIENVGKTARHHTFFEMLGNFSIGDYFKREAIHWAWEFLTSEKWIGFDPERLSVTVHPEDEEAYNIWRNEIGLPEERIIRLEGNFWDIGEGPSGPNTEIFYDRGEAFGNDPNDPELYPGGENDRYLEVWNLVFSQFNHNPDGTYTPLPKKNIDTGMGLERMCSILQDVPTNFETDLFMPIIRATEQIAGEQYGKDPNKDVAFKVIADHIRAVTFAVGDGALPSNEGRGYVLRRLLRRAVRYAKQIGIDRPFMYELVPVVGEIMQDYYPEVKEKADFIARVIRTEEERFHETLHEGLAILAEVMEKAKKQGSTVIPGEEAFRLYDTYGFPLELTEEYAAEAGMSVDHAGFEREMERQRERARAARQDVDSMQVQGGVLGDIKDESRFVGYDELVVSSTVIAIIKDGQLVEEVGTGEEAQIIVDVTPFYAESGGQIADQGVFEGETGTAVVKDVQKAPNGQHLHSIVVERGAVKKGDRYTARVDEVKRSQIVKNHTATHLLHQALKDVLGRHVNQAGSLVAPDRLRFDFTHFGQVKPDELERIEAIVNEQIWKSIPVDIFYKPLEEAKAMGAMALFGEKYGDIVRVVKVGDYSLELCGGCHVPNTAAIGLFKIVSESGIGAGTRRIEAVTGEAAYRFMSEQLALLQEAAQKLKTSPRELNARLDGLFAELRQLQRENESLAARLAHMEAEHLTRQVKEVGGVPVLAAKVQANDMNQLRAMADDLKQKLGTAVIVLAAVQGGKVQLIAAVTDDLVKKGYHAGKLVKEVASRCGGGGGGRPDMAQAGGKDANKVGEALDYVETWVKSIS

SEQ ID NO: 93 - DNA, 1671 bp, Geobacillus subterraneus
atgaacattgtcggacaaatgaaagaacagctgaaagaggaaattcgccaggcggtgggaaaagccgggctggtggcggctgaggagctgccagaagtattgcttgaggtgccgcgcgaaaaggctcatggcgattattcgacgaatatcgccatgcagctcgcccgcatcgcgaaaaagccaccgcgggcaatcgccgaagccatcgttgaaaagtttgacgccgagcgtgtttcggtggcgcgcatcgaggtagccggcccagggtttattaacttttacatggacaatcgctatttgacagcggttgtgccggcgattttgcaagcgggccaagcgtatggcgagtcgaatgtcggcaaaggggaaaaagtgcaagtcgagttcgtctcggctaacccgaccggcaacttgcatttaggtcatgctcgcggtgcggcggttggcgattcacttagcaatattttggcgaaagccggattcgatgtgacgcgtgaatattacattaatgatgccggcaaacaaatttataacttggcgaaatcagtcgaagcccgctatttccaagcgctcggtaccgatatgccgctgccggaggacggctattacggtgacgacatcgtggaaatcggcaaaaagctcgccgatgaatatggcgatcggttcgtccatgtggacgaagaagaacgactcgcctttttccgcgaatacggcctccgttatgagctcgacaaaattaaaaacgatttggctgccttccgcgttccatttgacgtttggtattcggaaacatcgctttatgagagcggcaaaatcgatgaggcgctctcaacgctgcgtgagcgcggttacatttacgaacaggacggagccacatggtttcgttcgacggcgtttggcgatgacaaagaccgtgtgttaatcaagcaagacggaacgtatacgtatttgcttccggacatcgcttaccatcaagataagctgcggcgtgggttcacgaagctaatcaacgtctggggagcggatcatcatggctacatcccgcgcatgaaagcggcgatcgctgcgctcggctacgatccagaagcgctcgaggtcgaaattatccaaatggtgaacttataccaaaacggcgagcgcgtcaaaatgagcaaacgtactggcaaagcggtgacgatgcgcgagctgatggaagaagtcggcgtcgatgctgtccgctacttcttcgctatgcgttcgggcgatacgcatctcgattttgatatggacttggctgttgcccagtcgaatgaaaacccggtctactatgtccaatatgcacatgcccgcgtctcaagcattctccgtcaagcaaaagagcatcaactgtcgtatgaaggcgacgtcgatcttcatcatctcgtggaaacagaaaaagaaatcgagctgctcaaagcgcttggcgacttcccggacgttgtcgctgaggcggccttgaaacggatgccacatcgcgtcaccgcctatgcgtttgatttggcgtcggcgctccacagcttttacaatgcggaaaaagtgcttgacctagaccagatcgaaaaaacgaaagctcgtctcgcgcttgtcaaggcggtgcaaatcacgctgcaaaacgctctagcgttaatcggcgtctcagcgccggaacaaatg

SEQ ID NO: 94 - protein, 557 aa, Geobacillus subterraneus
MNIVGQMKEQLKEEIRQAVGKAGLVAAEELPEVLLEVPREKAHGDYSTNIAMQLARIAKKPPRAIAEAIVEKFDAERVSVARIEVAGPGFINFYMDNRYLTAVVPAILQAGQAYGESNVGKGEKVQVEFVSANPTGNLHLGHARGAAVGDSLSNILAKAGFDVTREYYINDAGKQIYNLAKSVEARYFQALGTDMPLPEDGYYGDDIVEIGKKLADEYGDRFVHVDEEERLAFFREYGLRYELDKIKNDLAAFRVPFDVWYSETSLYESGKIDEALSTLRERGYIYEQDGATWFRSTAFGDDKDRVLIKQDGTYTYLLPDIAYHQDKLRRGFTKLINVWGADHHGYIPRMKAAIAALGYDPEALEVEIIQMVNLYQNGERVKMSKRTGKAVTMRELMEEVGVDAVRYFFAMRSGDTHLDFDMDLAVAQSNENPVYYVQYAHARVSSILRQAKEHQLSYEGDVDLHHLVETEKEIELLKALGDFPDVVAEAALKRMPHRVTAYAFDLASALHSFYNAEKVLDLDQIEKTKARLALVKAVQITLQNALALIGVSAPEQM

SEQ ID NO: 95 - DNA, 1323 bp, Geobacillus subterraneus
atggacgtgtcgattattggagggaatgtgtacgtgaaaacgacgattgctgaagtgaaccaatatgtaggtcaagaagtcacgatcggcgcttggttggcgaacaagcgctcgagcggaaaaatcgcctttttacagctgcgtgatgggactggctttattcaaggtgtagttgaaaaagcgaacgtctcagaagaggtatttcaacgtgcgaaaacgctgacgcaagaaacgtcgctctatgtgaccggcacggtgcgcgtcgacgagcgttcaccgttcggttatgagctttcggtgacgaacatacaggtcatcaatgaagcggtcgattatccgattacgccaaaagaacacggtgtcgagtttttaatggatcatcgtcacctttggcttcgttcgcggcgccaacatgcgatcatgaaaatccgcaacgaattgatccgtgcgacgtatgagttttttaacgaacgtggcttcgtcaaagtcgatgcgccgattttgactggcagcgcaccggaaggaacgaccgagctgttccatacgaagtattttgacgaggatgcctatttatcgcaaagcggccagctatatatggaagcagcagccatggcgctcggtaaagtgttttcgttcggtccgacattccgtgccgaaaagtcgaaaacgcgccgccatttgatcgaattttggatgatcgagcctgaaatggcgttttacgaatttgaagacaatttgcggctgcaagaagagtatgtctcttatctcgtacagtcggtgcttagccgttgccaacttgagctcgggcgccttggacgcgacgtcaccaagcttgagcttgtcaagccgccgtttccgcgtctaacgtatgacgaagcgatcaagctgctgcatgacaaagggtttaccgatatcgaatggggcgatgacttcggtgcgccgcatgagacagccatcgctgaaagcttcgacaagccggtgtttatcactcactacccgacgtcgttaaagccgttttatatgcagccagatccgaaccgtccggacgtcgtgctatgtgctgatttaatcgcgccggagggatacggggagattatcggcggttccgagcgcattcatgattatgagctgctcaagcagcgtctcgaggagcatcatttgccgcttgaagcatatgaatggtatttagatttgcgcaaatacggttccgtgccgcactccggattcgggctcggcctcgagcgaacggttgcttggatttgcggcgttgagcatgtacgcgagacgatcccgtttccgcggttgctcaaccgtctatacccg

SEQ ID NO: 96 - protein, 441 aa, Geobacillus subterraneus
MDVSIIGGNVYVKTTIAEVNQYVGQEVTIGAWLANKRSSGKIAFLQLRDGTGFIQGVVEKANVSEEVFQRAKTLTQETSLYVTGTVRVDERSPFGYELSVTNIQVINEAVDYPITPKEHGVEFLMDHRHLWLRSRRQHAIMKIRNELIRATYEFFNERGFVKVDAPILTGSAPEGTTELFHTKYFDEDAYLSQSGQLYMEAAAMALGKVFSFGPTFRAEKSKTRRHLIEFWMIEPEMAFYEFEDNLRLQEEYVSYLVQSVLSRCQLELGRLGRDVTKLELVKPPFPRLTYDEAIKLLHDKGFTDIEWGDDFGAPHETAIAESFDKPVFITHYPTSLKPFYMQPDPNRPDVVLCADLIAPEGYGEIIGGSERIHDYELLKQRLEEHHLPLEAYEWYLDLRKYGSVPHSGFGLGLERTVAWICGVEHVRETIPFPRLLNRLYP

SEQ ID NO: 97 - DNA, 1359 bp, Geobacillus subterraneus
atgtttcaaacacttgagcttcgtcataaagtggcgaaggcggtgcgcaactttttagacggcgaacgctttttagaagtggagacgccaatgttgacgaaaagcacaccggaaggggcgcgcgattatttagtgccaagccgcgttcatccgggggaattttacgccttgccgcagtcgccgcaaatttttaagcagcttttgatggtcggcggttttgaacgctattaccaaatcactcgttgcttccgcgatgaagatttgcgcgctgaccgccagccagagtttacgcaaattgacattgaaatgtcgtttgtcgaccaagaagacatcatcgatttaaccgaacggatgatggcggcggtcgtcaaagcaactaaagggattgacattccgcgcccatttccacgcatcacgtatgacgaagcgatgagccgttacggttccgataagccggacgtacgttttggccttgagcttgtcgatgtgtcggaagcggtccgcggctccgcgtttcaagtgttcgcccgcgccgttgagcaaggtggtcaagtgaaggcaatcaacgtaaaaggagcggcgagccgttattcgcgtaaagacattgacgcgttagcggagtttgccggccgctacggagcgaaagggctcgcttggttaaaagttgaaggcggggagctgaaagggccgatcgccaagtttttcgtcgatgatgagcaaacagcgctgcgccagctgcttgctgccgaagatggggatttgctgttgtttgttgctgacgagaaggcgattgtcgcggcggctcttggtgcgttgcggttaaagctcggcaaagagcttggcttgatcgatgaaacgaagctcgcttttttatgggtaacagattggccgcttttagagtacgacgaagaagaaggccgctattacgccgcccaccatccgtttacgatgccggtgcgtgacgatatcccgctgcttgagacaaacccaggcgctgttcgggcgcaggcgtatgatttagtgttaaacggctatgagcttggcggcggttcgctccgtatttttgagcgcgatgtacaagaaaaaatgttccgcgctctaggatttgaccaggaagaggcgcgccgccagtttggcttcctgcttgaggcgtttgaatatggcactccgccgcatggcggtatcgccctcggcctcgatcgacttgtgatgctcttagctgggcgcacaaacttgcgcgatacgatcgccttcccgaaaactgcgagcgccagctgcctgcttactgaagcgccgggaccggtcagtgaaaaacaactgaaagagttgcatttggctgtggtgcttcccgaccagcaa

SEQ ID NO: 98 - protein, 453 aa, Geobacillus subterraneus
MFQTLELRHKVAKAVRNFLDGERFLEVETPMLTKSTPEGARDYLVPSRVHPGEFYALPQSPQIFKQLLMVGGFERYYQITRCFRDEDLRADRQPEFTQIDIEMSFVDQEDIIDLTERMMAAVVKATKGIDIPRPFPRITYDEAMSRYGSDKPDVRFGLELVDVSEAVRGSAFQVFARAVEQGGQVKAINVKGAASRYSRKDIDALAEFAGRYGAKGLAWLKVEGGELKGPIAKFFVDDEQTALRQLLAAEDGDLLLFVADEKAIVAAALGALRLKLGKELGLIDETKLAFLWVTDWPLLEYDEEEGRYYAAHHPFTMPVRDDIPLLETNPGAVRAQAYDLVLNGYELGGGSLRIFERDVQEKMFRALGFDQEEARRQFGFLLEAFEYGTPPHGGIALGLDRLVMLLAGRTNLRDTIAFPKTASASCLLTEAPGPVSEKQLKELHLAVVLPDQQ

SEQ ID NO: 99 - DNA, 1419 bp, Geobacillus subterraneus
atgaaaggaagagcgaatatgagcagtatccgactttataatacgttgacgcgaaaaaaggaaacgtttgagccgctcgaaccgaacaaagtgaaaatgtatgtatgtggcccgacggtctataattatattcatatcggcaatgctcgcgccgctatcgtctttgatacgatccgccgttatttagagttccgcggttatgatgtgacgtatgtatccaactttactgatgtcgacgacaagctaatcagggcggcccgcgagcttggtgagagcgtgccggcgatcgccgagcggtttattgaggcgtattttgaggacattgaggcgctcggctgcaaaaaagcagatatccatccgcgcgtgacggaaaatatcgaaacgattatcgaattcattcaagcgctcattgacaaaggctatgcgtacgaagtcgatggtgacgtatactatcggacgcgcaagtttgatggctacggcaaattgtcgcatcagtcgatcgatgagctacaagcgggggcgcgcatcgaagttggggaaaagaaagatgatccactcgattttgctctttggaaagcagcgaaagaaggagagatttcttgggacagcccatgggggaaagggcggcccggctggcatatcgaatgttcagcgatggcgcgcaaatatttaggagatacgatcgacattcatgctggcggccaagacttaacgtttccacaccatgaaaacgaaattgcccaatcggaagcactgaccggcaaaccgtttgcgaaatattggctgcacaatgggtatttaaatattaacaatgaaaaaatgtccaagtcgcttggcaactttgtacttgttcacgatatcatccggcagattgacccacaagtgttgcgtttctttatgctgtcggtgcactatcgccacccgatcaactatagcgaggagctgcttgagagcgctcggcgtggtctcgaacgcttgaggacagcatacggtaatttgcagcaccggcttggggcgagcacgaacttaaccgataacgacggcgagtggctttcgcgcctcgcggatatccgcgcctcgttcattcgtgaaatggacgatgatttcaacacagcaaacggcattgcggtcttgttcgagctcgccaaacaagcgaacttgtatttgcaggagaaaacgacatccgagaatgtcattcacgcgtttttgcgcgaatttgagcagctgatggatgtactcggccttactttgaaacaagaggagttgcttgacgaagaaattgaggcgctgatccgccagcgcaatgaagcgcggaaaaatcgtgactttgccttagccgaccgcatccgcgacgagttgaaagcaaaaaatatcattttggaagatacgccgcaagggacgagatggaaacggggatcg

SEQ ID NO: 100 - protein, 473 aa, Geobacillus subterraneus
MKGRANMSSIRLYNTLTRKKETFEPLEPNKVKMYVCGPTVYNYIHIGNARAAIVFDTIRRYLEFRGYDVTYVSNFTDVDDKLIRAARELGESVPAIAERFIEAYFEDIEALGCKKADIHPRVTENIETIIEFIQALIDKGYAYEVDGDVYYRTRKFDGYGKLSHQSIDELQAGARIEVGEKKDDPLDFALWKAAKEGEISWDSPWGKGRPGWHIECSAMARKYLGDTIDIHAGGQDLTFPHHENEIAQSEALTGKPFAKYWLHNGYLNINNEKMSKSLGNFVLVHDIIRQIDPQVLRFFMLSVHYRHPINYSEELLESARRGLERLRTAYGNLQHRLGASTNLTDNDGEWLSRLADIRASFIREMDDDFNTANGIAVLFELAKQANLYLQEKTTSENVIHAFLREFEQLMDVLGLTLKQEELLDEEIEALIRQRNEARKNRDFALADRIRDELKAKNIILEDTPQGTRWKRGS

SEQ ID NO: 101 - DNA, 1488 bp, Geobacillus subterraneus
atggaattggaggtttggacgatggcaaaaaacgtgcgcgtgcgctatgcgccgagcccgactggccatttgcatatcggtggggcacggacagcgctgtttaactatttgtttgcccgccattacggcggaaaaatgatcgtccgcatcgaagatacggatattgaacggaacgttgaaggcggcgaagagtcgcagcttgaaaacttaaaatggcttggcatcgattatgacgaatcgattgataaggacggcggatatgggccgtatcgtcagacggaacggctcgatatctatcggaagtatgtgaacgagctgcttgaacaagggcatgcgtataaatgtttttgtacaccggaagagctcgagcgggaacgtgaggagcaacgggcggcaggtattgctgctccgcaatacagcggcaaatgccgccatttaacgccggagcaagttgccgagcttgaagcacaaggaaaaccgtatacgatccgcttgaaagtgccggaagggaaaacgtatgaagtagatgatttagtgcgcggtaaagtgacgtttgaatcgaaagacatcggcgattgggtcattgtgaaggcgaacggtattccgacgtacaactttgccgttgtcattgatgaccatttgatggaaatcagccatgtgttccgcggtgaggagcatttatccaacacgccgaaacagctaatggtgtacgaatatttcggttgggagccaccgcaattcgcccatatgacattgattgtcaacgagcagcggaaaaagctatccaagcgcgatgaatcgattatccagttcgtgtcgcaatataaagagctcggctatttgccggaggcgatgttcaactttttcgcccttcttggctggtcgccggaaggagaagaagaaatttttacgaaggacgagctcatccgcatttttgatgtcgcccggctgtcgaaatcgccgtcgatgtttgatacgaaaaagctgacatggatgaacaaccaatatatcaaaaagctggatctcgacaggcttgtcgagctggcgttgccgcatttagtgaaagccggacgcctgccggcagatatgagtgatgagcagcggcaatgggcacgcgatttgattgccttgtaccaagagcaaatgagctacggtgcggagatcgtttcgctgtccgagctgttctttaaagaagaagtcgaatacgaagacgaagcccgccaagtgctcgccgaagaacaagtaccggatgtgctctccgcctttttggcgaatgtgcgtgagcttgagccgtttacggcggatgagattaaagcagcgatcaaagcagtgcaaaaatcgacagggcaaaaaggcaagaagctgtttatgccgattcgcgccgcagtgactgggcaaacacacggaccggaactgccgtttgccatccaactgcttggcaaacaaaaggtgattgaacggctcgaacgggcactgcatgaaaaattt

SEQ ID NO: 102 - protein, 496 aa, Geobacillus subterraneus
MELEVWTMAKNVRVRYAPSPTGHLHIGGARTALFNYLFARHYGGKMIVRIEDTDIERNVEGGEESQLENLKWLGIDYDESIDKDGGYGPYRQTERLDIYRKYVNELLEQGHAYKCFCTPEELEREREEQRAAGIAAPQYSGKCRHLTPEQVAELEAQGKPYTIRLKVPEGKTYEVDDLVRGKVTFESKDIGDWVIVKANGIPTYNFAVVIDDHLMEISHVFRGEEHLSNTPKQLMVYEYFGWEPPQFAHMTLIVNEQRKKLSKRDESIIQFVSQYKELGYLPEAMFNFFALLGWSPEGEEEIFTKDELIRIFDVARLSKSPSMFDTKKLTWMNNQYIKKLDLDRLVELALPHLVKAGRLPADMSDEQRQWARDLIALYQEQMSYGAEIVSLSELFFKEEVEYEDEARQVLAEEQVPDVLSAFLANVRELEPFTADEIKAAIKAVQKSTGQKGKKLFMPIRAAVTGQTHGPELPFAIQLLGKQKVIERLERALHEKF

SEQ ID NO: 103 - DNA, 1398 bp, Geobacillus subterraneus
atggaggaggatgatgacatggctgcaacaatggaagaaatcgttgcccacgccaagcatcgcggcttcgtgtttccggggtcggaaatttacggtgggctggcgaacacatgggattacggtccgctcggtgtcgagctgaaaaataacattaaacgggcgtggtggaaaaagttcgtccaagaatcgccacacaatgtcggtttggacgctgccattttaatgaacccaaaaacgtgggaagcatccggccatttaggcaacttcaacgatccgatggtcgactgcaaacagtgtaaagcgcgtcatcgcgccgacaagctgattgagcaggcacttgaagaaaaaggaattgagatggtcgttgacggtttgccgcttgccaagatggaagagcttatccgtgaatacgacatcgcttgtccagaatgcggcagtcgtgactttacgaacgtgcgtcagtttaatttaatgttcaaaacataccaaggtgtcaccgaatcaagcgctaacgaaatttatttgcgcccggagacggcccaaggtatttttgtcaactttaaaaacgtccagcgcacgatgcgcaaaaaattaccgtttggcatcgcgcaaatcggaaaaagtttccgcaacgaaattacgccagggaactttacgttccgcacacgtgaatttgaacaaatggagcttgagtttttctgcaaaccgggcgaagagctgaaatggttcgactactggaaacaattttgcaaggaatggctgttgtcgctcggcatgaacgaagaacatatccgcctgcgcgaccatacgaaagaagaattatcccactatagtaatgcgacgactgatatcgagtatcagttcccgttcggctggggcgagctctggggtattgcgtcgcgcaccgattacgacttaaaacagcatatggaacactccggtgaggatttccattatcttgaccaagaaacgaatgagcgctacatcccgtactgcattgagccgtcgctcggtgccgaccgtgtcacgctcgcgtttatgattgacgcctatgacgaggaagagctcgaagacggcacgacccggacagttatgcatttgcatccagcgcttgcgccgtacaaagcagctgtcttgccgttatcgaaaaagctgggtgacggagcgcgccgaatttatgaagagctcgcgaagcatttcatggtcgactacgatgaaacaggttcgattggcaagcggtatcgtcgtcaagatgaaatcggcacgccgttttgtatcacgtacgactttgagtccgagcaagatggccaagtaaccgttcgtgaccgtgacacgatggaacaagtgcggttgccgattggggagctcaaagcctttttggataaaaaaattgccttt

SEQ ID NO: 104 - protein, 466 aa, Geobacillus subterraneus
MEEDDDMAATMEEIVAHAKHRGFVFPGSEIYGGLANTWDYGPLGVELKNNIKRAWWKKFVQESPHNVGLDAAILMNPKTWEASGHLGNFNDPMVDCKQCKARHRADKLIEQALEEKGIEMVVDGLPLAKMEELIREYDIACPECGSRDFTNVRQFNLMFKTYQGVTESSANEIYLRPETAQGIFVNFKNVQRTMRKKLPFGIAQIGKSFRNEITPGNFTFRTREFEQMELEFFCKPGEELKWFDYWKQFCKEWLLSLGMNEEHIRLRDHTKEELSHYSNATTDIEYQFPFGWGELWGIASRTDYDLKQHMEHSGEDFHYLDQETNERYIPYCIEPSLGADRVTLAFMIDAYDEEELEDGTTRTVMHLHPALAPYKAAVLPLSKKLGDGARRIYEELAKHFMVDYDETGSIGKRYRRQDEIGTPFCITYDFESEQDGQVTVRDRDTMEQVRLPIGELKAFLDKKIAF

SEQ ID NO: 105 - DNA, 1278 bp, Geobacillus subterraneus
atggcttttcaaattccaagagggacacaagatttattaccgggtgaaacggaaaaatggcaatatgtcgaacaagtggcccgcgacctgtgtagacggtacggctatgaagaaatacggacgccgatttttgaacatacggagctgtttttacgtggcgttggtgatacgaccgatatcgtccaaaaagagatgtacacgtttgaagacaaagggggccgtgcgttgacgctccgtccggaaggaaccgcaccggtcgtgcgggcgttcgtcgagcataagctgtacggcagcccgaatcagccggtcaagttgtattatgcgggaccaatgttccgttatgagcggccggaagccggacggttccgccaattcgtccagtttggtgttgaggcaattggcagcagtgatccggcgattgacgccgaggtgatggcgttagcgatgcatatttataaggcgcttggtttaaaacacatccggctcgtaatcaacagtttaggcgatgtagacagccgccgggcgcatcgcgaagcgcttgtccgccatttttctgaccgcattcatgaactgtgcccggactgtcaggcgcggcttgagacgaatccgctccgcattctcgattgtaaaaaggaccgcgatcatgaactgatggcgtcagcaccgtcgattttagactatttgaatgacgaatcgcgcgcgtattttgagaaggtgaagcaatatttaacgatgcttgacatcccgtttgtcattgactcgcggctcgtgcgcggcctcgattattacaaccatacgacgtttgaaattatgagcgaggctgaaggattcggcgcagcggcgactctttgcggcggcggacgctataacgggcttgtgcaagaaattggcggcccggaaacgcctggcatcggctttgcgttaagcattgaacggctgctggcggcgcttgaagcggaagggattgaactgccgatccatcgaggaatcgattgctatgttgtcgctgtcggtgagcgggcaaaagatgaaactgtccgcctcgtttacgaattgcgccgtgccggcctgcgtgtggagcaagactatttaggtcgaaaaatgaaggcacagctgaaggcagctgaccgtcttggcgcatcattcgttgccatcatcggcgacgaggagctggaaaaacagacagcagctgtgaaacacatggcgagcggcgagcaaactgatgtgccgcttggagagttggcgtcctttttaatagaacgaacaaaacgggaggag

SEQ ID NO: 106 - protein, 426 aa, Geobacillus subterraneus
MAFQIPRGTQDLLPGETEKWQYVEQVARDLCRRYGYEEIRTPIFEHTELFLRGVGDTTDIVQKEMYTFEDKGGRALTLRPEGTAPVVRAFVEHKLYGSPNQPVKLYYAGPMFRYERPEAGRFRQFVQFGVEAIGSSDPAIDAEVMALAMHIYKALGLKHIRLVINSLGDVDSRRAHREALVRHFSDRIHELCPDCQARLETNPLRILDCKKDRDHELMASAPSILDYLNDESRAYFEKVKQYLTMLDIPFVIDSRLVRGLDYYNHTTFEIMSEAEGFGAAATLCGGGRYNGLVQEIGGPETPGIGFALSIERLLAALEAEGIELPIHRGIDCYVVAVGERAKDETVRLVYELRRAGLRVEQDYLGRKMKAQLKAADRLGASFVAIIGDEELEKQTAAVKHMASGEQTDVPLGELASFLIERTKREE

SEQ ID NO: 107 - DNA, 2772 bp, Geobacillus subterraneus
atggactacaaagagacgctgctcatgccgcaaacggagttcccgatgcgtggcaacttgccgaagcgggagccggaaatgcaaaaaaaatgggaggaaatggacatttaccggaaagtgcaggagcggacgaaaggacggccgctgtttgtgctgcacgacggcccgccatacgccaacggtgatattcatatgggccatgcattaaataaaattttaaaagatattatcgtccgctacaagtcgatgagcggcttttgtgcgccgtatgtgcctggctgggatacacatggcttaccgattgaaacggcactgacgaagcaaggtgtcgaccgcaaatcgatgagtgtcgctgagttccgcaagctgtgcgaacaatacgcgtatgagcaaatcgacaaccagcgccaacagtttaaacggctcggggtgcggggcgattgggacaacccgtacattacgctcaagccggaatacgaagcccagcaaattaaagtgttcggtgaaatggcgaaaaaagggctcatttataaagggctgaagccggtgtattggtcgccgtcgagcgaatcggcgctcgccgaagcggaaatcgaatataaagacaaacggtcgccgtcgatttatgtcgcgttcccagttaaagatggtaaaggtgtgcttcaaggggatgaacgaatcgtcatttggacgacgacaccgtggacgattccagcgaacttggcgatcgccgttcacccggatttggactactatattgtcgaagcaaacgggcaaaaatacgttgttgctgcggccttggcggaatcggtagcgaaagaagtcggctgggaggcatggtccgtcgtcaaaacggtaaaaggaaaagaacttgagtacgtagtcgccaaacatccgttttacgagcgcgactcgcttgtcgtctgcggcgagcacgtcacgaccgacgccggtaccggctgcgttcatacggcaccaggacacggggaagacgactttatcgtcggacaaaaatacgggcttccggttctttgcccggttgatgagcgcggctatatgacagaagaagcgcctggatttgcagggatgttttacgacgaggcgaacaaagcgattacacaaaagctcgaggaagttggagcgctccttaagctcagcttcattacccactcgtatccgcatgattggcggacgaagcaaccgacaattttccgagcgacgacacaatggtttgcctccattgataaaattcgtgatcaacttcttgatgccatcaaggaaacgaaatgggtgccagaatggggagaaatccgcatccataacatggtgcgcgaccgcggtgactggtgcatctcccgccaacgcgcttggggcgtgccaattccggtcttttacggcgaaaacggcgagccgatcatcacagatgagacgatcgagcacgtgtcaaacctattccgccagtacggctcgaatgtttggtttgagcgtgaggcgaaagacttattgccggaaggattcacccatccgtccagcccgaacggcctctttacgaaagagacggatattatggacgtctggtttgactccggttcgtcgcatcaagccgtgcttgttgaacgcgatgacctagagcgtccggctgatttatacttagaaggatctgaccaatatcgcggctggtttaactcgtcgctgtctacagccgttgccgtcaccggaaaagcaccgtataaaggggtgttaagccatggcttcgttttagacggcgaagggcgaaaaatgagcaaatcgctcggcaacgtcgtcgtgccggccaaagtcatggaacagctcggtgccgacattttacgcctttgggtcgcctcggttgactatcaggcggatgtacgcatttccgataacattttaaaacaagtgtccgaagtgtatcggaaaatccgcaatacgttccgctttatgctcggcaacttgtttgattttgacccgaatcaaaacgctgtgccggttggggagcttggcgaagtcgatcgctacatgttagcgaaattaaataaactcatcgctaaagtgaaaaaggcgtatgacagctatgattttgctgctgtttatcatgagatgaaccatttctgcaccgtcgagttaagcgcattttatttggatatggcgaaagacattttgtacatcgaagcggccgattgtcgtgcccgccgtgcggtgcagacggtgctgtatgaaacggttgtcgccttggcgaagctcattgcgccgattttgccgcacacggccgatgaagtgtgggagcatatcccgaaccggaaagagcaagtggaaagcgtccagctcaccgacatgccggagtcaatggccatcgatggtgaagaagcgctgcttgcgaaatgggatgcgtttatggatgtacgagatgacattttaaaagcgctcgagaatgcgcgtaatgaaaaagtgatcggtaagtcgctcacggcgagcgtcactgtttacccgaaagacgaagtgcgggcgcttttggcttcgatcaacgaggacttgcgccaacttctcatcgtttccgcgttttcggtcgccgatgaatcgtatgacgccgcgccagccgaagcagaacggctcaaccatgtggccgtcatcgttcgcccggcggaaggtgagacgtgcgaacgttgctggacggtgacaccggacgtcggacgcgatgagtcccacccgacgctttgtccgcgctgcgcacatattgtgaacgaacattattcggca

SEQ ID NO: 108 - protein, 924 aa, Geobacillus subterraneus
MDYKETLLMPQTEFPMRGNLPKREPEMQKKWEEMDIYRKVQERTKGRPLFVLHDGPPYANGDIHMGHALNKILKDIIVRYKSMSGFCAPYVPGWDTHGLPIETALTKQGVDRKSMSVAEFRKLCEQYAYEQIDNQRQQFKRLGVRGDWDNPYITLKPEYEAQQIKVFGEMAKKGLIYKGLKPVYWSPSSESALAEAEIEYKDKRSPSIYVAFPVKDGKGVLQGDERIVIWTTTPWTIPANLAIAVHPDLDYYIVEANGQKYVVAAALAESVAKEVGWEAWSVVKTVKGKELEYVVAKHPFYERDSLVVCGEHVTTDAGTGCVHTAPGHGEDDFIVGQKYGLPVLCPVDERGYMTEEAPGFAGMFYDEANKAITQKLEEVGALLKLSFITHSYPHDWRTKQPTIFRATTQWFASIDKIRDQLLDAIKETKWVPEWGEIRIHNMVRDRGDWCISRQRAWGVPIPVFYGENGEPIITDETIEHVSNLFRQYGSNVWFEREAKDLLPEGFTHPSSPNGLFTKETDIMDVWFDSGSSHQAVLVERDDLERPADLYLEGSDQYRGWFNSSLSTAVAVTGKAPYKGVLSHGFVLDGEGRKMSKSLGNVVVPAKVMEQLGADILRLWVASVDYQADVRISDNILKQVSEVYRKIRNTFRFMLGNLFDFDPNQNAVPVGELGEVDRYMLAKLNKLIAKVKKAYDSYDFAAVYHEMNHFCTVELSAFYLDMAKDILYIEAADCRARRAVQTVLYETVVALAKLIAPILPHTADEVWEHIPNRKEQVESVQLTDMPESMAIDGEEALLAKWDAFMDVRDDILKALENARNEKVIGKSLTASVTVYPKDEVRALLASINEDLRQLLIVSAFSVADESYDAAPAEAERLNHVAVIVRPAEGETCERCWTVTPDVGRDESHPTLCPRCAHIVNEHYSA

SEQ ID NO: 109 - DNA, 2433 bp, Geobacillus subterraneus
atgaggaggagtgcgacgatgagtttcaaccatcgcgaaattgagaaaaagtggcaggattattgggaacagcataaaacgttccgcaccccggatgaaagcgataaaccgaagttttacgtgttggatatgtttccgtatccgtctggcgctggcttgcacgtcggccatccggaagggtatacggcgactgatattttggcgcgcatgaagcggatgcaagggtacaatgtccttcacccgatggggtgggacgcgttcggattgccggcagaacaatatgcgctcgataccggcaacgacccggccgaatttacgcaaaaaaacatcgacaacttccgccggcaaattaagtcgcttggtttttcgtatgactgggatcgggaaattaacacgactgatccgaactattacaaatggacgcaatggattttcttgaagctgtatgaaaaagggctcgcctacatggacgaagtaccggtcaactggtgtccggcgcttggcaccgtgctggcgaacgaagaagtcatcaacggccggagcgagcgcggtgggcatccggtcatccgcaagccaatgcggcaatggatgctgaaaattaccgcctatgccgaccggctgctcgaagatttggaggagcttgactggccggaaagcattaaagaaatgcaacgcaactggatcggccgttcggaaggagcggaaattgagtttgctgtcgacggccatgacgagtcgttcacggtatttacgacgcggccagatacgctgtttggcgccacgtacgcagtgttggctccggaacatccgcttgttgagaaaattacaacgccggagcaaaaaccagccgttgatgcttacttaaaagaagtgcaaagcaaaagcgacctcgagcgcaccgacttggcgaaagaaaaaacaggcgtgttcactggtgcgtacgccatccatccagttaccggcgacaagctgccgatttggatcgccgattacgtgttgatgggctacggcactggggcgatcatggctgtaccggcgcatgatgagcgcgactacgagtttgcgaaaacattcaacttgccgatcaaagaagtcgttgccggcgggaatgtcgaaaacgagccgtacactggcgacggggagcacatcaactctgagtttttgaacggcttgaacaaacaagaagcgatcgaaaaaatgatcgcctggcttgaagaaaacggaaaaggacaaaagaaagtgtcgtaccggctgcgcgactggttgtttagccgccaacgctactggggtgagccgattccggtcatccattgggaagatgggacgatgacgacggtgccggaagaagaattgccgcttgtcttgccgaaaacggatgaaattaaaccgtcgggaacgggtgaatcgccgctcgccaacatcgaagaatgggtcaatgttgtcgatccgaaaaccgggaaaaaagggcggcgtgaaacaaacacgatgccgcaatgggcgggaagctgctggtattatttgcgctacatcgacccgcataacgacaaacagctcgccgatccggaaaagttgaaacaatggctgccggttgacgtctacatcggcggggcggagcatgcggtcttgcacttgctgtacgctcgcttctggcataaagtgttgtacgaccttggcatcgtgccgacgaaagagccgttccaaaagctgtttaaccaagggatgatcttaggcgaaaacaatgaaaaaatgagcaaatcgaaaggcaatgtcgtcaacccggatgatatcgtcgagagccatggcgcggatacgttgcggctgtatgaaatgtttatggggccgcttgaagcgtcgatcgcctggtcgacgaaagggcttgacggagcgcgccgtttcttagagcgcgtctggcgtctgtttgtcaccgaagatggtcaactgaacccgaacatcgttgacgagccagcgaacgatacgctcgagcgcgtctaccatcaaacggtgaaaaaagtgacggaagactacgaagcgctgcgcttcaacaccgccatttcgcagctgatggtgttcattaacgaagcgtataaagcggagcagatgaaaaaagaatatatggaagggttcgtcaagctcttatcgccggtttgcccgcatattggcgaagagctctggcaaaagctcggccatactgacaccatcgcctatgaaccatggccgacatatgacgaagcgaaactcgtcgaagatgtcgttgaaatcgtgatccaaatcaacggcaaagtgcgggcgaaactgaacgtgccggcggacttatcgaaagaggcgctagaagaacgggcgctcgccgatgaaaaaattaaagagcagcttgcagggaaaacggtgcgtaaggtgatcactgtccctggtaagctcgtcaatatcgtcgccaac

SEQ ID NO: 110 - protein, 811 aa, Geobacillus subterraneus
MRRSATMSFNHREIEKKWQDYWEQHKTFRTPDESDKPKFYVLDMFPYPSGAGLHVGHPEGYTATDILARMKRMQGYNVLHPMGWDAFGLPAEQYALDTGNDPAEFTQKNIDNFRRQIKSLGFSYDWDREINTTDPNYYKWTQWIFLKLYEKGLAYMDEVPVNWCPALGTVLANEEVINGRSERGGHPVIRKPMRQWMLKITAYADRLLEDLEELDWPESIKEMQRNWIGRSEGAEIEFAVDGHDESFTVFTTRPDTLFGATYAVLAPEHPLVEKITTPEQKPAVDAYLKEVQSKSDLERTDLAKEKTGVFTGAYAIHPVTGDKLPIWIADYVLMGYGTGAIMAVPAHDERDYEFAKTFNLPIKEVVAGGNVENEPYTGDGEHINSEFLNGLNKQEAIEKMIAWLEENGKGQKKVSYRLRDWLFSRQRYWGEPIPVIHWEDGTMTTVPEEELPLVLPKTDEIKPSGTGESPLANIEEWVNVVDPKTGKKGRRETNTMPQWAGSCWYYLRYIDPHNDKQLADPEKLKQWLPVDVYIGGAEHAVLHLLYARFWHKVLYDLGIVPTKEPFQKLFNQGMILGENNEKMSKSKGNVVNPDDIVESHGADTLRLYEMFMGPLEASIAWSTKGLDGARRFLERVWRLFVTEDGQLNPNIVDEPANDTLERVYHQTVKKVTEDYEALRFNTAISQLMVFINEAYKAEQMKKEYMEGFVKLLSPVCPHIGEELWQKLGHTDTIAYEPWPTYDEAKLVEDVVEIVIQINGKVRAKLNVPADLSKEALEERALADEKIKEQLAGKTVRKVITVPGKLVNIVAN

SEQ ID NO: 111 - DNA, 1482 bp, Geobacillus subterraneus
atgagccatgaagaattgaacgaccaattgcgtgtccgccgggaaaagttaaaaaaaatcgaagagctaggtgtcgacccgtttggcaaacggttcgagcgcacgcataaagcagaagagctgtttaaactgtacggcgatttgtccaaagaagaacttgaagatcagcaaattgaagtcgctgtcgccggccgcattatgacgaaacgcggtaaaggaaaagcaggatttgctcacattcaagacgtcacagggcaaattcaaatttatgtccgccaagacgatgtcggtgaacagcaatatgagctgtttaaaatctctgaccttggtgatatcgtcggtgtgcgcggcactatgttcaaaacaaaagtcggcgagctttccatcaaagtgtcatcatatgaatttttaacaaaagcattgcgtccattgccggaaaaataccatggtttaaaggacgtcgaacaacgttaccgccaacgttatctcgacttaactatgaatccgcaaagtaagcagacgtttatcacccgtagtctcattattcaatcgatgcggcgttatctcgacagccaaggttatttggaagtcgaaacaccgatgatgcacgccatagcaggtggtgcggctgcacgtccgtttattacgcaccataatgcccttgatatgacactttatatgcgaatcgccatcgaactccatttaaaacggctcatcgtcggcggtttggaaaaagtgtatgaaatcggacgcgtcttccggaatgaggggatttccacccgtcacaatccggagtttacgatgcttgaactgtacgaggcatatgccgacttccgtgacatcatgaaattgacagaaaacttaattgctcacattgccacggaagtgcttggcacgacgaaaattcaatacggcgaacataccgtcgatttaacgcctgaatggcggcgacttcatatggtcgatgcgattaaagaatacgtcggcgttgatttctggcggcacatggacgacgaggaagcgcgggcgttggcgaaagaacatggggtcgaaatcgccccgcacatgacgtttggtcatatcgtcaatgaattttttgaacaaaaagtcgagtcgcaactcatccaaccgacgttcatttatggccaccctgtcgaaatttcgccgttagctaagaaaaacccggacgatccacgctttaccgatcgatttgagctatttatcgttggacgtgaacatgcgaacgcgtttacggaactaaacgatccgatcgaccagcgccaacgtttcgaagcacagttgaaagaacgtgaacaagggaacgatgaagcgcacgaaatggacgaagatttcctcgaagcgctcgagtacggtatgcctccaacaggcggactcggcatcggcgttgaccgtctagtcatgctcttgactaactctccgtccattcgggatgtgttactcttcccgcaaatgcgtcataaa

SEQ ID NO: 112 - protein, 494 aa, Geobacillus subterraneus
MSHEELNDQLRVRREKLKKIEELGVDPFGKRFERTHKAEELFKLYGDLSKEELEDQQIEVAVAGRIMTKRGKGKAGFAHIQDVTGQIQIYVRQDDVGEQQYELFKISDLGDIVGVRGTMFKTKVGELSIKVSSYEFLTKALRPLPEKYHGLKDVEQRYRQRYLDLTMNPQSKQTFITRSLIIQSMRRYLDSQGYLEVETPMMHAIAGGAAARPFITHHNALDMTLYMRIAIELHLKRLIVGGLEKVYEIGRVFRNEGISTRHNPEFTMLELYEAYADFRDIMKLTENLIAHIATEVLGTTKIQYGEHTVDLTPEWRRLHMVDAIKEYVGVDFWRHMDDEEARALAKEHGVEIAPHMTFGHIVNEFFEQKVESQLIQPTFIYGHPVEISPLAKKNPDDPRFTDRFELFIVGREHANAFTELNDPIDQRQRFEAQLKEREQGNDEAHEMDEDFLEALEYGMPPTGGLGIGVDRLVMLLTNSPSIRDVLLFPQMRHK

SEQ ID NO: 113 - DNA, 1950 bp, Geobacillus subterraneus
atggagaaaaagacgttttatttgacgacgccgatttattatccgagcgacaaattgcacatcggccatgcttatacaacagtggcgggggatacgctagcgcgctataaacggatgcgcggttacgatgttatgtatttgacgggaaccgatgagcacgggcaaaaaattcaacgcaaggcggaggaaaaaggagtaacgccgcagcaatatgtcgatgagatcgtcgctggcattcaggagctatggaaaaagctcgacatttcttatgacgatttcatccgtacaacgcaggagcggcataaaaaagtagtcgaaaagattttcgcgcgtcttgtcgaacaaggggatatttatttaggtgaatatgaaggatggtattgcacgccatgcgaatcgttttacactgagcgacagcttgtcgacggcaactgcccggactgtggtcgtccggttgaaaaagtgaaagagcagtcgtactttttccgaatgagcaaatacgtcgaccgtttgcttcaatattatgaggaaaatccagatttcatccagccggaatcgcggaaaaacgaaatgattaacaattttattaagccggggcttgaagatttagctgtgtcgcggacgacgtttgactggggcattaaagtgccgggcgatccgaaacatgtcatttacgtctggattgacgcgcttgccaactatattacagcgctcggttacggcacggacaatgatgaaaagttccgcaaatattggccggccgatgtccatttagtcggcaaggaaatcatccgctttcatacgatttattggccgattatgctcatggcgcttgacttgccgctgccgaaaaaagtattcggtcatggctggctgctcatgaaagacgggaaaatgtcgaaatcgaaaggcaatgtcgttgacccggtgacgttgatcgatcgatacggactcgatgcgcttcgttattatttactcagggaagtgccgttcggttctgacggcgtattcacgccggaaggatttattgagcgcatcaactacgatttagccaatgacctaggcaatttattgaatcgtacagtagcgatgattaagaaatattttgatggggtgattccgccgtaccgcggtccgaaaacgccgtttgacgaagagctggtacaaacggcgcgtgaggtggtccgtcagtatgaggaagcgatggaacggatggagttttccgttgcccttgcttcggtttggcaactgattggccggacgaacaaatacattgatgagacgcagccatgggtattggccaaagatgaaagcaaacgggaagagcttgcttctgtcatgacccacctagccgagtcgctccgccatacggcagtgctgttgcagccgtttttgacacgcacgccagagcgcatttttgcccagctcggcattgccgaccgttcattaaaagagtgggatagcttgtacgagttcgggctcattccggaaggaacaaacgtgcaaaaaggagaaccactgttcccgcgccttgatattgaagcggaagtcgagtacattaaggcgcatatgcaaggcggcaagccggcggtggaacccgttaaagaggagaagcaagcggctgagacggccgaaatctcaattgatgagtttgccaaagttgacttgcgcgttgctgaagtcgtgcatgctgaacggatgaaaaacgccaataagctgttgaagctccaacttgatcttggcggcgagaaacggcaagtcatctctggtatcgctgaattttacaaaccagaggaactcatcggcaaaaaggtcatttgcgtcgccaatttaaaaccggccaaactgcgcggtgagtggtcggaaggaatgattttggccggcggtaacggcggagagttttcactggcgaccgtcgatcaacatgtgccaaacggaacaaaaattaaa

SEQ ID NO: 114 - protein, 650 aa, Geobacillus subterraneus
MEKKTFYLTTPIYYPSDKLHIGHAYTTVAGDTLARYKRMRGYDVMYLTGTDEHGQKIQRKAEEKGVTPQQYVDEIVAGIQELWKKLDISYDDFIRTTQERHKKVVEKIFARLVEQGDIYLGEYEGWYCTPCESFYTERQLVDGNCPDCGRPVEKVKEQSYFFRMSKYVDRLLQYYEENPDFIQPESRKNEMINNFIKPGLEDLAVSRTTFDWGIKVPGDPKHVIYVWIDALANYITALGYGTDNDEKFRKYWPADVHLVGKEIIRFHTIYWPIMLMALDLPLPKKVFGHGWLLMKDGKMSKSKGNVVDPVTLIDRYGLDALRYYLLREVPFGSDGVFTPEGFIERINYDLANDLGNLLNRTVAMIKKYFDGVIPPYRGPKTPFDEELVQTAREVVRQYEEAMERMEFSVALASVWQLIGRTNKYIDETQPWVLAKDESKREELASVMTHLAESLRHTAVLLQPFLTRTPERIFAQLGIADRSLKEWDSLYEFGLIPEGTNVQKGEPLFPRLDIEAEVEYIKAHMQGGKPAVEPVKEEKQAAETAEISIDEFAKVDLRVAEVVHAERMKNANKLLKLQLDLGGEKRQVISGIAEFYKPEELIGKKVICVANLKPAKLRGEWSEGMILAGGNGGEFSLATVDQHVPNGTKIK

SEQ ID NO: 115 - DNA, 1074 bp, Geobacillus subterraneus
atgagggacgggtttttttattttgttagaggagggattggcgtgaaagaacggttgcatgagcttgaacgagaagcgcttgaaaaaattgaacaagctggcgatttaaaagcgctcaacgatgtgcgtgtcgcctatttaggcaaaaaagggccgattaccgaagtgctgcgcggcatgggagcattgccgtcagaagagcgtccgaaaattggtgcgcttgccaatgaggtaagagaggcgatccaaaaggcgctcgaagcaaaacaaacgaaactggaagaagaagaagtcgagcggaagttggcggctgaagcgatcgatgtgacgcttccgggccgtccggtgaaactggggaatcctcatccgctgacgcgcgtcatcgaggaaattgaagatttgtttatcggcatgggctatacggtcgccgaaggtccggaagtcgagaccgattattacaattttgaggcgctcaatttgccgaaaggacacccggcccgcgatatgcaagattcgttttatattacggaagaaattctgcttcgcacccacacgtcgccgatgcaggcacggacgatggaaaaacatcgcgggcgcggtccggtaaaaatcatttgcccggggaaagtgtatcgccgcgataccgatgatgcgacccattcacatcagtttacgcaaattgaaggattggttgttgaccgcaacatccggatgagcgatttaaaagggacgctgcgcgaatttgcccgcaagctgttcggtgaagggcgcgacatccgttttcgtccgagctttttcccgtttaccgagccttcagtcgaggtcgatgtgtcctgcttccgctgcgaagggcacggctgcagcgtttgcaaaggtacgggctggattgaaattttaggcgctggcatggtgcacccgaacgtgcttgagatggccggctttgattcgaaaacgtataccggatttgcgttcggcatggggccggagcggatcgcgatgttgaaatacggcattgatgacatccgccatttctatcagaacgatcttcgtttcttgcaacaatttttgcgtgtc

SEQ ID NO: 116 - protein, 358 aa, Geobacillus subterraneus
MRDGFFYFVRGGIGVKERLHELEREALEKIEQAGDLKALNDVRVAYLGKKGPITEVLRGMGALPSEERPKIGALANEVREAIQKALEAKQTKLEEEEVERKLAAEAIDVTLPGRPVKLGNPHPLTRVIEEIEDLFIGMGYTVAEGPEVETDYYNFEALNLPKGHPARDMQDSFYITEEILLRTHTSPMQARTMEKHRGRGPVKIICPGKVYRRDTDDATHSHQFTQIEGLVVDRNIRMSDLKGTLREFARKLFGEGRDIRFRPSFFPFTEPSVEVDVSCFRCEGHGCSVCKGTGWIEILGAGMVHPNVLEMAGFDSKTYTGFAFGMGPERIAMLKYGIDDIRHFYQNDLRFLQQFLRV

SEQ ID NO: 117 - DNA, 2412 bp, Geobacillus subterraneus
atgctcgtttcttatcgttggctaggcgaatacgtcgatttgacgggcgtgacggcggaacaactcgctgatcgcattacaaaaagcggcattgaagtcgagcgggttgaagcgcttgagcggggaatgaaaggagtcgtcatcggccatgtgctcgaatgcgagccacacccaaacgccgataaactgcggaaatgtcttgttgatcttggcgaaggagagccggtgcaaatcatttgcggtgccccgaacgtcgccaaggggcaaaaagttgctgtagcgaaagttggagcgagactgccgggcaattttaaaatcaaacgggcgaagctgcgcggcgaagagtcgaacggcatgatttgctcgctccaagaactcggtgttgaaacaaaagtcgtgccgaaagaatacgccgaaggcattttcgtcttcccaagcgacgcgccggtcggcgctgatgcgcttgaatggctcggcttgcacgatgaagtgctcgaactcgccttgacgccgaatcgcgccgattgcttaagcatgcttggcgttgcctacgaagtcgctgcgattctcggccgcgatgtgaagttgccggaaacggcggtgaacgaaaatgaagaaagcgtccatgactacatttctgtccgtgtcgaggcgccggaagacaatccgctgtacgccggacggatcgtgaaaaacgtccaaatcggcccgtcgccgctttggatgcaagcgcgcttgatggcggccggcattcgtccacacaacaatgttgtcgatatcaccaactacattttgcttgagtacggccagccgcttcacgcgtttgactacgaccgtctcggttcgaaggagatcgtcgttcgtcgtgccaaggcgggagaaatgatcgtgacgcttgacgatgtcgagcggaagctgactgaagatcatctcgtcatcacaaacggccgtgagccggtcgccttagccggtgtgatgggcggagcgaactcggaagtgcaggatgacacgaaaacagtgttcatcgaagccgcgtattttacgagcccggtcatccgccaggcggtgaaagaccacgggttgcgcagcgaagcgagcacccggtttgaaaaagggattgatccggcgcggacgaaagaagcgctcgagcgcgctgctgctttgatggcagaatacgccggcggcgaggtcgtcagcggtatcgtggaagctaatacatggaaagaagagccggttgtcgtaacggtggcgctggaacgcatcaacggcgtcctcggcacagcgatgacgaaagaggaagtagctggcattctttcaaacttgcaattctcgtttacggaagataatggaacgtttacaatccatgttccatcgcgccgccgcgatattacgatcgaagaagatattatcgaggaagtcgcccgtttgtatggctacgaccatttgccagcgactttgccggtggccgaagcaaaaccgggcgagttgacaccgtaccaagcgaaacgccgccgtgtccgccgctatttcgaaggcgcgggcttgttccaggcgatcacgtattcgcttaccagtccggacaaagcgacgcggtttgctttggagacaaccgaaccagtccgcttggcgttgccgatgagtgaggagcggagcgttctccggcaaagcttggtgccgcatttgctcgaagcggcgagctacaaccgtgcccgccaagttgagaacgtcgcgctatatgaaatcggctctgtctatttgtccaagggggaaaatgtccaaccggcggaaaaagaacggctcgccggcgtcatcaccggtttatggcatgcccacctttggcaaggagagaaaaaagcagctgatttctatgttgcaaaaggcgtgcttgacggcttgttcgccctgcttgggctgtctgatcgcatcagctaccgtccggcgaagcgtgctgatttgcatctggggcggacagcggagattgtgcttgacggcaaagagatcggctttgtcggccagctccatccggctgtacaaaaagagtacgatttgaaagaaacgtatgtctttgaactcgccttcgctgagctactgaatacagaaggcgaaacgatccgttacgagtcgattccgcgcttcccgtcagtcgtgcgcgacatcgctttagtcgtcgacgacaatgtcgaagcaggtgctctcaagcaggcgatcgccgaagcggggaacccgctattaaaagacgtggccctctttgacgtctataaaggcgaccgtctgccggccgggaaaaaatcgctcgccttctcgctccgctactacgatccggaacggacgctcactgatgaggaagttactgccgtccatgaacgggttttggcagcggtcgaggagcagtttggcgcggtgttgcgcggg

SEQ ID NO: 118 - protein, 804 aa, Geobacillus subterraneus
MLVSYRWLGEYVDLTGVTAEQLADRITKSGIEVERVEALERGMKGVVIGHVLECEPHPNADKLRKCLVDLGEGEPVQIICGAPNVAKGQKVAVAKVGARLPGNFKIKRAKLRGEESNGMICSLQELGVETKVVPKEYAEGIFVFPSDAPVGADALEWLGLHDEVLELALTPNRADCLSMLGVAYEVAAILGRDVKLPETAVNENEESVHDYISVRVEAPEDNPLYAGRIVKNVQIGPSPLWMQARLMAAGIRPHNNVVDITNYILLEYGQPLHAFDYDRLGSKEIVVRRAKAGEMIVTLDDVERKLTEDHLVITNGREPVALAGVMGGANSEVQDDTKTVFIEAAYFTSPVIRQAVKDHGLRSEASTRFEKGIDPARTKEALERAAALMAEYAGGEVVSGIVEANTWKEEPVVVTVALERINGVLGTAMTKEEVAGILSNLQFSFTEDNGTFTIHVPSRRRDITIEEDIIEEVARLYGYDHLPATLPVAEAKPGELTPYQAKRRRVRRYFEGAGLFQAITYSLTSPDKATRFALETTEPVRLALPMSEERSVLRQSLVPHLLEAASYNRARQVENVALYEIGSVYLSKGENVQPAEKERLAGVITGLWHAHLWQGEKKAADFYVAKGVLDGLFALLGLSDRISYRPAKRADLHLGRTAEIVLDGKEIGFVGQLHPAVQKEYDLKETYVFELAFAELLNTEGETIRYESIPRFPSVVRDIALVVDDNVEAGALKQAIAEAGNPLLKDVALFDVYKGDRLPAGKKSLAFSLRYYDPERTLTDEEVTAVHERVLAAVEEQFGAVLRG

SEQ ID NO: 119 - DNA, 1755 bp, Geobacillus subterraneus
atgacattcaaaaattcttcctataatgaaagagagaaaacgaggtggctattgatgagacaaagtcaagggtttattccgacattgcgcgaagtgccggcggacgcggaagtgaaaagccatcagctcctgttgcgggccggcttcgtccgccaaagcgcaagcggcgtctacacgtttttgccgctcgggcaacgtgttttgcaaaaagtggaagcgattattcgtgaggagatgaatcgcgccggagcattggagcttctcatgcctgctttgcagccggctgagctttggcagcagtccgggcgctggtattcgtatggaccggagctcatgcgcctgaaagaccgtcacgagcgcgatttcgttctcggaccgacacacgaagagatgattactacgatcgttcgcgatgaagtgaaaacgtataagcggctgccgcttatcttgtatcaaattcaaacgaaattccgtgatgaaaaacgtccgcgtttcgggctgttgcgcggtcgcgagttcatcatgaaagatgcgtattcattccacacatcgcaggaaagtttggacgaaacgtacaataaaatgtatgaagcgtacgcgaacattttccgccgctgcggcttaaatttccgcgctgtcattgctgactccggagcgatgggcggcaaagatacgcacgagtttatggtgctgtctgatattggcgaggatacgatcgcttattccgatgcgtccgactatgcggccaacattgaaatggcaccggtcgtcactacgtatgaaaaaagcagtgagccgctggtggaactgaaaaaagtggcgaccccggagcaaaaaacgattgctgaagttgcttcgtatttgcaagtagcaccggaacgttgcattaaatcgcttttatttaacgttgatggccgctacgtgctcgttctggtgcgcggcgatcatgaagcgaatgatgtgaaagtgaaaaatgtgcttgatgcgactgtcgtggagctggcgacaccggaagaaacagcacgagtgatgaactgcccggttggttcgctcggcccgattggcgtcagcgaagaggtgacgattatcgccgatcatgctgtcgcggcgatcgtaaacggcgtctgcggcgccaatgaggaaggataccattatacgggtgtcaatccagaccgcgattttgccgtcagtcaatatgcggatttgcgtttcgtccaagaaggcgacccttctccggatggcaacgggacgatccgcttcgctcgtggcattgaagttggacatgtgtttaagctcggtacgaaatatagcgaggcgatgaacgccgtttacctcgacgaaaatggtcggacacagacgatgattatgggttgctacggcattggcgtctctaggctcgttgcggcgatcgccgagcagttcgccgatgagaacgggcttgtatggccggtttcggtcgcaccgtttcacgttcatttgctgacggcgaacgcgaaaagcgatgaacagcgcatgctggctgaagagtggtacgaaaaactcggacaggccggatttgacgtgttgtatgatgaccgtccggaacgggccggggtgaagtttgccgacagcgatttgatcggcatcccgctccgcgtcaccgttggcaagcgggcaagtgaaggtgtggtcgaagtaaaagttcggaaaacaggcgagacgtttgacgtgccggtcggtgagctgatcgaaacagtgcgccgtcttttgcaagga

SEQ ID NO: 120 - protein, 585 aa, Geobacillus subterraneus
MTFKNSSYNEREKTRWLLMRQSQGFIPTLREVPADAEVKSHQLLLRAGFVRQSASGVYTFLPLGQRVLQKVEAIIREEMNRAGALELLMPALQPAELWQQSGRWYSYGPELMRLKDRHERDFVLGPTHEEMITTIVRDEVKTYKRLPLILYQIQTKFRDEKRPRFGLLRGREFIMKDAYSFHTSQESLDETYNKMYEAYANIFRRCGLNFRAVIADSGAMGGKDTHEFMVLSDIGEDTIAYSDASDYAANIEMAPVVTTYEKSSEPLVELKKVATPEQKTIAEVASYLQVAPERCIKSLLFNVDGRYVLVLVRGDHEANDVKVKNVLDATVVELATPEETARVMNCPVGSLGPIGVSEEVTIIADHAVAAIVNGVCGANEEGYHYTGVNPDRDFAVSQYADLRFVQEGDPSPDGNGTIRFARGIEVGHVFKLGTKYSEAMNAVYLDENGRTQTMIMGCYGIGVSRLVAAIAEQFADENGLVWPVSVAPFHVHLLTANAKSDEQRMLAEEWYEKLGQAGFDVLYDDRPERAGVKFADSDLIGIPLRVTVGKRASEGVVEVKVRKTGETFDVPVGELIETVRRLLQG

SEQ ID NO: 121 - DNA, 1296 bp, Geobacillus subterraneus
atggtggataaggaggtaaagcgaatgctggatgtgaaattactacgcacccaatttcaagaggtgaaagaaaaactgctgcagcgcggcgacgacttggccaacatcgaccggtttgagcagcttgataaagagcgtcgtcgtttgatcgctcaggtggaggagttaaaaagcaagcgcaatgaggtgtcgcaacaaattgctgtcttaaagcgtgaaaaaaaggacgccgagtcgttgatcgtcgaaatgcgcgaagtcggcgaccgcattaaacaaatggacgagcaaattcgccaacttgaagaagagctcgacagccttctgttatcgattccgaatgtaccgcatgagtcagtgccagtcggtcagtcggaagaagataatgtcgaagtgcgaagatggggggaaccgcgttcgttctcgttcgaaccgaagccacattgggacattgctgaccaactcggtttgctcgattttgagcgggctgccaaagtggcaggaagtcggtttgtgttttacaaaggactaggggctcgtcttgagcgggcattaatcaactttatgctcgacatccatctcgatgaatttggctatcaagaggtgttgccgccatacttagtgaaccgggcgagcatgatcggaacagggcaattgccaaaatttgcggaagacgcgttccacttggacaatgaagactattttctcattccaacagcggaagtgcctgtgacgaatttgcatcgcgatgaaattttaacggctgatgacttgccgctttactatgcggcttacagcgcgtgcttccgcgccgaagctggctcggctggccgtgacacgcgggggctcatccgccagcaccaattcaataaagtggagctcgtcaagttcgtcaagccggaggattcatatgacgagttggaaaaattgacgcaccaagccgaaacgatcctgcaacggctcggacttccgtatcgcgtcgtagccttgtgtacaggggatctgggattttcagcggcgaagacgtatgatattgaggtgtggctgccaagctatggaacgtatcgggaaatttcgtcgtgcagcaactttgaggcgttccaggcgcgccgagctaatatccgcttccgtcgcgagccgaaagcaaagccagaatatgtgcatacgctaaacggttcggggctagccatcggccgcacggttgctgccattttggaaaactaccaacaagaagacggatcggtcgtcatcccggaagcgctccgtccatatatggggaatcgggatgtcattcgc

SEQ ID NO: 122 - protein, 432 aa, Geobacillus subterraneus
MVDKEVKRMLDVKLLRTQFQEVKEKLLQRGDDLANIDRFEQLDKERRRLIAQVEELKSKRNEVSQQIAVLKREKKDAESLIVEMREVGDRIKQMDEQIRQLEEELDSLLLSIPNVPHESVPVGQSEEDNVEVRRWGEPRSFSFEPKPHWDIADQLGLLDFERAAKVAGSRFVFYKGLGARLERALINFMLDIHLDEFGYQEVLPPYLVNRASMIGTGQLPKFAEDAFHLDNEDYFLIPTAEVPVTNLHRDEILTADDLPLYYAAYSACFRAEAGSAGRDTRGLIRQHQFNKVELVKFVKPEDSYDELEKLTHQAETILQRLGLPYRVVALCTGDLGFSAAKTYDIEVWLPSYGTYREISSCSNFEAFQARRANIRFRREPKAKPEYVHTLNGSGLAIGRTVAAILENYQQEDGSVVIPEALRPYMGNRDVIR

SEQ ID NO: 123 - DNA, 1947 bp, Geobacillus subterraneus
atgccagacgttattcgcattacgttcccggacggggcgaaaaaggagtttccgagcggaacgtcaactgaggacatcgctgcctcgatcagtccgggattgaagaaaaaagcgattgccgggaaactgaacggccggtttgttgatttacgcacgccgcttcaagaagacggcgagcttgtcattattacccaggacatgcctgaggcacttgatattttgcgtcatagcaccgcccatttaatggcgcaagcgatcaagcggctgtatgacaacgtcaagcttggcgtcggcccggtcattgaaaacggcttctactatgatattgatatggaacataagctgacgccggatgatttgccgaaaattgaggcggaaatgcgcaaaatcgtaaaggaaaatcttgacgttgttcgcaaagaggtgagccgtgacgaggcgattcgcctgtatgaaaaaattggtgatcacttgaaactggagctcatcaacgatattccggaaggcgagacgatttccatttacgagcaaggcgagtttttcgatctttgtcggggtgtgcacgtgccgtcgaccgggaaaatcaaagagttcaagctgctcagcatctcgggggcctactggcgcggtgacagcaacaacaaaatgctgcagcgtatttacggtacggcgtttttcaaaaaagaagatctggaccattatttgcagttgctcgaagaggcgaaagagcgcgatcatcgcaaattgggcaaagagcttgagctatttacgacatcacaaaaagtcggacaaggactgccgctttggttgccgaaaggggcgacgatccgtcgcttgattgaacggtacattgtcgataaagaaatcgcccttggttatgatcatgtatatacgccggtgctcggcagtgtggagctgtataaaacctcaggacactgggaccattataaagaaaacatgttcccaccgatggaaatggataacgaagagctcgtgctgcggccgatgaactgcccgcaccatatgatgatttataaaagcaagcttcatagctaccgtgagctgccgatccgcatcgccgagctcggcacgatgcatcgctacgaaatgtccggggcgcttactggactgcagcgtgtccgcggcatgacgctcaacgacgcccatattttcgtgcgcccggatcaaattaaagacgagtttaagcgcgtcgttaatttgattttggaagtatacaaagactttgggctggacgaatattcgttccgcctgtcgtaccgcgacccacaagataaagaaaaatattacgacgacgacgagatgtgggaaaaggcgcaacgcatgctgcgcgaggcgatggatgaacttggcctcgattactacgaagcggaaggggaagcagcgttttacggaccgaagctcgatgtgcaagtgcgcacggcactcggcaaagatgagacgctgtcgactgtacagcttgacttcctcttgccggagcggtttgacttaacatatatcggcgaagatggaaaaccgcaccgcccggtcgtcatccaccgcggcgttgtttccacgatggaacggtttgtcgccttcttgatcgaagaatacaaaggggcatttccaacgtggctcgccccggtgcaagtggaagtcatcccggtatcgtcggaagcccatctcgattatgcgtatgaagtgaaacaagcgctgcaagtaaacggcttccgcgtcgaagtcgacgaacgggatgaaaaaatcggctataaaatccgcgaagcgcaaatgcaaaaaattccttatatgctcgttgtcggcgacaaagaagcggccgagcgagcggtcaacgtccgccgctacggtgaaaaagaaagcgagactgtggcgcttgacaagtttatcgcgatgctagaagaagatgtgcggcaaaaacgagtgaaaaaacga

SEQ ID NO: 124 - protein, 649 aa, Geobacillus subterraneus
MPDVIRITFPDGAKKEFPSGTSTEDIAASISPGLKKKAIAGKLNGRFVDLRTPLQEDGELVIITQDMPEALDILRHSTAHLMAQAIKRLYDNVKLGVGPVIENGFYYDIDMEHKLTPDDLPKIEAEMRKIVKENLDVVRKEVSRDEAIRLYEKIGDHLKLELINDIPEGETISIYEQGEFFDLCRGVHVPSTGKIKEFKLLSISGAYWRGDSNNKMLQRIYGTAFFKKEDLDHYLQLLEEAKERDHRKLGKELELFTTSQKVGQGLPLWLPKGATIRRLIERYIVDKEIALGYDHVYTPVLGSVELYKTSGHWDHYKENMFPPMEMDNEELVLRPMNCPHHMMIYKSKLHSYRELPIRIAELGTMHRYEMSGALTGLQRVRGMTLNDAHIFVRPDQIKDEFKRVVNLILEVYKDFGLDEYSFRLSYRDPQDKEKYYDDDEMWEKAQRMLREAMDELGLDYYEAEGEAAFYGPKLDVQVRTALGKDETLSTVQLDFLLPERFDLTYIGEDGKPHRPVVIHRGVVSTMERFVAFLIEEYKGAFPTWLAPVQVEVIPVSSEAHLDYAYEVKQALQVNGFRVEVDERDEKIGYKIREAQMQKIPYMLVVGDKEAAERAVNVRRYGEKESETVALDKFIAMLEEDVRQKRVKKR

SEQ ID NO: 125 - DNA, 984 bp, Geobacillus subterraneus
atgaaaaccattttttctggcattcagccaagcggcgtcattacccttggcaactacattggtgcgatgcgacaatttgtcgaactgcagcatgagtacaactgctatttttgcattgtcgaccaacatgccattactgttccgcaaaatccgaacgaactgcaacaaaacattcgccgtctcgctgccttatatttggcagtcggcatcgatcctaaacaggcgacgctgttcgttcaatcggaggtgccggcgcacgcccaagcggcttggatgctgcaatgcatcgtctatatcggcgaactggagcggatgacgcagtttaaagacaaatcagccggtaaagaggcggtcagtgccgggttgctcacgtatccaccgcttatggcagccgacattttgctttacaacacggacattgtcccagtcggcgaagaccaaaagcagcacatcgagctgacgcgcgatttagctgagcgcttcaacaaacggtacggcgagctgttcactatcccggaagcgcgcatcccgaaaatcggcgcccgcattatgtcgcttaccgatccgacgaaaaaaatgagcaaatctgacccaaacccgaaatcgtttattacgctgcttgacgacgccaaaacgattgaaaagaaaattaaaagtgctgtgaccgattcagaaggaacgattcgctatgacaaggaagcgaaaccgggcatttcgaacttgctcaacatttattcgattttatcgggtcagccgattgacgaacttgagcggcaatacgaaggaaaaggatacggggtctttaaatccgatttggcccaagtggtcattgaaacgctccaaccgatccaagagcggtattatcattggctcgaaagtgaagagctcgaccgcgtcctagacgaaggggcggaaaaagcgaaccgtgtcgcctcggaaatggtgcgcaaaatggaacaagccatggggcttgggcggcgtcgg

SEQ ID NO: 126 - protein, 328 aa, Geobacillus subterraneus
MKTIFSGIQPSGVITLGNYIGAMRQFVELQHEYNCYFCIVDQHAITVPQNPNELQQNIRRLAALYLAVGIDPKQATLFVQSEVPAHAQAAWMLQCIVYIGELERMTQFKDKSAGKEAVSAGLLTYPPLMAADILLYNTDIVPVGEDQKQHIELTRDLAERFNKRYGELFTIPEARIPKIGARIMSLTDPTKKMSKSDPNPKSFITLLDDAKTIEKKIKSAVTDSEGTIRYDKEAKPGISNLLNIYSILSGQPIDELERQYEGKGYGVFKSDLAQVVIETLQPIQERYYHWLESEELDRVLDEGAEKANRVASEMVRKMEQAMGLGRRR

SEQ ID NO: 127 - DNA, 1257 bp, Geobacillus subterraneus
atgaacctgcttgaagaactgcaatggcgcggacttgtcaatcaaacgacggatgaggatgggcttcgaaagctcctgaatgaggagaaggtgacgctttattgcgggtttgacccgacagcagacagcttgcatatcggccatttggtcacgatcatgaccttgcgtcgtttccaacaggcggggcatcaaccgatcgccttagtcggcggcgccaccgggttgatcggcgatccgagtggcagaaaaagcgagcgcacgctcaacgccaaggagacggtcgagacgtggagcgcccgaatcaaagcgcaactcgagcggtttcttgattttgaggctgagagcaatccagcgaaaatcaaaaacaactacgactggatcgggccgcttgatgtcatctcgtttttgcgtgacatcggcaagcatttcagcgtcaattacatgcttgcgaaagaatcggtgcagtcgcgcattgaaatgggcatttcgtttaccgagttcagctatatgatgctgcaggcgtacgacttcctcaacttgtacgaaacggaaggttgccgactacaaatcggtggcagcgaccaatggggcaacatcacggcggggcttgagctcatccgcagaacgaaaggtgaggcgaaagcatttggtttgacggttccgctcgtgacgaaagccgatgggacgaagttcggaaaaacggaaagcggcgcggtttggctcgatccggaaaaaacgtcgccgtatgagttttaccagttctggatcaacaccgatgaccgcgatgtgatccgttacttaaaatatttcacgttcttgacaaaagaagagatcgacgcgcttgaacaagagctgcgcgaagcgccggagaagcgggtggcgcaaaaaacgcttgcttccgaagtgacgaagctcgtgcatggcgaagaggcgctcaatcaagcgattcgtatttcagaagcactctttagcggcgacattgccgaactgacggctgcggaaatcgagcaagggtttaaaaacgtgccgtcgtttgtccatgaaggaggcgacgtcccgctcgtcgagctgctcgtagctgccggcatctcgccatcgaagcggcaggcgcgcgaagatgttcaaaacggtgcgatttatgtcaacggcgagcgcatccaagatgtcggcgctgtcttaacggccgaacaccgtttggaagggcggtttaccgtgatccgccgcggcaagaagaagtattatttaatccgctacgct

SEQ ID NO: 128 - protein, 419 aa, Geobacillus subterraneus
MNLLEELQWRGLVNQTTDEDGLRKLLNEEKVTLYCGFDPTADSLHIGHLVTIMTLRRFQQAGHQPIALVGGATGLIGDPSGRKSERTLNAKETVETWSARIKAQLERFLDFEAESNPAKIKNNYDWIGPLDVISFLRDIGKHFSVNYMLAKESVQSRIEMGISFTEFSYMMLQAYDFLNLYETEGCRLQIGGSDQWGNITAGLELIRRTKGEAKAFGLTVPLVTKADGTKFGKTESGAVWLDPEKTSPYEFYQFWINTDDRDVIRYLKYFTFLTKEEIDALEQELREAPEKRVAQKTLASEVTKLVHGEEALNQAIRISEALFSGDIAELTAAEIEQGFKNVPSFVHEGGDVPLVELLVAAGISPSKRQAREDVQNGAIYVNGERIQDVGAVLTAEHRLEGRFTVIRRGKKKYYLIRYA

SEQ ID NO: 129 - DNA, 2727 bp, Geobacillus subterraneus
atgaaaggggcttttttgcttgcctatcggacggttgatcctgtaggcaacacagccattgtttatcacatgaaggagggaataaaagtggcacagcatgaagtgtcgatgccgccaaaatacgatcaccgcgctgttgaagcggggcgctatgactggtggctgaaaggcaagttttttgaaacgaccggcgatccggacaaacaaccgtttacgatcgttatcccaccgccgaacgtcacaggcaaactgcatttgggccatgcgtgggatacgacgctgcaagacatcattacgcgcatgaagcggatgcaagggtatgatgtcctatggcttccgggtatggaccatgccggcatcgccacccaggcgaaagtggaagaaaaattgcgccaacaaggactgtcccgctacgatttaggacgggaaaaatttttggaagaaacgtggaaatggaaagaagaatatgccggccatatccgcagccaatgggcaaaattagggctcggcctcgattacacgcgcgagcggtttacgcttgatgaagggctgtcaaaagccgtacgcgaagtgttcgtctcgctttaccggaaagggctcatttaccgcggtgaatacattatcaactgggatccggcgaccaaaaccgccttgtccgacatcgaggtcatttacaaggaagtgaaaggtgcgctttatcatttgcgctatccgctcgctgacggctcgggctacattgaagtagcgacaacccgtccagaaacgatgctcggtgacacggccgtcgcggttcatccggatgacgagcggtataaacacttgatcggcaagatggtgaaattgccaatcgttggccgggaaattccgatcatcgctgatgagtatgtcgatatggaattcggttccggcgcggtaaaaattacaccggcacacgatccgaacgactttgaagttggcaaccgccacaacttgccgcgcattctcgtcatgaacgaagacggtacaatgaacgaaaacgcattgcaatatcaagggcttgaccggtttgaatgccggaagcaaatcgtccgtgatttacaagagcaaggcgtcctctttaaaattgaggaacacgtccactcggtcgggcacagtgaacggagcggcgccgttgttgaaccgtatttgtcgacacaatggttcgtaaaaatgaagccgctcgcggaagctgccatcaagatgcagcaaacagaaggaaaagtgcaatttgtgccggagcggtttgaaaaaacgtacttgcactggcttgagaacattcgcgactggtgcatttcgcgtcagctttggtgggggcaccgcattccggcgtggtaccataaagaaacgggtgaaatttacgtcgaccacgagccgccggcagacattgaaaattgggagcaagacccggatgtgcttgatacatggttcagctcggcactctggccgttctccacaatggggtggccggatacggaagcgccggactacaagcgctattacccgaccgatgtgcttgtcaccggctatgacatcattttcttctgggtgtcgcgcatgattttccaagggcttgagttcactgggaagagaccgtttaaagatgtgttgatccacggcctcgtccgcgacgctcaaggaagaaaaatgagcaagtcgctcggcaacggtgtcgacccgatggatgtcattgaccaatacggcgccgatgcgctccgctacttcctagcgaccggtagctcgccaggacaagatttgcgctttagcacggaaaaagttgaggcgacgtggaattttgctaacaaaatttggaacgcttcacgtttcgccttaatgaacatgggcggcatgacatatgaggagctcgatttgagcggcgaaaaaacggtcgccgaccattggattttaacgcgcttaaatgaaacgatcgacacggtgacgaagctcgccgacaaatacgagtttggtgaagtcggtcgcacgttgtacaactttatttgggacgatttgtgcgactggtacattgaaatggcgaagctgccgctttacggcgatgatgagacagcgaaaaagacgacgcgttcagttttagcgtatgtgcttgacaatacgatgcgcttgttgcatccattcatgccgttcattaccgaggaaatttggcaaaacttgccgcatgacggcgaatcgattaccgttgcctcgtggccgcaagtgcgtccggagctgtcaaacgaagaagcggcggaagaaatgcggatgctcgttgacattatccgcgcggtccgaaacgttcgtgccgaagtcaatacgccgccgagcaaaccgattgcgctctacattaagacaaaagacgaacaagtgcgcgcagcgcttatgaaaaaccgcgcttatctcgaacggttctgcaatccgagcgaattgatcattgacacggatgttccggcgccagaaaaagcgatgactgctgtcgtcacaggggcagagctcattttgccgcttgaaggactcatcaatatcgaagaagaaatcaagcggcttgagaaagagctcgacaaatggaacaaagaagtcgagcgtgtcgaaaagaaactggcgaacgaaggctttttggcaaaagcgccggctcatgtcgtcgaggaagagcggcgcaagcggcaagattacatcgaaaaacgcgaagcagtgaaagcgcgtcttgccgagttgaaacgg

SEQ ID NO: 130 - protein, 909 aa, Geobacillus subterraneus
MKGAFLLAYRTVDPVGNTAIVYHMKEGIKVAQHEVSMPPKYDHRAVEAGRYDWWLKGKFFETTGDPDKQPFTIVIPPPNVTGKLHLGHAWDTTLQDIITRMKRMQGYDVLWLPGMDHAGIATQAKVEEKLRQQGLSRYDLGREKFLEETWKWKEEYAGHIRSQWAKLGLGLDYTRERFTLDEGLSKAVREVFVSLYRKGLIYRGEYIINWDPATKTALSDIEVIYKEVKGALYHLRYPLADGSGYIEVATTRPETMLGDTAVAVHPDDERYKHLIGKMVKLPIVGREIPIIADEYVDMEFGSGAVKITPAHDPNDFEVGNRHNLPRILVMNEDGTMNENALQYQGLDRFECRKQIVRDLQEQGVLFKIEEHVHSVGHSERSGAVVEPYLSTQWFVKMKPLAEAAIKMQQTEGKVQFVPERFEKTYLHWLENIRDWCISRQLWWGHRIPAWYHKETGEIYVDHEPPADIENWEQDPDVLDTWFSSALWPFSTMGWPDTEAPDYKRYYPTDVLVTGYDIIFFWVSRMIFQGLEFTGKRPFKDVLIHGLVRDAQGRKMSKSLGNGVDPMDVIDQYGADALRYFLATGSSPGQDLRFSTEKVEATWNFANKIWNASRFALMNMGGMTYEELDLSGEKTVADHWILTRLNETIDTVTKLADKYEFGEVGRTLYNFIWDDLCDWYIEMAKLPLYGDDETAKKTTRSVLAYVLDNTMRLLHPFMPFITEEIWQNLPHDGESITVASWPQVRPELSNEEAAEEMRMLVDIIRAVRNVRAEVNTPPSKPIALYIKTKDEQVRAALMKNRAYLERFCNPSELIIDTDVPAPEKAMTAVVTGAELILPLEGLINIEEEIKRLEKELDKWNKEVERVEKKLANEGFLAKAPAHVVEEERRKRQDYIEKREAVKARLAELKR

SEQ ID NO: 131 - DNA, 963 bp, Geobacillus subterraneus
atgctgatgacgaacattgtctttatgggaacgcctgattttgcggtgccggttttacggcagctgcttgatgacgggtatcgggttgttgccgttgttacgcagccggacaagccgaaagggcgaaagcgcgagcttgttccgccccccgttaaggtcgaggcgcaaaaacacggcatcccggtattgcaaccgacgaaaattcgtgaaccggaacaatacgaacaagtgctggcgtttgcgcctgacttgatcgtgaccgcggcatttggacaaattttgcctaaggctctgcttgacgctcccaaatatggctgcattaatgttcacgcctcgcttcttcccgagctgcgcggcggtgcgccgatccattatgccatttggcaagggaaaacgaaaacaggtgtcacgattatgtatatggcggaaaagttggatgccggcgacatgttgacgcaagtcgaagtgccgattgaagaaaccgataccgtcggcacactgcatgataaattgagcgctgccggggctaaactattatcagaaacgctcccgcttttattggaaggtaaccttgcgcctattccgcaagaggaagagaaagcgacatatgctccgaatatccggcgtgaacaagagcggattgactgggcgcagcctggtgaggcgatttacaaccatatccgtgcttttcatccgtggccggttacgtatacgacatacgacgggaacgtttggaaaatctggtggggcgaaaaagtgccggcgccaagcttagcgtcgccaggcacgattttatcgcttgaggaagacggcatcgtcgtcgccaccggcagtgagacggccattaaaattactgaattgcagccggccggcaaaaagcgaatggcggccagcgagtttttgcgcggtgctggcagccggcttgcggtcggcacgaagctaggagagaacaatgaacgtacg

SEQ ID NO: 132 - protein, 321 aa, Geobacillus subterraneus
MLMTNIVFMGTPDFAVPVLRQLLDDGYRVVAVVTQPDKPKGRKRELVPPPVKVEAQKHGIPVLQPTKIREPEQYEQVLAFAPDLIVTAAFGQILPKALLDAPKYGCINVHASLLPELRGGAPIHYAIWQGKTKTGVTIMYMAEKLDAGDMLTQVEVPIEETDTVGTLHDKLSAAGAKLLSETLPLLLEGNLAPIPQEEEKATYAPNIRREQERIDWAQPGEAIYNHIRAFHPWPVTYTTYDGNVWKIWWGEKVPAPSLASPGTILSLEEDGIVVATGSETAIKITELQPAGKKRMAASEFLRGAGSRLAVGTKLGENNERT

SEQ ID NO: 133 - protein, 358 aa, Geobacillus stearothermophilus
MFDRLEAVEQRYEKLNELLMEPDVINDPKKLRDYSKEQADLGETVQTYREYKSVREQLAEAKAMLEEKLEPELREMVKEEIGELEEREEALVEKLKVLLLPKDPNDEKNVIMEIRAAAGGEEAALFAGDLYRMYTRYAESQGWKTEVIEASPTGLGGYKEIIFMINGKGAYSKLKFENGAHRVQRVPETESGGRIHTSTATVACLPEMEEIEVEINEKDIRVDTFASSGPGGQSVNTTMSAVRLTHIPTGIVVTCQDEKSQIKNKEKAMKVLRARIYDKYQQEARAEYDQTRKQAVGTGDRSERIRTYNFPQNRVTDHRIGLTIQKLDQVPDGHLDEIIEALILDDQAKKLEQANDAS

SEQ ID NO: 134 - protein, 255 aa, Aequorea victoria
MRGSHHHHHHGSSKGEELFTGVVPILVELDGDVNGHKFSVRGEGEGDATNGKLTLKFICTTGKLPVPWPTLVTTLTYGVLCFSRYPDHMKRHDFFKSAMPEGYVQERTISFKDDGTYKTRAEVKFEGDTLVNRIELKGIDFKEDGNILGHKLEYNFNSHNVYITADKQKNGIKAYFKIRHNVEDGSVQLADHYQQNTPIGDGPVLLPDNHYLSTQSVLSKDPNEKRDHMVLLEDVTAAGITHGMDELYKGSEPEA

SEQ ID NO: 135 - protein, 225 aa, Aequorea victoria
MELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGI

SEQ ID NO: 136 - protein, 883 aa, T7 Bacteriophage
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA